SEQUENCE LISTING (572 sequences)

SEQ ID NO: 1 - RNA, 20 nt, Artificial Sequence
  Synthetic: nucleobase sequence
ggugaauaguauaacaauau

SEQ ID NO: 2 - RNA, 20 nt, Artificial Sequence
  Synthetic: nucleobase sequence
auguuguuauaguauccacc

SEQ ID NO: 3 - RNA, 45 nt, Artificial Sequence
  Synthetic: nucleobase sequence
ggugaauaguauaacaauaugcuaaauguuguuauaguauccacc

SEQ ID NO: 4 - RNA, 20 nt, Artificial Sequence
  Synthetic: nucleobase sequence
ggugaagaggagaacaauau

SEQ ID NO: 5 - RNA, 20 nt, Artificial Sequence
  Synthetic: nucleobase sequence
auguuguucucgucuccacc

SEQ ID NO: 6 - RNA, 45 nt, Artificial Sequence
  Synthetic: nucleobase sequence
ggugaagaggagaacaauaugcuaaauguuguucucgucuccacc

SEQ ID NO: 7 - RNA, 25 nt, Artificial Sequence
  Synthetic: nucleobase sequence
ggugucgagaagaggagaacaauau

SEQ ID NO: 8 - RNA, 25 nt, Artificial Sequence
  Synthetic: nucleobase sequence
auguuguucucgucuccucgacacc

SEQ ID NO: 9 - RNA, 55 nt, Artificial Sequence
  Synthetic: nucleobase sequence
ggugucgagaagaggagaacaauaugcuaaauguuguucucgucuccucgacacc

SEQ ID NO: 10 - RNA, 22 nt, Artificial Sequence
  Synthetic: nucleobase sequence
ggguggaauaguauaacaauau

SEQ ID NO: 11 - RNA, 22 nt, Artificial Sequence
  Synthetic: nucleobase sequence
auguuguuauaguaucccaccu

SEQ ID NO: 12 - RNA, 49 nt, Artificial Sequence
  Synthetic: nucleobase sequence
ggguggaauaguauaacaauaugcuaaauguuguuauaguaucccaccu

SEQ ID NO: 13 - RNA, 20 nt, Artificial Sequence
  Synthetic: nucleobase sequence
guggaauaguauaacaauau

SEQ ID NO: 14 - RNA, 20 nt, Artificial Sequence
  Synthetic: nucleobase sequence
auguuguuauaguaucccac

SEQ ID NO: 15 - RNA, 45 nt, Artificial Sequence
  Synthetic: nucleobase sequence
guggaauaguauaacaauaugcuaaauguuguuauaguaucccac

SEQ ID NO: 16 - RNA, 25 nt, Artificial Sequence
  Synthetic: nucleobase sequence
ggugucgagaauaguauaacaauau

SEQ ID NO: 17 - RNA, 25 nt, Artificial Sequence
  Synthetic: nucleobase sequence
auguuguuauaguauccucgacacc

SEQ ID NO: 18 - RNA, 55 nt, Artificial Sequence
  Synthetic: nucleobase sequence
ggugucgagaauaguauaacaauaugcuaaauguuguuauaguauccucgacacc

SEQ ID NO: 19 - RNA, 22 nt, Artificial Sequence
  Synthetic: nucleobase sequence
ggguggaauaguauaacaauau

SEQ ID NO: 20 - RNA, 22 nt, Artificial Sequence
  Synthetic: nucleobase sequence
auguuguuauaguaucccaccu

SEQ ID NO: 21 - RNA, 49 nt, Artificial Sequence; identical to SEQ ID NO: 12
  Synthetic: nucleobase sequence

SEQ ID NO: 22 - RNA, 18 nt, Artificial Sequence
  Synthetic: nucleobase sequence
ggguggaauaguauacca

SEQ ID NO: 23 - RNA, 18 nt, Artificial Sequence
  Synthetic: nucleobase sequence
ugguauaguaucccaccu

SEQ ID NO: 24 - RNA, 40 nt, Artificial Sequence
  Synthetic: nucleobase sequence
ggguggaauaguauaccauucgugguauaguaucccaccu

SEQ ID NO: 25 - RNA, 20 nt, Artificial Sequence
  Synthetic: nucleobase sequence
guggguggaauaguauacca

SEQ ID NO: 26 - RNA, 20 nt, Artificial Sequence
  Synthetic: nucleobase sequence
ugguauaguaucccaccuac

SEQ ID NO: 27 - RNA, 44 nt, Artificial Sequence
  Synthetic: nucleobase sequence
guggguggaauaguauaccauucgugguauaguaucccaccuac

SEQ ID NO: 28 - RNA, 19 nt, Artificial Sequence
  Synthetic: nucleobase sequence
uggguggaauaguauacca

SEQ ID NO: 29 - RNA, 19 nt, Artificial Sequence
  Synthetic: nucleobase sequence
ugguauaguaucccaccua

SEQ ID NO: 30 - RNA, 42 nt, Artificial Sequence
  Synthetic: nucleobase sequence
uggguggaauaguauaccauucgugguauaguaucccaccua

SEQ ID NO: 31 - RNA, 17 nt, Artificial Sequence
  Synthetic: nucleobase sequence
gguggaauaguauacca

SEQ ID NO: 32 - RNA, 17 nt, Artificial Sequence
  Synthetic: nucleobase sequence
ugguauaguaucccacc

SEQ ID NO: 33 - RNA, 38 nt, Artificial Sequence
  Synthetic: nucleobase sequence
gguggaauaguauaccauucgugguauaguaucccacc

SEQ ID NO: 34 - RNA, 16 nt, Artificial Sequence
  Synthetic: nucleobase sequence
guggaauaguauacca

SEQ ID NO: 35 - RNA, 16 nt, Artificial Sequence
  Synthetic: nucleobase sequence
ugguauaguaucccac

SEQ ID NO: 36 - RNA, 36 nt, Artificial Sequence
  Synthetic: nucleobase sequence
guggaauaguauaccauucgugguauaguaucccac

SEQ ID NO: 37 - RNA, 45 nt, Artificial Sequence; identical to SEQ ID NO: 3
  Synthetic: GluR2 ADAR-recruiting domain

SEQ ID NO: 38 - RNA, 45 nt, Artificial Sequence; identical to SEQ ID NO: 6
  Synthetic: GluR2 ADAR-recruiting domain

SEQ ID NO: 39 - RNA, 55 nt, Artificial Sequence; identical to SEQ ID NO: 9
  Synthetic: GluR2 ADAR-recruiting domain

SEQ ID NO: 40 - RNA, 29 nt, Artificial Sequence
  Synthetic: GluR2 ADAR-recruiting domain
ssggagaagaggagaaaaagaaaggggaa

SEQ ID NO: 41 - RNA, 49 nt, Artificial Sequence; identical to SEQ ID NO: 12
  Synthetic: GluR2 ADAR-recruiting domain

SEQ ID NO: 42 - RNA, 45 nt, Artificial Sequence; identical to SEQ ID NO: 15
  Synthetic: GluR2 ADAR-recruiting domain

SEQ ID NO: 43 - RNA, 55 nt, Artificial Sequence; identical to SEQ ID NO: 18
  Synthetic: GluR2 ADAR-recruiting domain

SEQ ID NO: 44 - RNA, 49 nt, Artificial Sequence; identical to SEQ ID NO: 12
  Synthetic: GluR2 ADAR-recruiting domain

SEQ ID NO: 45 - RNA, 40 nt, Artificial Sequence; identical to SEQ ID NO: 24
  Synthetic: GluR2 ADAR-recruiting domain

SEQ ID NO: 46 - RNA, 44 nt, Artificial Sequence; identical to SEQ ID NO: 27
  Synthetic: GluR2 ADAR-recruiting domain

SEQ ID NO: 47 - RNA, 42 nt, Artificial Sequence; identical to SEQ ID NO: 30
  Synthetic: GluR2 ADAR-recruiting domain

SEQ ID NO: 48 - RNA, 38 nt, Artificial Sequence; identical to SEQ ID NO: 33
  Synthetic: GluR2 ADAR-recruiting domain

SEQ ID NO: 49 - RNA, 36 nt, Artificial Sequence; identical to SEQ ID NO: 36
  Synthetic: GluR2 ADAR-recruiting domain

SEQ ID NO: 50 - DNA, 19 bp, Artificial Sequence
  Synthetic: MS2 ADAR-recruiting domain
acatgaggatcacccatgt

SEQ ID NO: 51 - DNA, 20 bp, Artificial Sequence
  Synthetic: SERPINA1 fwd
agcacctggaaaatgaactc

SEQ ID NO: 52 - DNA, 21 bp, Artificial Sequence
  Synthetic: SERPINA1 rvs
tttttttgcgatgcaatttcc

SEQ ID NO: 53 - DNA, 50 bp, Artificial Sequence
  Synthetic: KB-018-001
cuaaaaacauggccccagcagcuucagucccuuuctcgucgauggucagc

SEQ ID NO: 54 - DNA, 45 bp, Artificial Sequence
  Synthetic: KB-018-002
aacauggccccagcagcuucagucccuuuctcgucgauggucagc

SEQ ID NO: 55 - DNA, 40 bp, Artificial Sequence
  Synthetic: KB-018-003
ggccccagcagcuucagucccuuuctcgucgauggucagc

SEQ ID NO: 56 - DNA, 35 bp, Artificial Sequence
  Synthetic: KB-018-004
cagcagcuucagucccuuuctcgucgauggucagc

SEQ ID NO: 57 - DNA, 31 bp, Artificial Sequence
  Synthetic: KB-018-005
agcuucagucccuuuctcgucgauggucagc

SEQ ID NO: 58 - DNA, 29 bp, Artificial Sequence
  Synthetic: KB-018-006
cuucagucccuuuctcgucgauggucagc

SEQ ID NO: 59 - DNA, 25 bp, Artificial Sequence
  Synthetic: KB-018-007
cagucccuuuctcgucgauggucag

SEQ ID NO: 60 - DNA, 23 bp, Artificial Sequence
  Synthetic: KB-018-008
agucccuuuctcgucgaugguca

SEQ ID NO: 61 - RNA, 50 nt, Artificial Sequence
  Synthetic: KB-018-026
cuaaaaacauggccccagcagcuucagucccuuucucgucgauggucagc

SEQ ID NO: 62 - RNA, 45 nt, Artificial Sequence
  Synthetic: KB-018-364
aacauggccccagcagcuucagucccuuucucgucgauggucagc

SEQ ID NO: 63 - RNA, 40 nt, Artificial Sequence
  Synthetic: KB-018-365
ggccccagcagcuucagucccuuucucgucgauggucagc

SEQ ID NO: 64 - RNA, 35 nt, Artificial Sequence
  Synthetic: KB-018-366
cagcagcuucagucccuuucucgucgauggucagc

SEQ ID NO: 65 - RNA, 31 nt, Artificial Sequence
  Synthetic: KB-018-367
agcuucagucccuuucucgucgauggucagc

SEQ ID NO: 66 - RNA, 50 nt, Artificial Sequence; identical to SEQ ID NO: 61
  Synthetic: KB-018-074

SEQ ID NO: 67 - RNA, 50 nt, Artificial Sequence; identical to SEQ ID NO: 61
  Synthetic: KB-018-075

SEQ ID NO: 68 - DNA, 50 bp, Artificial Sequence; identical to SEQ ID NO: 53
  Synthetic: KB-018-015

SEQ ID NO: 69 - RNA, 50 nt, Artificial Sequence; identical to SEQ ID NO: 61
  Synthetic: KB-018-016

SEQ ID NO: 70 - RNA, 50 nt, Artificial Sequence; identical to SEQ ID NO: 61
  Synthetic: KB-018-017

SEQ ID NO: 71 - RNA, 50 nt, Artificial Sequence; identical to SEQ ID NO: 61
  Synthetic: KB-018-093

SEQ ID NO: 72 - DNA, 50 bp, Artificial Sequence; identical to SEQ ID NO: 53
  Synthetic: KB-018-018

SEQ ID NO: 73 - DNA, 50 bp, Artificial Sequence; identical to SEQ ID NO: 53
  Synthetic: KB-018-019

SEQ ID NO: 74 - DNA, 50 bp, Artificial Sequence; identical to SEQ ID NO: 53
  Synthetic: KB-018-020

SEQ ID NO: 75 - RNA, 50 nt, Artificial Sequence; identical to SEQ ID NO: 61
  Synthetic: KB-018-021

SEQ ID NO: 76 - RNA, 50 nt, Artificial Sequence; identical to SEQ ID NO: 61
  Synthetic: KB-018-022

SEQ ID NO: 77 - RNA, 50 nt, Artificial Sequence; identical to SEQ ID NO: 61
  Synthetic: KB-018-023

SEQ ID NO: 78 - RNA, 50 nt, Artificial Sequence; identical to SEQ ID NO: 61
  Synthetic: KB-018-010

SEQ ID NO: 79 - DNA, 50 bp, Artificial Sequence; identical to SEQ ID NO: 53
  Synthetic: KB-018-024

SEQ ID NO: 80 - DNA, 50 bp, Artificial Sequence; identical to SEQ ID NO: 53
  Synthetic: KB-018-025

SEQ ID NO: 81 - RNA, 50 nt, Artificial Sequence; identical to SEQ ID NO: 61
  Synthetic: KB-018-026

SEQ ID NO: 82 - DNA, 50 bp, Artificial Sequence; identical to SEQ ID NO: 53
  Synthetic: KB-018-027

SEQ ID NO: 83 - RNA, 50 nt, Artificial Sequence; identical to SEQ ID NO: 61
  Synthetic: KB-018-028

SEQ ID NO: 84 - RNA, 50 nt, Artificial Sequence; identical to SEQ ID NO: 61
  Synthetic: KB-018-029

SEQ ID NO: 85 - RNA, 50 nt, Artificial Sequence; identical to SEQ ID NO: 61
  Synthetic: KB-018-030

SEQ ID NO: 86 - skipped

SEQ ID NO: 87 - protein, 16 aa, Artificial Sequence
  Synthetic: MTS-containing peptide
AAVALLPAVLLALLAP

SEQ ID NO: 88 - protein, 11 aa, Artificial Sequence
  Synthetic: RFGF analogue
AALLPVLLAAP

SEQ ID NO: 89 - protein, 13 aa, Human Immunodeficiency Virus
  misc_feature: HIV Tat protein
GRKKRRQRRRPPQ

SEQ ID NO: 90 - protein, 16 aa, Drosophila sp.
  misc_feature: Drosophila Antennapedia protein
RQIKIWFQNRRMKWKK

SEQ ID NO: 91 - RNA, 50 nt, Artificial Sequence; identical to SEQ ID NO: 61
  Synthetic: KB-018-031

SEQ ID NO: 92 - RNA, 50 nt, Artificial Sequence; identical to SEQ ID NO: 61
  Synthetic: KB-018-032

SEQ ID NO: 93 - RNA, 50 nt, Artificial Sequence; identical to SEQ ID NO: 61
  Synthetic: KB-018-033

SEQ ID NO: 94 - RNA, 50 nt, Artificial Sequence; identical to SEQ ID NO: 61
  Synthetic: KB-018-203

SEQ ID NO: 95 - DNA, 50 bp, Artificial Sequence; identical to SEQ ID NO: 53
  Synthetic: KB-018-204

SEQ ID NO: 96 - RNA, 50 nt, Artificial Sequence; identical to SEQ ID NO: 61
  Synthetic: KB-018-697

SEQ ID NO: 97 - RNA, 50 nt, Artificial Sequence; identical to SEQ ID NO: 61
  Synthetic: KB-018-698

SEQ ID NO: 98 - RNA, 50 nt, Artificial Sequence; identical to SEQ ID NO: 61
  Synthetic: KB-018-699

SEQ ID NO: 99 - RNA, 50 nt, Artificial Sequence
  Synthetic: KB-018-704
cuaaaaacauggccccagcagcuucagucccuuucgcgucgauggucagc

SEQ ID NO: 100 - RNA, 50 nt, Artificial Sequence; identical to SEQ ID NO: 99
  Synthetic: KB-018-705

SEQ ID NO: 101 - RNA, 50 nt, Artificial Sequence; identical to SEQ ID NO: 99
  Synthetic: KB-018-706

SEQ ID NO: 102 - RNA, 50 nt, Artificial Sequence; identical to SEQ ID NO: 99
  Synthetic: KB-018-707

SEQ ID NO: 103 - RNA, 50 nt, Artificial Sequence; identical to SEQ ID NO: 99
  Synthetic: KB-018-708

SEQ ID NO: 104 - RNA, 50 nt, Artificial Sequence
  Synthetic: KB-018-709
  misc_feature (38)..(38): n is inosine
cuaaaaacauggccccagcagcuucagucccuuucgcnucgauggucagc

SEQ ID NO: 105 - RNA, 50 nt, Artificial Sequence
  Synthetic: KB-018-710
  misc_feature (36)..(36): n is inosine
  misc_feature (38)..(38): n is inosine
cuaaaaacauggccccagcagcuucagucccuuucncnucgauggucagc

SEQ ID NO: 106 - RNA, 50 nt, Artificial Sequence
  Synthetic: KB-018-711
  misc_feature (36)..(36): n is inosine
cuaaaaacauggccccagcagcuucagucccuuucncgucgauggucagc

SEQ ID NO: 107 - RNA, 50 nt, Artificial Sequence; identical to SEQ ID NO: 106
  Synthetic: KB-018-712
  misc_feature (36)..(36): n is inosine

SEQ ID NO: 108 - RNA, 50 nt, Artificial Sequence; identical to SEQ ID NO: 106
  Synthetic: KB-018-713
  misc_feature (36)..(36): n is inosine

SEQ ID NO: 109 - DNA, 50 bp, Artificial Sequence; identical to SEQ ID NO: 53
  Synthetic: KB-018-714

SEQ ID NO: 110 - RNA, 50 nt, Artificial Sequence; identical to SEQ ID NO: 99
  Synthetic: KB-018-715

SEQ ID NO: 111 - RNA, 50 nt, Artificial Sequence; identical to SEQ ID NO: 61
  Synthetic: KB-018-716

SEQ ID NO: 112 - RNA, 50 nt, Artificial Sequence; identical to SEQ ID NO: 106
  Synthetic: KB-018-717
  misc_feature (36)..(36): n is inosine

SEQ ID NO: 113 - RNA, 50 nt, Artificial Sequence; identical to SEQ ID NO: 99
  Synthetic: KB-018-723

SEQ ID NO: 114 - DNA, 50 bp, Artificial Sequence; identical to SEQ ID NO: 53
  Synthetic: KB-018-001

SEQ ID NO: 115 - DNA, 50 bp, Artificial Sequence; identical to SEQ ID NO: 53
  Synthetic: KB-018-205

SEQ ID NO: 116 - DNA, 50 bp, Artificial Sequence; identical to SEQ ID NO: 53
  Synthetic: KB-018-206

SEQ ID NO: 117 - DNA, 50 bp, Artificial Sequence; identical to SEQ ID NO: 53
  Synthetic: KB-018-207

SEQ ID NO: 118 - DNA, 50 bp, Artificial Sequence; identical to SEQ ID NO: 53
  Synthetic: KB-018-208

SEQ ID NO: 119 - DNA, 50 bp, Artificial Sequence; identical to SEQ ID NO: 53
  Synthetic: KB-018-209

SEQ ID NO: 120 - DNA, 50 bp, Artificial Sequence; identical to SEQ ID NO: 53
  Synthetic: KB-018-210

SEQ ID NO: 121 - DNA, 50 bp, Artificial Sequence; identical to SEQ ID NO: 53
  Synthetic: KB-018-211

SEQ ID NO: 122 - RNA, 50 nt, Artificial Sequence; identical to SEQ ID NO: 61
  Synthetic: KB-018-212

SEQ ID NO: 123 - DNA, 50 bp, Artificial Sequence; identical to SEQ ID NO: 53
  Synthetic: KB-018-213

SEQ ID NO: 124 - DNA, 50 bp, Artificial Sequence; identical to SEQ ID NO: 53
  Synthetic: KB-018-214

SEQ ID NO: 125 - DNA, 51 bp, Artificial Sequence
  Synthetic: KB-018-215
cuaaaaacauggccccagcagcuucagucccuuuctgcgucgauggucagc

SEQ ID NO: 126 - RNA, 51 nt, Artificial Sequence
  Synthetic: KB-018-216
cuaaaaacauggccccagcagcuucagucccuuucugcgucgauggucagc

SEQ ID NO: 127 - DNA, 50 bp, Artificial Sequence; identical to SEQ ID NO: 53
  Synthetic: KB-018-001

SEQ ID NO: 128 - DNA, 50 bp, Artificial Sequence; identical to SEQ ID NO: 53
  Synthetic: KB-018-590

SEQ ID NO: 129 - DNA, 50 bp, Artificial Sequence; identical to SEQ ID NO: 53
  Synthetic: KB-018-217

SEQ ID NO: 130 - DNA, 50 bp, Artificial Sequence; identical to SEQ ID NO: 53
  Synthetic: KB-018-591

SEQ ID NO: 131 - DNA, 50 bp, Artificial Sequence; identical to SEQ ID NO: 53
  Synthetic: KB-018-592

SEQ ID NO: 132 - DNA, 50 bp, Artificial Sequence; identical to SEQ ID NO: 53
  Synthetic: KB-018-593

SEQ ID NO: 133 - DNA, 50 bp, Artificial Sequence; identical to SEQ ID NO: 53
  Synthetic: KB-018-594

SEQ ID NO: 134 - DNA, 50 bp, Artificial Sequence; identical to SEQ ID NO: 53
  Synthetic: KB-018-595

SEQ ID NO: 135 - DNA, 50 bp, Artificial Sequence; identical to SEQ ID NO: 53
  Synthetic: KB-018-596

SEQ ID NO: 136 - DNA, 50 bp, Artificial Sequence; identical to SEQ ID NO: 53
  Synthetic: KB-018-001

SEQ ID NO: 137 - RNA, 50 nt, Artificial Sequence; identical to SEQ ID NO: 61
  Synthetic: KB-018-600

SEQ ID NO: 138 - DNA, 50 bp, Artificial Sequence; identical to SEQ ID NO: 53
  Synthetic: KB-018-218

SEQ ID NO: 139 - DNA, 50 bp, Artificial Sequence; identical to SEQ ID NO: 53
  Synthetic: KB-018-601

SEQ ID NO: 140 - RNA, 50 nt, Artificial Sequence; identical to SEQ ID NO: 61
  Synthetic: KB-018-602

SEQ ID NO: 141 - DNA, 50 bp, Artificial Sequence; identical to SEQ ID NO: 53
  Synthetic: KB-018-603

SEQ ID NO: 142 - RNA, 50 nt, Artificial Sequence; identical to SEQ ID NO: 61
  Synthetic: KB-018-604

SEQ ID NO: 143 - RNA, 50 nt, Artificial Sequence; identical to SEQ ID NO: 61
  Synthetic: KB-018-605

SEQ ID NO: 144 - RNA, 50 nt, Artificial Sequence; identical to SEQ ID NO: 61
  Synthetic: KB-018-606

SEQ ID NO: 145 - DNA, 50 bp, Artificial Sequence; identical to SEQ ID NO: 53
  Synthetic: KB-018-001

SEQ ID NO: 146 - RNA, 50 nt, Artificial Sequence; identical to SEQ ID NO: 61
  Synthetic: KB-018-617

SEQ ID NO: 147 - DNA, 50 bp, Artificial Sequence; identical to SEQ ID NO: 53
  Synthetic: KB-018-219

SEQ ID NO: 148 - DNA, 50 bp, Artificial Sequence; identical to SEQ ID NO: 53
  Synthetic: KB-018-618

SEQ ID NO: 149 - RNA, 50 nt, Artificial Sequence; identical to SEQ ID NO: 61
  Synthetic: KB-018-619

SEQ ID NO: 150 - DNA, 50 bp, Artificial Sequence; identical to SEQ ID NO: 53
  Synthetic: KB-018-620

SEQ ID NO: 151 - RNA, 50 nt, Artificial Sequence; identical to SEQ ID NO: 61
  Synthetic: KB-018-621

SEQ ID NO: 152 - RNA, 50 nt, Artificial Sequence; identical to SEQ ID NO: 61
  Synthetic: KB-018-622

SEQ ID NO: 153 - RNA, 50 nt, Artificial Sequence; identical to SEQ ID NO: 61
  Synthetic: KB-018-623

SEQ ID NO: 154 - DNA, 50 bp, Artificial Sequence; identical to SEQ ID NO: 53
  Synthetic: KB-018-001

SEQ ID NO: 155 - RNA, 50 nt, Artificial Sequence
  Synthetic: KB-018-634
cuaaaaacauggccccagcagcuucagucccuuucscgucgauggucagc

SEQ ID NO: 156 - DNA, 50 bp, Artificial Sequence
  Synthetic: KB-018-220
cuaaaaacauggccccagcagcuucagucccuuuctsgucgauggucagc

SEQ ID NO: 157 - DNA, 50 bp, Artificial Sequence
  Synthetic: KB-018-635
cuaaaaacauggccccagcagcuucagucccuuuctcsucgauggucagc

SEQ ID NO: 158 - RNA, 50 nt, Artificial Sequence
  Synthetic: KB-018-636
cuaaaaacauggccccagcagcuucagucccuuucsssucgauggucagc

SEQ ID NO: 159 - DNA, 50 bp, Artificial Sequence
  Synthetic: KB-018-637
cuaaaaacauggccccagcagcuucagucccuuuctssucgauggucagc

SEQ ID NO: 160 - RNA, 50 nt, Artificial Sequence
  Synthetic: KB-018-638
cuaaaaacauggccccagcagcuucagucccuuucssgucgauggucagc

SEQ ID NO: 161 - RNA, 50 nt, Artificial Sequence
  Synthetic: KB-018-639
cuaaaaacauggccccagcagcuucagucccuuucscsucgauggucagc

SEQ ID NO: 162 - RNA, 50 nt, Artificial Sequence; identical to SEQ ID NO: 161
  Synthetic: KB-018-640

SEQ ID NO: 163 - DNA, 50 bp, Artificial Sequence; identical to SEQ ID NO: 53
  Synthetic: KB-018-001

SEQ ID NO: 164 - RNA, 50 nt, Artificial Sequence; identical to SEQ ID NO: 155
  Synthetic: KB-018-641

SEQ ID NO: 165 - DNA, 50 bp, Artificial Sequence; identical to SEQ ID NO: 156
  Synthetic: KB-018-642

SEQ ID NO: 166 - DNA, 50 bp, Artificial Sequence; identical to SEQ ID NO: 157
  Synthetic: KB-018-643

SEQ ID NO: 167 - RNA, 50 nt, Artificial Sequence; identical to SEQ ID NO: 158
  Synthetic: KB-018-644

SEQ ID NO: 168 - DNA, 50 bp, Artificial Sequence; identical to SEQ ID NO: 159
  Synthetic: KB-018-645

SEQ ID NO: 169 - RNA, 50 nt, Artificial Sequence; identical to SEQ ID NO: 160
  Synthetic: KB-018-646

SEQ ID NO: 170 - RNA, 50 nt, Artificial Sequence; identical to SEQ ID NO: 161
  Synthetic: KB-018-647

SEQ ID NO: 171 - RNA, 50 nt, Artificial Sequence; identical to SEQ ID NO: 161
  Synthetic: KB-018-648

SEQ ID NO: 172 - DNA, 50 bp, Artificial Sequence; identical to SEQ ID NO: 53
  Synthetic: KB-018-001

SEQ ID NO: 173 - RNA, 50 nt, Artificial Sequence; identical to SEQ ID NO: 155
  Synthetic: KB-018-650

SEQ ID NO: 174 - DNA, 50 bp, Artificial Sequence; identical to SEQ ID NO: 156
  Synthetic: KB-018-651

SEQ ID NO: 175 - DNA, 50 bp, Artificial Sequence; identical to SEQ ID NO: 157
  Synthetic: KB-018-652

SEQ ID NO: 176 - RNA, 50 nt, Artificial Sequence; identical to SEQ ID NO: 158
  Synthetic: KB-018-653

SEQ ID NO: 177 - DNA, 50 bp, Artificial Sequence; identical to SEQ ID NO: 159
  Synthetic: KB-018-654

SEQ ID NO: 178 - RNA, 50 nt, Artificial Sequence; identical to SEQ ID NO: 160
  Synthetic: KB-018-655

SEQ ID NO: 179 - RNA, 50 nt, Artificial Sequence; identical to SEQ ID NO: 161
  Synthetic: KB-018-656

SEQ ID NO: 180 - RNA, 50 nt, Artificial Sequence; identical to SEQ ID NO: 161
  Synthetic: KB-018-657

SEQ ID NO: 181 - DNA, 50 bp, Artificial Sequence
  Synthetic: KB-018-724
cuaaaaacauggccccagcagcuucagucccuuuctcgtcgauggucagc

SEQ ID NO: 182 - DNA, 50 bp, Artificial Sequence; identical to SEQ ID NO: 181
  Synthetic: KB-018-725

SEQ ID NO: 183 - DNA, 50 bp, Artificial Sequence; identical to SEQ ID NO: 53
  Synthetic: KB-018-726

SEQ ID NO: 184 - DNA, 50 bp, Artificial Sequence; identical to SEQ ID NO: 181
  Synthetic: KB-018-727

SEQ ID NO: 185 - DNA, 50 bp, Artificial Sequence; identical to SEQ ID NO: 53
  Synthetic: KB-018-728

SEQ ID NO: 186 - DNA, 50 bp, Artificial Sequence; identical to SEQ ID NO: 53
  Synthetic: KB-018-001

SEQ ID NO: 187 - DNA, 50 bp, Artificial Sequence; identical to SEQ ID NO: 53
  Synthetic: KB-018-451

SEQ ID NO: 188 - DNA, 50 bp, Artificial Sequence; identical to SEQ ID NO: 53
  Synthetic: KB-018-452

SEQ ID NO: 189 - DNA, 50 bp, Artificial Sequence; identical to SEQ ID NO: 53
  Synthetic: KB-018-453

SEQ ID NO: 190 - DNA, 50 bp, Artificial Sequence; identical to SEQ ID NO: 53
  Synthetic: KB-018-454

SEQ ID NO: 191 - DNA, 50 bp, Artificial Sequence; identical to SEQ ID NO: 53
  Synthetic: KB-018-455

SEQ ID NO: 192 - DNA, 50 bp, Artificial Sequence; identical to SEQ ID NO: 53
  Synthetic: KB-018-456

SEQ ID NO: 193 - DNA, 50 bp, Artificial Sequence; identical to SEQ ID NO: 53
  Synthetic: KB-018-457

SEQ ID NO: 194 - DNA, 50 bp, Artificial Sequence; identical to SEQ ID NO: 53
  Synthetic: KB-018-458

SEQ ID NO: 195 - DNA, 50 bp, Artificial Sequence; identical to SEQ ID NO: 53
  Synthetic: KB-018-459

SEQ ID NO: 196 - DNA, 50 bp, Artificial Sequence; identical to SEQ ID NO: 53
  Synthetic: KB-018-460

SEQ ID NO: 197 - DNA, 50 bp, Artificial Sequence; identical to SEQ ID NO: 53
  Synthetic: KB-018-461

SEQ ID NO: 198 - DNA, 50 bp, Artificial Sequence; identical to SEQ ID NO: 53
  Synthetic: KB-018-462

SEQ ID NO: 199 - DNA, 50 bp, Artificial Sequence; identical to SEQ ID NO: 53
  Synthetic: KB-018-463

SEQ ID NO: 200 - DNA, 50 bp, Artificial Sequence; identical to SEQ ID NO: 53
  Synthetic: KB-018-464

SEQ ID NO: 201 - DNA, 50 bp, Artificial Sequence; identical to SEQ ID NO: 53
  Synthetic: KB-018-465

SEQ ID NO: 202 - DNA, 50 bp, Artificial Sequence; identical to SEQ ID NO: 53
  Synthetic: KB-018-466

SEQ ID NO: 203 - DNA, 50 bp, Artificial Sequence; identical to SEQ ID NO: 53
  Synthetic: KB-018-467

SEQ ID NO: 204 - DNA, 50 bp, Artificial Sequence; identical to SEQ ID NO: 53
  Synthetic: KB-018-468

SEQ ID NO: 205 - DNA, 50 bp, Artificial Sequence; identical to SEQ ID NO: 53
  Synthetic: KB-018-469

SEQ ID NO: 206 - DNA, 50 bp, Artificial Sequence; identical to SEQ ID NO: 53
  Synthetic: KB-018-470

SEQ ID NO: 207 - DNA, 50 bp, Artificial Sequence; identical to SEQ ID NO: 53
  Synthetic: KB-018-471

SEQ ID NO: 208 - DNA, 50 bp, Artificial Sequence; identical to SEQ ID NO: 53
  Synthetic: KB-018-472

SEQ ID NO: 209 - DNA, 50 bp, Artificial Sequence; identical to SEQ ID NO: 53
  Synthetic: KB-018-473

SEQ ID NO: 210 - DNA, 50 bp, Artificial Sequence; identical to SEQ ID NO: 53
  Synthetic: KB-018-474

SEQ ID NO: 211 - DNA, 50 bp, Artificial Sequence; identical to SEQ ID NO: 53
  Synthetic: KB-018-475

SEQ ID NO: 212 - DNA, 50 bp, Artificial Sequence; identical to SEQ ID NO: 53
  Synthetic: KB-018-476

SEQ ID NO: 213 - DNA, 50 bp, Artificial Sequence; identical to SEQ ID NO: 53
  Synthetic: KB-018-477

SEQ ID NO: 214 - DNA, 50 bp, Artificial Sequence; identical to SEQ ID NO: 53
  Synthetic: KB-018-478

SEQ ID NO: 215 - DNA, 50 bp, Artificial Sequence; identical to SEQ ID NO: 53
  Synthetic: KB-018-479

SEQ ID NO: 216 - DNA, 50 bp, Artificial Sequence; identical to SEQ ID NO: 53
  Synthetic: KB-018-480

SEQ ID NO: 217 - DNA, 50 bp, Artificial Sequence; identical to SEQ ID NO: 53
  Synthetic: KB-018-481

SEQ ID NO: 218 - DNA, 50 bp, Artificial Sequence; identical to SEQ ID NO: 53
  Synthetic: KB-018-482

SEQ ID NO: 219 - DNA, 50 bp, Artificial Sequence; identical to SEQ ID NO: 53
  Synthetic: KB-018-483

SEQ ID NO: 220 - DNA, 50 bp, Artificial Sequence; identical to SEQ ID NO: 53
  Synthetic: KB-018-484

SEQ ID NO: 221 - DNA, 50 bp, Artificial Sequence; identical to SEQ ID NO: 53
  Synthetic: KB-018-485

SEQ ID NO: 222 - DNA, 50 bp, Artificial Sequence; identical to SEQ ID NO: 53
  Synthetic: KB-018-486

SEQ ID NO: 223 - DNA, 50 bp, Artificial Sequence; identical to SEQ ID NO: 53
  Synthetic: KB-018-487

SEQ ID NO: 224 - DNA, 50 bp, Artificial Sequence; identical to SEQ ID NO: 53
  Synthetic: KB-018-488

SEQ ID NO: 225 - DNA, 50 bp, Artificial Sequence; identical to SEQ ID NO: 53
  Synthetic: KB-018-489

SEQ ID NO: 226 - DNA, 50 bp, Artificial Sequence; identical to SEQ ID NO: 53
  Synthetic: KB-018-490

SEQ ID NO: 227 - DNA, 50 bp, Artificial Sequence; identical to SEQ ID NO: 53
  Synthetic: KB-018-491

SEQ ID NO: 228 - DNA, 50 bp, Artificial Sequence; identical to SEQ ID NO: 53
  Synthetic: KB-018-492

SEQ ID NO: 229 - DNA, 50 bp, Artificial Sequence; identical to SEQ ID NO: 53
  Synthetic: KB-018-493

SEQ ID NO: 230 - DNA, 50 bp, Artificial Sequence; identical to SEQ ID NO: 53
  Synthetic: KB-018-494

SEQ ID NO: 231 - DNA, 50 bp, Artificial Sequence; identical to SEQ ID NO: 53
  Synthetic: KB-018-495

SEQ ID NO: 232 - DNA, 50 bp, Artificial Sequence; identical to SEQ ID NO: 53
  Synthetic: KB-018-496

SEQ ID NO: 233 - DNA, 50 bp, Artificial Sequence; identical to SEQ ID NO: 53
  Synthetic: KB-018-497

SEQ ID NO: 234 - DNA, 50 bp, Artificial Sequence; identical to SEQ ID NO: 53
  Synthetic: KB-018-001

SEQ ID NO: 235 - DNA, 45 bp, Artificial Sequence; identical to SEQ ID NO: 54
  Synthetic: KB-018-378

SEQ ID NO: 236 - DNA, 50 bp, Artificial Sequence; identical to SEQ ID NO: 53
  Synthetic: KB-018-1261

SEQ ID NO: 237 - DNA, 50 bp, Artificial Sequence; identical to SEQ ID NO: 53
  Synthetic: KB-018-1262

SEQ ID NO: 238 - DNA, 50 bp, Artificial Sequence; identical to SEQ ID NO: 53
  Synthetic: KB-018-1263

SEQ ID NO: 239 - DNA, 50 bp, Artificial Sequence; identical to SEQ ID NO: 53
  Synthetic: KB-018-1264

SEQ ID NO: 240 - DNA, 50 bp, Artificial Sequence; identical to SEQ ID NO: 53
  Synthetic: KB-018-1289

SEQ ID NO: 241 - DNA, 50 bp, Artificial Sequence; identical to SEQ ID NO: 53
  Synthetic: KB-018-001

SEQ ID NO: 242 - DNA, 50 bp, Artificial Sequence; identical to SEQ ID NO: 53
  Synthetic: KB-018-403

SEQ ID NO: 243 - DNA, 50 bp, Artificial Sequence; identical to SEQ ID NO: 53
  Synthetic: KB-018-404

SEQ ID NO: 244 - DNA, 50 bp, Artificial Sequence; identical to SEQ ID NO: 53
  Synthetic: KB-018-405

SEQ ID NO: 245 - DNA, 50 bp, Artificial Sequence; identical to SEQ ID NO: 53
  Synthetic: KB-018-406

SEQ ID NO: 246 - DNA, 50 bp, Artificial Sequence; identical to SEQ ID NO: 53
  Synthetic: KB-018-407

SEQ ID NO: 247 - DNA, 50 bp, Artificial Sequence; identical to SEQ ID NO: 53
  Synthetic: KB-018-408

SEQ ID NO: 248 - DNA, 50 bp, Artificial Sequence; identical to SEQ ID NO: 53
  Synthetic: KB-018-409

SEQ ID NO: 249 - DNA, 50 bp, Artificial Sequence; identical to SEQ ID NO: 53
  Synthetic: KB-018-410

SEQ ID NO: 250 - DNA, 50 bp, Artificial Sequence; identical to SEQ ID NO: 53
  Synthetic: KB-018-411

SEQ ID NO: 251 - DNA, 50 bp, Artificial Sequence; identical to SEQ ID NO: 53
  Synthetic: KB-018-412

SEQ ID NO: 252 - DNA, 50 bp, Artificial Sequence; identical to SEQ ID NO: 53
  Synthetic: KB-018-413

SEQ ID NO: 253 - DNA, 50 bp, Artificial Sequence; identical to SEQ ID NO: 53
  Synthetic: KB-018-414

SEQ ID NO: 254 - DNA, 50 bp, Artificial Sequence; identical to SEQ ID NO: 53
  Synthetic: KB-018-415

SEQ ID NO: 255 - DNA, 50 bp, Artificial Sequence; identical to SEQ ID NO: 53
  Synthetic: KB-018-416

SEQ ID NO: 256 - DNA, 50 bp, Artificial Sequence; identical to SEQ ID NO: 53
  Synthetic: KB-018-417

SEQ ID NO: 257 - DNA, 50 bp, Artificial Sequence; identical to SEQ ID NO: 53
  Synthetic: KB-018-418

SEQ ID NO: 258 - DNA, 50 bp, Artificial Sequence; identical to SEQ ID NO: 53
  Synthetic: KB-018-419

SEQ ID NO: 259 - DNA, 50 bp, Artificial Sequence; identical to SEQ ID NO: 53
  Synthetic: KB-018-420

SEQ ID NO: 260 - DNA, 50 bp, Artificial Sequence; identical to SEQ ID NO: 53
  Synthetic: KB-018-421

SEQ ID NO: 261 - DNA, 50 bp, Artificial Sequence; identical to SEQ ID NO: 53
  Synthetic: KB-018-422

SEQ ID NO: 262 - DNA, 50 bp, Artificial Sequence; identical to SEQ ID NO: 53
  Synthetic: KB-018-423

SEQ ID NO: 263 - DNA, 50 bp, Artificial Sequence; identical to SEQ ID NO: 53
  Synthetic: KB-018-424

SEQ ID NO: 264 - DNA, 50 bp, Artificial Sequence; identical to SEQ ID NO: 53
  Synthetic: KB-018-425

SEQ ID NO: 265 - DNA, 50 bp, Artificial Sequence; identical to SEQ ID NO: 53
  Synthetic: KB-018-426

SEQ ID NO: 266 - DNA, 50 bp, Artificial Sequence; identical to SEQ ID NO: 53
  Synthetic: KB-018-427

SEQ ID NO: 267 - DNA, 50 bp, Artificial Sequence; identical to SEQ ID NO: 53
  Synthetic: KB-018-428

SEQ ID NO: 268 - DNA, 50 bp, Artificial Sequence; identical to SEQ ID NO: 53
  Synthetic: KB-018-429

SEQ ID NO: 269 - DNA, 50 bp, Artificial Sequence; identical to SEQ ID NO: 53
  Synthetic: KB-018-430

SEQ ID NO: 270 - DNA, 50 bp, Artificial Sequence; identical to SEQ ID NO: 53
  Synthetic: KB-018-431

SEQ ID NO: 271 - DNA, 50 bp, Artificial Sequence; identical to SEQ ID NO: 53
  Synthetic: KB-018-432

SEQ ID NO: 272 - DNA, 50 bp, Artificial Sequence; identical to SEQ ID NO: 53
  Synthetic: KB-018-433

SEQ ID NO: 273 - DNA, 50 bp, Artificial Sequence; identical to SEQ ID NO: 53
  Synthetic: KB-018-434

SEQ ID NO: 274 - DNA, 50 bp, Artificial Sequence; identical to SEQ ID NO: 53
  Synthetic: KB-018-435

SEQ ID NO: 275 - DNA, 50 bp, Artificial Sequence; identical to SEQ ID NO: 53
  Synthetic: KB-018-436

SEQ ID NO: 276 - RNA, 50 nt, Artificial Sequence; identical to SEQ ID NO: 61
  Synthetic: KB-018-437

SEQ ID NO: 277 - DNA, 50 bp, Artificial Sequence; identical to SEQ ID NO: 53
  Synthetic: KB-018-438

SEQ ID NO: 278 - DNA, 50 bp, Artificial Sequence; identical to SEQ ID NO: 53
  Synthetic: KB-018-439

SEQ ID NO: 279 - DNA, 50 bp, Artificial Sequence; identical to SEQ ID NO: 53
  Synthetic: KB-018-440

SEQ ID NO: 280 - DNA, 50 bp, Artificial Sequence; identical to SEQ ID NO: 53
  Synthetic: KB-018-441

SEQ ID NO: 281 - DNA, 50 bp, Artificial Sequence; identical to SEQ ID NO: 53
  Synthetic: KB-018-442

SEQ ID NO: 282 - DNA, 50 bp, Artificial Sequence; identical to SEQ ID NO: 53
  Synthetic: KB-018-443

SEQ ID NO: 283 - DNA, 50 bp, Artificial Sequence; identical to SEQ ID NO: 53
  Synthetic: KB-018-444

SEQ ID NO: 284 - DNA, 50 bp, Artificial Sequence; identical to SEQ ID NO: 53
  Synthetic: KB-018-445

SEQ ID NO: 285 - DNA, 50 bp, Artificial Sequence; identical to SEQ ID NO: 53
  Synthetic: KB-018-446

SEQ ID NO: 286 - DNA, 50 bp, Artificial Sequence; identical to SEQ ID NO: 53
  Synthetic: KB-018-447

SEQ ID NO: 287 - DNA, 50 bp, Artificial Sequence; identical to SEQ ID NO: 53
  Synthetic: KB-018-448

SEQ ID NO: 288 - DNA, 50 bp, Artificial Sequence; identical to SEQ ID NO: 53
  Synthetic: KB-018-449

SEQ ID NO: 289 - DNA, 50 bp, Artificial Sequence; identical to SEQ ID NO: 53
  Synthetic: KB-018-450

SEQ ID NO: 290 - RNA, 50 nt, Artificial Sequence; identical to SEQ ID NO: 61
  Synthetic: KB-018-700

SEQ ID NO: 291 - RNA, 50 nt, Artificial Sequence; identical to SEQ ID NO: 61
  Synthetic: KB-018-701

SEQ ID NO: 292 - RNA, 50 nt, Artificial Sequence; identical to SEQ ID NO: 61
  Synthetic: KB-018-702

SEQ ID NO: 293 - RNA, 50 nt, Artificial Sequence; identical to SEQ ID NO: 61
  Synthetic: KB-018-703

SEQ ID NO: 294 - RNA, 50 nt, Artificial Sequence; identical to SEQ ID NO: 61
  Synthetic: KB-018-718

SEQ ID NO: 295 - DNA, 50 bp, Artificial Sequence; identical to SEQ ID NO: 53
  Synthetic: KB-018-719

SEQ ID NO: 296 - RNA, 50 nt, Artificial Sequence; identical to SEQ ID NO: 61
  Synthetic: KB-018-720

SEQ ID NO: 297 - RNA, 50 nt, Artificial Sequence; identical to SEQ ID NO: 61
  Synthetic: KB-018-721

SEQ ID NO: 298 - RNA, 50 nt, Artificial Sequence; identical to SEQ ID NO: 61
  Synthetic: KB-018-437

SEQ ID NO: 299 - DNA, 50 bp, Artificial Sequence; identical to SEQ ID NO: 53
  Synthetic: KB-018-438

SEQ ID NO: 300 - DNA, 50 bp, Artificial Sequence; identical to SEQ ID NO: 53
  Synthetic: KB-018-439

SEQ ID NO: 301 - RNA, 50 nt, Artificial Sequence; identical to SEQ ID NO: 61
  Synthetic: KB-018-722

SEQ ID NO: 302 - DNA, 50 bp, Artificial Sequence; identical to SEQ ID NO: 53
  Synthetic: KB-018-001

SEQ ID NO: 303 - DNA, 50 bp, Artificial Sequence; identical to SEQ ID NO: 53
  Synthetic: KB-018-1265

SEQ ID NO: 304 - DNA, 50 bp, Artificial Sequence; identical to SEQ ID NO: 53
  Synthetic: KB-018-1266

SEQ ID NO: 305 - DNA, 50 bp, Artificial Sequence; identical to SEQ ID NO: 53
  Synthetic: KB-018-1267

SEQ ID NO: 306 - DNA, 50 bp, Artificial Sequence; identical to SEQ ID NO: 53
  Synthetic: KB-018-1268

SEQ ID NO: 307 - RNA, 50 nt, Artificial Sequence; identical to SEQ ID NO: 61
  Synthetic: KB-018-697

SEQ ID NO: 308 - RNA, 50 nt, Artificial Sequence; identical to SEQ ID NO: 61
  Synthetic: KB-018-699

SEQ ID NO: 309 - DNA, 50 bp, Artificial Sequence; identical to SEQ ID NO: 53
  Synthetic: KB-018-001

SEQ ID NO: 310 - DNA, 50 bp, Artificial Sequence; identical to SEQ ID NO: 53
  Synthetic: KB-018-1269

SEQ ID NO: 311 - DNA, 50 bp, Artificial Sequence; identical to SEQ ID NO: 53
  Synthetic: KB-018-1270

SEQ ID NO: 312 - DNA, 50 bp, Artificial Sequence; identical to SEQ ID NO: 53
  Synthetic: KB-018-1271

SEQ ID NO: 313 - DNA, 50 bp, Artificial Sequence; identical to SEQ ID NO: 53
  Synthetic: KB-018-1272

SEQ ID NO: 314 - DNA, 50 bp, Artificial Sequence; identical to SEQ ID NO: 53
  Synthetic: KB-018-1290

SEQ ID NO: 315 - RNA, 50 nt, Artificial Sequence; identical to SEQ ID NO: 61
  Synthetic: KB-018-1273

SEQ ID NO: 316 - RNA, 50 nt, Artificial Sequence; identical to SEQ ID NO: 61
  Synthetic: KB-018-1274

SEQ ID NO: 317 - DNA, 50 bp, Artificial Sequence; identical to SEQ ID NO: 53
  Synthetic: KB-018-001

SEQ ID NO: 318 - DNA, 50 bp, Artificial Sequence
  Synthetic: KB-018-012
cuaaaaacauggccccagcagcuucagucccuuucttgucgauggucagc

SEQ ID NO: 319 - DNA, 50 bp, Artificial Sequence
  Synthetic: KB-018-174
cuaaaaacauggccccagcagcuucagucccuuuctggucgauggucagc

SEQ ID NO: 320 - DNA, 50 bp, Artificial Sequence
  Synthetic: KB-018-175
cuaaaaacauggccccagcagcuucagucccuuuctagucgauggucagc

SEQ ID NO: 321 - RNA, 50 nt, Artificial Sequence; identical to SEQ ID NO: 99
  Synthetic: KB-018-176

SEQ ID NO: 322 - RNA, 50 nt, Artificial Sequence; identical to SEQ ID NO: 106
  Synthetic: KB-018-177
  misc_feature (36)..(36): n is inosine

SEQ ID NO: 323 - RNA, 50 nt, Artificial Sequence
  Synthetic: KB-018-178
cuaaaaacauggccccagcagcuucagucccuuucacgucgauggucagc

SEQ ID NO: 324 - RNA, 50 nt, Artificial Sequence
  Synthetic: KB-018-179
cuaaaaacauggccccagcagcuucagucccuuucccgucgauggucagc

SEQ ID NO: 325 - DNA, 50 bp, Artificial Sequence
  Synthetic: KB-018-180
cuaaaaacauggccccagcagcuucagucccuuuctcaucgauggucagc

SEQ ID NO: 326 - DNA, 50 bp, Artificial Sequence
  Synthetic: KB-018-181
cuaaaaacauggccccagcagcuucagucccuuuctccucgauggucagc

SEQ ID NO: 327 - DNA, 50 bp, Artificial Sequence
  Synthetic: KB-018-182
cuaaaaacauggccccagcagcuucagucccuuuctctucgauggucagc

SEQ ID NO: 328 - RNA, 50 nt, Artificial Sequence
  Synthetic: KB-018-183
cuaaaaacauggccccagcagcuucagucccuuucgccucgauggucagc

SEQ ID NO: 329 - DNA, 50 bp, Artificial Sequence; identical to SEQ ID NO: 53
  Synthetic: KB-018-001

SEQ ID NO: 330 - DNA, 50 bp, Artificial Sequence
  Synthetic: KB-018-110
cuaaaaacauggccccagcagcuucaguccccuuctcgucgauggucagc

SEQ ID NO: 331 - DNA, 50 bp, Artificial Sequence
  Synthetic: KB-018-559
cuaaaaacauggccccagcagcuucaguccguuuctcgucgauggucagc

SEQ ID NO: 332 - DNA, 50 bp, Artificial Sequence
  Synthetic: KB-018-560
cuaaaaacauggccccagcagcuucagucgcuuuctcgucgauggucagc

SEQ ID NO: 333 - DNA, 50 bp, Artificial Sequence
  Synthetic: KB-018-561
cuaaaaacauggccccagcagcuucagugccuuuctcgucgauggucagc

SEQ ID NO: 334 - DNA, 50 bp, Artificial Sequence
  Synthetic: KB-018-111
cuaaaaacauggccccagcagcuucagccccuuuctcgucgauggucagc

SEQ ID NO: 335 - DNA, 50 bp, Artificial Sequence
  Synthetic: KB-018-562
cuaaaaacauggccccagcagcuucacucccuuuctcgucgauggucagc

SEQ ID NO: 336 - DNA, 50 bp, Artificial Sequence
  Synthetic: KB-018-563
cuaaaaacauggccccagcagcuucugucccuuuctcgucgauggucagc

SEQ ID NO: 337 - DNA, 50 bp, Artificial Sequence
  Synthetic: KB-018-564
cuaaaaacauggccccagcagcuugagucccuuuctcgucgauggucagc

SEQ ID NO: 338 - DNA, 50 bp, Artificial Sequence
  Synthetic: KB-018-112
cuaaaaacauggccccagcagcuccagucccuuuctcgucgauggucagc

SEQ ID NO: 339 - DNA, 50 bp, Artificial Sequence
  Synthetic: KB-018-113
cuaaaaacauggccccagcagccucagucccuuuctcgucgauggucagc

SEQ ID NO: 340 - DNA, 50 bp, Artificial Sequence
  Synthetic: KB-018-565
cuaaaaacauggccccagcagguucagucccuuuctcgucgauggucagc

SEQ ID NO: 341 - DNA, 50 bp, Artificial Sequence
  Synthetic: KB-018-566
cuaaaaacauggccccagcaccuucagucccuuuctcgucgauggucagc

SEQ ID NO: 342 - DNA, 50 bp, Artificial Sequence
  Synthetic: KB-018-172
cuaaaaacauggccccagccgcuucagucccuuuctcgucgauggucagc

SEQ ID NO: 343 - DNA, 50 bp, Artificial Sequence
  Synthetic: KB-018-567
cuaaaaacauggccccaggagcuucagucccuuuctcgucgauggucagc

SEQ ID NO: 344 - DNA, 50 bp, Artificial Sequence
  Synthetic: KB-018-568
cuaaaaacauggccccaccagcuucagucccuuuctcgucgauggucagc

SEQ ID NO: 345 - DNA, 50 bp, Artificial Sequence
  Synthetic: KB-018-569
cuaaaaacauggccccugcagcuucagucccuuuctcgucgauggucagc

SEQ ID NO: 346 - DNA, 50 bp, Artificial Sequence
  Synthetic: KB-018-570
cuaaaaacauggcccgagcagcuucagucccuuuctcgucgauggucagc

SEQ ID NO: 347 - DNA, 50 bp, Artificial Sequence
  Synthetic: KB-018-571
cuaaaaacauggccgcagcagcuucagucccuuuctcgucgauggucagc

SEQ ID NO: 348 - DNA, 50 bp, Artificial Sequence
  Synthetic: KB-018-572
cuaaaaacauggcgccagcagcuucagucccuuuctcgucgauggucagc

SEQ ID NO: 349 - DNA, 50 bp, Artificial Sequence
  Synthetic: KB-018-573
cuaaaaacaugggcccagcagcuucagucccuuuctcgucgauggucagc

SEQ ID NO: 350 - DNA, 50 bp, Artificial Sequence
  Synthetic: KB-018-574
cuaaaaacaugcccccagcagcuucagucccuuuctcgucgauggucagc

SEQ ID NO: 351 - DNA, 50 bp, Artificial Sequence
  Synthetic: KB-018-575
cuaaaaacaucgccccagcagcuucagucccuuuctcgucgauggucagc

SEQ ID NO: 352 - DNA, 50 bp, Artificial Sequence
  Synthetic: KB-018-114
cuaaaaacacggccccagcagcuucagucccuuuctcgucgauggucagc

SEQ ID NO: 353 - DNA, 50 bp, Artificial Sequence
  Synthetic: KB-018-576
cuaaaaacuuggccccagcagcuucagucccuuuctcgucgauggucagc

SEQ ID NO: 354 - DNA, 50 bp, Artificial Sequence
  Synthetic: KB-018-577
cuaaaaagauggccccagcagcuucagucccuuuctcgucgauggucagc

SEQ ID NO: 355 - DNA, 50 bp, Artificial Sequence
  Synthetic: KB-018-578
cuaaaaucauggccccagcagcuucagucccuuuctcgucgauggucagc

SEQ ID NO: 356 - DNA, 50 bp, Artificial Sequence
  Synthetic: KB-018-579
cuaaauacauggccccagcagcuucagucccuuuctcgucgauggucagc

SEQ ID NO: 357 - DNA, 50 bp, Artificial Sequence; identical to SEQ ID NO: 53
  Synthetic: KB-018-001

SEQ ID NO: 358 - RNA, 50 nt, Artificial Sequence; identical to SEQ ID NO: 61
  Synthetic: KB-018-093

SEQ ID NO: 359 - DNA, 50 bp, Artificial Sequence
  Synthetic: KB-018-108
cuaaaaacauggccccagcagcuucagugccucuctcgucgauggucagc

SEQ ID NO: 360 - DNA, 50 bp, Artificial Sequence
  Synthetic: KB-018-094
cuaaaaacauggccccagcaacuugagucccuuuctcgucgauggucagc

SEQ ID NO: 361 - RNA, 50 nt, Artificial Sequence
  Synthetic: KB-018-095
cuaaaaacauggccccagcaacuugagucccuuucucgucgauggucagc

SEQ ID NO: 362 - RNA, 50 nt, Artificial Sequence; identical to SEQ ID NO: 361
  Synthetic: KB-018-096

SEQ ID NO: 363 - RNA, 50 nt, Artificial Sequence; identical to SEQ ID NO: 361
  Synthetic: KB-018-097

SEQ ID NO: 364 - DNA, 39 bp, Artificial Sequence
  Synthetic: KB-018-098
auggccccagcaacuugagucccuuuctcgucgaugguc

SEQ ID NO: 365 - RNA, 39 nt, Artificial Sequence
  Synthetic: KB-018-099
auggccccagcaacuugagucccuuucucgucgaugguc

SEQ ID NO: 366 - DNA, 40 bp, Artificial Sequence
  Synthetic: KB-018-100
cauggccccagcggcuccagucccuuuctcgucgaugguc

SEQ ID NO: 367 - RNA, 40 nt, Artificial Sequence
  Synthetic: KB-018-101
cauggccccagcggcuccagucccuuucucgucgaugguc

SEQ ID NO: 368 - DNA, 50 bp, Artificial Sequence
  Synthetic: KB-018-109
cuaaaaacauggccccaggagccucagucccuuuctcgucgauggucagc

SEQ ID NO: 369 - DNA, 50 bp, Artificial Sequence
  Synthetic: KB-018-102
cuaaaaacauggcccgagccgcuucagucccuuuctcgucgauggucagc

SEQ ID NO: 370 - RNA, 50 nt, Artificial Sequence
  Synthetic: KB-018-103
cuaaaaacauggcccgagccgcuucagucccuuucucgucgauggucagc

SEQ ID NO: 371 - RNA, 50 nt, Artificial Sequence; identical to SEQ ID NO: 370
  Synthetic: KB-018-104

SEQ ID NO: 372 - RNA, 50 nt, Artificial Sequence; identical to SEQ ID NO: 370
  Synthetic: KB-018-105

SEQ ID NO: 373 - DNA, 43 bp, Artificial Sequence
  Synthetic: KB-018-106
aaacauggcccgagccgcuucagucccuuuctcgucgaugguc

SEQ ID NO: 374 - RNA, 43 nt, Artificial Sequence
  Synthetic: KB-018-107
aaacauggcccgagccgcuucagucccuuucucgucgaugguc

SEQ ID NO: 375 - DNA, 50 bp, Artificial Sequence; identical to SEQ ID NO: 369
  Synthetic: KB-018-102

SEQ ID NO: 376 - RNA, 50 nt, Artificial Sequence; identical to SEQ ID NO: 370
  Synthetic: KB-018-195

SEQ ID NO: 377 - DNA, 50 bp, Artificial Sequence; identical to SEQ ID NO: 369
  Synthetic: KB-018-196

SEQ ID NO: 378 - DNA, 50 bp, Artificial Sequence; identical to SEQ ID NO: 369
  Synthetic: KB-018-197

SEQ ID NO: 379 - RNA, 50 nt, Artificial Sequence; identical to SEQ ID NO: 370
  Synthetic: KB-018-198

SEQ ID NO: 380 - DNA, 50 bp, Artificial Sequence; identical to SEQ ID NO: 369
  Synthetic: KB-018-199

SEQ ID NO: 381 - RNA, 50 nt, Artificial Sequence; identical to SEQ ID NO: 370
  Synthetic: KB-018-200

SEQ ID NO: 382 - RNA, 50 nt, Artificial Sequence; identical to SEQ ID NO: 370
  Synthetic: KB-018-368

SEQ ID NO: 383 - RNA, 50 nt, Artificial Sequence; identical to SEQ ID NO: 370
  Synthetic: KB-018-201

SEQ ID NO: 384 - DNA, 50 bp, Artificial Sequence; identical to SEQ ID NO: 369
  Synthetic: KB-018-202

SEQ ID NO: 385 - DNA, 50 bp, Artificial Sequence; identical to SEQ ID NO: 369
  Synthetic: KB-018-243

SEQ ID NO: 386 - DNA, 50 bp, Artificial Sequence; identical to SEQ ID NO: 369
  Synthetic: KB-018-221

SEQ ID NO: 387 - DNA, 50 bp, Artificial Sequence; identical to SEQ ID NO: 369
  Synthetic: KB-018-597

SEQ ID NO: 388 - DNA, 50 bp, Artificial Sequence; identical to SEQ ID NO: 369
  Synthetic: KB-018-598

SEQ ID NO: 389 - DNA, 50 bp, Artificial Sequence; identical to SEQ ID NO: 369
  Synthetic: KB-018-599

SEQ ID NO: 390 - DNA, 50 bp, Artificial Sequence; identical to SEQ ID NO: 369
  Synthetic: KB-018-222

SEQ ID NO: 391 - RNA, 50 nt, Artificial Sequence; identical to SEQ ID NO: 370
  Synthetic: KB-018-607

SEQ ID NO: 392 - RNA, 50 nt, Artificial Sequence; identical to SEQ ID NO: 370
  Synthetic: KB-018-608

SEQ ID NO: 393 - RNA, 50 nt, Artificial Sequence; identical to SEQ ID NO: 370
  Synthetic: KB-018-609

SEQ ID NO: 394 - DNA, 50 bp, Artificial Sequence; identical to SEQ ID NO: 369
  Synthetic: KB-018-223

SEQ ID NO: 395 - RNA, 50 nt, Artificial Sequence; identical to SEQ ID NO: 370
  Synthetic: KB-018-624

SEQ ID NO: 396 - RNA, 50 nt, Artificial Sequence; identical to SEQ ID NO: 370
  Synthetic: KB-018-625

SEQ ID NO: 397 - RNA, 50 nt, Artificial Sequence; identical to SEQ ID NO: 370
  Synthetic: KB-018-626

SEQ ID NO: 398 - DNA, 50 bp, Artificial Sequence
  Synthetic: KB-018-224
cuaaaaacauggcccgagccgcuucagucccuuuctsgucgauggucagc

SEQ ID NO: 399 - DNA, 50 bp, Artificial Sequence; identical to SEQ ID NO: 398
  Synthetic: KB-018-649

SEQ ID NO: 400 - DNA, 50 bp, Artificial Sequence; identical to SEQ ID NO: 398
  Synthetic: KB-018-658

SEQ ID NO: 401 - RNA, 50 nt, Artificial Sequence
  Synthetic: KB-018-188
  misc_feature (36)..(36): n is inosine
cuaaaaacauggcccgagccgcuucagucccuuucncgucgauggucagc

SEQ ID NO: 402 - DNA, 50 bp, Artificial Sequence; identical to SEQ ID NO: 369
  Synthetic: KB-018-102

SEQ ID NO: 403 - DNA, 50 bp, Artificial Sequence
  Synthetic: KB-018-184
cuaaaaacauggcccgagccgcuucagucccuuucttgucgauggucagc

SEQ ID NO: 404 - DNA, 50 bp, Artificial Sequence
  Synthetic: KB-018-185
cuaaaaacauggcccgagccgcuucagucccuuuctggucgauggucagc

SEQ ID NO: 405 - DNA, 50 bp, Artificial Sequence
  Synthetic: KB-018-186
cuaaaaacauggcccgagccgcuucagucccuuuctagucgauggucagc

SEQ ID NO: 406 - RNA, 50 nt, Artificial Sequence
  Synthetic: KB-018-187
cuaaaaacauggcccgagccgcuucagucccuuucgcgucgauggucagc

SEQ ID NO: 407 - RNA, 50 nt, Artificial Sequence; identical to SEQ ID NO: 401
  Synthetic: KB-018-188
  misc_feature (36)..(36): n is inosine

SEQ ID NO: 408 - RNA, 50 nt, Artificial Sequence
  Synthetic: KB-018-189
cuaaaaacauggcccgagccgcuucagucccuuucacgucgauggucagc

SEQ ID NO: 409 - RNA, 50 nt, Artificial Sequence
  Synthetic: KB-018-190
cuaaaaacauggcccgagccgcuucagucccuuucccgucgauggucagc

SEQ ID NO: 410 - DNA, 50 bp, Artificial Sequence
  Synthetic: KB-018-191
cuaaaaacauggcccgagccgcuucagucccuuuctcaucgauggucagc

SEQ ID NO: 411 - DNA, 50 bp, Artificial Sequence
  Synthetic: KB-018-192
cuaaaaacauggcccgagccgcuucagucccuuuctccucgauggucagc

SEQ ID NO: 412 - DNA, 50 bp, Artificial Sequence
  Synthetic: KB-018-193
cuaaaaacauggcccgagccgcuucagucccuuuctctucgauggucagc

SEQ ID NO: 413 - RNA, 50 nt, Artificial Sequence
  Synthetic: KB-018-194
cuaaaaacauggcccgagccgcuucagucccuuucgccucgauggucagc

SEQ ID NO: 414 - DNA, 50 bp, Artificial Sequence; identical to SEQ ID NO: 369
  Synthetic: KB-018-102

SEQ ID NO: 415 - DNA, 50 bp, Artificial Sequence; identical to SEQ ID NO: 369
  Synthetic: KB-018-225

SEQ ID NO: 416 - DNA, 50 bp, Artificial Sequence; identical to SEQ ID NO: 369
  Synthetic: KB-018-226

SEQ ID NO: 417 - DNA, 50 bp, Artificial Sequence; identical to SEQ ID NO: 369
  Synthetic: KB-018-227

SEQ ID NO: 418 - DNA, 50 bp, Artificial Sequence; identical to SEQ ID NO: 369
  Synthetic: KB-018-228

SEQ ID NO: 419 - DNA, 50 bp, Artificial Sequence; identical to SEQ ID NO: 369
  Synthetic: KB-018-229

SEQ ID NO: 420 - DNA, 50 bp, Artificial Sequence; identical to SEQ ID NO: 369
  Synthetic: KB-018-230

SEQ ID NO: 421 - DNA, 50 bp, Artificial Sequence; identical to SEQ ID NO: 369
  Synthetic: KB-018-231

SEQ ID NO: 422 - DNA, 50 bp, Artificial Sequence; identical to SEQ ID NO: 369
  Synthetic: KB-018-232

SEQ ID NO: 423 - DNA, 50 bp, Artificial Sequence; identical to SEQ ID NO: 369
  Synthetic: KB-018-233

SEQ ID NO: 424 - DNA, 50 bp, Artificial Sequence; identical to SEQ ID NO: 369
  Synthetic: KB-018-234

SEQ ID NO: 425 - DNA, 43 bp, Artificial Sequence; identical to SEQ ID NO: 373
  Synthetic: KB-018-106

SEQ ID NO: 426 - DNA, 43 bp, Artificial Sequence; identical to SEQ ID NO: 373
  Synthetic: KB-018-235

SEQ ID NO: 427 - DNA, 43 bp, Artificial Sequence; identical to SEQ ID NO: 373
  Synthetic: KB-018-236

SEQ ID NO: 428 - DNA, 43 bp, Artificial Sequence; identical to SEQ ID NO: 373
  Synthetic: KB-018-237

SEQ ID NO: 429 - DNA, 50 bp, Artificial Sequence; identical to SEQ ID NO: 53
  Synthetic: KB-018-001

SEQ ID NO: 430 - DNA, 50 bp, Artificial Sequence; identical to SEQ ID NO: 53
  Synthetic: KB-018-748

SEQ ID NO: 431 - DNA, 50 bp, Artificial Sequence; identical to SEQ ID NO: 53
  Synthetic: KB-018-749

SEQ ID NO: 432 - RNA, 50 nt, Artificial Sequence; identical to SEQ ID NO: 61
  Synthetic: KB-018-750

SEQ ID NO: 433 - DNA, 50 bp, Artificial Sequence; identical to SEQ ID NO: 53
  Synthetic: KB-018-751

SEQ ID NO: 434 - RNA, 50 nt, Artificial Sequence; identical to SEQ ID NO: 61
  Synthetic: KB-018-752

SEQ ID NO: 435 - DNA, 50 bp, Artificial Sequence
  Synthetic: KB-018-753
ctaaaaacauggccccagcagcuucagucccuuuctcgucgatggucagc

SEQ ID NO: 436 - DNA, 50 bp, Artificial Sequence; identical to SEQ ID NO: 53
  Synthetic: KB-018-754

SEQ ID NO: 437 - DNA, 43 bp, Artificial Sequence
  Synthetic: KB-018-755
cauggccccagcagcuucagucccuuuctcgucgauggucagc

SEQ ID NO: 438 - DNA, 40 bp, Artificial Sequence
  Synthetic: KB-018-756
cauggccccagcagcuucagucccuuuctcgucgatggtc

SEQ ID NO: 439 - DNA, 50 bp, Artificial Sequence; identical to SEQ ID NO: 369
  Synthetic: KB-018-102

SEQ ID NO: 440 - DNA, 50 bp, Artificial Sequence; identical to SEQ ID NO: 369
  Synthetic: KB-018-757

SEQ ID NO: 441 - DNA, 50 bp, Artificial Sequence; identical to SEQ ID NO: 369
  Synthetic: KB-018-758

SEQ ID NO: 442 - DNA, 50 bp, Artificial Sequence; identical to SEQ ID NO: 369
  Synthetic: KB-018-759

SEQ ID NO: 443 - DNA, 45 bp, Artificial Sequence; identical to SEQ ID NO: 54
  Synthetic: KB-018-380

SEQ ID NO: 444 - DNA, 50 bp, Artificial Sequence; identical to SEQ ID NO: 53
  Synthetic: KB-018-381

SEQ ID NO: 445 - DNA, 50 bp, Artificial Sequence; identical to SEQ ID NO: 53
  Synthetic: KB-018-382

SEQ ID NO: 446 - DNA, 50 bp, Artificial Sequence; identical to SEQ ID NO: 53
  Synthetic: KB-018-383

SEQ ID NO: 447 - DNA, 50 bp, Artificial Sequence; identical to SEQ ID NO: 53
  Synthetic: KB-018-384

SEQ ID NO: 448 - DNA, 50 bp, Artificial Sequence; identical to SEQ ID NO: 53
  Synthetic: KB-018-385

SEQ ID NO: 449 - DNA, 50 bp, Artificial Sequence; identical to SEQ ID NO: 53
  Synthetic: KB-018-386

SEQ ID NO: 450 - DNA, 50 bp, Artificial Sequence; identical to SEQ ID NO: 53
  Synthetic: KB-018-373

SEQ ID NO: 451 - DNA, 50 bp, Artificial Sequence; identical to SEQ ID NO: 53
  Synthetic: KB-018-001

SEQ ID NO: 452 - DNA, 50 bp, Artificial Sequence; identical to SEQ ID NO: 53
  Synthetic: KB-018-373

SEQ ID NO: 453 - RNA, 50 nt, Artificial Sequence; identical to SEQ ID NO: 61
  Synthetic: KB-018-374

SEQ ID NO: 454 - RNA, 50 nt, Artificial Sequence; identical to SEQ ID NO: 61
  Synthetic: KB-018-375

SEQ ID NO: 455 - DNA, 45 bp, Artificial Sequence; identical to SEQ ID NO: 54
  Synthetic: KB-018-376

SEQ ID NO: 456 - DNA, 45 bp, Artificial Sequence; identical to SEQ ID NO: 54
  Synthetic: KB-018-377

SEQ ID NO: 457 - DNA, 45 bp, Artificial Sequence; identical to SEQ ID NO: 54
  Synthetic: KB-018-378

SEQ ID NO: 458 - DNA, 45 bp, Artificial Sequence; identical to SEQ ID NO: 54
  Synthetic: KB-018-379

SEQ ID NO: 459 - DNA, 50 bp, Artificial Sequence; identical to SEQ ID NO: 53
  Synthetic: KB-018-001

SEQ ID NO: 460 - DNA, 50 bp, Artificial Sequence
  Synthetic: KB-018-397
ucagucccuuuctcgucgauggucagcacagccuuaugcacggccuugga

SEQ ID NO: 461 - DNA, 50 bp, Artificial Sequence; identical to SEQ ID NO: 360
  Synthetic: KB-018-094

SEQ ID NO: 462 - DNA, 50 bp, Artificial Sequence; identical to SEQ ID NO: 369
  Synthetic: KB-018-102

SEQ ID NO: 463 - DNA, 50 bp, Artificial Sequence
  Synthetic: KB-018-398
ucagucccuuuctcgucgauggucaacaccgccuuaugcacggccuugga

SEQ ID NO: 464 - DNA, 50 bp, Artificial Sequence
  Synthetic: KB-018-399
ucagucccuuuctcgucgauggucagcaccgccguaugcacggccuugga

SEQ ID NO: 465 - DNA, 50 bp, Artificial Sequence
  Synthetic: KB-018-400
ucagucccuuuctcgucgauggucagcacaaccugaugcacggccuugga

SEQ ID NO: 466 - DNA, 65 bp, Artificial Sequence
  Synthetic: KB-018-034
guggaauaguauaacaauaugcuaaauguuguuauaguaucccaccuuuctcgucgauggucagc

SEQ ID NO: 467 - DNA, 65 bp, Artificial Sequence; identical to SEQ ID NO: 466
  Synthetic: KB-018-035

SEQ ID NO: 468 - DNA, 65 bp, Artificial Sequence
  Synthetic: KB-018-036
guggaauaguauaacaauaugcuaaauguuguuauaguaucccacccuuuctcgucgauggucag

SEQ ID NO: 469 - DNA, 65 bp, Artificial Sequence; identical to SEQ ID NO: 468
  Synthetic: KB-018-037

SEQ ID NO: 470 - DNA, 64 bp, Artificial Sequence
  Synthetic: KB-018-038
guggaauaguauaacaauaugcuaaauguuguuauaguaucccaccuuuctcgucgauggucag

SEQ ID NO: 471 - DNA, 64 bp, Artificial Sequence
  Synthetic: KB-018-039
guggaauaguauaacaauaugcuaaauguuguuauaguaucccacccuuuctcgucgaugguca

SEQ ID NO: 472 - DNA, 63 bp, Artificial Sequence
  Synthetic: KB-018-040
guggaauaguauaacaauaugcuaaauguuguuauaguaucccaccuuuctcgucgaugguca

SEQ ID NO: 473 - DNA, 63 bp, Artificial Sequence
  Synthetic: KB-018-041
guggaauaguauaacaauaugcuaaauguuguuauaguaucccacccuuuctcgucgaugguc

SEQ ID NO: 474 - DNA, 62 bp, Artificial Sequence
  Synthetic: KB-018-042
guggaauaguauaacaauaugcuaaauguuguuauaguaucccaccuuuctcgucgaugguc

SEQ ID NO: 475 - DNA, 62 bp, Artificial Sequence; identical to SEQ ID NO: 474
  Synthetic: KB-018-043

SEQ ID NO: 476 - DNA, 62 bp, Artificial Sequence
  Synthetic: KB-018-044
guggaauaguauaacaauaugcuaaauguuguuauaguaucccacccuuuctcgucgauggu

SEQ ID NO: 477 - DNA, 62 bp, Artificial Sequence; identical to SEQ ID NO: 476
  Synthetic: KB-018-045

SEQ ID NO: 478 - DNA, 62 bp, Artificial Sequence
  Synthetic: KB-018-115
guggaauaguauaacaauaugcuaaauguuguuauaguaucccacuctcgucgauggucagc

SEQ ID NO: 479 - DNA, 63 bp, Artificial Sequence
  Synthetic: KB-018-116
guggaauaguauaacaauaugcuaaauguuguuauaguaucccacuuctcgucgauggucagc

SEQ ID NO: 480 - DNA, 64 bp, Artificial Sequence
  Synthetic: KB-018-117
guggaauaguauaacaauaugcuaaauguuguuauaguaucccacuuuctcgucgauggucagc

SEQ ID NO: 481 - DNA, 65 bp, Artificial Sequence; identical to SEQ ID NO: 466
  Synthetic: KB-018-035

SEQ ID NO: 482 - DNA, 66 bp, Artificial Sequence
  Synthetic: KB-018-118
guggaauaguauaacaauaugcuaaauguuguuauaguaucccacccuuuctcgucgauggucagc

SEQ ID NO: 483 - DNA, 67 bp, Artificial Sequence
  Synthetic: KB-018-119
guggaauaguauaacaauaugcuaaauguuguuauaguaucccaccccuuuctcgucgauggucagc

SEQ ID NO: 484 - DNA, 68 bp, Artificial Sequence
  Synthetic: KB-018-120
guggaauaguauaacaauaugcuaaauguuguuauaguaucccacucccuuuctcgucgauggucagc

SEQ ID NO: 485 - DNA, 69 bp, Artificial Sequence
  Synthetic: KB-018-121
guggaauaguauaacaauaugcuaaauguuguuauaguaucccacgucccuuuctcgucgauggucagc

SEQ ID NO: 486 - DNA, 59 bp, Artificial Sequence
  Synthetic: KB-018-122
guggaauaguauaacaauaugcuaaauguuguuauaguaucccacccuuuctcgucgau

SEQ ID NO: 487 - DNA, 60 bp, Artificial Sequence
  Synthetic: KB-018-123
guggaauaguauaacaauaugcuaaauguuguuauaguaucccacccuuuctcgucgaug

SEQ ID NO: 488 - DNA, 61 bp, Artificial Sequence
  Synthetic: KB-018-124
guggaauaguauaacaauaugcuaaauguuguuauaguaucccacccuuuctcgucgaugg

SEQ ID NO: 489 - DNA, 62 bp, Artificial Sequence; identical to SEQ ID NO: 476
  Synthetic: KB-018-045

SEQ ID NO: 490 - DNA, 63 bp, Artificial Sequence; identical to SEQ ID NO: 473
  Synthetic: KB-018-125

SEQ ID NO: 491 - DNA, 64 bp, Artificial Sequence; identical to SEQ ID NO: 471
  Synthetic: KB-018-126

SEQ ID NO: 492 - DNA, 65 bp, Artificial Sequence; identical to SEQ ID NO: 468
  Synthetic: KB-018-037

SEQ ID NO: 493 - DNA, 66 bp, Artificial Sequence; identical to SEQ ID NO: 482
  Synthetic: KB-018-128

SEQ ID NO: 494 - DNA, 67 bp, Artificial Sequence
  Synthetic: KB-018-129
guggaauaguauaacaauaugcuaaauguuguuauaguaucccacccuuuctcgucgauggucagca

SEQ ID NO: 495 - DNA, 68 bp, Artificial Sequence
  Synthetic: KB-018-130
guggaauaguauaacaauaugcuaaauguuguuauaguaucccacccuuuctcgucgauggucagcac

SEQ ID NO: 496 - DNA, 69 bp, Artificial Sequence
  Synthetic: KB-018-131
guggaauaguauaacaauaugcuaaauguuguuauaguaucccacccuuuctcgucgauggucagcaca

SEQ ID NO: 497 - RNA, 63 nt, Artificial Sequence
  Synthetic: KB-018-081
ggugaagaggagaacaauaugcuaaauguuguucucgucuccaccccuuucucgucgaugguc

SEQ ID NO: 498 - RNA, 63 nt, Artificial Sequence; identical to SEQ ID NO: 497
  Synthetic: KB-018-082

SEQ ID NO: 499 - RNA, 63 nt, Artificial Sequence; identical to SEQ ID NO: 497
  Synthetic: KB-018-083

SEQ ID NO: 500 - DNA, 63 bp, Artificial Sequence
  Synthetic: KB-018-084
ggugaagaggagaacaauaugcuaaauguuguucucgucuccaccccuuuctcgucgaugguc

SEQ ID NO: 501 - RNA, 73 nt, Artificial Sequence
  Synthetic: KB-018-085
ggugucgagaagaggagaacaauaugcuaaauguuguucucgucuccucgacaccccuuucucgucgaugguc

SEQ ID NO: 502 - RNA, 73 nt, Artificial Sequence; identical to SEQ ID NO: 501
  Synthetic: KB-018-086

SEQ ID NO: 503 - RNA, 73 nt, Artificial Sequence; identical to SEQ ID NO: 501
  Synthetic: KB-018-087

SEQ ID NO: 504 - DNA, 73 bp, Artificial Sequence
  Synthetic: KB-018-088
ggugucgagaagaggagaacaauaugcuaaauguuguucucgucuccucgacaccccuuuctcgucgaugguc

SEQ ID NO: 505 - DNA, 95 bp, Artificial Sequence
  Synthetic: KB-018-089
ggugucgagaagaggagaacaauaugcuaaauguuguucucgucuccucgacacccauggccccagcagcuucagucccuuucucgucgatggtc

SEQ ID NO: 506 - DNA, 85 bp, Artificial Sequence
  Synthetic: KB-018-760
ggugaagaggagaacaauaugcuaaauguuguucucgucuccacccauggccccagcagcuucagucccuuuctcgucgatggtc

SEQ ID NO: 507 - DNA, 95 bp, Artificial Sequence
  Synthetic: KB-018-761
ggugucgagaagaggagaacaauaugcuaaauguuguucucgucuccucgacacccauggccccagcagcuucagucccuuuctcgucgatggtc

SEQ ID NO: 508 - DNA, 95 bp, Artificial Sequence; identical to SEQ ID NO: 507
  Synthetic: KB-018-1303

SEQ ID NO: 509 - DNA, 63 bp, Artificial Sequence; identical to SEQ ID NO: 473
  Synthetic: KB-018-041

SEQ ID NO: 510 - DNA, 63 bp, Artificial Sequence; identical to SEQ ID NO: 473
  Synthetic: KB-018-125

SEQ ID NO: 511 - DNA, 63 bp, Artificial Sequence; identical to SEQ ID NO: 500
  Synthetic: KB-018-132

SEQ ID NO: 512 - DNA, 63 bp, Artificial Sequence; identical to SEQ ID NO: 500
  Synthetic: KB-018-084

SEQ ID NO: 513 - DNA, 63 bp, Artificial Sequence; identical to SEQ ID NO: 500
  Synthetic: KB-018-133

SEQ ID NO: 514 - DNA, 63 bp, Artificial Sequence; identical to SEQ ID NO: 500
  Synthetic: KB-018-134

SEQ ID NO: 515 - DNA, 63 bp, Artificial Sequence; identical to SEQ ID NO: 500
  Synthetic: KB-018-135

SEQ ID NO: 516 - DNA, 73 bp, Artificial Sequence; identical to SEQ ID NO: 504
  Synthetic: KB-018-136

SEQ ID NO: 517 - DNA, 73 bp, Artificial Sequence; identical to SEQ ID NO: 504
  Synthetic: KB-018-088

SEQ ID NO: 518 - DNA, 73 bp, Artificial Sequence; identical to SEQ ID NO: 504
  Synthetic: KB-018-137

SEQ ID NO: 519 - DNA, 73 bp, Artificial Sequence; identical to SEQ ID NO: 504
  Synthetic: KB-018-138

SEQ ID NO: 520 - DNA, 73 bp, Artificial Sequence; identical to SEQ ID NO: 504
  Synthetic: KB-018-139

SEQ ID NO: 521 - DNA, 63 bp, Artificial Sequence; identical to SEQ ID NO: 473
  Synthetic: KB-018-125

SEQ ID NO: 522 - DNA, 63 bp, Artificial Sequence; identical to SEQ ID NO: 473
  Synthetic: KB-018-140

SEQ ID NO: 523 - DNA, 63 bp, Artificial Sequence; identical to SEQ ID NO: 500
  Synthetic: KB-018-141

SEQ ID NO: 524 - DNA, 73 bp, Artificial Sequence; identical to SEQ ID NO: 504
  Synthetic: KB-018-142

SEQ ID NO: 525 - DNA, 63 bp, Artificial Sequence; identical to SEQ ID NO: 473
  Synthetic: KB-018-125

SEQ ID NO: 526 - RNA, 63 nt, Artificial Sequence
  Synthetic: KB-018-146
guggaauaguauaacaauaugcuaaauguuguuauaguaucccacccuuucucgucgaugguc

SEQ ID NO: 527 - DNA, 63 bp, Artificial Sequence; identical to SEQ ID NO: 473
  Synthetic: KB-018-147

SEQ ID NO: 528 - DNA, 63 bp, Artificial Sequence; identical to SEQ ID NO: 473
  Synthetic: KB-018-148

SEQ ID NO: 529 - RNA, 63 nt, Artificial Sequence; identical to SEQ ID NO: 526
  Synthetic: KB-018-149

SEQ ID NO: 530 - DNA, 63 bp, Artificial Sequence; identical to SEQ ID NO: 473
  Synthetic: KB-018-150

SEQ ID NO: 531 - RNA, 63 nt, Artificial Sequence; identical to SEQ ID NO: 526
  Synthetic: KB-018-151

SEQ ID NO: 532 - RNA, 63 nt, Artificial Sequence; identical to SEQ ID NO: 526
  Synthetic: KB-018-152

SEQ ID NO: 533 - DNA, 63 bp, Artificial Sequence; identical to SEQ ID NO: 473
  Synthetic: KB-018-125

SEQ ID NO: 534 - RNA, 63 nt, Artificial Sequence; identical to SEQ ID NO: 526
  Synthetic: KB-018-153

SEQ ID NO: 535 - DNA, 63 bp, Artificial Sequence; identical to SEQ ID NO: 473
  Synthetic: KB-018-154

SEQ ID NO: 536 - DNA, 63 bp, Artificial Sequence; identical to SEQ ID NO: 473
  Synthetic: KB-018-155

SEQ ID NO: 537 - RNA, 63 nt, Artificial Sequence; identical to SEQ ID NO: 526
  Synthetic: KB-018-156

SEQ ID NO: 538 - DNA, 63 bp, Artificial Sequence; identical to SEQ ID NO: 473
  Synthetic: KB-018-157

SEQ ID NO: 539 - RNA, 63 nt, Artificial Sequence; identical to SEQ ID NO: 526
  Synthetic: KB-018-158

SEQ ID NO: 540 - RNA, 63 nt, Artificial Sequence; identical to SEQ ID NO: 526
  Synthetic: KB-018-159

SEQ ID NO: 541 - RNA, 63 nt, Artificial Sequence; identical to SEQ ID NO: 526
  Synthetic: KB-018-160

SEQ ID NO: 542 - RNA, 63 nt, Artificial Sequence; identical to SEQ ID NO: 526
  Synthetic: KB-018-161

SEQ ID NO: 543 - RNA, 63 nt, Artificial Sequence; identical to SEQ ID NO: 526
  Synthetic: KB-018-162

SEQ ID NO: 544 - RNA, 63 nt, Artificial Sequence; identical to SEQ ID NO: 526
  Synthetic: KB-018-163

SEQ ID NO: 545 - DNA, 63 bp, Artificial Sequence; identical to SEQ ID NO: 473
  Synthetic: KB-018-125

SEQ ID NO: 546 - DNA, 63 bp, Artificial Sequence; identical to SEQ ID NO: 473
  Synthetic: KB-018-164

SEQ ID NO: 547 - DNA, 63 bp, Artificial Sequence; identical to SEQ ID NO: 473
  Synthetic: KB-018-165

SEQ ID NO: 548 - DNA, 63 bp, Artificial Sequence; identical to SEQ ID NO: 473
  Synthetic: KB-018-166

SEQ ID NO: 549 - RNA, 63 nt, Artificial Sequence; identical to SEQ ID NO: 526
  Synthetic: KB-018-167

SEQ ID NO: 550 - DNA, 63 bp, Artificial Sequence; identical to SEQ ID NO: 473
  Synthetic: KB-018-168

SEQ ID NO: 551 - RNA, 63 nt, Artificial Sequence; identical to SEQ ID NO: 526
  Synthetic: KB-018-169

SEQ ID NO: 552 - RNA, 63 nt, Artificial Sequence; identical to SEQ ID NO: 526
  Synthetic: KB-018-170

SEQ ID NO: 553 - DNA, 64 bp, Artificial Sequence
  Synthetic: KB-018-171
guggaauaguauaacaauaugcuaaauguuguuauaguaucccacccuuuctgcgucgaugguc

SEQ ID NO: 554 - DNA, 66 bp, Artificial Sequence; identical to SEQ ID NO: 482
  Synthetic: KB-018-128

SEQ ID NO: 555 - RNA, 66 nt, Artificial Sequence
  Synthetic: KB-018-610
guggaauaguauaacaauaugcuaaauguuguuauaguaucccacccuuucucgucgauggucagc

SEQ ID NO: 556 - DNA, 66 bp, Artificial Sequence; identical to SEQ ID NO: 482
  Synthetic: KB-018-611

SEQ ID NO: 557 - DNA, 66 bp, Artificial Sequence; identical to SEQ ID NO: 482
  Synthetic: KB-018-612

SEQ ID NO: 558 - RNA, 66 nt, Artificial Sequence; identical to SEQ ID NO: 555
  Synthetic: KB-018-613

SEQ ID NO: 559 - DNA, 66 bp, Artificial Sequence; identical to SEQ ID NO: 482
  Synthetic: KB-018-614

SEQ ID NO: 560 - RNA, 66 nt, Artificial Sequence; identical to SEQ ID NO: 555
  Synthetic: KB-018-615

SEQ ID NO: 561 - RNA, 66 nt, Artificial Sequence; identical to SEQ ID NO: 555
  Synthetic: KB-018-616

SEQ ID NO: 562 - DNA, 66 bp, Artificial Sequence; identical to SEQ ID NO: 482
  Synthetic: KB-018-128

SEQ ID NO: 563 - RNA, 66 nt, Artificial Sequence; identical to SEQ ID NO: 555
  Synthetic: KB-018-627

SEQ ID NO: 564 - DNA, 66 bp, Artificial Sequence; identical to SEQ ID NO: 482
  Synthetic: KB-018-628

SEQ ID NO: 565 - DNA, 66 bp, Artificial Sequence; identical to SEQ ID NO: 482
  Synthetic: KB-018-629

SEQ ID NO: 566 - RNA, 66 nt, Artificial Sequence; identical to SEQ ID NO: 555
  Synthetic: KB-018-630

SEQ ID NO: 567 - DNA, 66 bp, Artificial Sequence; identical to SEQ ID NO: 482
  Synthetic: KB-018-631

SEQ ID NO: 568 - RNA, 66 nt, Artificial Sequence; identical to SEQ ID NO: 555
  Synthetic: KB-018-632

SEQ ID NO: 569 - RNA, 66 nt, Artificial Sequence; identical to SEQ ID NO: 555
  Synthetic: KB-018-633

SEQ ID NO: 570 - DNA, 65 bp, Artificial Sequence; identical to SEQ ID NO: 466
  Synthetic: KB-018-035

SEQ ID NO: 571 - DNA, 66 bp, Artificial Sequence
  Synthetic: KB-018-401
ucagucccuuuctcgucgaugcacccuaugauauuguuguaaaucguauaacaauaugauaaggug

SEQ ID NO: 572 - DNA, 66 bp, Artificial Sequence
  Synthetic: KB-018-402
ucagucccuuuctcgucgaugguggaauaguauaacaauaugcuaaauguuguuauaguaucccac

(19) United States
(12) Patent Application Publication
(10) Pub. No.: US 2021/0380980 A1
(43) Pub. Date: Dec. 9, 2021

(54) Methods and Compositions for the ADAR-Mediated Editing of SERPINA1

(71) Applicant: Korro Bio, Inc., Cambridge, MA (US)

(72) Inventors: Steven Robinette, Cambridge, MA (US); Andrew Fraley, Arlington, MA (US); Mallikarjuna Reddy Putta, Lexington, MA (US)

(73) Assignee: Korro Bio, Inc., Cambridge, MA (US)

(21) Appl. No.: 17/332,400

(22) Filed: May 27, 2021

Related U.S. Application Data
(60) Provisional application No. 62/704,793, filed on May 28, 2020.
(60) Provisional application No. 62/705,838, filed on Jul. 17, 2020.

(51) Int. Cl. C12N 15/113; A61K 31/7088; C12Q 1/6883; A61P 3/00

(52) U.S. Cl. CPC C12N 15/113; A61K 31/7088; C12Q 1/6883; A61P 3/00; C12N 2310/351; C12Q 2600/106; C12N 2310/346; C12N 2310/321; C12N 2310/322; C12N 2310/3231; C12N 2310/3341; C12N 2310/315; C12Q 2600/156

(57) ABSTRACT

The present invention relates to methods and compositions for editing a SERPINA1 polynucleotide, e.g., a SERPINA1 polynucleotide comprising a SNP associated with alpha 1 antitrypsin deficiency. The invention also relates to methods and compositions for treating or preventing alpha 1 antitrypsin deficiency in a subject.

### CROSS-REFERENCE TO RELATED APPLICATIONS

This application claims the benefit of priority of US Provisional Application No. 62/705,838, filed Jul. 17, 2020, and US Provisional Application No. 62/704,793, filed May 28, 2020, each of which is incorporated by reference herein in its entirety for any purpose.

### SEQUENCE LISTING

The present application contains a Sequence Listing in electronic format. The Sequence Listing is provided as a file entitled “01249-0002-00US_ST25.txt” created on Jul. 15, 2021, which is 122,880 bytes in size. The information in the electronic format of the sequence listing is incorporated herein by reference in its entirety.

### BACKGROUND OF THE INVENTION

The SERPINA1 gene encodes serine protease inhibitor alpha-1 antitrypsin (A1AT). A1AT protects tissues from certain inflammatory enzymes, including neutrophil elastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in liver, resulting in cirrhosis and liver failure.

A1AD may be caused by an E342K mutation, which is associated with SNP rs28929474(A). There exists an ongoing need for methods to selectively and efficiently edit the SERPINA1 gene and correct any pathogenic mutations in the gene in order to treat alpha 1 antitrypsin deficiency (A1AD) which may result in hepatic failure and/or emphysema.

### SUMMARY OF THE INVENTION

The present invention provides methods and compositions for editing a SERPINA1 polynucleotide and methods of treating or preventing a SERPIN1-associated disease, in a subject using a guide oligonucleotide capable of effecting an adenosine deaminase acting on RNA (ADAR)-mediated adenosine to inosine alteration in the target gene. In some embodiments, the deamination correcting the pathogenic mutation in the gene reverses a E342K mutation back to wild-type, reversing or slowing the SERPIN1-associated disease and any related symptoms experienced by the patient.
Embodiment 1. A method of editing a SERPINA1 polynucleotide comprising a single nucleotide polymorphism (SNP) associated with alpha 1 antitrypsin deficiency which may result in hepatic failure or emphysema, the method comprising contacting the SERPINA1 polynucleotide with a guide oligonucleotide capable of effecting an adenosine deaminase acting on RNA (ADAR)-mediated adenosine to inosine alteration of the SNP associated with alpha 1 antitrypsin deficiency, thereby editing the SERPINA1 polynucleotide.
Embodiment 2. The method of embodiment 1, wherein the SERPINA1 polynucleotide is contacted with the guide oligonucleotide in a cell.
Embodiment 3. The method of embodiment 2, wherein the cell endogenously expresses ADAR.
Embodiment 4. The method of embodiment 3, wherein the ADAR is a human ADAR.
Embodiment 5. The method of embodiment 4, wherein the ADAR is human ADAR1.
Embodiment 6. The method of embodiment 4, wherein the ADAR is human ADAR2.
Embodiment 7. The method of any one of embodiments 2-6, wherein the cell is selected from eukaryotic cell, a mammalian cell, and a human cell.
Embodiment 8. The method of any one of embodiments 2-7, wherein the cell is in vivo.
Embodiment 9. The method of any one of embodiments 2-7, wherein the cell is ex vivo.
Embodiment 10. A method of treating alpha 1 antitrypsin deficiency in a subject in need thereof, the method comprising
   a) identifying a subject with a single nucleotide polymorphism (SNP) associated with alpha 1 antitrypsin deficiency in a SERPINA1 polynucleotide;
   b) contacting the SERPINA1 polynucleotide in a cell of the subject with a guide oligonucleotide capable of effecting an adenosine deaminase acting on RNA (ADAR)-mediated adenosine to inosine alteration of the SNP associated with alpha 1 antitrypsin deficiency, thereby treating the subject.
Embodiment 11. A method of treating alpha 1 antitrypsin deficiency in a subject in need thereof, the method comprising
   a) identifying a subject with a single nucleotide polymorphism (SNP) associated with alpha 1 antitrypsin deficiency in a SERPINA1 polynucleotide;
   b) contacting the SERPINA1 polynucleotide in a cell with a guide oligonucleotide capable of effecting an adenosine deaminase acting on RNA (ADAR)-mediated adenosine to inosine alteration of the SNP associated with alpha 1 antitrypsin deficiency, and
   c) administering the cell to the subject, thereby treating the subject.
Embodiment 12. The method of embodiment 11, wherein the cell is autologous, allogenic, or xenogenic to the subject.
Embodiment 13. The method of any one of embodiments 10-12, wherein the subject is a human subject.
Embodiment 14. The method of any one of embodiments 1-13, wherein the guide oligonucleotide comprises a nucleic acid sequence complementary to a SERPINA1 mRNA sequence comprising the SNP associated with alpha 1 antitrypsin deficiency.
Embodiment 15. The method of any one of embodiments 1-14, wherein the oligonucleotide further comprises one or more adenosine deaminase acting on RNA (ADAR)-recruiting domains.
Embodiment 16. The method of any one of embodiments 1-15, wherein the SERPINA1 polynucleotide encodes a SERPINA1 protein comprising a pathogenic amino acid comprising a lysine at position 342 resulting from the SNP.
Embodiment 17. The method of embodiment 16, wherein the adenosine to inosine alteration substitutes the pathogenic amino acid with a wild type amino acid.
Embodiment 18. The method of embodiment 17, wherein the wild type amino acid at position 342 comprises a glutamic acid.
Embodiment 19. The method of any one of embodiments 1-18, wherein the guide oligonucleotide comprises the structure:

[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of
X¹, X², or X³ is an alternative nucleotide.
Embodiment 20. The method of any one of embodiments 1-19, wherein the guide oligonucleotide comprises the structure:

[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of
X¹, X², or X³ has the structure of any one of Formula I-V:

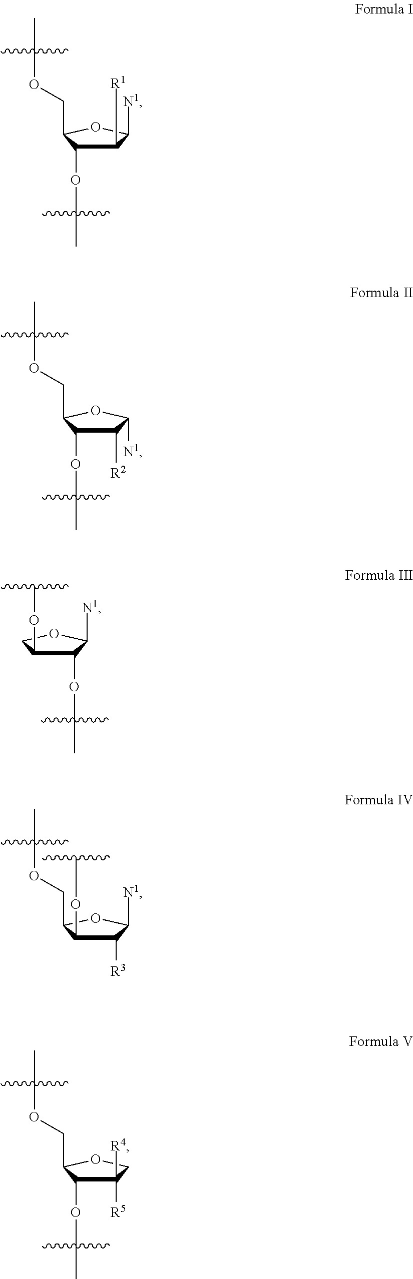

wherein N¹ is hydrogen or a nucleobase;
is R¹ hydroxy, halogen, or C₁-C₆ alkoxy;
R² is hydrogen, hydroxy, halogen, or C₁-C₆ alkoxy;
R³ is hydrogen, hydroxy, halogen, or C₁-C₆ alkoxy;
R⁴ is hydrogen, hydroxy, halogen, or C₁-C₆ alkoxy; and
R⁵ is hydrogen, hydroxy, halogen, or C₁-C₆ alkoxy.
Embodiment 21. The method of embodiment 20, wherein R⁴ is hydrogen and R⁵ is not hydrogen or hydroxy, R⁵ is hydrogen and R⁴ is not hydrogen, or R⁵ is hydroxy and R⁴ is not hydrogen.
Embodiment 22. The method of embodiment 20 or embodiment 21, wherein at least 80% of the nucleotides of [Aₘ] and/or [Bₙ] include a nucleobase, a sugar, and an internucleoside linkage.
Embodiment 23. The method of any one of embodiments 20 to 22, wherein R¹ is hydroxy, halogen, or OCH₃.
Embodiment 24. The method of any one of embodiments 20 to 23, wherein R² is hydrogen.
Embodiment 25. The method of any one of embodiments 20 to 24, wherein at least one of X¹, X², or X³ has the structure of Formula I, Formula II, or Formula V; and none of X¹, X², or X³ has the structure of Formula IV or Formula III.
Embodiment 26. The method of any one of embodiments 20 to 25, wherein at least one of X¹, X², or X³ has the structure of Formula I or Formula II; and none of X¹, X², or X³ has the structure of Formula III, Formula IV, or Formula V.
Embodiment 27. The method of any one of embodiments 20 to 26, wherein the halogen is fluoro.
Embodiment 28. The method of any one of embodiments 20 to 27, wherein at least one of X¹, X², and X³ has the structure of Formula I, wherein R¹ is fluoro and N¹ is a nucleobase. Embodiment 29. The method of embodiment 28, wherein X¹ has the structure of Formula I, wherein R¹ is fluoro and N¹ is a nucleobase.
Embodiment 30. The method of embodiment 28 or 29, wherein X² has the structure of Formula I, wherein R¹ is fluoro and N¹ is a nucleobase.
Embodiment 31. The method of any one of embodiments 28 to 30, wherein X³ has the structure of Formula I, wherein R¹ is fluoro and N¹ is a nucleobase.
Embodiment 32. The method of any one of embodiments 20 to 27, wherein at least one of X¹, X², and X³ has the structure of Formula I, wherein R¹ is hydroxy and N¹ is a nucleobase.

Embodiment 33. The method of embodiment 32, wherein X¹ has the structure of Formula I, wherein R¹ is hydroxy and N¹ is a nucleobase.
Embodiment 34. The method of embodiment 32 or 33, wherein X² has the structure of Formula I, wherein R¹ is hydroxy and N¹ is a nucleobase.
Embodiment 35. The method of any one of embodiments 32 to 34, wherein X³ has the structure of Formula I, wherein R¹ is hydroxy and N¹ is a nucleobase.
Embodiment 36. The method of any one of embodiments 20 to 27, wherein at least one of X¹, X², and X³ has the structure of Formula I, wherein R¹ is methoxy and N¹ is a nucleobase.
Embodiment 37. The method of embodiment 36, wherein X¹ has the structure of Formula I, wherein R¹ is methoxy and N¹ is a nucleobase; and each of X² and X³ is a deoxyribonucleotide or a ribonucleotide.
Embodiment 38. The method of embodiment 36 or 37, wherein X² has the structure of Formula I, wherein R¹ is methoxy and N¹ is a nucleobase.
Embodiment 39. The method of any one of embodiments 36 to 38, wherein X³ has the structure of Formula I, wherein R¹ is methoxy and N¹ is a nucleobase.
Embodiment 40. The method of any one of embodiments 20 to 27, wherein at least one of X¹, X², and X³ has the structure of Formula II, wherein R² is hydrogen and N¹ is a nucleobase.
Embodiment 41. The method of embodiment 40, wherein X² has the structure of Formula II, wherein R² is hydrogen and N¹ is a nucleobase.
Embodiment 42. The method of any one of embodiments 20 to 25, wherein at least one of X¹ and X² has the structure of Formula V.
Embodiment 43. The method of embodiment 42, wherein X² has the structure of Formula V, wherein R⁴ is hydrogen and R⁵ is hydrogen.
Embodiment 44. The method of embodiment 42, wherein X² has the structure of Formula V, wherein R⁴ is hydrogen and R⁵ is hydroxy.
Embodiment 45. The method of embodiment 42, wherein X¹ has the structure of Formula V, wherein R⁴ is hydrogen and R⁵ is hydrogen.
Embodiment 46. The method of embodiment 42, wherein X¹ has the structure of Formula V, wherein R⁴ is hydrogen and R⁵ is hydroxy.
Embodiment 47. The method of embodiment 42, wherein X² has the structure of Formula V, wherein R⁴ is hydrogen and R⁵ is methoxy.
Embodiment 48. The method of any one of embodiments 20 to 47, wherein when X¹ has the structure of any one of Formulas I to V, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X² has the structure of any one of Formulas I to V, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X³ has the structure of any one of Formulas I to V, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X² each have the structure of any one of Formulas i to V, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X³ each have the structure of any one of Formulas I to V, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; and when X² and X³ each have the structure of any one of Formulas I to V, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide.
Embodiment 49. The method of embodiment 48, wherein when X¹ has the structure of any one of Formulas I to V, each of X² and X³ is, independently, a ribonucleotide, a 2′-F-nucleotide, 2′-O-methoxyethyl-nucleotide, or a deoxyribonucleotide; when X² has the structure of any one of Formulas I to V, each of X¹ and X³ is, independently, a ribonucleotide, a 2′-F-nucleotide, 2′-O-methoxyethyl-nucleotide, or a deoxyribonucleotide; when X³ has the structure of any one of Formulas I to V, each of X¹ and X² is, independently, a ribonucleotide, a 2′-F-nucleotide, 2′-O-methoxyethyl-nucleotide, or a deoxyribonucleotide; when X¹ and X² each have the structure of any one of Formulas I to V, X³ is a ribonucleotide, a 2′-F-nucleotide, 2′-O-methoxyethyl-nucleotide, or a deoxyribonucleotide; when X¹ and X³ each have the structure of any one of Formulas I to V, X² is a ribonucleotide, a 2′-F-nucleotide, 2′-O-methoxyethyl-nucleotide, or a deoxyribonucleotide; and when X² and X³ each have the structure of any one of Formulas I to V, X¹ is a ribonucleotide, a 2′-F-nucleotide, 2′-O-methoxyethyl-nucleotide, or a deoxyribonucleotide.
Embodiment 50. The method of embodiment 49, wherein when X¹ has the structure of any one of Formulas I to V, each of X² and X³ is a deoxyribonucleotide or a ribonucleotide; when X² has the structure of any one of Formulas I to V, each of X¹ and X³ is a deoxyribonucleotide or a ribonucleotide; when X³ has the structure of any one of Formulas I to V, each of X¹ and X² is a deoxyribonucleotide or a ribonucleotide; when X¹ and X² each have the structure of any one of Formulas I to V, X³ is a deoxyribonucleotide or a ribonucleotide; when X¹ and X³ each have the structure of any one of Formulas I to V, X² is a deoxyribonucleotide or a ribonucleotide; and when X² and X³ each have the structure of any one of Formulas I to V, X¹ is a deoxyribonucleotide or a ribonucleotide.
Embodiment 51. The method of any one of embodiments 20 to 40 and 42 to 50, wherein none of X¹, X², and X³ has the structure of Formula II, wherein N¹ is a nucleobase.
Embodiment 52. The method of embodiment 51, wherein none of X¹, X², and X³ has the structure of Formula II, wherein N¹ is a cytosine nucleobase.
Embodiment 53. The method of any one of embodiments 20 to 44 and 47 to 52, wherein X¹ comprises a uracil or thymine nucleobase.
Embodiment 54. The method of embodiment 53, wherein X¹ comprises a uracil nucleobase.
Embodiment 55. The method of any one of 20 to 44 and 47 to 52, wherein X¹ comprises a hypoxanthine nucleobase.
Embodiment 56. The method of any one of embodiments 20 to 44 and 47 to 52, wherein X¹ comprises a cytosine nucleobase.
Embodiment 57. The method of any one of embodiments 20 to 56, wherein X³ comprises a guanine nucleobase.
Embodiment 58. The method of any one of embodiments 20 to 56, wherein X³ comprises a hypoxanthine nucleobase.
Embodiment 59. The method of any one of embodiments 20 to 56, wherein X³ comprises an adenine nucleobase.
Embodiment 60. The method of any one of embodiments 20 to 42, 46, 47, and 48 to 59, wherein X² comprises a cytosine or 5-methylcytosine nucleobase.
Embodiment 61. The method of embodiment 60, wherein X² comprises a cytosine nucleobase.
Embodiment 62. The method of any one of embodiments 20 to 24, wherein X² has the structure of any one of Formula I-V.
Embodiment 63. The method of any one of embodiments 20 to 62, wherein X² is not a 2′-O-methyl-nucleotide.
Embodiment 64. The method of embodiment 63, wherein X¹, X², and X³ are not 2′-O-methyl-nucleotides.
Embodiment 65. The method of any one of embodiments 1-19, wherein the guide oligonucleotide comprises the structure:

[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², or X³ has the structure of any one of Formula VI-XI:

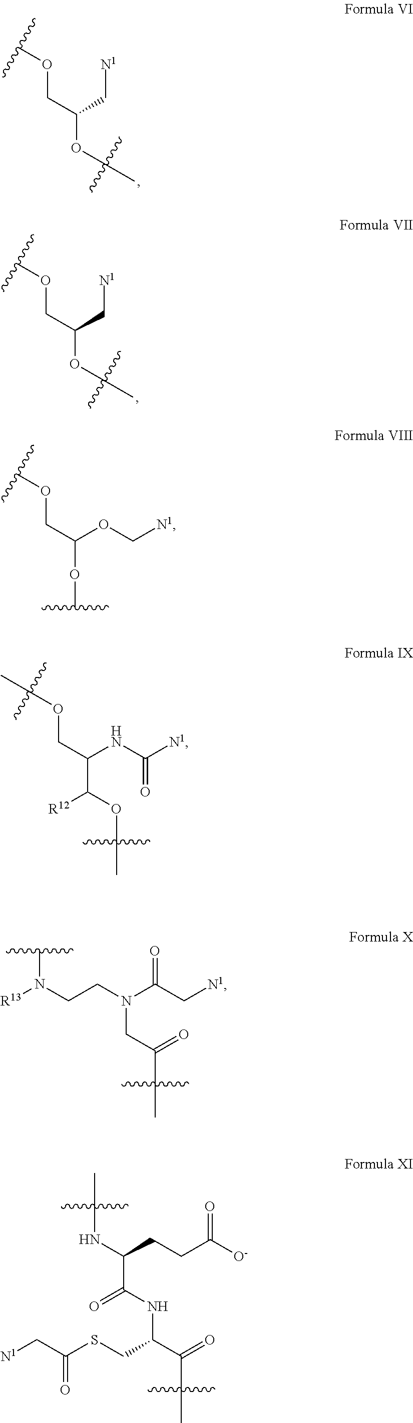

wherein N¹ is hydrogen or a nucleobase;
R¹² is hydrogen, hydroxy, fluoro, halogen, C₁-C₆ alkyl, C₁-C₆ heteroalkyl, or C₁-C₆ alkoxy;
R¹³ is hydrogen or C₁-C₆ alkyl,
wherein at least one of X¹, X², or X³ has the structure of any one of Formula VI-IX.
Embodiment 66. The method of embodiment 65, wherein at least 80% of the nucleotides of [Aₘ] and/or [Bₙ] include a nucleobase, a sugar, and an internucleoside linkage.
Embodiment 67. The method of embodiment 65 or 66, wherein R¹² is hydrogen, halogen, C₁-C₆ alkyl, or C₁-C₆ heteroalkyl.
Embodiment 68. The method of any one of embodiments 65 to 67, wherein the halogen is fluoro.
Embodiment 69. The method of any one of embodiments 65 to 68, wherein R¹² is hydrogen or C₁-C₆ alkyl;
Embodiment 70. The method of any one of embodiments 65 to 69, wherein R¹² is hydrogen.
Embodiment 71. The method of any one of embodiments 65 to 70, wherein at least one of X¹, X², and X³ has the structure of Formula VI, and N¹ is a nucleobase.
Embodiment 72. The method of embodiment 71, wherein X¹ has the structure of Formula VI, and N¹ is a nucleobase.
Embodiment 73. The method of embodiment 71 or 72, wherein X² has the structure of Formula VI, and N¹ is a nucleobase.
Embodiment 74. The method of any one of embodiments 65 to 70, wherein at least one of X¹, X², and X³ has the structure of Formula VII, and N¹ is a nucleobase.
Embodiment 75. The method of embodiment 74, wherein X¹ has the structure of Formula VII, and N¹ is a nucleobase.
Embodiment 76. The method of embodiment 74 or 75, wherein X² has the structure of Formula VII, and N¹ is a nucleobase.
Embodiment 77. The method of any one of embodiments 65 to 70, wherein at least one of X¹, X², and X³ has the structure of Formula IX, and N¹ is a nucleobase.
Embodiment 78. The method of embodiment 77, wherein X¹ has the structure of Formula IX, and N¹ is a nucleobase.
Embodiment 79. The method of embodiment 77 or 78, wherein X² has the structure of Formula IX, and N¹ is a nucleobase.
Embodiment 80. The method of any one of embodiments 65 to 70, wherein at least one of X¹, X², and X³ has the structure of Formula VIII, and N¹ is a nucleobase.
Embodiment 81. The method of embodiment 80, wherein X¹ has the structure of Formula VIII, and N¹ is a nucleobase.
Embodiment 82. The method of embodiment 80 or 81, wherein X² has the structure of Formula VIII, and N¹ is a nucleobase.
Embodiment 83. The method of any one of embodiments 65 to 72 and 74 to 82, wherein X² does not have the structure of Formula VI.
Embodiment 84. The method of any one of embodiments 65 to 83, wherein X³ does not have the structure of Formula VI.
Embodiment 85. The method of any one of embodiments 65 to 75 and 77 to 84, wherein X² does not have the structure of Formula VII.
Embodiment 86. The method of any one of embodiments 65 to 85, wherein X³ does not have the structure of Formula VII.
Embodiment 87. The method of any one of embodiments 65 to 78 and 80 to 86, wherein X² does not have the structure of Formula IX.
Embodiment 88. The method of any one of embodiments 65 to 70, wherein X² has the structure of Formula VI or Formula VII.
Embodiment 89. The method of any one of embodiments 65 to 88, wherein when X¹ has the structure of any one of Formulas VI to XI, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X² has the structure of any one of Formulas VI to XI, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X³ has the structure of any one of Formulas VI to XI, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X² each have the structure of any one of Formulas VI to XI, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X³ each have the structure of any one of Formulas VI to XI, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; and when X² and X³ each have the structure of any one of Formulas VI to XI, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide.
Embodiment 90. The method of embodiment 89, wherein when X¹ has the structure of any one of Formulas VI to XI, each of X² and X³ is, independently, a ribonucleotide, a 2′-F-nucleotide, 2′-O-methoxyethyl-nucleotide, or a deoxyribonucleotide; when X² has the structure of any one of Formulas VI to XI, each of X¹ and X³ is, independently, a ribonucleotide, a 2′-F-nucleotide, 2′-O-methoxyethyl-nucleotide, or a deoxyribonucleotide; when X³ has the structure of any one of Formulas VI to XI, each of X¹ and X² is, independently, a ribonucleotide, a 2′-F-nucleotide, 2′-O-methoxyethyl-nucleotide, or a deoxyribonucleotide; when X¹ and X² each have the structure of any one of Formulas VI to XI, X³ is a ribonucleotide, a 2′-F-nucleotide, 2′-O-methoxyethyl-nucleotide, or a deoxyribonucleotide; when X¹ and X³ each have the structure of any one of Formulas VI to XI, X² is a ribonucleotide, a 2′-F-nucleotide, 2′-O-methoxyethyl-nucleotide, or a deoxyribonucleotide; and when X² and X³ each have the structure of any one of Formulas VI to XI, X¹ is a ribonucleotide, a 2′-F-nucleotide, 2′-O-methoxyethyl-nucleotide, or a deoxyribonucleotide.
Embodiment 91. The method of embodiment 90, wherein when X¹ has the structure of any one of Formulas VI to XI, each of X² and X³ is a deoxyribonucleotide or a ribonucleotide; when X² has the structure of any one of Formulas VI to XI, each of X¹ and X³ is a deoxyribonucleotide or a ribonucleotide; when X³ has the structure of any one of Formulas VI to XI, each of X¹ and X² is a deoxyribonucleotide or a ribonucleotide; when X¹ and X² each have the structure of any one of Formulas VI to XI, X³ is a deoxyribonucleotide or a ribonucleotide; when X¹ and X³ each have the structure of any one of Formulas VI to XI, X² is a deoxyribonucleotide or a ribonucleotide; and when X² and X³ each have the structure of any one of Formulas VI to XI, X¹ is a deoxyribonucleotide or a ribonucleotide. Embodiment 92. The method of any one of embodiments 65 to 91, wherein X¹ comprises a hypoxanthine nucleobase. Embodiment 93. The method of any one of embodiments 65 to 91, wherein X¹ comprises a uracil nucleobase. Embodiment 94. The method of any one of embodiments 65 to 91, wherein X¹ comprises a cytosine nucleobase. Embodiment 95. The method of any one of embodiments 65 to 94, wherein X³ comprises a hypoxanthine nucleobase. Embodiment 96. The method of any one of embodiments 65 to 94, wherein X³ comprises a guanine nucleobase. Embodiment 97. The method of any one of embodiments 65 to 94, wherein X³ comprises a adenine nucleobase. Embodiment 98. The method of any one of embodiments 65 to 97, wherein X² comprises a cytosine nucleobase. Embodiment 99. The method of any one of embodiments 65 to 97, wherein X² comprises a uracil nucleobase.
Embodiment 100. The method of any one of embodiments 65 to 97, wherein X² does not include a nucleobase.
Embodiment 101. The method of any one of embodiments 65 to 100, wherein X² is not a 2′-O-methyl-nucleotide.
Embodiment 102. The method of any one of embodiments 65 to 101, wherein X¹, X², and X³ are not 2′-O-methyl-nucleotides.
Embodiment 103. The method of any one of embodiments 1-19, wherein the guide oligonucleotide comprises the structure:

[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², and X³ has the structure of any one of Formula XII-XV:

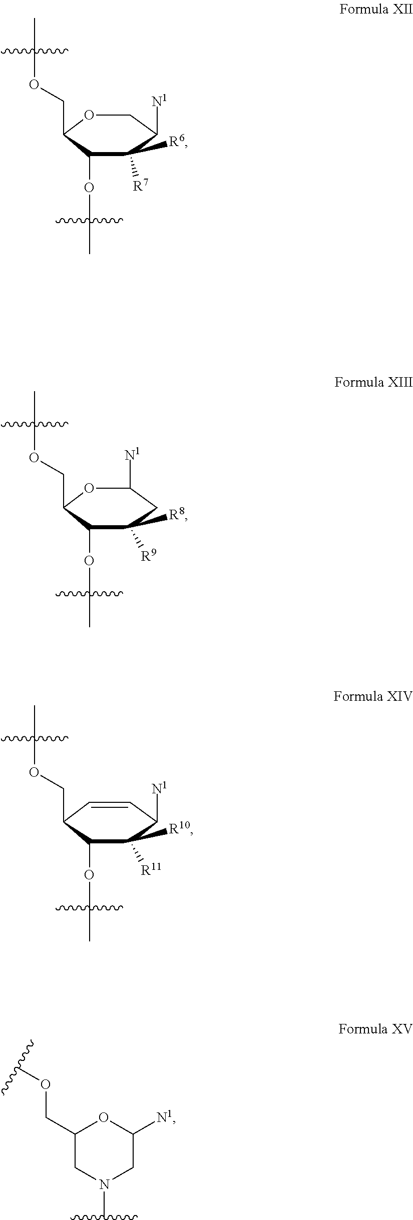

wherein N¹ is hydrogen or a nucleobase;
R⁶ is hydrogen, hydroxy, or halogen;
R⁷ is hydrogen, hydroxy, halogen, or C₁-C₆ alkoxy;
R⁸ is hydrogen or halogen;
R⁹ is hydrogen or hydroxy, halogen, or C₁-C₆ alkoxy;
R¹⁰ is hydrogen or halogen; and
R¹¹ is hydrogen or hydroxy, halogen, or C₁-C₆ alkoxy.
Embodiment 104. The method of embodiment 103, wherein at least 80% of the nucleotides of [Aₘ] and/or [Bₙ] include a nucleobase, a sugar, and an internucleoside linkage.
Embodiment 105. The method of embodiment 103 or 104, wherein halogen is fluoro.
Embodiment 106. The method of any one of embodiments 103 to 105, wherein C₁-C₆ alkoxy is OCH₃.
Embodiment 107. The method of any one of embodiments 103 to 106, wherein at least one of X¹, X², and X³ has the structure of Formula XIII, in which each of R⁸ and R⁹ is hydrogen.
Embodiment 108. The method of embodiment 107, wherein X¹ has the structure of Formula XIII, in which each of R⁸ and R⁹ is hydrogen.
Embodiment 109. The method of embodiment 107 or 108, wherein X² has the structure of Formula XIII, in which each of R⁸ and R⁹ is hydrogen.
Embodiment 110. The method of any one of embodiments 103 to 106, wherein X² has the structure of any one of Formula XII-XV.
Embodiment 111. The method of any one of embodiments 103 to 110, wherein when X¹ has the structure of any one of Formulas XII-XV, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X² has the structure of any one of Formulas XII-XV, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X³ has the structure of any one of Formulas XII-XV, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X² each have the structure of any one of Formulas XII-XV, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; when X¹ and X³ each have the structure of any one of Formulas XII-XV, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide; and when X² and X³ each have the structure of any one of Formulas XII-XV, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a deoxyribonucleotide.
Embodiment 112. The method of embodiment 111, wherein when X¹ has the structure of any one of Formulas XII-XV, each of X² and X³ is, independently, a ribonucleotide, a 2′-F-nucleotide, 2′-O-methoxyethyl-nucleotide, or a deoxyribonucleotide; when X² has the structure of any one of Formulas XII-XV, each of X¹ and X³ is, independently, a ribonucleotide, a 2′-F-nucleotide, 2′-O-methoxyethyl-nucleotide, or a deoxyribonucleotide; when X³ has the structure of any one of Formulas XII-XV, each of X¹ and X² is, independently, a ribonucleotide, a 2′-F-nucleotide, 2′-O-methoxyethyl-nucleotide, or a deoxyribonucleotide; when X¹ and X² each have the structure of any one of Formulas XII-XV, X³ is a ribonucleotide, a 2′-F-nucleotide, 2′-O-methoxyethyl-nucleotide, or a deoxyribonucleotide; when X¹ and X³ each have the structure of any one of Formulas XII-XV, X² is a ribonucleotide, a 2′-F-nucleotide, 2′-O-methoxyethyl-nucleotide, or a deoxyribonucleotide; and when X² and X³ each have the structure of any one of Formulas XII-XV, X¹ is a ribonucleotide, a 2′-F-nucleotide, 2′-O-methoxyethyl-nucleotide, or a deoxyribonucleotide.
Embodiment 113. The method of embodiment 112, wherein when X¹ has the structure of any one of Formulas XII-XV, each of X² and X³ is a deoxyribonucleotide or a ribonucleotide; when X² has the structure of any one of Formulas XII-XV, each of X¹ and X³ is a deoxyribonucleotide or a ribonucleotide; when X³ has the structure of any one of Formulas XII-XV, each of X¹ and X² is a deoxyribonucleotide or a ribonucleotide; when X¹ and X² each have the structure of any one of Formulas XII-XV, X³ is a deoxyribonucleotide or a ribonucleotide; when X¹ and X³ each have the structure of any one of Formulas XII-XV, X² is a deoxyribonucleotide or a ribonucleotide; and when X² and X³ each have the structure of any one of Formulas XII-XV, X¹ is a deoxyribonucleotide or a ribonucleotide.
Embodiment 114. The method of any one of embodiments 103 to 113, wherein X¹ includes a hypoxanthine nucleobase.
Embodiment 115. The method of any one of embodiments 103 to 113, wherein X¹ includes a uracil nucleobase.
Embodiment 116. The method of any one of embodiments 103 to 113, wherein X¹ includes a cytosine nucleobase.
Embodiment 117. The method of any one of embodiments 103 to 116, wherein X³ includes a hypoxanthine nucleobase.
Embodiment 118. The method of any one of embodiments 103 to 116, wherein X³ includes an adenine nucleobase.
Embodiment 119. The method of any one of embodiments 103 to 118, wherein X² includes a cytosine nucleobase.
Embodiment 120. The method of any one of embodiments 103 to 118, wherein X² includes a uracil nucleobase.
Embodiment 121. The method of any one of embodiments 103 to 118, wherein X² does not include a nucleobase.
Embodiment 122. The method of any one of embodiments 103 to 121, wherein X² is not a 2′-O-methyl-nucleotide.
Embodiment 123. The method of any one of embodiments 103 to 122, wherein X¹, X², and X³ are not 2′-O-methyl-nucleotides.
Embodiment 124. The method of any one of embodiments 19 to 123, wherein [Aₘ] comprises at least one nuclease resistant nucleotide.
Embodiment 125. The method of any one of embodiments 19 to 124, wherein [Aₘ] comprises at least one 2′-O-C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one constrained ethyl (cEt)-nucleotide, at least one LNA-nucleotide, and/or at least one deoxyribonucleotide.
Embodiment 126. The method of embodiment 125, wherein [Aₘ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 127. The method of any one of embodiments 20 to 126, wherein [Aₘ] comprises at least five terminal 2′-O-methyl-nucleotides.
Embodiment 128. The method of any one of embodiments 20 to 127, wherein [Aₘ] comprises at least one phosphorothioate linkage.
Embodiment 129. The method of any one of embodiments 20 to 128, wherein [Aₘ] comprises at least four terminal phosphorothioate linkages.
Embodiment 130. The method of embodiment 128 or 129, wherein at least one phosphorothioate linkage is stereopure.
Embodiment 131. The method of any one of embodiments 20 to 130, wherein [Bₙ] comprises at least one nuclease resistant nucleotide.
Embodiment 132. The method of any one of embodiments 20 to 131, wherein [Bₙ] comprises at least one at least one 2′-O-C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 133. The method of embodiment 132, wherein [Bₙ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 134. The method of any one of embodiments 20 to 132, wherein [Bₙ] comprises at least five terminal 2′-O-methyl-nucleotides.
Embodiment 135. The method of any one of embodiments 20 to 134, wherein [Bₙ] comprises at least one phosphorothioate linkage.
Embodiment 136. The method of any one of embodiments 20 to 135, wherein [Bₙ] comprises at least four terminal phosphorothioate linkages.
Embodiment 137. The method of embodiment 135 or embodiment 136, wherein at least one phosphorothioate linkage is stereopure.
Embodiment 138. The method of any one of embodiments 20 to 137, wherein at least 20% of the nucleotides of [Aₘ] and [Bₙ] combined are 2′-O-methyl-nucleotides.
Embodiment 139. The method of any one of embodiments 20 to 138, wherein the oligonucleotide further comprises a 5′-cap structure.
Embodiment 140. The method of any one of embodiments 20 to 139, wherein the oligonucleotide comprises at least one alternative nucleobase.
Embodiment 141. The method of any one of embodiments 20 to 140, wherein the 5′-terminal nucleotide is a 2′-amino-nucleotide.
Embodiment 142. The method of any one of embodiments 20 to 141, wherein A and B combined consist of 18 to 80 nucleotides.
Embodiment 143. The method of any one of embodiments 20 to 142, wherein m is 5 to 40.
Embodiment 144. The method of any one of embodiments 20 to 143, wherein n is 5 to 40.
Embodiment 145. The method of embodiment 20, wherein m and n are each, independently, an integer from 5 to 40; at least one of X¹, X², and X³ has the structure of Formula I, wherein R¹ is fluoro, hydroxy, or methoxy and N¹ is a nucleobase, or the structure of Formula V, wherein R⁴ is hydrogen and R⁵ is hydrogen; each of X¹, X², and X³ that does not have the structure of Formula I or Formula V is a deoxyribonucleotide or a ribonucleotide; [Aₘ] and [Bₙ] each comprise at least five terminal 2′-O-methyl-nucleotides and at least four terminal phosphorothioate linkages; and at least 20% of the nucleotides of [Aₘ] and [Bₙ] combined are 2′-O-methyl-nucleotides.
Embodiment 146. The method of embodiment 65, wherein m and n are each, independently, an integer from 5 to 40; at least one of X¹, X², and X³ has the structure of Formula VI, Formula VII, Formula VIII, or Formula IX, wherein N¹ is a nucleobase and each of X¹, X², and X³ that does not have the structure of Formula VI, Formula VII, Formula VIII, or Formula IX is a deoxyribonucleotide or a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides.
Embodiment 147. The method of embodiment 103, wherein m and n are each, independently, an integer from 5 to 40; at least of X¹, X², and X³ has the structure of Formula XIII, wherein R⁸ and R⁹ are each hydrogen, and each of X¹, X² and X³ that does not have the structure of Formula XII is a deoxyribonucleotide or a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides.
Embodiment 148. The method of any one of embodiments 19-147, wherein when the oligonucleotide is hybridized to a SERPINA1 mRNA, X² is aligned with A at SNP rs28929474.
Embodiment 149. The method of any one of embodiments 1-148, wherein the guide oligonucleotide is capable of effecting ADAR-mediated adenosine to inosine alteration of the A at SNP rs28929474.
Embodiment 150. The method of any one of embodiments 10-149, wherein the treating comprises preventing, reversing, or slowing at least one symptom of A1AD selected from liver damage, hepatic failure, cirrhosis, jaundice, excessive break down of elastin in the lungs, emphysema, and COPD.
Embodiment 151. The method of any one of the preceding embodiments, wherein the guide oligonucleotide comprises or consists of an oligonucleotide selected from Tables 5-19.
Embodiment 152. An oligonucleotide capable of effecting an adenosine deaminase acting on RNA (ADAR)-mediated adenosine to inosine alteration or a target RNA, wherein the oligonucleotide comprises the structure:

[Aₘ]-X¹-X²-X³-X⁴-[Bₙ]
wherein each of A and B is a nucleotide;
m and n are each, independently, an integer from 1 to 50;
X¹, X², and X³ are each a deoxyribonucleotide and X⁴ is a 2′-fluoronucleotide, wherein when the oligonucleotide is hybridized to the target RNA, X² is opposite the adenosine that is to be deaminated to inosine.
Embodiment 153. An oligonucleotide comprising the structure:

[Aₘ]-X¹-X²-X³-X⁴-[Bₙ]
wherein each of A and B is a nucleotide;
m and n are each, independently, an integer from 1 to 50;
X¹, X², and X³ are each, independently, a nucleotide, and X⁴ is selected from a 2′-O-methylnucleotide and a 2′-fluoronucleotide;
wherein at least one of X¹, X², or X³ has the structure of any one of Formula I-V:

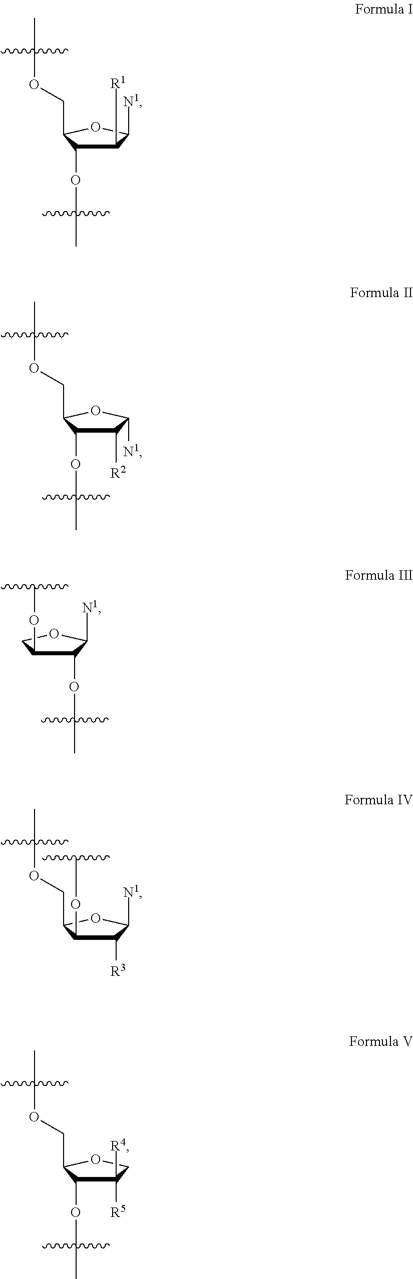

wherein N¹ is hydrogen or a nucleobase;
R¹ is hydroxy, halogen, or C₁-C₆ alkoxy;
R² is hydrogen, hydroxy, halogen, or C₁-C₆ alkoxy;
R³ is hydrogen, hydroxy, halogen, or C₁-C₆ alkoxy;
R⁴ is hydrogen, hydroxy, halogen, or C₁-C₆ alkoxy; and
R⁵ is hydrogen, hydroxy, halogen, or C₁-C₆ alkoxy.
Embodiment 154. The oligonucleotide of embodiment 153, wherein the oligonucleotide capable of effecting an adenosine deaminase acting on RNA (ADAR)-mediated adenosine to inosine alteration or a target RNA.
Embodiment 155. The oligonucleotide of embodiment 153 or 154, wherein X² is opposite the adenosine that is to be deaminated to inosine.
Embodiment 156. The oligonucleotide of any one of embodiments 153 to 155, wherein R⁴ is hydrogen and R⁵ is not hydrogen or hydroxy, R⁵ is hydrogen and R⁴ is not hydrogen, or R⁵ is hydroxy and R⁴ is not hydrogen.
Embodiment 157. The oligonucleotide of any one of embodiments 153 to 156, wherein R¹ is hydroxy, halogen, or OCH₃.
Embodiment 158. The oligonucleotide of any one of embodiments 153 to 157, wherein R² is hydrogen.
Embodiment 159. The oligonucleotide of any one of embodiments 153 to 158, wherein at least one of X¹, X², or X³ has the structure of Formula I, Formula II, or Formula V; and none of X¹, X², or X³ has the structure of Formula IV or Formula III.
Embodiment 160. The oligonucleotide of any one of embodiments 153 to 159, wherein at least one of X¹, X², or X³ has the structure of Formula I or Formula II; and none of X¹, X², or X³ has the structure of Formula III, Formula IV, or Formula V.
Embodiment 161. The oligonucleotide of any one of embodiments 153 to 160, wherein the halogen is fluoro.
Embodiment 162. The oligonucleotide of any one of embodiments 153 to 161, wherein at least one of X¹, X², and X³ has the structure of Formula I, wherein R¹ is fluoro and N¹ is a nucleobase.
Embodiment 163. The oligonucleotide of embodiment 162, wherein X¹ has the structure of Formula I, wherein R¹ is fluoro and N¹ is a nucleobase.
Embodiment 164. The oligonucleotide of embodiment 162 or 163, wherein X² has the structure of Formula I, wherein R¹ is fluoro and N¹ is a nucleobase.
Embodiment 165. The oligonucleotide of any one of embodiments 162 to 164, wherein X³ has the structure of Formula I, wherein R¹ is fluoro and N¹ is a nucleobase.
Embodiment 166. The oligonucleotide of any one of embodiments 153 to 161, wherein at least one of X¹, X², and X³ has the structure of Formula I, wherein R¹ is hydroxy and N¹ is a nucleobase.
Embodiment 167. The oligonucleotide of embodiment 166, wherein X¹ has the structure of Formula I, wherein R¹ is hydroxy and N¹ is a nucleobase.
Embodiment 168. The oligonucleotide of embodiment 166 or 167, wherein X² has the structure of Formula I, wherein R¹ is hydroxy and N¹ is a nucleobase.
Embodiment 169. The oligonucleotide of any one of embodiments 166 to 168, wherein X³ has the structure of Formula I, wherein R¹ is hydroxy and N¹ is a nucleobase.
Embodiment 170. The oligonucleotide of any one of embodiments 153 to 161, wherein at least one of X¹, X², and X³ has the structure of Formula I, wherein R¹ is methoxy and N¹ is a nucleobase.
Embodiment 171. The oligonucleotide of embodiment 170, wherein X¹ has the structure of Formula I, wherein R¹ is methoxy and N¹ is a nucleobase; and each of X² and X³ is a deoxyribonucleotide or a ribonucleotide.
Embodiment 172. The oligonucleotide of embodiment 170 or 171, wherein X² has the structure of Formula I, wherein R¹ is methoxy and N¹ is a nucleobase.
Embodiment 173. The oligonucleotide of any one of embodiments 170 to 172, wherein X³ has the structure of Formula I, wherein R¹ is methoxy and N¹ is a nucleobase.
Embodiment 174. The oligonucleotide of any one of embodiments 153 to 161, wherein at least one of X¹, X², and X³ has the structure of Formula II, wherein R² is hydrogen and N¹ is a nucleobase.
Embodiment 175. The oligonucleotide of embodiment 174, wherein X² has the structure of Formula II, wherein R² is hydrogen and N¹ is a nucleobase.
Embodiment 176. The oligonucleotide of any one of embodiments 153 to 159, wherein at least one of X¹ and X² has the structure of Formula V.
Embodiment 177. The oligonucleotide of embodiment 176, wherein X² has the structure of Formula V, wherein R⁴ is hydrogen and R⁵ is hydrogen.
Embodiment 178. The oligonucleotide of embodiment 176, wherein X² has the structure of Formula V, wherein R⁴ is hydrogen and R⁵ is hydroxy.
Embodiment 179. The oligonucleotide of embodiment 176, wherein X¹ has the structure of Formula V, wherein R⁴ is hydrogen and R⁵ is hydrogen.
Embodiment 180. The oligonucleotide of embodiment 176, wherein X¹ has the structure of Formula V, wherein R⁴ is hydrogen and R⁵ is hydroxy.
Embodiment 181. The oligonucleotide of embodiment 176, wherein X² has the structure of Formula V, wherein R⁴ is hydrogen and R⁵ is methoxy.
Embodiment 182. The oligonucleotide of any one of embodiments 153-181, wherein each of X¹, X², or X³ that does not have the structure of any one of Formula I-V is a deoxyribonucleotide.
Embodiment 183. The oligonucleotide of any one of embodiments 153 to 173 and 176 to 182, wherein none of X¹, X², and X³ has the structure of Formula II, wherein N¹ is a nucleobase.
Embodiment 184. The oligonucleotide of embodiment 183, wherein none of X¹, X², and X³ has the structure of Formula II, wherein N¹ is a cytosine nucleobase.
Embodiment 185. The oligonucleotide of any one of embodiments 153 to 178 and 181 to 184, wherein X¹ comprises a uracil or thymine nucleobase.
Embodiment 186. The oligonucleotide of embodiment 185, wherein X¹ comprises a uracil nucleobase.
Embodiment 187. The oligonucleotide of any one of 153 to 178 and 181 to 184, wherein X¹ comprises a hypoxanthine nucleobase.
Embodiment 188. The oligonucleotide of any one of embodiments 153 to 178 and 181 to 184, wherein X¹ comprises a cytosine nucleobase.
Embodiment 189. The oligonucleotide of any one of embodiments 153 to 188, wherein X³ comprises a guanine nucleobase.
Embodiment 190. The oligonucleotide of any one of embodiments 153 to 188, wherein X³ comprises a hypoxanthine nucleobase.
Embodiment 191. The oligonucleotide of any one of embodiments 153 to 188, wherein X³ comprises an adenine nucleobase.
Embodiment 192. The oligonucleotide of any one of embodiments 153 to 176, 179, 180, and 182 to 191, wherein X² comprises a cytosine or 5-methylcytosine nucleobase.
Embodiment 193. The oligonucleotide of embodiment 192, wherein X² comprises a cytosine nucleobase.
Embodiment 194. The oligonucleotide of any one of embodiments 153 to 158, wherein X¹ has the structure of any one of Formula I-V.
Embodiment 195. The oligonucleotide of any one of embodiments 153 to 194, wherein X² is not a 2′-O-methyl-nucleotide.
Embodiment 196. The oligonucleotide of embodiment 196, wherein X¹, X¹, and X³ are not 2′-O-methyl-nucleotides.
Embodiment 197. The oligonucleotide of any one of embodiments 152 to 196, wherein at least 80% of the nucleotides of [Aₘ] and/or [Bₙ] include a nucleobase, a sugar, and an internucleoside linkage.
Embodiment 198. The oligonucleotide of any one of embodiments 152 to 197, wherein [Aₘ] comprises at least one nuclease resistant nucleotide.
Embodiment 199. The oligonucleotide of any one of embodiments 152 to 198, wherein [Aₘ] comprises at least one 2′-O-C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one constrained ethyl (cEt)-nucleotide, at least one LNA-nucleotide, and/or at least one deoxyribonucleotide.
Embodiment 200. The oligonucleotide of embodiment 199, wherein [Aₘ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 201. The oligonucleotide of any one of embodiments 152 to 200, wherein [Aₘ] comprises at least five terminal 2′-O-methyl-nucleotides.
Embodiment 202. The oligonucleotide of any one of embodiments 152 to 201, wherein [Aₘ] comprises at least one phosphorothioate linkage.
Embodiment 203. The oligonucleotide of any one of embodiments 152 to 202, wherein [Aₘ] comprises at least four terminal phosphorothioate linkages.
Embodiment 204. The oligonucleotide of embodiment 202 or embodiment 203, wherein at least one phosphorothioate linkage is stereopure.
Embodiment 205. The oligonucleotide of any one of embodiments 152 to 204, wherein [Bₙ] comprises at least one nuclease resistant nucleotide.
Embodiment 206. The oligonucleotide of any one of embodiments 152 to 205, wherein [Bₙ] comprises at least one 2′-O-C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 207. The oligonucleotide of embodiment 206, wherein [Bₙ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one deoxyribonucleotide.
Embodiment 208. The oligonucleotide of any one of embodiments 152 to 207, wherein [Bₙ] comprises at least five terminal 2′-O-methyl-nucleotides.
Embodiment 209. The oligonucleotide of any one of embodiments 152 to 208, wherein [Bₙ] comprises at least one phosphorothioate linkage.
Embodiment 210. The oligonucleotide of any one of embodiments 152 to 209, wherein [Bₙ] comprises at least four terminal phosphorothioate linkages.
Embodiment 211. The oligonucleotide of embodiment 209 or embodiment 210, wherein at least one phosphorothioate linkage is stereopure.
Embodiment 212. The oligonucleotide of any one of embodiments 152 to 208, wherein at least 20% of the nucleotides of [Aₘ] and [Bₙ] combined are 2′-O-methyl-nucleotides.
Embodiment 213. The oligonucleotide of any one of embodiments 152 to 212, wherein the oligonucleotide further comprises a 5′-cap structure.
Embodiment 214. The oligonucleotide of any one of embodiments 152 to 213, wherein the oligonucleotide comprises at least one alternative nucleobase.
Embodiment 215. The oligonucleotide of any one of embodiments 152 to 214, wherein the 5′-terminal nucleotide is a 2′-amino-nucleotide.
Embodiment 216. The oligonucleotide of any one of embodiments 152 to 215, wherein A and B combined consist of 18 to 80 nucleotides.
Embodiment 217. The oligonucleotide of any one of embodiments 152 to 216, wherein m is 5 to 40.
Embodiment 218. The oligonucleotide of any one of embodiments 152 to 217, wherein n is 5 to 40.
Embodiment 219. The oligonucleotide of any one of embodiments 152 to 218, wherein the oligonucleotide further comprises one or more adenosine deaminase acting on RNA (ADAR)-recruiting domains.
Embodiment 220. The oligonucleotide of any one of embodiments 152 to 219, wherein the oligonucleotide capable of effecting an adenosine deaminase acting on RNA (ADAR)-mediated adenosine to inosine alteration of a SNP associated with alpha 1 antitrypsin deficiency.
Embodiment 221. The oligonucleotide of embodiment 220, wherein the oligonucleotide comprises a nucleic acid sequence complementary to a SERPINA1 mRNA sequence comprising the SNP associated with alpha 1 antitrypsin deficiency.
Embodiment 222. The oligonucleotide of embodiment 221, wherein the SNP is rs28929474(A).
Embodiment 223. The oligonucleotide of embodiment 222, wherein the SERPINA1 mRNA encodes a SERPINA1 protein comprising a pathogenic amino acid comprising a lysine at position 342 resulting from the SNP.
Embodiment 224. The oligonucleotide of embodiment 222 or embodiment 223, wherein when the oligonucleotide is hybridized to a SERPINA1 mRNA, X² is aligned with A at SNP rs28929474.
Embodiment 225. The oligonucleotide of any one of embodiments 220 to 224, wherein the oligonucleotide is capable of effecting ADAR-mediated adenosine to inosine alteration of the A at SNP rs28929474.
Embodiment 226. A conjugate comprising an oligonucleotide of any one of embodiments 152 to 225 conjugated to a targeting moiety.
Embodiment 227. The conjugate of embodiment 226, wherein the targeting moiety is a lipid, a sterol, a carbohydrate, and/or a peptide.
Embodiment 228. A complex comprising:
   an oligonucleotide of any one of embodiments 152 to 225 or a conjugate of embodiment 226 or 227; and
   an mRNA,
   wherein the oligonucleotide or conjugate and mRNA are hybridized to each other and the complex comprises a first mismatch at an adenosine of the mRNA.
Embodiment 229. The complex of embodiment 228, wherein the complex includes a second mismatch that is four nucleotides 5′ to the first mismatch.
Embodiment 230. The complex of embodiment 228 or 229, wherein the complex includes one, two, three, four, five, six, seven, or eight mismatches.
Embodiment 231. The complex of any one of embodiments 228 to 230, wherein the mRNA comprises an adenosine which may be deaminated to produce a therapeutic result.
Embodiment 232. The complex of any one of embodiments 228 to 230, wherein the mRNA comprises a guanosine to adenosine mutation compared to the corresponding natural mRNA.
Embodiment 233. The complex of embodiment 232, wherein the guanosine to adenosine mutation is a missense or nonsense mutation.
Embodiment 234. The complex of any one of embodiments 228 to 233, wherein the first mismatch is at an adenosine in a start codon of the mRNA.
Embodiment 235. The complex of any one of embodiments 228 to 233, wherein the first mismatch is at an adenosine in a stop codon of the mRNA.
Embodiment 236. The complex of embodiment 235, wherein the stop codon is a premature stop codon.
Embodiment 237. A method of producing a complex of any one of embodiments 228 to 236, the method comprising contacting a cell with an oligonucleotide of any one of embodiments 152 to 225 or a conjugate of embodiment 226 or 227.
Embodiment 238. A method for deamination of an adenosine in an mRNA, the method comprising contacting a cell with an oligonucleotide of any one of embodiments 152 to 225 or a conjugate of embodiment 226 or 227.
Embodiment 239. A method of treating a disorder in a subject in need thereof, the method comprising administering to the subject an effective amount of an oligonucleotide of any one of embodiments 152 to 226 or a conjugate of embodiment 226 or 227.
Embodiment 240. A method of editing a SERPINA1 polynucleotide comprising a single nucleotide polymorphism (SNP) associated with alpha 1 antitrypsin deficiency which may result in hepatic failure or emphysema, the method comprising contacting the SERPINA1 polynucleotide with the oligonucleotide of any one of embodiments 220 to 225 or the conjugate of embodiment 226 or embodiment 227, thereby editing the SERPINA1 polynucleotide.
Embodiment 241. The method of embodiment 240, wherein the SERPINA1 polynucleotide is contacted with the guide oligonucleotide in a cell.
Embodiment 242. The method of embodiment 241, wherein the cell endogenously expresses ADAR.
Embodiment 243. The method of embodiment 242, wherein the ADAR is a human ADAR.
Embodiment 244. The method of embodiment 243, wherein the ADAR is human ADAR1.
Embodiment 245. The method of embodiment 243, wherein the ADAR is human ADAR2.
Embodiment 246. The method of any one of embodiments 241-245, wherein the cell is selected from eukaryotic cell, a mammalian cell, and a human cell.
Embodiment 247. The method of any one of embodiments 241-246, wherein the cell is in vivo.
Embodiment 248. The method of any one of embodiments 241-246, wherein the cell is ex vivo.
Embodiment 249. A method of treating alpha 1 antitrypsin deficiency in a subject in need thereof, the method comprising
   identifying a subject with a single nucleotide polymorphism (SNP) associated with alpha 1 antitrypsin deficiency in a SERPINA1 polynucleotide;
   contacting the SERPINA1 polynucleotide in a cell of the subject with the oligonucleotide of any one of embodiments 220 to 225 or the conjugate of embodiment 226 or embodiment 227, thereby treating the subject.
Embodiment 250. A method of treating alpha 1 antitrypsin deficiency in a subject in need thereof, the method comprising
   identifying a subject with a single nucleotide polymorphism (SNP) associated with alpha 1 antitrypsin deficiency in a SERPINA1 polynucleotide;
   contacting the SERPINA1 polynucleotide in a cell with the oligonucleotide of any one of embodiments 220 to 225 or the conjugate of embodiment 226 or embodiment 227, and administering the cell to the subject, thereby treating the subject.
Embodiment 251. The method of embodiment 250, wherein the cell is autologous, allogenic, or xenogenic to the subject.
Embodiment 252. The method of any one of embodiments 249-251, wherein the subject is a human subject.
Embodiment 253. The method of any one of embodiments 240-252, wherein the guide oligonucleotide comprises a nucleic acid sequence complementary to a SERPINA1 mRNA sequence comprising the SNP associated with alpha 1 antitrypsin deficiency.
Embodiment 254. The method of any one of embodiments 240-253, wherein the oligonucleotide further comprises one or more adenosine deaminase acting on RNA (ADAR)-recruiting domains.
Embodiment 255. The method of any one of embodiments 240-254, wherein the SERPINA1 polynucleotide encodes a SERPINA1 protein comprising a pathogenic amino acid comprising a lysine at position 342 resulting from the SNP.
Embodiment 256. The method of embodiment 255, wherein the adenosine to inosine alteration substitutes the pathogenic amino acid with a wild type amino acid.
Embodiment 257. The method of embodiment 256, wherein the wild type amino acid at position 342 comprises a glutamic acid.

### BRIEF DESCRIPTION OF THE DRAWING

FIG. 1 shows the sensitivity of target mRNA editing at two different concentrations of guide oligonucleotides comprising various nucleotide modifications at the +2 and −2 positions. For each set of bars, the top bar is 100 nM guide oligonucleotide and the bottom bar is 10 nM guide oligonucleotide.

### DETAILED DESCRIPTION OF THE INVENTION

The present invention provides methods of editing a SERPINA1 polynucleotide, e.g., a SERPINA1 polynucleotide comprising a single nucleotide polymorphism (SNP) associated with alpha 1 antitrypsin deficiency, and methods for treating or preventing a SERPIN1-associated disease and its symptoms, e.g., alpha 1 antitrypsin deficiency, in a subject using a guide oligonucleotide capable of effecting an adenosine deaminase acting on RNA (ADAR)-mediated adenosine to inosine alteration in the target gene, e.g., an ADAR-mediated adenosine to inosine alternation of the SNP associated with alpha 1 antitrypsin deficiencies. In some embodiments, the deamination correcting the pathogenic mutation in the gene reverses the E342K mutation, restoring the glutamic acid and reversing and/or slowing liver and/or lung symptoms caused by the alpha 1 antitrypsin deficiency.

The following detailed description discloses methods for editing a SERPINA1 polynucleotide using a guide oligonucleotide capable of effecting an ADAR-mediated adenosine to inosine alteration, how to make and use compositions containing the guide oligonucleotides capable of effecting an ADAR-mediated adenosine to inosine alteration, as well as compositions, uses, and methods for treating subjects having a SERPIN1-associated disease that would benefit from editing the sequence of a SERPINA1 gene.

### I. Definitions.

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The term “or” is used herein to mean, and is used interchangeably with, the term “and/or,” unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means +10%. In certain embodiments, about means +5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, an oligonucleotide with “no more than 5 unmodified nucleotides” has 5, 4, 3, 2, 1, or 0 unmodified nucleotides. When “no more than” is present before a series of numbers or a range, it is understood that “no more than” can modify each of the numbers in the series or range.

As used herein, a “SERPINA1” or “serpin family A member 1” refers to the well-known gene and protein. SERPINA1 is also known as PI, A1A, AAT, PI1, A1AT, nNIF, PRO2275, and alpha1AT. The most common pathogenic AAT variant is Z (Glu342Lys) which causes AAT to misfold and polymerise within hepatocytes and other AAT-producing cells. The sequence of a human SERPINA1 mRNA transcript can be found at National Center for Biotechnology Information (NCBI) RefSeq accession number NM_000295.5, and the encoded protein sequence for A1AT precursor is found at NP_000286.3. Additional examples of SERPINA1 mRNA sequences are readily available using publicly available databases, e.g., GenBank, UniProt, and OMIM.

An “SERPIN1-associated disease,” as used herein, is intended to include any disease associated with the SERPINA1 gene or protein. Such a disease may be caused, for example, by the SERPINA1 gene mutation, by excess production of the SERPINA1 protein, by abnormal cleavage of the SERPINA1 protein, instability of SERPINA1 tetramers, by abnormal interactions between SERPINA1 and other proteins or other endogenous or exogenous substances. In some embodiments, the “SERPIN1-associated disease” is alpha 1 antitrypsin deficiency (A1AD). Nonlimiting exemplary symptoms of A1AD include cirrhosis, hepatic encephalopathy, ascites, jaundice, COPD, emphysema, bronchiectasis, asthma, shortness of breath after physical activity, weight loss, vision changes, fatigue, repeated respiratory infections, rapid heartbeat when standing, and/or a barrel-shaped chest. In some embodiments, A1AD leads to hepatic failure and/or or emphysema and/or COPD.

As used herein, the term “single nucleotide polymorphisms (SNP),” refers to a variation at a single position in a DNA sequence among individuals. If more than 1% of a population does not carry the same nucleotide at a specific position in the DNA sequence, then this variation can be classified as an SNP. If an SNP occurs within a gene, then the gene is described as having more than one allele. In these cases, SNPs may lead to variations in the amino acid sequence. For example, at a specific base position in the human genome, the C nucleotide can appear in most individuals, but in a minority of individuals, the position is occupied by an A. This means that there is an SNP at this specific position, and the two possible nucleotide variations, C or A, are the two alleles for this position.

SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant is a variation in a single nucleotide without any limitations of frequency and can arise in somatic cells. A somatic single nucleotide variation can also be called a single-nucleotide alteration.

Although a particular SNP may not cause a disorder, some SNPs are associated with certain diseases. These associations allow for the use of specific SNPs to evaluate an individual's genetic predisposition to develop a disease. In addition, if certain SNPs are known to be associated with a trait, then examination of certain stretches of DNA near these SNPs will help identify the gene or genes responsible for the trait.

As used herein, the phrase “SNP associated with alpha 1 antitrypsin deficiency” refers to any SNPs that are associated with the onset or development of alpha 1 antitrypsin deficiencies. Exemplary SNPs associated with alpha 1 antitrypsin deficiency may include, but are not limited to, any single nucleotide changes in the SERPINA1 polynucleotide that result in a pathogenic amino acid at positions 342 of the SERPINA1 protein. In some embodiments, the SNP associated with alpha 1 antitrypsin deficiency is rs28929474, which is sometimes referred to as the “Pi-Z” allele. SNP rs28929474(A) is associated with the Glu342Lys pathological variant, which leads to A1AD.

The term “pathogenic amino acid” refers to any amino acid that is not a wild-type amino acid in a protein and which leads to a pathogenesis.

The terms “pathogenic mutation”, “pathogenic variant”, “disease causing mutation”, “disease causing variant”, or “deleterious mutation”, refers to a genetic alteration or mutation that increases an individual's susceptibility or predisposition to a certain disease or disorder. In some embodiments, the pathogenic mutation comprises at least one wild-type amino acid substituted by at least one pathogenic amino acid in a protein encoded by a gene.

The term “adenosine deaminase”, as used herein, refers to a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). In some embodiments, the adenosine deaminase catalyzes the hydrolytic deamination of adenine or adenosine in ribonucleic acid (RNA). The adenosine deaminases may be from any organism, such as a human, chimpanzee, gorilla, monkey, cow, dog, rat, or mouse. In some embodiments, the adenosine deaminase is from a bacterium, such as E. coli, S. aureus, S. typhi, S. putrefaciens, H. influenzae, or C. crescentus. In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase. For example, deaminase domains are described in International PCT Application Nos. PCT/2017/045381 (WO 2018/027078) and PCT/US2016/058344 (WO 2017/070632), each of which is incorporated herein by reference for its entirety. Also see Komor, A.C., et al., Nature 533, 420-424 (2016); Gaudelli, N. M., et al., Nature 551, 464-471 (2017); Komor, A. C., et al., Science Advances 3:eaao4774 (2017), and Rees, H. A., et al., Nat Rev Genet. 2018; 19(12):770-788. doi: 10.1038/s41576-018-0059-1, the entire contents of which are hereby incorporated by reference.

As used herein, the term “Adenosine deaminases acting on RNA (ADAR)” refers to editing enzymes which can recognize certain structural motifs of double-stranded RNA (dsRNA), bind to dsRNA and convert adenosine to inosine through deamination, resulting in recoding of amino acid codons that may lead to changes to the encoded protein and its function. The nucleobases surrounding the editing site, especially the one immediately 5′ of the editing site and one immediately 3′ to the editing site, which together with the editing site are termed the triplet, play an important role in the deamination of adenosine. A preference for U at the 5′ position and G at the 3′ position relative to the editing site, was revealed from the analysis of yeast RNAs efficiently edited by overexpressed human ADAR2 and ADAR1. (See Wang et al., (2018) Biochemistry, 57: 1640-1651; Eifler et al., (2013) Biochemistry, 52: 7857-7869, and Eggington et al., (2011) Nat. Commun., 319: 1-9.) There are three known ADAR proteins expressed in humans, ADAR1, ADAR2, and ADAR3. ADAR1 and ADAR2 are expressed throughout the body, whereas ADAR3 is expressed only in the brain. ADAR1 and ADAR2 are catalytically active, while ADAR3 is thought to be inactive. Recruiting ADAR to specific sites of selected transcripts and deamination of adenosine regardless of neighboring bases holds great promise for the treatment of disease.

As used herein, the term “ADAR-recruiting domain” refers to nucleotide sequences that may be part of the oligonucleotides of the present invention and which are able to recruit an ADAR enzyme. For example, such recruiting domains may form stem-loop structures that act as recruitment and binding regions for the ADAR enzyme. Oligonucleotides including such ADAR-recruiting domains may be referred to as “axiomer AONs” or “self-looping AONs.” The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein. An ADAR-recruiting domain may be a nucleotide sequence based on a natural substrate (e.g., the GluR2 receptor pre-mRNA; such as a GluR2 ADAR-recruiting domain), a Z-DNA structure, or a domain known to recruit another protein which is part of an ADAR fusion protein, e.g., an MS2 ADAR-recruiting domain known to be recognized by the dsRNA binding regions of ADAR. A stem-loop structure of an ADAR-recruiting domain can be an intermolecular stem-loop structure, formed by two separate nucleic acid strands, or an intramolecular stem loop structure, formed within a single nucleic acid strand.

As used herein, the term “Z-DNA” refers to a left-handed conformation of the DNA double helix or RNA stem loop structures. Such DNA or dsRNA helices wind to the left in a zigzag pattern (as opposed to the right, like the more commonly found B-DNA form). Z-DNA is a known high-affinity ADAR binding substrate and has been shown to bind to human ADAR1 enzyme.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide including a nucleotide bearing such replacement moiety. For example, without limitation, a nucleotide including hypoxanthine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, hypoxanthine. In another example, adenine and cytosine anywhere in the oligonucleotide can be replaced with guanine and uracil, respectively to form G-U wobble base pairing with the target mRNA. Sequences containing such replacement moieties are suitable for the compositions and methods featured in the invention.

The terms “nucleobase” and “base” include the purine (e.g., adenine and guanine) and pyrimidine (e.g., uracil, thymine, and cytosine) moiety present in nucleosides and nucleotides which form hydrogen bonds in nucleic acid hybridization. In the context of the present invention, the term nucleobase also encompasses alternative nucleobases which may differ from naturally-occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are, for example, described in Hirao et al (2012) Accounts of Chemical Research vol 45, page 2055 and Bergstrom (2009) Current Protocols in Nucleic Acid Chemistry Suppl. 37 Chapter 1, unit 4.1.

In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methylcytosine, 5-thiozolo-cytosine, 5-propynyl-cytosine, 5-propynyl-uracil, 5-bromouracil, 5-thiazolo-uracil, 2-thio-uracil, pseudouracil, 1-methylpseudouracil, 5-methoxyuracil, 2′-thio-thymine, hypoxanthine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g. A, T, G, C, or U, wherein each letter may optionally include alternative nucleobases of equivalent function.

A “sugar” or “sugar moiety,” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein refers to a monomeric unit of an oligonucleotide or polynucleotide that includes a nucleoside and an internucleoside linkage. The internucleoside linkage may or may not include a phosphate linkage. Similarly, “linked nucleosides” may or may not be linked by phosphate linkages. Many “alternative internucleoside linkages” are known in the art, including, but not limited to, phosphorothioate and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide” as used herein, refers to a nucleotide having an alternative nucleobase or an alternative sugar, and an internucleoside linkage, which may include alternative nucleoside linkages.

The term “nucleoside” refers to a monomeric unit of an oligonucleotide or a polynucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

The term “alternative nucleoside” refers to a nucleoside having an alternative sugar or an alternative nucleobase, such as those described herein.

The term “nuclease resistant nucleotide” as used herein refers to nucleotides which limit nuclease degradation of oligonucleotides. Nuclease resistant nucleotides generally increase stability of oligonucleotides by being poor substrates for the nucleases. Nuclease resistant nucleotides are known in the art, e.g., 2′-O-methyl-nucleotides and 2′-fluoro-nucleotides.

The terms “oligonucleotide” and “polynucleotide” as used herein, are defined as it is generally understood by the skilled person as a molecule including two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, and is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide of the invention may include one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

The phrases “an oligonucleotide that is capable of effecting an adenosine deaminase acting on RNA (ADAR)-mediated adenosine to inosine alteration” or “a guide oligonucleotide that is capable of effecting an ADAR-mediated adenosine to inosine alteration” refer to an oligonucleotide that is specific for a target sequence and is capable to be utilized for the deamination reaction of a specific adenosine in a target sequence through an ADAR-mediated pathway. The oligonucleotide may comprise a nucleic acid sequence complementary to a target sequence, e.g., a SERPINA1 mRNA sequence comprising the SNP associated with alpha 1 antitrypsin deficiency. In some embodiments, the oligonucleotides may comprise a nucleic acid sequence complementary to target mRNA with the exception of at least one mismatch. The oligonucleotide includes a mismatch opposite the target adenosine. In some embodiments, the oligonucleotides for use in the methods of the present invention do not include those used by any other gene editing technologies known in the art., e.g., CRISPR.

The oligonucleotide may be of any length, and may range from about 10-100 bases in length, e.g., about 15-100 bases in length or about 18-100 bases in length, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 bases in length, such as about 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-31, 15-30, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-31, 18-30, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-31, 19-30, 20-50, 20-49, 20-48, 20-47, 20-46, 20-45, 20-44,20-43, 20-42, 20-41, 20-40, 20-39, 20-38, 20-37, 20-36, 20-35, 20-34, 20-33, 20-32, 20-31, 20-31, 20-30, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-31, or 21-30 bases in length. Ranges and lengths intermediate to the above recited ranges and lengths are also contemplated to be part of the invention.

The term “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some embodiments of the invention the conjugate or oligonucleotide conjugate of the invention may optionally, include a linker region which is positioned between the oligonucleotide and the conjugate moiety. In some embodiments, the linker between the conjugate and oligonucleotide is biocleavable. Phosphodiester containing biocleavable linkers are described in more detail in WO 2014/076195 (herein incorporated by reference).

“Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. Complementary sequences between an oligonucleotide and a target sequence as described herein, include base-pairing of the oligonucleotide or polynucleotide including a first nucleotide sequence to an oligonucleotide or polynucleotide including a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally no more than 5, 4, 3 or 2 mismatched base pairs upon hybridization for a duplex up to 30 base pairs, while retaining the ability to hybridize under the conditions most relevant to their ultimate application, e.g., deamination of an adenosine. “Substantially complementary” can also refer to a polynucleotide that is substantially complementary to a contiguous portion of the mRNA of interest (e.g., an mRNA having a target adenosine). For example, a polynucleotide is complementary to at least a part of the mRNA of interest if the sequence is substantially complementary to a non-interrupted portion of the mRNA of interest. In some embodiments, an oligonucleotide or portion of an oligonucleotide is at least 80%, at least 85%, at least 90%, at least 95%, at least 99%, or 100% complementary to a reference (e.g., target) sequence. In such embodiments, the percent complementarity is calculated over the length of the oligonucleotide or portion thereof.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide including the first nucleotide or nucleoside sequence to hybridize and form a duplex structure under certain conditions with an oligonucleotide or polynucleotide including the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). In some embodiments, the presently disclosed compositions can efficiently generate an“intended mutation”, such as a point mutation, in a nucleic acid (e.g., a nucleic acid within a genome of a subject) without generating a significant number of unintended mutations, such as unintended point mutations. In some embodiments, an intended mutation is a mutation that is generated by a specific guide oligonucleotide, specifically designed to generate the intended mutation. In general, mutations made or identified in a sequence (e.g., an amino acid sequence as described herein) are numbered in relation to a reference (or wild type) sequence, i.e., a sequence that does not contain the mutations. The skilled practitioner in the art would readily understand how to determine the position of mutations in amino acid and nucleic acid sequences relative to a reference sequence.

The term “contacting,” as used herein, includes contacting a target gene, e.g., SERPINA1 by any means. In some embodiments, a target gene is contacted with a guide oligonucleotide in a cell. Contacting a SERPINA1 polynucleotide in a cell with a guide oligonucleotide includes contacting the SERPINA1 polynucleotide in a cell in vitro with the guide oligonucleotide or contacting the SERPINA1 polynucleotide in a cell in vivo with the guide oligonucleotide.

Contacting a cell in vitro may be done, for example, by incubating the cell with the guide oligonucleotide. Contacting a cell in vivo may be done, for example, by injecting the guide oligonucleotide into or near the tissue where the cell is located, or by injecting the guide oligonucleotide agent into another area, e.g., the bloodstream or the subcutaneous space, such that the agent will subsequently reach the tissue where the cell to be contacted is located. For example, the guide oligonucleotide may contain and/or be coupled to a ligand that directs the oligonucleotide to a site of interest. Combinations of in vitro and in vivo methods of contacting are also possible. For example, a cell may also be contacted in vitro with a guide oligonucleotide and subsequently transplanted into a subject.

In one embodiment, contacting a cell with a guide oligonucleotide includes “introducing” or “delivering the oligonucleotide into the cell” by facilitating or effecting uptake or absorption into the cell. Absorption or uptake of a guide oligonucleotide can occur through unaided diffusive or active cellular processes, or by auxiliary agents or devices. Introducing a guide oligonucleotide into a cell may be in vitro and/or in vivo. For example, for in vivo introduction, oligonucleotides can be injected into a tissue site or administered systemically. In vitro introduction into a cell includes methods known in the art such as electroporation and lipofection. Further approaches are described herein below and/or are known in the art.

As used herein, “lipid nanoparticle” or “LNP” is a vesicle including a lipid layer encapsulating a pharmaceutically active molecule, such as a nucleic acid molecule, e.g., an oligonucleotide. LNP refers to a stable nucleic acid-lipid particle. LNPs typically contain a cationic, ionizable lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it may. Liposomes also include “sterically stabilized” liposomes, a term which, as used herein, refers to liposomes including one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (MA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “level” is meant a level or activity of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2-fold, about 3-fold, about 3.5-fold, about 4.5-fold, about 5-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder; a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “administration” refers to the administration of a composition (e.g., a compound or a preparation that includes a compound as described herein) to a subject or system. Administration to an animal subject (e.g., to a human) may be by any appropriate route, such as the one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “effective amount,” “therapeutically effective amount,” and “a “sufficient amount” of an agent that results in a therapeutic effect (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a disorder, it is an amount of the agent that is sufficient to achieve a treatment response as compared to the response obtained without administration. The amount of a given agent will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent is an amount which results in a beneficial or desired result in a subject as compared to a control. As defined herein, a therapeutically effective amount of an agent may be readily determined by one of ordinary skill by routine methods known in the art. Dosage regimen may be adjusted to provide the optimum therapeutic response.

“Prophylactically effective amount,” as used herein, is intended to include the amount of an oligonucleotide that, when administered to a subject having or predisposed to have a disorder, is sufficient to prevent or ameliorate the disease or one or more symptoms of the disease. Ameliorating the disease includes slowing the course of the disease or reducing the severity of later-developing disease. The “prophylactically effective amount” may vary depending on the oligonucleotide, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount (either administered in a single or in multiple doses) of an oligonucleotide that produces some desired local or systemic effect at a reasonable benefit/risk ratio applicable to any treatment. Oligonucleotides employed in the methods of the present invention may be administered in a sufficient amount to produce a reasonable benefit/risk ratio applicable to such treatment.

A prophylactically effective amount may also refer to, for example, an amount sufficient to, when administered to the subject, including a human, to delay the onset of one or more of the disorders described herein by at least 120 days, for example, at least 6 months, at least 12 months, at least 2 years, at least 3 years, at least 4 years, at least 5 years, at least 10 years or more, when compared with the predicted onset.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

When a particular substituent may be present multiple times in the same structure, each instance of the substituent may be independently selected from the list of possible definitions for that substituent.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “hydroxy,” as used herein, represents an —OH group.

The alkyl, heteroalkyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide 35 of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control

The details of one or more embodiments of the invention are set forth in the description below. Other features, objects, and advantages of the invention will be apparent from the description and from the claims.

### II. Methods of the Invention.

The present invention provides methods of editing a SERPINA1 polynucleotide, e.g., a SERPINA1 polynucleotide comprising a single nucleotide polymorphism (SNP) associated with alpha 1 antitrypsin deficiency (for example, hepatic failure and/or emphysema), and methods for treating or preventing a SERPINA1-associated disease, e.g., alpha 1 antitrypsin deficiency, in a subject. The methods include contacting the SERPINA1 polynucleotide with a guide oligonucleotide capable of effecting an adenosine deaminase acting on RNA (ADAR)-mediated adenosine to inosine alteration of the SNP associated with alpha 1 antitrypsin deficiency

The invention is used to make desired changes in a target sequence, e.g., a SERPINA1 polynucleotide comprising a SNP associated with alpha 1 antitrypsin deficiency, in a cell or a subject by site-directed editing of nucleotides through the use of an oligonucleotide that is capable of effecting an adenosine deaminase acting on RNA (ADAR)-mediated adenosine to inosine alteration of the SNP. As a result, the target sequence is edited through an adenosine deamination reaction mediated by ADAR, converting adenosines into inosine. In some embodiments, because I is recognized as G, the deamination correcting the pathogenic mutation in the gene reverses the E342K mutation back to wild-type, reversing or slowing symptoms associated with A1AD experienced by the patient

The changes may be in 5′ or 3′ untranslated regions of a target RNA, in splice sites, in exons (changing amino acids in protein translated from the target RNA, changing codon usage or splicing behavior by changing exonic splicing silencers or enhancers, and/or introducing or removing start or stop codons), in introns (changing splicing by altering intronic splicing silencers or intronic splicing enhancers, branch points) and in general in any region affecting RNA stability, structure or functioning. The target RNA sequence may comprise a mutation that one may wish to correct or alter, such as a transition or a transversion.

RNA editing enzymes are known in the art. In some embodiments, the RNA editing enzyme is the adenosine deaminase acting on RNA (ADARs), such as hADARI and hADAR2 in humans or human cells.

Adenosine deaminases acting on RNA (ADARs) catalyze adenosine (A) to inosine (I) editing of RNA that possesses double-stranded (ds) structure. A-to-I RNA editing results in nucleotide substitution, because I is recognized as G instead of A both by ribosomes and by RNA polymerases. A-to-I substitution can also cause dsRNA destabilization, as I:U mismatch base pairs are less stable than A:U base pairs. A-to-I editing occurs with both viral and cellular RNAs, and affects a broad range of biological processes. These include virus growth and persistence, apoptosis and embryogenesis, neurotransmitter receptor and ion channel function, pancreatic cell function, and post-transcriptional gene regulation by microRNAs. Biochemical processes that provide a framework for understanding the physiologic changes following ADAR-catalyzed A-to-I (=G) editing events include mRNA translation by changing codons and hence the amino acid sequence of proteins; pre-mRNA splicing by altering splice site recognition sequences; RNA stability by changing sequences involved in nuclease recognition; genetic stability in the case of RNA virus genomes by changing sequences during viral RNA replication; and RNA-structure-dependent activities such as microRNA production or targeting or protein-RNA interactions.

Three human ADAR genes are known, of which two encode active deaminases (ADAR1 and ADAR2). Human ADAR3 (hADAR3) has been described in the prior art, but reportedly has no deaminase activity. Alternative promoters together with alternative splicing give rise to two protein size forms of ADAR1: an interferon-inducible ADAR1-p150 deaminase that binds dsRNA and Z-DNA, and a constitutively expressed ADAR1-p110 deaminase. ADAR2, like ADAR1-p110, is constitutively expressed and binds dsRNA. It is known that only the longer isoform of ADAR1 is capable of binding to the Z-DNA structure that can be comprised in the recruiting portion of the oligonucleotide construct according to the invention. Consequently, the level of the 150 kDa isoform present in the cell may be influenced by interferon, particularly interferon-gamma (IFN-gamma). hADARI is also inducible by TNF-alpha. This provides an opportunity to develop combination therapy, whereby interferon-gamma or TNF-alpha and oligonucleotide constructs comprising Z-DNA as recruiting portion according to the invention are administered to a patient either as a combination product, or as separate products, either simultaneously or subsequently, in any order. Certain disease conditions may already coincide with increased IFN-gamma or TNF-alpha levels in certain tissues of a patient, creating further opportunities to make editing more specific for diseased tissues.

Recruiting ADAR to specific sites of selected transcripts and deamination of adenosine regardless of neighboring bases holds great promise for the treatment of disease. In some embodiments, the oligonucleotide that is capable of effecting an adenosine deaminase acting on RNA (ADAR)-mediated adenosine to inosine alteration of the SNP, e.g., a guide oligonucleotide as described herein, further comprises an ADAR-recruiting domain. In some embodiments, the ADAR-recruiting domain comprises nucleotide sequences that may be covalently linked to the oligonucleotides for use in the methods of the present invention and may form stem-loop structures that act as recruitment and binding regions for the ADAR enzyme. Oligonucleotides including such ADAR-recruiting domains may be referred to as “axiomer AONs” or “self-looping AONs.” The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein. An ADAR-recruiting domain may be a nucleotide sequence based on a natural substrate (e.g., the GluR2 receptor pre-mRNA; such as a GluR2 ADAR-recruiting domain), a Z-DNA structure, or a domain known to recruit another protein which is part of an ADAR fusion protein, e.g., an MS2 ADAR-recruiting domain known to be recognized by the dsRNA binding regions of ADAR. A stem-loop structure of an ADAR-recruiting domain can be an intermolecular stem-loop structure, formed by two separate nucleic acid strands, or an intramolecular stem loop structure, formed within a single nucleic acid strand.

In some embodiments, the ADAR is endogenously expressed in a cell. The cell is selected from the group consisting of a bacterial cell, a eukaryotic cell, a mammalian cell, and a human cell. In principle the invention can be used with cells from any mammalian species, but it is preferably used with a human cell.

The oligonucleotide capable of effecting an adenosine deaminase acting on RNA (ADAR)-mediated adenosine to inosine alteration of the SNP, e.g., a guide oligonucleotide as described herein, comprises a nucleic acid sequence complementary to the SERPINA1 mRNA encoding the SNP associated with alpha 1 antitrypsin deficiency. In some embodiments, the guide oligonucleotides are complementary to target mRNA with the exception of at least one mismatch. The oligonucleotide includes a mismatch opposite the target adenosine.

Once the oligonucleotide hybridizes to the target mRNA sequence, it forms a double-stranded RNA structure, which can be recognized by ADAR, and facilitates the recruitment of ADAR to the target sequence. As a result, ADAR can catalyze the deamination reaction of the specific adenosine in the SNP associated with alpha 1 antitrypsin deficiency into an inosine.

Upon successful editing by the methods of the invention, the rs28929474 (A) allele is deaminated and converted to the rs28929474(G) allele by ADAR, and this ADAR-mediated adenosine to inosine alteration substitutes the pathogenic amino acid, serine, at position 342 of the SERPINA1 protein with a wild type amino acid, glycine, thereby removing the pathogenic or disease causing mutation in SERPINA1 protein.

The methods of the present invention can be used with cells from any organ, e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject. Such cells include but are not limited to hepatocytes, hepatocyte like cells, and/or alveolar type II cells.

The methods of the invention can also be used with mammalian cells which are not naturally present in an organism e.g. with a cell line or with an embryonic stem (ES) cell. The methods of the invention can be used with various types of stem cells, including pluripotent stem cells, totipotent stem cells, embryonic stem cells, induced pluripotent stem cells, etc.

The cells can be located in vitro or in vivo. One advantage of the invention is that it can be used with cells in situ in a living organism, but it can also be used with cells in culture. In some embodiments, cells are treated ex vivo and are then introduced into a living organism (e.g. re-introduced into an organism from whom they were originally derived). In some embodiments, the cell is contacted in vivo. In other embodiments, the cell is ex vivo.

The methods of invention can also be used to edit target RNA sequences in cells within a so-called organoid. Organoids are self-organized three-dimensional tissue structures derived from stem cells. Such cultures can be crafted to replicate much of the complexity of an organ, or to express selected aspects of it like producing only certain types of cells (Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. Thus, according to another preferred embodiment, the invention may be practised on organoids grown from tissue samples taken from a patient (e.g. from their gastrointestinal tract; see Sala et al. J Surg Res. 2009; 156(2):205-12, and Sato et al. Gastroenterology 2011; 141: 1762-72). Upon RNA editing in accordance with the invention, the organoids, or stem cells residing within the organoids, may be used to transplant back into the patient to ameliorate organ function.

In some embodiments, the cells to be treated have a genetic mutation. The mutation may be heterozygous or homozygous. The invention can be used to modify point mutations, for example, to correct a G to A mutation. In other embodiments, the cells to be treated do not have a genetic mutation. The invention can be used to create point mutations, for example, to generate a A to G mutation.

Accordingly, the invention is not limited to correcting mutations, as it may instead be useful to change a wild-type sequence into a mutated sequence by applying oligonucleotides according to the invention. One example where it may be advantageous to modify a wild-type adenosine is to bring about skipping of an exon, for example by modifying an adenosine that happens to be a branch site required for splicing of said exon. Another example is where the adenosine defines or is part of a recognition sequence for protein binding or is involved in secondary structure defining the stability of the mRNA. In some embodiments, however, the invention is used in the opposite way by introducing a disease-associated mutation into a cell line or an animal, in order to provide a useful research tool for the disease in question. As an example of creating a disease model for research purposes, an oligonucleotide sequence described herein provides for the recruitment of editing activity in a human cell to create a mutation in SERPINA1, e.g., a SERPINA1 mutation, that forms the basis for the onset of alpha 1 antitrypsin deficiency. As a result, the invention can be used to provide research tools for diseases, to introduce new mutations which are less deleterious than an existing mutation.

A mutation to be reverted through RNA editing may have arisen on the level of the chromosome or some other form of DNA, such as mitochondrial DNA, or RNA, including pre-mRNA, ribosomal RNA or mitochondrial RNA. A change to be made may be in a target RNA of a pathogen, including fungi, yeasts, parasites, kinetoplastids, bacteria, phages, viruses etc, with which the cell or subject has been infected. Subsequently, the editing may take place on the RNA level on a target sequence inside such cell, subject or pathogen. Certain pathogens, such as viruses, release their nucleic acid, DNA or RNA into the cell of the infected host (cell). Other pathogens reside or circulate in the infected host. The oligonucleotide constructs of the invention may be used to edit target RNA sequences residing in a cell of the infected eukaryotic host, or to edit a RNA sequence inside the cell of a pathogen residing or circulating in the eukaryotic host, as long as the cells where the editing is to take place contain an editing entity compatible with the oligonucleotide construct administered thereto.

Without wishing to be bound be theory, the RNA editing through ADAR1 and ADAR2 is thought to take place on pre-mRNAs in the nucleus, during transcription or splicing. Editing of mitochondrial RNA codons or non-coding sequences in mature mRNAs is not excluded.

Deamination of an adenosine using the oligonucleotides disclosed herein includes any level of adenosine deamination, e.g., at least 1 deaminated adenosine within a target sequence (e.g., at least, 1, 2, 3, or more deaminated adenosines in a target sequence).

Adenosine deamination may be assessed by a decrease in an absolute or relative level of adenosines within a target sequence compared with a control level. The control level may be any type of control level that is utilized in the art, e.g., pre-dose baseline level, or a level determined from a similar subject, cell, or sample that is untreated or treated with a control (such as, e.g., buffer only control or inactive agent control).

Because the enzymatic activity of ADAR converts adenosines to inosines, adenosine deamination can alternatively be assessed by an increase in an absolute or relative level of inosines within a target sequence compared with a control level. Similarly, the control level may be any type of control level that is utilized in the art, e.g., pre-dose baseline level, or a level determined from a similar subject, cell, or sample that is untreated or treated with a control (such as, e.g., buffer only control or inactive agent control).

The levels of adenosines and/or inosines within a target sequence can be assessed using any of the methods known in the art for determining the nucleotide composition of a polynucleotide sequence. For example, the relative or absolute levels of adenosines or inosines within a target sequence can be assessed using nucleic acid sequencing technologies including but not limited to Sanger sequencing methods, Next Generation Sequencing (NGS; e.g., pyrosequencing, sequencing by reversible terminator chemistry, sequencing by ligation, and real-time sequencing) such as those offered on commercially available platforms (e.g., Illumina, Qiagen, Pacific Biosciences, Thermo Fisher, Roche, and Oxford Nanopore Technologies). Clonal amplification of target sequences for NGS may be performed using real-time polymerase chain reaction (also known as qPCR) on commercially available platforms from Applied Biosystems, Roche, Stratagene, Cepheid, Eppendorf, or Bio-Rad Laboratories. Additionally or alternatively, emulsion PCR methods can be used for amplification of target sequences using commercially available platforms such as Droplet Digital PCR by Bio-Rad Laboratories.

In certain embodiments, surrogate markers can be used to detect adenosine deamination within a target sequence. For example, effective treatment of a subject having a genetic disorder involving G-to-A mutations with an oligonucleotide of the present disclosure, as demonstrated by an acceptable diagnostic and monitoring criteria can be understood to demonstrate a clinically relevant adenosine deamination. In certain embodiments, the methods include a clinically relevant adenosine deamination, e.g., as demonstrated by a clinically relevant outcome after treatment of a subject with an oligonucleotide of the present disclosure.

Adenosine deamination in a gene of interest may be manifested by an increase or decrease in the levels of mRNA expressed by a first cell or group of cells (such cells may be present, for example, in a sample derived from a subject) in which a gene of interest is transcribed and which has or have been treated (e.g., by contacting the cell or cells with an oligonucleotide of the present disclosure, or by administering an oligonucleotide of the invention to a subject in which the cells are or were present) such that the expression of the gene of interest is increased or decreased, as compared to a second cell or group of cells substantially identical to the first cell or group of cells but which has not or have not been so treated (control cell(s) not treated with an oligonucleotide or not treated with an oligonucleotide targeted to the gene of interest). The degree of increase or decrease in the levels of mRNA of a gene of interest may be expressed in terms of:

$$\frac{( mRNA in control cells ) - ( mRNA in treated cells )}{( mRNA in control cells )} \times 100 \%$$

In other embodiments, change in the levels of a gene may be assessed in terms of a reduction of a parameter that is functionally linked to the expression of a gene of interest, e.g., protein expression of the gene of interest or signaling downstream of the protein. A change in the levels of the gene of interest may be determined in any cell expressing the gene of interest, either endogenous or heterologous from an expression construct, and by any assay known in the art.

A change in the level of expression of a gene of interest may be manifested by an increase or decrease in the level of the protein produced by the gene of interest that is expressed by a cell or group of cells (e.g., the level of protein expressed in a sample derived from a subject). As explained above, for the assessment of mRNA suppression, the change in the level of protein expression in a treated cell or group of cells may similarly be expressed as a percentage of the level of protein in a control cell or group of cells.

A control cell or group of cells that may be used to assess the change in the expression of a gene of interest includes a cell or group of cells that has not yet been contacted with an oligonucleotide of the present disclosure. For example, the control cell or group of cells may be derived from an individual subject (e.g., a human or animal subject) prior to treatment of the subject with an oligonucleotide.

The level of mRNA of a gene of interest that is expressed by a cell or group of cells may be determined using any method known in the art for assessing mRNA expression. In one embodiment, the level of expression of a gene of interest in a sample is determined by detecting a transcribed polynucleotide, or portion thereof, e.g., mRNA of the gene of interest. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA of the gene of interest may be detected using methods the described in PCT Publication WO2012/177906, the entire contents of which are hereby incorporated herein by reference. In some embodiments, the level of expression of the gene of interest is determined using a nucleic acid probe. The term “probe,” as used herein, refers to any molecule that is capable of selectively binding to a specific sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of a gene of interest is determined by quantitative fluorogenic RT-PCR (i.e., the TAQMAN™ System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of nucleic acids of the gene of interest.

The level of protein produced by the expression of a gene of interest may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

### Methods of Treatment

The present invention also includes methods of treating or preventing a SERPINA1-associated disease or disorder, e.g., alpha 1 antitrypsin deficiency. For example, the methods of the invention may be used to treat or prevent any SERPIN1-associated disorders which may be caused by a guanosine to adenosine mutation, the introduction of a premature stop codon, or expression of an undesired protein. In some embodiments, the oligonucleotides for use in the methods of the invention, when introduced to a cell or a subject, can result in correction of a guanosine to adenosine mutation. In some embodiments, the oligonucleotides for use in the methods of the invention can result in turning off of a premature stop codon so that a desired protein is expressed. In some embodiments, the oligonucleotides for use in the methods of the invention can result in inhibition of expression of an undesired protein.

In one aspect, the present invention is directed to a method of treating an alpha 1 antitrypsin deficiency in a subject in need thereof. In some embodiments, the method comprises identifying a subject with a single nucleotide polymorphism (SNP) associated with alpha 1 antitrypsin deficiency in a SERPINA1 polynucleotide; and contacting the SERPINA1 polynucleotide in a cell of the subject with a guide oligonucleotide capable of effecting an adenosine deaminase acting on RNA (ADAR)-mediated adenosine to inosine alteration of the SNP associated with alpha 1 antitrypsin deficiency, thereby treating the subject.

In another aspect, the present invention is directed to a method of treating an alpha 1 antitrypsin deficiency, in a subject in need thereof. In some embodiments, the method comprises identifying a subject with a single nucleotide polymorphism (SNP) associated with alpha 1 antitrypsin deficiency in a SERPINA1 polynucleotide; contacting the SERPINA1 polynucleotide in a cell with a guide oligonucleotide capable of effecting an adenosine deaminase acting on RNA (ADAR)-mediated adenosine to inosine alteration of the SNP associated with alpha 1 antitrypsin deficiency, and administering the cell to the subject, thereby treating the subject.

In some embodiments, a method provided herein prevents, reverses, or slows the buildup of mutant A1AT in the liver in a subject with A1AD. In some embodiments, a method provided herein prevents, reverses, or slows liver damage associated with A1AD. In some embodiments, a method provided herein prevents, reverses, or slows hepatic failure in a subject with A1AD. In some embodiments, a method provided herein prevents, reverses, or slows development of cirrhosis in a subject with A1AD. In some embodiments, a method provided herein prevents, reverses, or slows development of jaundice in a subject with A1AD. In some embodiments, a method provided herein prevents, reverses, or slows development of ascites (i.e., abnormal accumulation of fluids in the abdominal cavity). In some embodiments, a method provided herein prevents, reverses, or slows development of hepatic encephalopathy.

In some embodiments, a method provided herein prevents, reverses, or slows the excessive break down of elastin in the lungs in a subject with A1AD. In some embodiments, a method provided herein prevents, reverses, or slows development of emphysema in a subject with A1AD. In some embodiments, a method provided herein prevents, reverses, or slows development of COPD in a subject with A1AD. In some embodiments, a method provided herein prevents or reduces the number of respiratory infections in a subject with A1AD. In some embodiments, a method provided herein prevents, reverses, or slows development of asthma. In some embodiments, a method provided herein prevents, reverses, or slows development of bronchiectasis.

In some embodiments, the subject is a human subject.

The methods of the invention thus may include a step of identifying a subject with a single nucleotide polymorphism (SNP) associated with an alpha 1 antitrypsin deficiency in a SERPINA1 polynucleotide. Specifically, the methods of the invention include a step of identifying the presence of the desired nucleotide change or SNPs in the target RNA sequence, thereby verifying that the target RNA sequence has the disease causing mutations to be corrected or editted. This step will typically involve sequencing of the relevant part of the target RNA sequence, or a cDNA copy thereof (or a cDNA copy of a splicing product thereof, in case the target RNA is a pre-mRNA), and the sequence change can thus be easily verified. Alternatively, the modifications may be assessed on the level of the protein (length, glycosylation, function or the like), or by some functional read-out.

The methods disclosed herein also include contacting the SERPINA1 polynucleotides with a single nucleotide polymorphism (SNP) associated with alpha 1 antitrypsin deficiency in a cell or a subject (including a subject identified as being in need of such treatment, or a subject suspected of being at risk of disease and in need of such treatment) with a guide oligonucleotide capable of effecting an adenosine deaminase acting on RNA (ADAR)-mediated adenosine to inosine alteration of the SNP associated with alpha 1 antitrypsin deficiency, as described herein.

The guide oligonucleotides for use in the methods of the invention are designed to specifically target the SERPINA1 gene of a subject (e.g., a human patient) in need thereof, and are capable of effecting an ADAR-mediated adenosine to inosine alteration in the SNPs associated with alpha 1 antitrypsin deficiency in the SERPINA1 gene. In some embodiments, the guide oligonucleotides are capable of recruiting the ADAR to the target mRNA, which then catalyze deamination of target adenosines in the target mRNA. Such treatment will be suitably introduced to a subject, particularly a human subject, suffering from, having, susceptible to, or at risk for developing an alpha 1 antitrypsin deficiency. In some embodiments, this deamination correcting the pathogenic mutation in the gene reverses the E342K mutation back to wild-type, reducing alpha 1 antitrypsin deficiency, and reversing or slowing the hepatic and/or emphysema related symptoms. The compositions disclosed herein may be also used in the treatment of any other disorders in which an alpha 1 antitrypsin deficiency may be implicated.

In one embodiment, the invention provides a method of monitoring treatment progress. The method includes the step of determining a level of diagnostic marker (Marker) (e.g., SNP associated with alpha 1 antitrypsin deficiency) or diagnostic measurement (e.g., screen, assay) in a subject suffering from or susceptible to developing alpha 1 antitrypsin deficiency, or symptoms associated with alpha 1 antitrypsin deficiency in which the subject has been administered a therapeutic amount of a composition disclosed herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In some embodiments, cells are obtained from the subject and contacted with an oligonucleotide composition of the invention as provided herein. In some embodiments, the cell is autologous, allogenic, or xenogenic to the subject. In some embodiments, cells removed from a subject and contacted ex vivo with an oligonucleotide composition of the invention are re-introduced into the subject, optionally after the desired genomic modification has been effected or detected in the cells.

In some embodiments, the oligonucleotide for use in the methods of the present disclosure is introduced to a subject such that the oligonucleotide is delivered to a specific site within the subject. The change in the expression of the gene of interest may be assessed using measurements of the level or change in the level of mRNA or protein produced by the gene of interest in a sample derived from a specific site within the subject.

In other embodiments, the oligonucleotide is introduced into the cell or the subject in an amount and for a time effective to result in one of (or more, e.g., two or more, three or more, four or more of: (a) decrease the number of adenosines within a target sequence of the gene of interest, (b) decrease the number of pathogenic mutations in the target protein, e.g., SERPINA1, (c) delayed onset of alpha 1 antitrypsin deficiency, (d) increased survival of subject, (e) recovery or change in protein function, and (f) reduction in one or more of symptoms related to alpha 1 antitrypsin deficiency, such as emphysema or hepatic failure.

Treating disorders associated with G-to-A mutations can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a compound or pharmaceutically acceptable salt of a compound described herein. A decrease in the mortality rate of a population may also be measured, for example, by calculating for a population the average number of disease-related deaths per unit time following completion of a first round of treatment with a compound or pharmaceutically acceptable salt of a compound described herein.

A. Methods of Administration

The delivery of an oligonucleotide for use in the methods of the invention to a cell e.g., a cell within a subject, such as a human subject (e.g., a subject in need thereof, such as a subject having alpha 1 antitrypsin deficiency) can be achieved in a number of different ways. For example, delivery may be performed by contacting a cell with an oligonucleotide of the invention either in vitro or in vivo. In vivo delivery may also be performed directly by administering a composition including an oligonucleotide to a subject. Alternatively, in vivo delivery may be performed indirectly by administering one or more vectors that encode and direct the expression of the oligonucleotide. Combinations of in vitro and in vivo methods of contacting a cell are also possible. Contacting a cell may be direct or indirect. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest, for example, the liver and/or lungs.

Contacting of a cell with an oligonucleotide may be done in vitro or in vivo. Known methods can be adapted for use with an oligonucleotide of the invention (see e.g., Akhtar S. and Julian R L., (1992) Trends Cell. Biol. 2(5):139-144 and W094/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the invention. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the invention are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

i. Membranous Molecular Assembly Delivery Methods

Oligonucleotides for use in the methods of the invention can also be delivered using a variety of membranous molecular assembly delivery methods including polymeric, biodegradable microparticle, or microcapsule delivery devices known in the art. For example, a colloidal dispersion system may be used for targeted delivery an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate ADAR-mediated RNA editing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. No. 4,897,355; U.S. Pat. No. 5,171,678; Bangham etal., (1965) M. Mol. Biol. 23:238; Olson etal., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. No. 5,283,185; U.S. Pat. No. 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems including non-ionic surfactant and cholesterol. Non-ionic liposomal formulations including NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes may also be sterically stabilized liposomes, including one or more specialized lipids that result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes including one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes including sphingomyelin. Liposomes including 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotides (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that include positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotides into the skin. In some implementations, liposomes are used for delivering oligonucleotides to epidermal cells and also to enhance the penetration of oligonucleotides into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems including non-ionic surfactant and cholesterol. Non-ionic liposomal formulations including Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with oligonucleotide are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable to the present invention.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotide for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

ii. Lipid Nanoparticle-Based Delivery Methods

Oligonucleotides for use in the methods of in the invention may be fully encapsulated in a lipid formulation, e.g., a lipid nanoparticle (LNP), or other nucleic acid-lipid particles. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipid include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

B. Combination Therapies

A method of the invention can be used alone or in combination with an additional therapeutic agent, e.g., other agents that treat the same disorder, e.g., alpha 1 antitrypsin deficiency or symptoms associated therewith, or in combination with other types of therapies to the disorder. In combination treatments, the dosages of one or more of the therapeutic compounds may be reduced from standard dosages when administered alone. For example, doses may be determined empirically from drug combinations and permutations or may be deduced by isobolographic analysis. Dosages of the compounds when combined should provide a therapeutic effect.

In some embodiments, the second therapeutic agent is a medication to prevent slow, or reverse liver damage. In some embodiments, the second therapeutic agent is Sulphasalazine. In some embodiments, the second therapeutic agent is acetylcysteine. In some embodiments, the second therapeutic agent is an agent that controls associated complications providing time for the liver to heal.

In some embodiments, the second therapeutic agent is a medication to prevent, slow, or reverse emphysema and/or COPD. In some embodiments, the second therapeutic agent is an antibiotic, bronchodilator, cough medicine, a flu vaccine and/or pneumococcal vaccine. In some embodiments, the second therapeutic agent is azithromycin, clarithromycin, cefuroxime, cefpodoxime, cefdinir, telithromycin, doxycycline, or trimethoprim/sulfamethoxazole. In some embodiments, the second therapeutic agent is albuterol, levalbuterol, or ipratropium. In some embodiments, the second therapeutic agent is aclidinium, arformotero, formoterol, glycopyrrolate, indacaterol, olodaterol, revefenacin, salmeterol, tiotropium, or umeclidinium.

The second agent may also be a therapeutic agent which is a non-drug treatment to prevent, slow, or reverse liver damage. In some embodiments, the second therapeutic agent is a revised diet and increased exercise prescribed to assist with recovery of liver functions. In other embodiments, the second therapeutic agent is a liver transplant.

The second agent may also be a therapeutic agent which is a non-drug treatment to prevent, slow, or reverse emphysema and/or COPD. In some embodiments, the second therapeutic agent is a lifestyle change the requires removal of lung irritants from living environments. In some embodiments, the second therapeutic agent is breathing exercises to strengthen the breathing muscles. In some embodiments, the second therapeutic agent is oxygen therapy. In some embodiments, the second therapeutic agent is a revised diet and increased exercise prescribed to strengthen the body generally. In other embodiments, the second therapeutic agent is surgery to remove damaged lung tissue or to provide a lung transplant.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### III. Compositions for Use in the Methods of the Invention

The compositions for use in the methods of the present invention, i.e., methods of editing a SERPINA1 polynucleotide, e.g., a SERPINA1 polynucleotide comprising a single nucleotide polymorphism (SNP) associated with alpha 1 antitrypsin deficiency (which may result in hepatic failure or emphysema), and methods for treating or preventing a SERPINA1-associated disease, e.g., alpha 1 antitrypsin deficiency, in a subject, include a guide oligonucleotide capable of effecting an adenosine deaminase acting on RNA (ADAR)-mediated adenosine to inosine alteration of the SNP associated with alpha 1 antitrypsin deficiency.

The oligonucleotides, or guide oligonucleotides, for use in the methods of the invention may be utilized to deaminate target adenosines on a specific mRNA, e.g., an adenosine which may be deaminated to produce a therapeutic result, e.g., in a subject in need thereof.

Examples of modifications resulting from deamination of target adenosines within a target codon are provided in Tables 1 and 2 below.

TABLE 1

| | Amino Acid | | Amino Acid |
|---|---|---|---|
| | Encoded by | | Encoded by |
| Target Codon | Target Codon | Modified Codon | Modified Codon |
| AAA | Lys | IAA | Glu |
| | | AIA | Arg |
| | | IIA | Gly |
| | | AII | Arg |
| | | IAI | Glu |
| | | III | Gly |
| AAC | Asn | IAC | Asp |
| | | AIC | Ser |
| | | IIC | Gly |
| AAG | Lys | IAG | Glu |
| | | AIG | Arg |
| | | IIG | Gly |
| AAU | Arg | IAU | Asp |
| | | AIU | Ser |
| | | IIU | Gly |
| ACA | Thr | ICA | Ala |
| | | ICI | Ala |
| ACC | Thr | ICC | Ala |
| ACG | Thr | ICG | Ala |
| ACU | Thr | ICU | Ala |
| AGA | Arg | IGA | Gly |
| | | IGI | Gly |
| AGC | Ser | IGC | Gly |
| AGG | Arg | IGG | Gly |
| AGU | Ser | IGU | Gly |
| AUA | Ile | IUA | Asp |
| | | AUI | Met |
| | | IUI | Val |
| AUC | Ile | IUC | Val |
| AUG | Met | IUG | Val |
| AUU | Ile | IUU | Val |
| CAA | Gln | CIA | Arg |
| | | CII | Arg |
| CAC | His | CIC | Arg |
| CAG | Gln | CIG | Arg |
| CAU | His | CIU | Arg |
| GAA | Glu | GIA | Gly |
| | | GII | Gly |
| GAC | Asp | GIC | Gly |
| GAG | Glu | GIG | Gly |
| GAU | Asp | GIU | Gly |
| UAA | Stop | UII | Trp |
| UGA | Stop | UGI | Trp |
| UAC | Tyr | UTC | Cys |
| UAG | Stop | UIG | Trp |
| UAU | Tyr | UIU | Cys |

TABLE 2
Target Codon Base Composition and Resulting Modified Codon

| Target Codon | Modified Codon |
|---|---|
| AAA | AIA |
| AAC | AIC |
| AAG | AIG |
| AAU | AIU |
| CAA | CIA |
| CAC | CIC |
| CAG | CIG |
| CAU | CIU |
| GAA | GIA |
| GAC | GIC |
| GAG | GIG |
| GAU | GIU |
| UAA | UIA |
| UAC | UIC |
| UAG | UIG |
| UAU | UIU |

Because the deamination of the adenosine to an inosine may result in a protein that is no longer suffering from the mutated A at the target position, the identification of the deamination into inosine may be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. When the presence of a target adenosine causes aberrant splicing, the read-out may be the assessment of whether the aberrant splicing is still taking place, or not, or less. On the other hand, when the deamination of a target adenosine is wanted to introduce a splice site, then similar approaches can be used to check whether the required type of splicing is indeed taking place. A very suitable manner to identify the presence of an inosine after deamination of the target adenosine is of course RT-PCR and sequencing, using methods that are well-known to the person skilled in the art.

In general, mutations in any target RNA that can be reversed using oligonucleotide constructs according to the invention are G-to-A mutations, and oligonucleotide constructs can be designed accordingly. Mutations that may be targeted using oligonucleotide constructs according to the invention also include C to A, U to A (T to A on the DNA level) in the case of recruiting adenosine deaminases. Although RNA editing in the latter circumstances may not necessarily revert the mutation to wild-type, the edited nucleotide may give rise to an improvement over the original mutation. For example, a mutation that causes an in frame stop codon—giving rise to a truncated protein, upon translation—may be changed into a codon coding for an amino acid that may not be the original amino acid in that position, but that gives rise to a (full length) protein with at least some functionality, at least more functionality than the truncated protein.

### Oligonucleotide Agents

The oligonucleotides for use in the methods of the present invention are complementary to target mRNA sequence, e.g., SERPINA1, comprising the SNP associated with a disease, e.g., alpha 1 antitrypsin deficiency (which may result in hepatic failure or emphysema). In some embodiments, the guide oligonucleotides are complementary to target mRNA with the exception of at least one mismatch. The oligonucleotide includes a mismatch opposite the target adenosine.

The guide oligonucleotides are also capable of recruiting adenosine deaminase acting on RNA (ADAR) enzymes to deaminate selected adenosines on the target mRNA. In some embodiments, the oligonucleotide further comprises one or more ADAR-recruiting domains. In some embodiments, only one adenosine is deaminated. In some embodiments, 1, 2, or 3 adenosines are deaminated.

The oligonucleotides for use in the methods of the invention may further include modifications (e.g., alternative nucleotides) to increase stability and/or increase deamination efficiency.

### A. Oligonucleotides

In one embodiment, one or more of the nucleotides of the oligonucleotide of the invention, is naturally-occurring, and does not include, e.g., chemical modifications and/or conjugations known in the art and described herein. In another embodiment, one or more of the nucleotides of an oligonucleotide of the invention, is chemically modified to enhance stability or other beneficial characteristics (e.g., alternative nucleotides). Without being bound by any particular theory, it is believed that certain modification can increase nuclease resistance and/or serum stability, or decrease immunogenicity. For example, polynucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides of the invention may be linked to one another through naturally-occurring phosphodiester bonds, or may be modified to be covalently linked through phosphorothiorate, 3′ -methylenephosphonate, 5′-methylenephosphonate, 3′-phosphoamidate, 2′-5′ phosphodiester, guanidinium, S-methylthiourea, or peptide bonds.

In some embodiments, one or more of the nucleotides of the oligonucleotide of the invention has the structure of any one of Formula I-V:

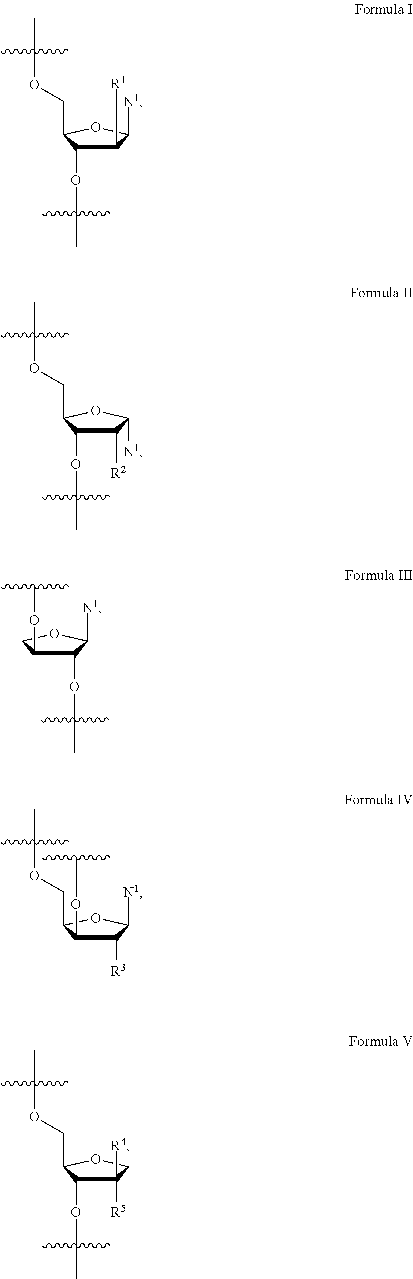

In some embodiments, one or more of the nucleotides of the oligonucleotide of the invention has the structure of any one of Formula I, e.g., has the structure:

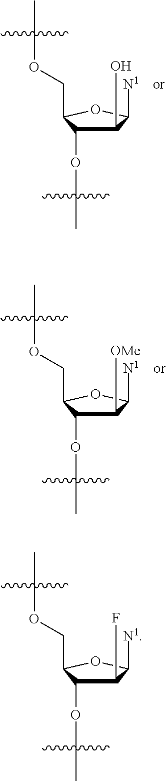

In some embodiments, one or more of the nucleotides of the oligonucleotide of the invention has the structure of any one of Formula II, e.g., has the structure:

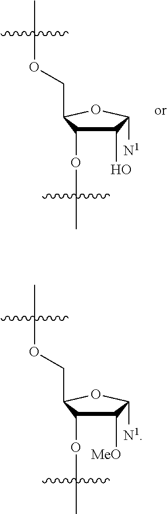

In some embodiments, one or more of the nucleotides of the oligonucleotide of the invention has the structure of any one of Formula III.

In some embodiments, one or more of the nucleotides of the oligonucleotide of the invention has the structure of any one of Formula IV, e.g., has the structure:

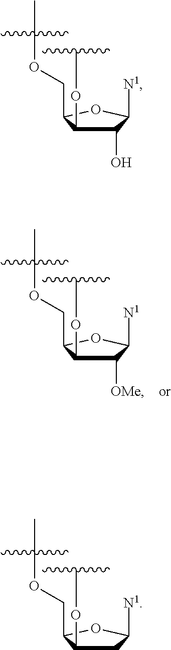

In some embodiments, one or more of the nucleotides of the oligonucleotide of the invention has the structure of any one of Formula V, e.g., has the structure:

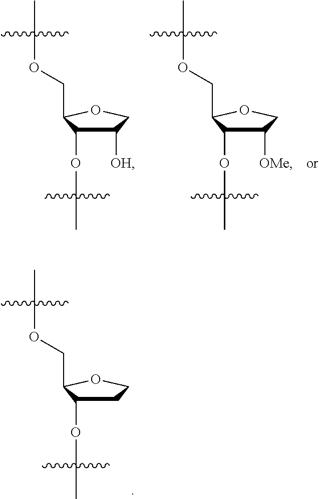

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments, the oligonucleotides of the present invention include the structure:

[Aₘ]-X¹-X²-X³-[Bₙ]

wherein each of A and B is a nucleotide; m and n are each, independently, an integer from 5 to 40; and at least one of X¹, X², and X³ has the structure of any one of Formulas I-V. In some embodiments, at least one of X¹, X², and X³ has the structure of Formula I, wherein R¹ is fluoro, hydroxy, or methoxy and N¹ is a nucleobase, or the structure of Formula V, wherein R⁴ is hydrogen and R⁵ is hydrogen; each of X¹, X², and X³ that does not have the structure of Formula I is a deoxyribonucleotide or a ribonucleotide; [Aₘ] and [Bₙ] each include at least five terminal 2′-O-methyl-nucleotides and at least four terminal phosphorothioate linkages, and at least 20% of the nucleotides of [Aₘ] and [Bₙ] combined are 2′-O-methyl-nucleotides.

In some embodiments the oligonucleotides of the present invention include the structure:

[Aₘ]-X¹-X²-X³-X⁴-[Bₙ]
wherein each of A and B is a nucleotide, and m and n are each, independently, an integer from 1 to 50; X¹, X², and X³ are each, independently, a nucleotide, and X⁴ is selected from a 2′-O-methylnucleotide and a 2′-fluoronucleotide; and where at least one of X¹, X², or X³ has the structure of any one of Formula I-V above, wherein N¹ is hydrogen or a nucleobase, R¹ is hydroxy, halogen, or C₁-C₆ alkoxy, R² is hydrogen, hydroxy, halogen, or C₁-C₆ alkoxy, R³ is hydrogen, hydroxy, halogen, or C₁-C₆ alkoxy, R⁴ is hydrogen, hydroxy, halogen, or C₁-C₆ alkoxy, and R⁵ is hydrogen, hydroxy, halogen, or C₁-C₆ alkoxy.

In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X² includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X² includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, X² includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X² includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X¹ includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, one or more of the nucleotides of the oligonucleotide of the invention has the structure of any one of Formula VI-XI:

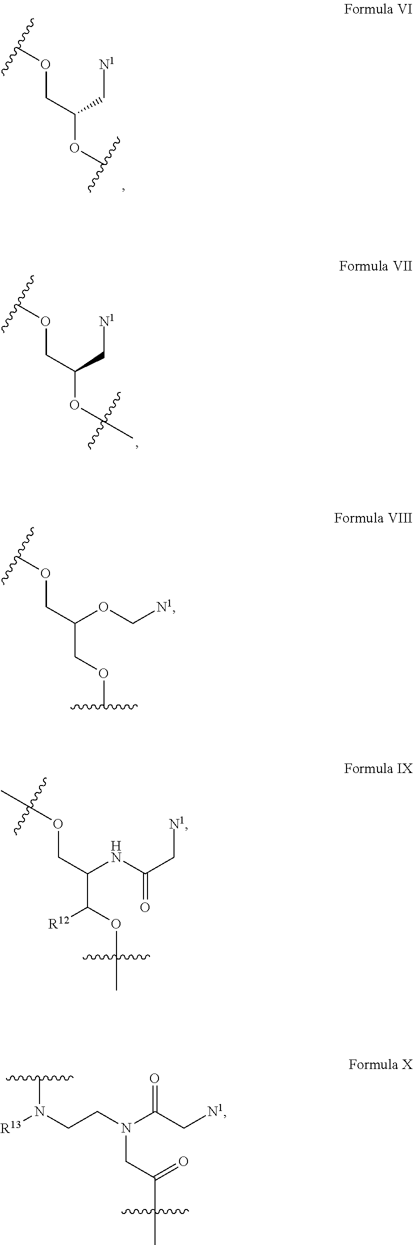

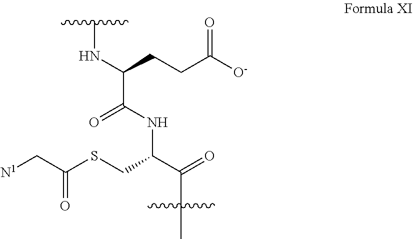

In some embodiments, one or more of the nucleotides of the oligonucleotide of the invention has the structure of any one of Formula VI.

In some embodiments, one or more of the nucleotides of the oligonucleotide of the invention has the structure of any one of Formula VII.

In some embodiments, one or more of the nucleotides of the oligonucleotide of the invention has the structure of any one of Formula VIII.

In some embodiments, one or more of the nucleotides of the oligonucleotide of the invention has the structure of any one of Formula IX, e.g., has the structure:

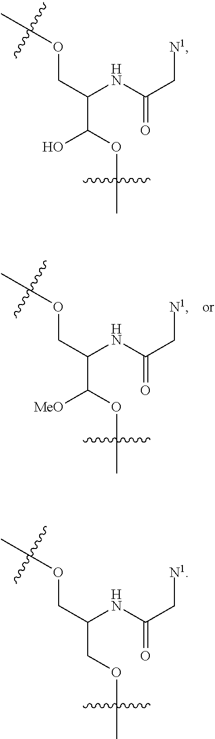

In some embodiments, one or more of the nucleotides of the oligonucleotide of the invention has the structure of any one of Formula X, e.g., has the structure:

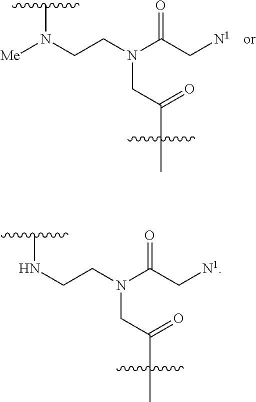

In some embodiments, one or more of the nucleotides of the oligonucleotide of the invention has the structure of any one of Formula XI, e.g., has the structure:

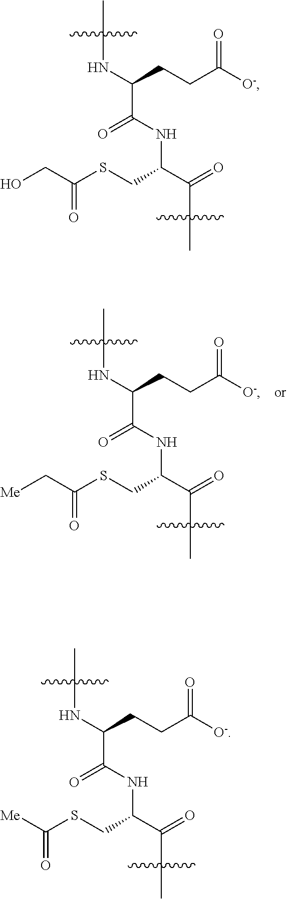

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments of the invention, the oligonucleotides of the present invention include the structure:

[Aₘ]-X¹-X²-X³-[Bₙ]

wherein each of A and B is a nucleotide; m and n are each, independently, an integer from 5 to 40; at least one of X¹, X², and X³ has the structure of any one of Formula VI-XI. In some embodiments, at least one of X¹, X², and X³ has the structure of Formula VI, Formula VII, Formula VIII, or Formula IX, wherein N¹ is a nucleobase and each of X¹, X², and X³ that does not have the structure of Formula VI, Formula VII, Formula VIII, or Formula IX is a deoxyribonucleotide or a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides. In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X¹ includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X¹ includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X¹ includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X¹ includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, one or more of the nucleotides of the oligonucleotide of the invention has the structure of any one of Formula XII-XV:

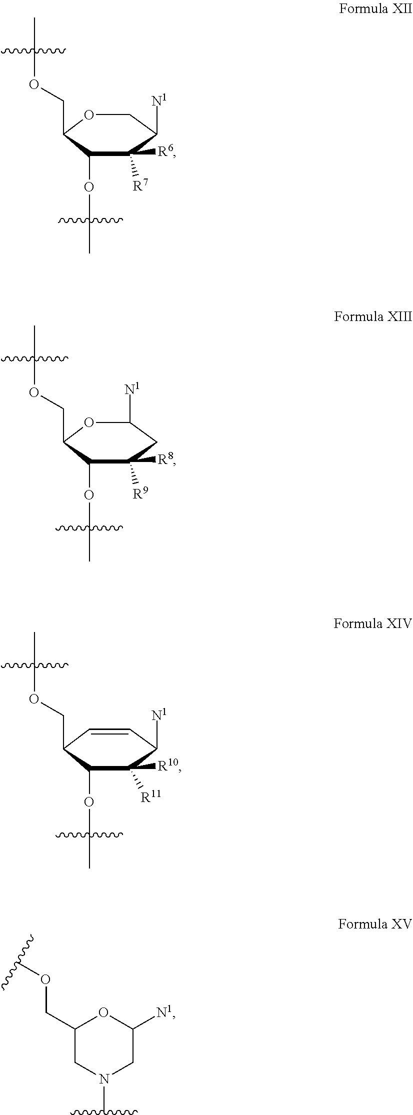

In some embodiments, one or more of the nucleotides of the oligonucleotide of the invention has the structure of any one of Formula XII, e.g., has the structure:

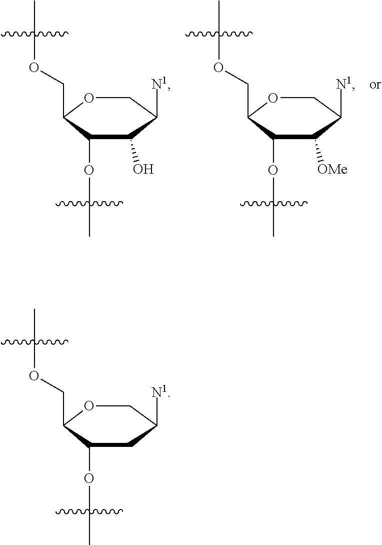

In some embodiments, one or more of the nucleotides of the oligonucleotide of the invention has the structure of any one of Formula XIII, e.g., has the structure:

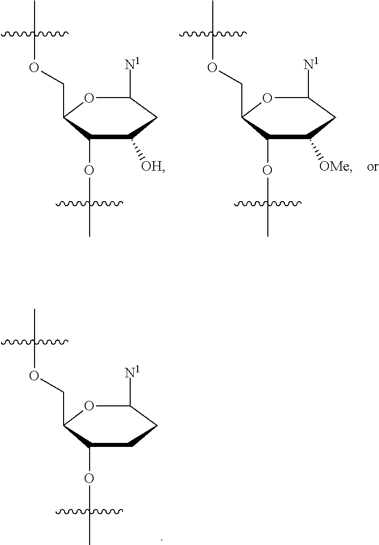

In some embodiments, one or more of the nucleotides of the oligonucleotide of the invention has the structure of any one of Formula XIV, e.g., has the structure:

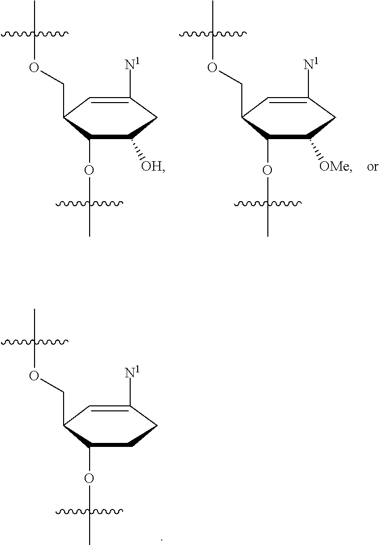

In some embodiments, one or more of the nucleotides of the oligonucleotide of the invention has the structure of any one of Formula XV.

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments, the oligonucleotides of the present invention include the structure:

[Aₘ]-X¹-X²-X³-[Bₙ]

wherein each of A and B is a nucleotide; m and n are each, independently, an integer from 5 to 40; at least of X¹, X², and X³ has the structure of any one of Formula XII-XV. In some embodiments, at least of X¹, X², and X³ has the structure of Formula XIII, wherein R⁸ and R⁹ are each hydrogen, and each of X¹, X² and X³ that does not have the structure of Formula XIII is a deoxyribonucleotide or a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides.

In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X² includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X² includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, X² includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X² includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X² includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, oligonucleotides of the present invention are capable of effecting an adenosine deaminase acting on RNA (ADAR)-mediated adenosine to inosine alteration or a target RNA, wherein the oligonucleotide includes the structure:

[Aₘ]-X¹-X²-X³-X⁴-[Bₙ]

wherein each of A and B is a nucleotide, m and n are each, independently, an integer from 1 to 50; X¹, X², and X³ are each a deoxyribonucleotide and X⁴ is a 2′-fluoronucleotide; and wherein when the oligonucleotide is hybridized to the target RNA, X² is opposite the adenosine that is to be deaminated to inosine.

In some embodiments, the oligonucleotides for use in the methods of the present invention include a recruitment domain for the ADAR enzyme (e.g., an ADAR-recruiting domain). In some embodiments, the ADAR-recruiting domain is a stem-loop structure. Such oligonucleotides may be referred to as “axiomer AONs” or “self-looping AONs.” The recruitment portion acts in recruiting a natural ADAR enzyme present in the cell to the dsRNA formed by hybridization of the target sequence with the targeting portion. The recruitment portion may be a stem-loop structure mimicking either a natural substrate (e.g. the glutamate ionotropic receptor AMPA type subunit 2 (GluR2) receptor; such as a GluR2 ADAR-recruiting domain) or a Z-DNA structure known to be recognized by the dsRNA binding regions of ADAR enzymes (e.g., a Z-DNA ADAR-recruiting domain). As GluR2 and Z-DNA ADAR-recruiting domains are high affinity binding partners to ADAR, there is no need for conjugated entities or presence of modified recombinant ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion may be a step loop structure described in WO 2016/097212, US 2018/0208924, Merkle et al. Nature Biotechnology, 37: 133-8 (2019), Katrekar et al. Nature Methods, 16(3): 239-42 (2019), Fukuda et al. Scientific Reports, 7: 41478 (2017), the stem-loop structures of the ADAR recruitment portion of which are herein incorporated by reference. In some embodiments, the oligonucleotides include one or more ADAR-recruiting domains (e.g., 1 or 2 ADAR-recruiting domains). In some embodiments, the ADAR-recruiting domain is at the 5′ end of the oligonucleotide. In other embodiments, the ADAR-recruiting domain is at the 3′ end of said oligonucleotide. In some embodiments, the oligonucleotide includes a first ADAR-recruiting domain and a second ADAR-recruiting domain. the first ADAR-recruiting domain is at the 5′ end of said oligonucleotide, and the second ADAR-recruiting domain is at the 3′ end of said oligonucleotide.

In some embodiments, the oligonucleotide includes the structure of Formula XVI:

C-L₁-D-L₂-[Aₘ]-X¹-X²-X³-[Bₙ] Formula XVI,

wherein [Aₘ]-X¹-X²-X³-[Bₙ] is the oligonucleotide of any one of formulas I-XV; C is a single-stranded oligonucleotide of 10-50 linked nucleosides in length; L₁ is a loop region; and D is a single-stranded oligonucleotide of 10-50 linked nucleosides in length; L₂ is an optional linker; wherein the oligonucleotide includes a duplex structure formed by C and D of between 10-50 linked nucleosides in length, wherein the duplex structure includes at least one mismatch between nucleotides of C and nucleotides of D, and wherein C or D includes at least one alternative nucleobase.

In some embodiments, C and D include at least one alternative nucleobase. In other embodiments, L₁ includes linked nucleosides. In yet another embodiment, L₁ consists of linked nucleosides. In some embodiments, L₁ includes at least one alternative nucleobase, at least one alternative internucleoside linkage, and/or at least one alternative sugar moiety. In some embodiments, C or D includes at least one alternative internucleoside linkage and/or at least one alternative sugar moiety. In some embodiments, C and D each independently includes at least one alternative internucleoside linkage and/or at least one alternative sugar moiety.

In some embodiments, the oligonucleotide includes the structure of Formula XVII:

C-L₁-D-L₂-[Aₘ]-X¹-X²-X³-[Bₙ] Formula XVII,

wherein [Aₘ]-X¹-X²-X³-[Bₙ] is the oligonucleotide of any one of Formulas I-XV; C is a single-stranded oligonucleotide of 10-50 linked nucleosides in length; L₁ is a loop region that does not consist of linked nucleosides; and D is a single-stranded oligonucleotide of 10-50 linked nucleosides in length; L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, L₁ has the structure of Formula XVIII:

F¹-(G¹)ⱼ-(H¹)ₖ-(G²)ₘ-(I)-(G³)ₙ-(H²)ₚ-(G⁴)_{q}-F² Formula XVIII,

wherein F¹ is a bond between the loop region and C; F² is a bond between D and [Aₘ] or between D and, optionally, the linker; G¹, G², G³, and G⁴ each, independently, is selected from optionally substituted C₁-C₂ alkyl, optionally substituted C1-C3 heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; j, k, m, n, p, and q are each, independently, 0 or 1; and I is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking F¹-(G¹)ⱼ-(H¹)ₖ-(G²)ₘ-(I)-(G³)ₙ-(H²)ₚ-(G⁴)_{q}-F².

In some embodiments, L₁ includes a carbohydrate-containing linking moiety.

In some embodiments, C or D each includes at least one alternative nucleobase, at least one alternative internucleoside linkage, and/or at least one alternative sugar moiety. In some embodiments, C and D each includes at least one alternative nucleobase, at least one alternative internucleoside linkage, and/or at least one alternative sugar moiety.

In some embodiments, the oligonucleotide includes the structure of Formula XIX:

C-L₁-D-L₂-[Aₘ]-X¹-X²-X³-[Bₙ] Formula XIX,

wherein [Aₘ]-X¹-X²-X³-[Bₙ] is the oligonucleotide of any one of formulas I to XV; C is a single-stranded oligonucleotide of 10-50 linked nucleosides in length; L₁ is a loop region including at least one alternative nucleobase or at least one alternative internucleoside linkage; and D is a single-stranded oligonucleotide of 10-50 linked nucleosides in length; L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, L₁ includes at least one alternative nucleobase and at least one alternative internucleoside linkage.

In some embodiments, the oligonucleotide includes the structure of Formula XX:

C-L₁-D-L₂-[Aₘ]-X¹-X²-X³-[Bₙ] Formula XX,

wherein [Aₘ]-X¹-X²-X³-[Bₙ] is the oligonucleotide of any one of formulas I to XV; C is a single-stranded oligonucleotide of 10-50 linked nucleosides in length; L₁ is a loop region including at least one alternative sugar moiety, wherein the alternative sugar moiety is selected from the group consisting of a 2′-O-C₁-C₆ alkyl-sugar moiety, a 2′-amino-sugar moiety, a 2′-fluoro-sugar moiety, a 2′-O-MOE sugar moiety, an arabino nucleic acid (ANA) sugar moiety, a deoxyribose sugar moiety, and a bicyclic nucleic acid; D is a single-stranded oligonucleotide of 10-50 linked nucleosides in length; and L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, the bicyclic sugar moiety is selected from an oxy-LNA sugar moiety (also referred to as an “LNA sugar moiety”), a thio-LNA sugar moiety, an amino-LNA sugar moiety, a cEt sugar moiety, and an ethylene-bridged (ENA) sugar moiety. In some embodiments, the ANA sugar moiety is a 2′-fluoro-ANA sugar moiety.

In some embodiments, C or D includes at least one alternative nucleobase, at least one alternative internucleoside linkage, and/or at least one alternative sugar moiety. In some embodiments, C and D each includes at least one alternative nucleobase, at least one alternative internucleoside linkage, and/or at least one alternative sugar moiety. In some embodiments, C is complementary to at least 5 contiguous nucleobases of D. In some embodiments, at least 80% (e.g., at least 85%, at least 90%, at least 95%) of the nucleobases of C are complementary to the nucleobases of D.

In some embodiments, C includes a nucleobase sequence having at least 80% sequence identity to a nucleobase sequence set forth in any one of SEQ ID NO. 1, 4, 7, 10, 13, 16, 19, 22, 25, 28, 31, and 34.

In some embodiments, D includes a nucleobase sequence having at least 80% sequence identity to a nucleobase sequence set forth in any one of SEQ ID NOs. 2, 5, 8, 11, 14, 17, 20, 23, 26, 29, 32, and 35.

In some embodiments, C-L₁-D includes a nucleobase sequence having at least 80% sequence identity to a nucleobase sequence set forth in any one of SEQ ID NOs. 3, 6, 9, 12, 15, 18, 21, 24, 27, 30, 33, and 36.

In some embodiments, the at least one alternative nucleobase is selected from the group consisting of 5-methylcytosine, 5-hydroxycytosine, 5-methoxycytosine, N4-methylcytosine, N3-Methylcytosine, N4-ethylcytosine, pseudoisocytosine, 5-fluorocytosine, 5-bromocytosine, 5-iodocytosine, 5-aminocytosine, 5-ethynylcytosine, 5-propynylcytosine, pyrrolocytosine, 5-aminomethylcytosine, 5-hydroxymethylcytosine, naphthyridine, 5-methoxyuracil, pseudouracil, dihydrouracil, 2-thiouracil, 4-thiouracil, 2-thiothymine, 4-thiothymine, 5,6-dihydrothymine, 5-halouracil, 5-propynyluracil, 5-aminomethyluracil, 5-hydroxymethyluracil, hypoxanthine, 7-deazaguanine, 8-aza-7-deazaguanine, 7-aza-2,6-diaminopurine, thienoguanine, N1-methylguanine, N2-methylguanine, 6-thioguanine, 8-methoxyguanine, 8-allyloxyguanine, 7-aminomethyl-7-deazaguanine, 7-methylguanine, imidazopyridopyrimidine, 7-deazaadenine, 3-deazaadenine, 8-aza-7-deazaadenine, 8-aza-7-deazaadenine, N1-methyladenine, 2-methyladenine, N6-methyladenine, 7-methyladenine, 8-methyladenine, or 8-azidoadenine.

In some embodiments, the at least one alternative nucleobase is selected from the group consisting of 2-amino-purine, 2,6-diamino-purine, 3-deaza-adenine, 7-deaza-adenine, 7-methyl-adenine, 8-azido-adenine, 8-methyl-adenine, 5-hydroxymethyl-cytosine, 5-methyl-cytosine, pyrrolo-cytosine, 7-aminomethyl-7-deaza-guanine, 7-deaza-guanine, 7-methyl-guanine, 8-aza-7-deaza-guanine, thieno-guanine, hypoxanthine, 4-thio-uracil, 5-methoxy-uracil, dihydro-uracil, or pseudouracil.

In some embodiments, the at least one alternative internucleoside linkage is selected from the group consisting of a phosphorothioate internucleoside linkage, a 2′-alkoxy internucleoside linkage, and an alkyl phosphate internucleoside linkage. In some embodiments, the at least one alternative internucleoside linkage is at least one phosphorothioate internucleoside linkage.

In some embodiments, the at least one alternative sugar moiety is selected from the group consisting of a 2′-O-alkyl-sugar moiety, a 2′-O-methyl-sugar moiety, a 2′-amino-sugar moiety, a 2′-fluoro-sugar moiety, a 2′-O-MOE sugar moiety, an ANA sugar moiety deoxyribose sugar moiety, and a bicyclic nucleic acid. In some embodiments, the bicyclic sugar moiety is selected from an oxy-LNA sugar moiety, a thio-LNA sugar moiety, an amino-LNA sugar moiety, a cEt sugar moiety, and an ethylene-bridged (ENA) sugar moiety. In some embodiments, the ANA sugar moiety is a 2′-fluoro-ANA sugar moiety. In some embodiments, the at least one alternative sugar moiety is a 2′-O-methyl-sugar moiety, a 2′-fluoro-sugar moiety, or a 2′-O-MOE sugar moiety.

In some embodiments, the at least one mismatch is a paired A to C mismatch, a paired G to G mismatch, or a paired C to A mismatch. In some embodiments, the oligonucleotide includes at least two mismatches between nucleotides of C and nucleotides of D.

In some embodiments, the at least two mismatches are separated by at least three linked nucleosides. In some embodiments, the at least two mismatches are separated by three linked nucleosides.

In some embodiments, the at least one mismatch includes a nucleoside having an alternative nucleobase. In some embodiments, the alternative nucleobase has the structure:

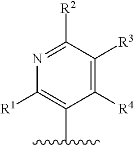

wherein R¹ is hydrogen, trifluoromethyl, optionally substituted amino, hydroxyl, or optionally substituted C₁-C₆ alkoxy; R² is hydrogen, optionally substituted amino, or optionally substituted C₁-C₆ alkyl; and R³ and R⁴ are, independently, hydrogen, halogen, or optionally substituted C₁-C₆ alkyl, or a salt thereof.

In one embodiment, the oligonucleotides of the invention include those including an ADAR-recruiting domain having a structure of Formula XXXIV:

C-L₁-D, Formula XXXIV,

wherein C is a single-stranded oligonucleotide of about 10-50 linked nucleosides in length (e.g., about 10, 15, 20, 25, 30, 35, 40, 45, 46, 47, 48, 49, or 50 linked nucleosides in length), L₁ is a loop region, and D is a single-stranded oligonucleotide of about 10-50 linked nucleosides in length (e.g., about 10, 15, 20, 25, 30, 35, 40, 45, 46, 47, 48, 49, or 50 linked nucleosides in length).

In some embodiments, C includes a region that is complementary to D such that the two strands hybridize and form a duplex under suitable conditions. Generally, the duplex structure is between 5 and 50 linked nucleosides in length, e.g., between, 5-49, 5-45, 5-40, 5-35, 5-30, 5-25, 5-20, 5-15, 5-10, 5-6, 8-50, 8-45, 8-40, 8-35, 8-30, 8-25, 8-20, 8-15, 8-10, 15-50, 15-45, 15-40, 15-35, 15-30, 15-25, 15-20, 15-16, 20-50, 20-45, 20-40, 20-35, 20-30, 20-25, 25-50, 25-45, 25-40, 25-35, or 25-30 linked nucleosides in length. Ranges and lengths intermediate to the above-recited ranges and lengths are also contemplated to be part of the invention. In some embodiments, C is complementary to at least 5 contiguous nucleobases (e.g., 5, 10, 15, 20, 25, 30, or more contiguous nucleobases) of D, and the oligonucleotide forms a duplex structure of between 10-50 linked nucleosides in length (e.g., at least 10, 15, 20, 25, 30, 35, 40, 45, 46, 47, 48, 49, or 50 linked nucleosides in length).

In some embodiments, the duplex structure includes at least one mismatch between nucleotides of C and nucleotides of D (e.g., at least 1, 2, 3, 4, or 5 mismatches). In some embodiments, the mismatch is a paired A to C mismatch. In some embodiments, the A nucleoside of the A to C mismatch is on the C strand and the C nucleoside of the A to C mismatch is on the D strand. In some embodiments, the A nucleoside of the A to C mismatch is on the D strand and the C nucleoside of the A to C mismatch is on the C strand. In other embodiments, the mismatch is a paired G-to-G mismatch. In still yet other embodiments, the mismatch is a paired C to A mismatch. In some embodiments, the C nucleoside of the C to A mismatch is on the C strand and the A nucleoside of the C to A mismatch is on the D strand. In some embodiments, the C nucleoside of the C to A mismatch is on the D strand and the A nucleoside of the C to A mismatch is on the C strand. In some embodiments, the mismatch is a paired i to I mismatch. In some embodiments, the mismatch is a paired i to G mismatch. In some embodiments, the I nucleoside of the i to G mismatch is on the C strand and the G nucleoside of the i to G mismatch is on the D strand. In some embodiments, the I nucleoside of the i to G mismatch is on the D strand and the G nucleoside of the i to G mismatch is on the C strand. In some embodiments, the mismatch is a paired G to I mismatch. In some embodiments, the G nucleoside of the G to I mismatch is on the C strand and the I nucleoside of the G to I mismatch is on the D strand. In some embodiments, the G nucleoside of the G to I mismatch is on the D strand and the I nucleoside of the G to I mismatch is on the C strand. In some embodiments, the mismatch includes a nucleoside having an alternative nucleobase. In some embodiments, the alternative nucleobase has the structure:

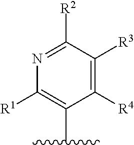

wherein R¹ is hydrogen, trifluoromethyl, optionally substituted amino, hydroxyl, or optionally substituted C₁-C₆ alkoxy; R² is hydrogen, optionally substituted amino, or optionally substituted C₁-C₆ alkyl; and R³ and R⁴ are, independently, hydrogen, halogen, or optionally substituted C₁-C₆ alkyl, or a salt thereof. In some embodiments, R¹ is a hydrogen bond donor group (e.g., a hydroxyl group, an amino group). In some embodiments, R¹ is a hydrogen bond accepting group (e.g., an alkoxy group).

In some embodiments, the duplex structure includes two mismatches. In some embodiments, the mismatches are at least three linked nucleosides apart. For example, when mismatches are “separated by 3 nucleotides,” the oligonucleotide includes the structure M₁-N₁-N₂-N₃-M₂, where M₁ is the first mismatch, N₁, N₂, and N₃ are paired nucleobases, and M₂ is the second mismatch. In some embodiments M₁ is a paired A to C mismatch and M₂ is a paired G-to-G mismatch.

In some embodiments, the loop region, L₁, includes linked nucleosides. In some embodiments, L₁ includes at least one alternative nucleobase, at least one alternative internucleoside linkage, and/or at least one alternative sugar moiety.

In other embodiments, the loop region has the structure of Formula XVIII:

F¹-(G¹)ⱼ-(H¹)ₖ-(G²)ₘ-(I)-(G³)ₙ-(H²)ₚ-(G⁴)_{q}-F² Formula XVIII,

wherein F¹ is a bond between the loop region and C; F² is a bond between D and a nucleotide or between D and, optionally, a linker; G¹, G², G³, and G⁴ each, independently, is selected from optionally substituted C1-C2 alkyl, optionally substituted C1-C3 heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; j, k, m, n, p, and q are each, independently, 0 or 1; and I is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking F¹-(G¹)ⱼ-(H¹)ₖ-(G²)ₘ-(I)-(G³)ₙ-(H²)ₚ-(G⁴)_{q}-F². In some embodiments, the linker is optional.

In some embodiments, the loop region, L₁ includes a carbohydrate-containing linking moiety.

In one embodiment, one or more of the nucleotides of the oligonucleotides of the invention, is naturally-occurring, and does not include, e.g., chemical modifications and/or conjugations known in the art and described herein. In another embodiment, one or more of the nucleotides of an oligonucleotide of the invention is chemically modified to enhance stability or other beneficial characteristics (e.g., alternative nucleotides). Without being bound by theory, it is believed that certain modification can increase nuclease resistance and/or serum stability, or decrease immunogenicity. For example, polynucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides of the invention may be linked to one another through naturally-occurring phosphodiester bonds, or may be modified to be covalently linked through phosphorothiorate, 3′ -methylenephosphonate, 5′ -methylenephosphonate, 3′-phosphoamidate, 2′-5′ phosphodiester, guanidinium, S-methylthiourea, or peptide bonds.

In some embodiments, C includes at least one alternative nucleobase, at least one alternative internucleoside linkage, and/or at least one alternative sugar moiety. In other embodiments, D includes at least one alternative nucleobase, at least one alternative internucleoside linkage, and/or at least one alternative sugar moiety. In some embodiments, both C and D each include at least one alternative nucleobase, at least one alternative internucleoside linkage, and/or at least one alternative sugar moiety.

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, an oligonucleotide of the invention can include no more than 5, 4, 3, 2, or 1 alternative nucleotides.

In one embodiment, the oligonucleotides of the invention include an ADAR-recruiting domain having the structure of Formula XXXIV, wherein C is a single-stranded oligonucleotide of 10-50 linked nucleosides in length, L₁ is a loop region, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, C or D includes at least one alternative nucleobase. In some embodiments, C and D each include at least one alternative nucleobase. In some embodiments, C and/or D, independently, further include at least one alternative internucleoside linkage and/or at least one alternative sugar moiety. In some embodiments, L₁ includes linked nucleotides. In other embodiments, L₁ consists of linked nucleosides. In some embodiments, L₁ includes at least one alternative nucleobase, at least one alternative internucleoside linkage, and/or at least one alternative sugar moiety.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region that does not consist of linked nucleosides, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, L₁ has the structure of Formula VIII, as described herein. In some embodiments, L₁ includes a carbohydrate-containing linking moiety. In some embodiments, C and/or D, independently, include at least one alternative nucleobase, at least one alternative internucleoside linkage, and/or at least one alternative sugar moiety.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region including at least one alternative nucleobase or at least one alternative internucleoside linkage, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, L₁ includes at least one alternative nucleobase and at least one alternative internucleoside linkage.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region including, at least one alternative sugar moiety that is not a 2′-O-methyl sugar moiety (e.g., the alternative sugar moiety is selected from the group consisting of a 2′-O-C₁-C₆ alkyl-sugar moiety, a 2′-amino-sugar moiety, a 2′-fluoro-sugar moiety, a 2′-O-MOE sugar moiety, an LNA sugar moiety, an arabino nucleic acid (ANA) sugar moiety, a 2′-fluoro-ANA sugar moiety, a deoxyribose sugar moiety, and a bicyclic nucleic acid), and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, C and/or D, independently, include at least one alternative nucleobase, at least one alternative internucleoside linkage, and/or at least one alternative sugar moiety.

In some embodiments, C includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 1, 4, 7, 10, 13, 16, 19, 22, 25, 28, 31, and 34, and D includes a nucleobase sequence complementary to the nucleobase sequence of C, wherein the sequence includes at least one mismatch as described herein. In other embodiments, D includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 2, 5, 8, 11, 14, 17, 20, 23, 26, 29, 32, and 35, and C includes a nucleobase sequence complementary to the nucleobase sequence of C, wherein the sequence includes at least one mismatch as described herein. In some embodiments, C-L₁-D includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 3, 6, 9, 12, 15, 18, 21, 24, 27, 30, 33, and 36, wherein the sequence includes at least one mismatch as described herein.

Nucleobase sequences of SEQ ID NOs. 1-36 are provided in Table 3:

TABLE 3

| | |
|---|---|
| GGUGAAUAGUAUAACAAUAU | SEQ ID NO. 1 |
| AUGUUGUUAUAGUAUCCACC | SEQ ID NO. 2 |
| GGUGAAUAGUAUAACAAUAUGCUAAAUGUUGUUA | SEQ ID NO. 3 |
| UAGUAUCCACC | |
| GGUGAAGAGGAGAACAAUAU | SEQ ID NO. 4 |
| AUGUUGUUCUCGUCUCCACC | SEQ ID NO. 5 |
| GGUGAAGAGGAGAACAAUAUGCUAAAUGUUGUUC | SEQ ID NO. 6 |
| UCGUCUCCACC | |
| GGUGUCGAGAAGAGGAGAACAAUAU | SEQ ID NO. 7 |
| AUGUUGUUCUCGUCUCCUCGACACC | SEQ ID NO. 8 |
| GGUGUCGAGAAGAGGAGAACAAUAUGCUAAAUGU | SEQ ID NO. 9 |
| UGUUCUCGUCUCCUCGACACC | |
| GGGUGGAAUAGUAUAACAAUAU | SEQ ID NO. 10 |
| AUGUUGUUAUAGUAUCCCACCU | SEQ ID NO. 11 |
| GGGUGGAAUAGUAUAACAAUAUGCUAAAUGUUGU | SEQ ID NO. 12 |
| UAUAGUAUCCCACCU | |
| GUGGAAUAGUAUAACAAUAU | SEQ ID NO. 13 |
| AUGUUGUUAUAGUAUCCCAC | SEQ ID NO. 14 |
| GUGGAAUAGUAUAACAAUAUGCUAAAUGUUGUUA | SEQ ID NO. 15 |
| UAGUAUCCCAC | |
| GGUGUCGAGAAUAGUAUAACAAUAU | SEQ ID NO. 16 |
| AUGUUGUUAUAGUAUCCUCGACACC | SEQ ID NO. 17 |
| GGUGUCGAGAAUAGUAUAACAAUAUGCUAAAUGU | SEQ ID NO. 18 |
| UGUUAUAGUAUCCUCGACACC | |
| GGGUGGAAUAGUAUAACAAUAU | SEQ ID NO. 19 |
| AUGUUGUUAUAGUAUCCCACCU | SEQ ID NO. 20 |
| GGGUGGAAUAGUAUAACAAUAUGCUAAAUGUUGU | SEQ ID NO. 21 |
| UAUAGUAUCCCACCU | |
| GGGUGGAAUAGUAUACCA | SEQ ID NO. 22 |
| UGGUAUAGUAUCCCACCU | SEQ ID NO. 23 |
| GGGUGGAAUAGUAUACCAUUCGUGGUAUAGUAUC | SEQ ID NO. 24 |
| CCACCU | |
| GUGGGUGGAAUAGUAUACCA | SEQ ID NO. 25 |
| UGGUAUAGUAUCCCACCUAC | SEQ ID NO. 26 |
| GUGGGUGGAAUAGUAUACCAUUCGUGGUAUAGUA | SEQ ID NO. 27 |
| UCCCACCUAC | |
| UGGGUGGAAUAGUAUACCA | SEQ ID NO. 28 |
| UGGUAUAGUAUCCCACCUA | SEQ ID NO. 29 |
| UGGGUGGAAUAGUAUACCAUUCGUGGUAUAGUAU | SEQ ID NO. 30 |
| CCCACCUA | |
| GGUGGAAUAGUAUACCA | SEQ ID NO. 31 |
| UGGUAUAGUAUCCCACC | SEQ ID NO. 32 |
| GGUGGAAUAGUAUACCAUUCGUGGUAUAGUAUCC | SEQ ID NO. 33 |
| CACC | |
| GUGGAAUAGUAUACCA | SEQ ID NO. 34 |
| UGGUAUAGUAUCCCAC | SEQ ID NO. 35 |
| GUGGAAUAGUAUACCAUUCGUGGUAUAGUAUCCC | SEQ ID NO. 36 |
| AC | |

It will be understood that, although the sequences in SEQ ID NOs. 1-36 are described as unmodified and/or un-conjugated sequences, the RNA of the oligonucleotides of the invention may include any one of the sequences set forth in SEQ ID NOs. 1-36 that is an alternative nucleoside and/or conjugated as described in detail below.

In some embodiments, the oligonucleotide of the invention may further include a 5′ cap structure. In some embodiments, the 5′ cap structure is a 2,2,7-trimethylguanosine cap.

An oligonucleotide of the invention can be synthesized by standard methods known in the art as further discussed below, e.g., by use of an automated DNA synthesizer, such as are commercially available from, for example, Biosearch, Applied Biosystems, Inc.

The oligonucleotide compound can be prepared using solution-phase or solid-phase organic synthesis or both. Organic synthesis offers the advantage that the oligonucleotide including unnatural or alternative nucleotides can be easily prepared. Single-stranded oligonucleotides of the invention can be prepared using solution-phase or solid-phase organic synthesis or both.

Further, it is contemplated that for any sequence identified herein, further optimization could be achieved by systematically either adding or removing linked nucleosides to generate longer or shorter sequences. Further still, such optimized sequences can be adjusted by, e.g., the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, and/or increasing interaction with RNA editing enzymes (e.g., ADAR)).

In some embodiments, the one or more ADAR-recruiting domains are GluR2 ADAR-recruiting domains. In some embodiments, the GluR2 ADAR-recruiting domain has the nucleotide sequence of SEQ ID NO. 37, as shown below in the 5′ to 3′ direction:

(SEQ ID NO. 37)
GGUGAAUAGUAUAACAAUAUGCUAAAUGUUGUUAUAGUAUCCACC

In some embodiments, the oligonucleotide includes the structure of Formula XXI, as shown below:

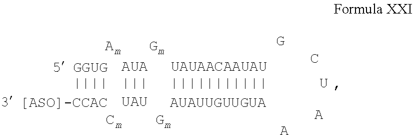

wherein [ASO] includes any of the oligonucleotides of the present invention, wherein m designates a mismatched nucleotide. In some embodiments, the GluR2 ADAR-recruiting domain has the nucleotide sequence of SEQ ID NO. 38, as shown below in the 5′ to 3′ direction:

GGUGAAGAGGAGAACAAUAUGCUAAAUGUUGUUCUCGUCUCCACC (SEQ ID NO. 38)

In some embodiments, the oligonucleotide includes the structure of Formula XXII, as shown below:

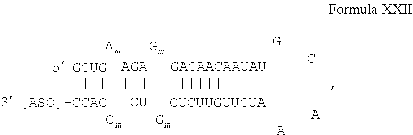

wherein [ASO] includes any of the oligonucleotides of the present invention, wherein m designates a mismatched nucleotide. In some embodiments, the GluR2 ADAR-recruiting domain has the nucleotide sequence of SEQ ID NO. 39, as shown below in the 5′ to 3′ direction:

GGUGUCGAGAAGAGGAGAACAAUAUGCUAAAUGUUGUUCUCGUCUCCUCGACACC (SEQ ID NO. 39)

In some embodiments, the oligonucleotide includes the structure of Formula XXIII, as shown below:

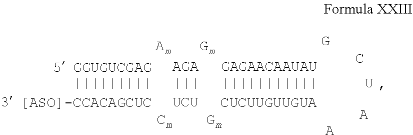

wherein [ASO] includes any of the oligonucleotides of the present invention, wherein m designates a mismatched nucleotide.

In some embodiments, the GluR2 ADAR-recruiting domain has the nucleotide sequence of SEQ ID NO. 40, as shown below in the 5′ to 3′ direction:

(SEQ ID NO. 40)
*s*s*G**GAGAAGAGGAGAA*AA*A*G**AAA*G**G*****G****
***GA*A**

wherein * is a 2′-O-methyl nucleotide and s is a phosphorothioate internucleoside linkage between two linked nucleotides. In some embodiments, the oligonucleotide includes the structure of Formula XXIV, as shown below:

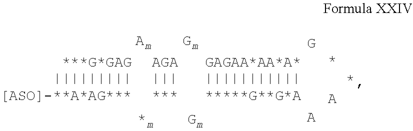

wherein [ASO] includes any one of the oligonucleotides presented herein, wherein * is a 2′-O-methyl nucleotide, wherein s is a phosphorothioate internucleoside linkage, wherein m designates a mismatched nucleotide. In some embodiments, the ADAR-recruiting domains further include at least one nuclease-resistant nucleotide (e.g., 2′-O-methyl nucleotide). In some embodiments, the ADAR-recruiting domains include at least one alternative internucleoside linkage (e.g., a phosphorothioate internucleoside linkage). In some embodiments, the GluR2 ADAR-recruiting domain has the nucleotide sequence of SEQ ID NO. 41, as shown below in the 5′ to 3′ direction:

(SEQ ID NO. 41)
GGGUGGAAUAGUAUAACAAUAUGCUAAAUGUUGUUAUAGUAUCCCACCU

In some embodiments, the oligonucleotide includes the structure of Formula XXV, as shown below:

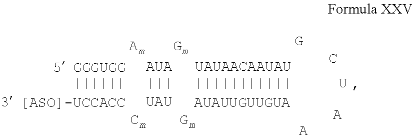

wherein [ASO] includes any of the oligonucleotides of the present invention, wherein m designates a mismatched nucleotide. In some embodiments, the GluR2 ADAR-recruiting domain has the nucleotide sequence of SEQ ID NO. 42, as shown below in the 5′ to 3′ direction:

(SEQ ID NO. 42)
GUGGAAUAGUAUAACAAUAUGCUAAAUGUUGUUAUAGUAUCCCAC

In some embodiments, the oligonucleotide includes the structure of Formula XXVI, as shown below:

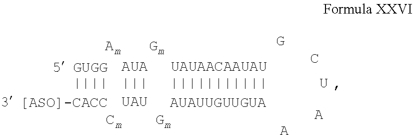

wherein [ASO] includes any of the oligonucleotides of the present invention, wherein m designates a mismatched nucleotide. In some embodiments, the GluR2 ADAR-recruiting domain has the nucleotide sequence of SEQ ID NO. 43, as shown below in the 5′ to 3′ direction:

GGUGUCGAGAAUAGUAUAACAAUAUGCUAAAUGUUGUUAUAGUAUCCUCGACACC (SEQ ID NO. 43)

In some embodiments, the oligonucleotide includes the structure of Formula XXVII, as shown below:

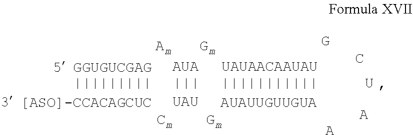

wherein [ASO] includes any of the oligonucleotides of the present invention, wherein m designates a mismatched nucleotide. In some embodiments, the GluR2 ADAR-recruiting domain has the nucleotide sequence of SEQ ID NO. 44, as shown below in the 5′ to 3′ direction:

GGGUGGAAUAGUAUAACAAUAUGCUAAAUGUUGUUAUAGUAUCCCACCU (SEQ ID NO. 44)

In some embodiments, the oligonucleotide includes the structure of Formula XXVIII, as shown below:

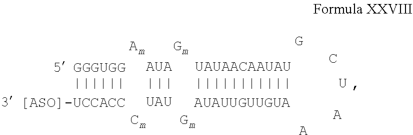

wherein [ASO] includes any of the oligonucleotides of the present invention, wherein m designates a mismatched nucleotide. In some embodiments, the GluR2 ADAR-recruiting domain has the nucleotide sequence of SEQ ID NO. 45, as shown below in the 5′ to 3′ direction:

(SEQ ID NO. 45)
GGGUGGAAUAGUAUACCAUUCGUGGUAUAGUAUCCCACCU

In some embodiments, the oligonucleotide includes the structure of Formula XXIX, as shown below:

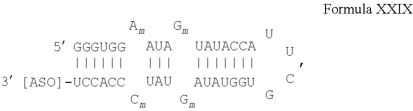

wherein [ASO] includes any of the oligonucleotides of the present invention, wherein m designates a mismatched nucleotide. In some embodiments, the GluR2 ADAR-recruiting domain has the nucleotide sequence of SEQ ID NO. 46, as shown below in the 5′ to 3′ direction:

GUGGGUGGAAUAGUAUACCAUUCGUGGUAUAGUAUCCCACCUAC (SEQ ID NO. 46)

In some embodiments, the oligonucleotide includes the structure of Formula XXX, as shown below:

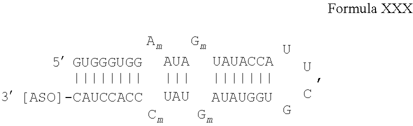

wherein [ASO] includes any of the oligonucleotides of the present invention, wherein m designates a mismatched nucleotide. In some embodiments, the GluR2 ADAR-recruiting domain has the nucleotide sequence of SEQ ID NO. 47, as shown below in the 5′ to 3′ direction:

UGGGUGGAAUAGUAUACCAUUCGUGGUAUAGUAUCCCACCUA (SEQ ID NO. 47)

In some embodiments, the oligonucleotide includes the structure of Formula XXXI, as shown below:

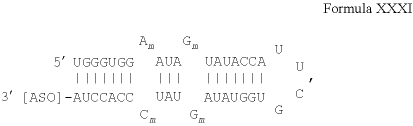

wherein [ASO] includes any of the oligonucleotides of the present invention, wherein m designates a mismatched nucleotide. In some embodiments, the GluR2 ADAR-recruiting domain has the nucleotide sequence of SEQ ID NO. 48, as shown below in the 5′ to 3′ direction:

(SEQ ID NO. 48)
GGUGGAAUAGUAUACCAUUCGUGGUAUAGUAUCCCACC

In some embodiments, the oligonucleotide includes the structure of Formula XXXII, as shown below:

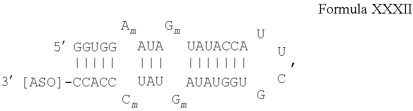

wherein [ASO] includes any of the oligonucleotides of the present invention, wherein m designates a mismatched nucleotide. In some embodiments, the GluR2 ADAR-recruiting domain has the nucleotide sequence of SEQ ID NO. 49, as shown below in the 5′ to 3′ direction:

GUGGAAUAGUAUACCAUUCGUGGUAUAGUAUCCCAC (SEQ ID NO. 49)

In some embodiments, the oligonucleotide includes the structure of Formula XXXIII, as shown below:

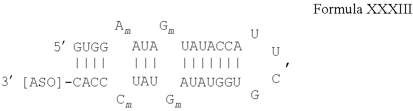

wherein [ASO] includes any of the oligonucleotides of the present invention, wherein m designates a mismatched nucleotide.

In some embodiments, the ADAR-recruiting domains are Z-DNA ADAR-recruiting domains. In some embodiments, the ADAR-recruiting domains are MS2 ADAR-recruiting domains. In some embodiments, an MS2 bacteriophage stem-loop structure may be used as an ADAR-recruiting domain (e.g., and MS2 ADAR-recruiting domain). MS2 stem-loops are known to bind the MS2 bacteriophage coat protein, which when fused to the deaminase domain of ADAR (e.g. an ADAR fusion protein) can be used for target-specific deamination. In some embodiments, the MS2 ADAR-recruiting domain has the nucleotide sequence of SEQ ID NO. 50, as shown below in the 5′ to 3′ direction:

(SEQ ID NO. 50)
ACATGAGGATCACCCATGT

In some embodiments, an ADAR fusion protein is administered to the cell or to the subject using an expression vector construct including a polynucleotide encoding an ADAR fusion protein. In some embodiments, the ADAR fusion protein includes a deaminase domain of ADAR fused to an MS2 bacteriophage coat protein. In some embodiments, the deaminase domain of ADAR is a deaminase domain of ADAR1. In some embodiments, the deaminase domain of ADAR is a deaminase domain of ADAR2. The ADAR fusion protein may be a fusion protein described in Katrekar et al. Nature Methods, 16(3): 239-42 (2019), the ADAR fusion protein of which is herein incorporated by reference

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase may also be an isonucleoside in which the nucleobase is moved from the Cl position of the sugar moiety to a different position (e.g. C2, C3, C4, or C5). Specific examples of oligonucleotide compounds useful in the embodiments described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can also be considered to be oligonucleosides. In some embodiments, an oligonucleotide will have a phosphorus atom in its internucleoside backbone.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides of the invention are described in, for example, in Nielsen et al., Science, 1991, 254, 1497-1500.

Some embodiments featured in the invention include oligonucleotides with phosphorothioate backbones and oligonucleotides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂— ] of the above-referenced U.S. Pat. No. 5,489,677, and the amide backbones of the above-referenced U.S. Pat. No. 5,602,240. In some embodiments, the oligonucleotides featured herein have morpholino backbone structures of the above-referenced U.S. Pat. No. 5,034,506. In other embodiments, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can also contain one or more substituted sugar moieties. The oligonucleotides, e.g., oligonucleotides, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)ₙCH₃]₂, where n and m are from 1 to about 10. In other embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-O-MOE) (Martin et al., Helv. Chin. Acta, 1995, 78:486-504) i.e., an alkoxy-alkoxy group. 2′-O-MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternatives include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can also be made at other positions on the nucleosides and nucleotides of an oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920. The entire contents of each of the foregoing are hereby incorporated herein by reference.

An oligonucleotide for use in the methods of the present invention can also include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytosine, 5-hydroxymethylcytosine, 5-formylcytosine, 5-carboxycytosine, pyrrolocytosine, dideoxycytosine, uracil, 5-methoxyuracil, 5-hydroxydeoxyuracil, dihydrouracil, 4-thiouracil, pseudouracil, 1-methyl-pseudouracil, deoxyuracil, 5-hydroxybutynl-2′-deoxyuracil, xanthine, hypoxanthine, 7-deaza-xanthine, thienoguanine, 8-aza-7-deazaguanine, 7-methylguanine, 7-deazaguanine, 6-aminomethyl-7-deazaguanine, 8-aminoguanine, 2,2,7-trimethylguanine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other embodiments, the sugar moiety in the nucleotide may be a ribose molecule, optionally having a 2′-O-methyl, 2′-O-MOE, 2′-F, 2′-amino, 2′-O-propyl, 2′-aminopropyl, or 2′-OH modification.

An oligonucleotide for use in the methods of the present invention can include one or more bicyclic sugar moieties. A “bicyclic sugar” is a furanosyl ring modified by the bridging of two atoms. A “bicyclic nucleoside” (“BNA”) is a nucleoside having a sugar moiety including a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleosides. A locked nucleoside is a nucleoside having a modified ribose moiety in which the ribose moiety includes an extra bridge connecting the 2′ and 4′ carbons. In other words, a locked nucleoside is a nucleoside including a bicyclic sugar moiety including a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the polynucleotides of the invention include without limitation nucleosides including a bridge between the 4′ and the 2′ ribosyl ring atoms. In certain embodiments, the polynucleotide agents of the invention include one or more bicyclic nucleosides including a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An oligonucleotide for use in the methods of the invention can also be modified to include one or more constrained ethyl nucleotides. As used herein, a “constrained ethyl nucleotide” or “cEt” is a locked nucleic acid including a bicyclic sugar moiety including a 4′-CH(CH3)-O-2′ bridge. In one embodiment, a constrained ethyl nucleotide is in the S conformation referred to herein as “S-cEt.”

An oligonucleotide for use in the methods of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide for use in the methods of the invention includes one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

The ribose molecule can also be replaced with non-sugars such as cyclohexene to produce cyclohexene nucleic acid (CeNA) or glycol to produce glycol nucleic acids (GNA).Potentially stabilizing modifications to the ends of nucleotide molecules can include N-(acetylaminocaproyl)-4-hydroxyprolinol (Hyp-C₆-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Exemplary oligonucleotides for use in the methods of the invention include sugar-modified nucleosides and may also include DNA or RNA nucleosides. In some embodiments, the oligonucleotide includes sugar-modified nucleosides and DNA nucleosides. Incorporation of alternative nucleosides into the oligonucleotide of the invention may enhance the affinity of the oligonucleotide for the target nucleic acid. In that case, the alternative nucleosides can be referred to as affinity enhancing alternative nucleotides.

In some embodiments, the oligonucleotide includes at least 1 alternative nucleoside, such as at least 2, at least 3, at least 4, at least 5, at least 6, at least 7, at least 8, at least 9, at least 10, at least 11, at least 12, at least 13, at least 14, at least 15 or at least 16 alternative nucleosides. In other embodiments, the oligonucleotides include from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In an embodiment, the oligonucleotide of the invention may include alternatives, which are independently selected from these three types of alternative (alternative sugar moiety, alternative nucleobase, and alternative internucleoside linkage), or a combination thereof. Preferably the oligonucleotide includes one or more nucleosides including alternative sugar moieties, e.g., 2′ sugar alternative nucleosides. In some embodiments, the oligonucleotide of the invention include the one or more 2′ sugar alternative nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, ANA, 2′-fluoro-ANA, and BNA (e.g., LNA) nucleosides. In some embodiments, the one or more alternative nucleoside is a BNA.

In some embodiments, at least 1 of the alternative nucleosides is a BNA (e.g., an LNA), such as at least 2, such as at least 3, at least 4, at least 5, at least 6, at least 7, or at least 8 of the alternative nucleosides are BNAs. In a still further embodiment, all the alternative nucleosides are BNAs.

In a further embodiment the oligonucleotide includes at least one alternative internucleoside linkage. In some embodiments, the internucleoside linkages within the contiguous nucleotide sequence are phosphorothioate or boronophosphate internucleoside linkages. In some embodiments, all the internucleotide linkages in the contiguous sequence of the oligonucleotide are phosphorothioate linkages. In some embodiments the phosphorothioate linkages are stereochemically pure phosphorothioate linkages. In some embodiments, the phosphorothioate linkages are Sp phosphorothioate linkages. In other embodiments, the phosphorothioate linkages are Rp phosphorothioate linkages.

In some embodiments, the oligonucleotide for use in the methods of the invention includes at least one alternative nucleoside which is a 2′-O-MOE-RNA, such as 2, 3, 4, 5, 6, 7, 8, 9, or 10 2′-O-MOE-RNA nucleoside units. In some embodiments, the 2′-O-MOE-RNA nucleoside units are connected by phosphorothioate linkages. In some embodiments, at least one of said alternative nucleoside is 2′-fluoro DNA, such as 2, 3, 4, 5, 6, 7, 8, 9, or 10 2′-fluoro-DNA nucleoside units. In some embodiments, the oligonucleotide of the invention includes at least one BNA unit and at least one 2′ substituted alternative nucleoside. In some embodiments of the invention, the oligonucleotide includes both 2′ sugar modified nucleosides and DNA units.

B. Oligonucleotide Conjugated to Ligands

Oligonucleotides for use in the methods of the invention may be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one embodiment, a ligand alters the distribution, targeting, or lifetime of an oligonucleotide agent into which it is incorporated. In some embodiments, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-i sopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic ionizable lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that include a number of phosphorothioate linkages are also known to bind to serum protein, thus short oligonucleotides, e.g., oligonucleotides of about 5 bases, 10 bases, 15 bases, or 20 bases, including multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides of the present invention, such as the ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid-based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In another aspect, the ligand is a moiety, e.g., a vitamin, which is taken up by a target cell, e.g., a proliferating cell. Exemplary vitamins include vitamin A, E, and K.

ii. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO. 87). An RFGF analogue (e.g., amino acid sequence AALLPVLLAAP (SEQ ID NO. 88) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ; SEQ ID NO. 89) and the Drosophila Antennapedia protein (RQIKIWFQNRRMKWKK; SEQ ID NO. 90) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an a-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some embodiments of the compositions and methods of the invention, an oligonucleotide further includes a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the invention is a monosaccharide.

In some embodiments, the carbohydrate conjugate further includes one or more additional ligands as described above, such as, but not limited to, a PK modulator and/or a cell permeation peptide.

Additional carbohydrate conjugates (and linkers) suitable for use in the present invention include those described in PCT Publication Nos. WO 2014/179620 and WO 2014/179627, the entire contents of each of which are incorporated herein by reference.

iv. Linkers

In some embodiments, the conjugate or ligand described herein can be attached to an oligonucleotide with various linkers that can be cleavable or non-cleavable.

Linkers typically include a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

a. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one embodiment, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another embodiment, a cleavable linker includes a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—. These candidates can be evaluated using methods analogous to those described above.

c. Acid Cleavable Linking Groups

In another embodiment, a cleavable linker includes an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another embodiment, a cleavable linker includes an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another embodiment, a cleavable linker includes a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In one embodiment, an oligonucleotide of the invention is conjugated to a carbohydrate through a linker. Linkers include bivalent and trivalent branched linker groups. Exemplary oligonucleotide carbohydrate conjugates with linkers of the compositions and methods of the invention include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an amino linker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### IV. Pharmaceutical Compositions

The present disclosure also includes pharmaceutical compositions and formulations which include the oligonucleotides of the disclosure. In one embodiment, provided herein are pharmaceutical compositions containing an oligonucleotide, e.g., a guide oligonucleotide, as described herein, and a pharmaceutically acceptable carrier. The pharmaceutical compositions containing the oligonucleotide are useful for treating a subject who would benefit from editing a target gene, e.g., a SERPINA1 polynucleotide with a SNP associated with alpha 1 antitrypsin deficiency.

The pharmaceutical compositions of the present disclosure can be administered in a number of ways depending upon whether local or systemic treatment is desired and upon the area to be treated. Administration can be oral, parental, topical (e.g., by a transdermal patch), intranasal, intratracheal, epidermal and transdermal.

Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. Parenteral administration may be by continuous infusion over a selected period of time.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the oligonucleotides featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides featured in the disclosure can be encapsulated within liposomes or can form complexes thereto, in particular to cationic liposomes. Alternatively, Oligonucleotides can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Useful solutions for oral or parenteral administration can be prepared by any of the methods well known in the pharmaceutical art, described, for example; in Remington's Pharmaceutical Sciences, 18th ed. (Mack Publishing Company, 1990). The parenteral preparation can be enclosed in ampoules, disposable syringes or multiple dose vials made of glass or plastic. Formulations also can include, for example, polyalkylene glycols such as polyethylene glycol, oils of vegetable origin, and hydrogenated naphthalenes. Other potentially useful parenteral carriers for these drugs include ethylene-vinyl acetate copolymer particles, osmotic pumps, implantable infusion systems, and liposomes.

Formulations of the present disclosure suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding; in a suitable machine; a mixture of the powdered drug and suitable carrier moistened with an inert liquid diluent.

Oral compositions generally include an inert diluent or an edible carrier. For the purpose of oral therapeutic administration, the active compound can be incorporated with excipients. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water; ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions; methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystal line form, for example, in the form of an aqueous microcrystalline suspension. Liposomal formulations or biodegradable polymer systems may also be used to present the drug for both intra-articular and ophthalmic administration.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used; such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where the active compound is to be used as part of a transplant procedure, it can be provided to the living tissue or organ to be transplanted prior to removal of tissue or organ from the donor. The compound can be provided to the donor host. Alternatively, or in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

The compositions of the present disclosure can also be prepared and formulated in additional formulations, such as emulsions or microemulsions, or be incorporated into a particle, e.g., a microparticle, which can be produced by spray-drying, or other methods including lyophilization, evaporation, fluid bed drying, vacuum drying, or a combination of these techniques. Penetration enhancers, e.g., surfactants, fatty acids, bile salts, chelating agents, and non-chelating non-surfactants, may be added in order to effect the efficient delvery of the compositions of the present disclosure, e.g., the delivery of the oligonucleotides, to the subject. Agents that enhance uptake of oligonucletide agents at the cellular level can also be added to the pharmaceutical and other compositions of the present disclosure, such as, cationic lipids, e.g., lipofectin, cationic glycerol derivatives, and polycationic molecules, e.g., polylysine.

The pharmaceutical composition of the present disclosure may also include a pharmaceutical carrier or excipient. A pharmarceutical carrier or excipient is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Toxicity and therapeutic efficacy of the compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). Compounds that exhibit high therapeutic indices are preferred. The data obtained from cell culture assays and animal studies can be used in formulating a range of dosage for use in humans.

The dosage of the compositions (e.g., a composition including an oligonucleotide) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine whether to administer the composition and tailor the appropriate dosage and/or therapeutic regimen of treatment with the composition based on the above factors. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount. In some embodiments, treatment of a subject with a therapeutically effective amount of a composition can include a single treatment or a series of treatments. In addition, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

The pharmaceutical compositions of the disclosure may be administered in dosages sufficient to edit a target gene, e.g., a SERPINA1 polynucleotide, and/or treat alpha 1 antitrypsin deficiency. In therapeutic use for treating, preventing, or combating alpha 1 antitrypsin deficiency in subjects, the compounds or pharmaceutical compositions thereof will be administered orally or parenterally at a dosage to obtain and maintain a concentration, that is, an amount, or blood-level or tissue level of active component in the animal undergoing treatment which will be effective. The term “effective amount” is understood to mean that the compound of the disclosure is present in or on the recipient in an amount sufficient to elicit biological activity. Generally, an effective amount of dosage of active component will be in the range of from about 1 μg/kg to about 100 mg/kg, preferably from about 10 μg/kg to about 10 mg/kg, more preferably from about 100 μg/kg to about 1 mg/kg of body weight per day.

### V. Kits

In certain aspects, the instant disclosure provides kits that include a pharmaceutical formulation including an oligonucleotide agent capable of effecting an adenosine deaminase acting on RNA (ADAR)-mediated adenosine to inosine alteration of a SNP associated with a disease, e.g., alpha 1 antitrypsin deficiency, and a package insert with instructions to perform any of the methods described herein.

In some embodiments, the kits include instructions for using the kit to edit a SERPINA1 polynucleotide comprising a SNP associated with alpha 1 antitrypsin deficiency. In other embodiments, the kits include instructions for using the kit to edit a SERPINA1 polynucleotide comprising a SNP associated with alpha 1 antitrypsin deficiency and to treat alpha 1 antitrypsin deficiency. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including an oligonucleotide agent capable of effecting an ADAR-mediated adenosine to inosine alteration of a SNP associated with a disease, e.g., alpha 1 antitrypsin deficiency, an additional therapeutic agent, and a package insert with instructions to perform any of the methods described herein.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres, as described herein. The kit can further include a delivery device, e.g., for delivery to the [central nervous system], such as needles, syringes, pumps, and package inserts with instructions for use.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1. Reversing an Amino Acid Substitution Mutation E342K in the SERPINA1 Transcript by Targeted A to I Editing for the Treatment of Alpha 1 Antitrypsin Deficiency

Human ADAR2 sequence (NM_001112.4), Human ADAR1p110 (NM_001111.5) and mutant SERPINA1 sequences (ORF only) were cloned into pcDNA3.1 plasmid under the control of the CMV promoter using BamHI and Xbal restriction sites (Quintara Bio, Berkeley, Calif.) and the correct insert was sequence verified. The plasmids are referred to as ADAR2/pcDNA3.1, ADAR1p110/pcDNA3.1, or SERPINA1/pcDNA3.1. For editing experiments, 2μg of ADAR2/pcDNA3.1 or ADAR1p110/pcDNA3.1 plasmid and bug of SERPINA1/pcDNA3.1 plasmid were transfected into 5×10⁶ HEK293T cells (ATCC) using 25 μL of Lipofectamine 3000 and 24 μL of P3000 (Life Technologies) per 10 cm dish. After 4 hours, the culture media was replenished with fresh warmed media (DMEM High Glucose; Life Technologies). 12-16 hours after transfection, the transfected HEK293T cells were transfected with guide oligonucleotides such that the final concentration in the each well was 100 nM. All transfections were carried out with Lipofectamine 3000 (0.4 μL/per well) in a 96-well format according to manufacturer's instructions. 12-16 hours after the second transfection, the cells were washed once with ice cold PBS and total mRNA isolation was performed using Dyna Beads mRNA Direct Kit (Life Technologies) adapted for KingFisher Flex Purification (Life Technologies) according to manufacturer's instructions. The samples were treated with TURBO DNase (Life Technologies) prior to elution. The resultant isolated mRNA was used for cDNA synthesis using SuperScript IV Vilo according to the manufacturer's instructions (Life Technologies). One μl of the cDNA was used as template for PCR (Platinum II Hot-Start PCR Master Mix; Life Technologies) using gene specific primers to generate an amplicon for Sanger sequencing (Table 4). Sanger sequencing was performed by Quintara Biosciences (Berkeley, Calif.). Adenosine to inosine editing yields were quantified by measuring the peak height of adenosine and guanosine and dividing the guanosine peak height by the total peak height measurements of adenosine and guanosine combined.

TABLE 4
Primer List

| Name | Sequence 5′-3′ | SEQ ID NO |
|---|---|---|
| SERPINA1 fwd | AGCACCTGGAAAATGAACTC | 51 |
| SERPINA1 rvs | TTTTTTTGCGATGCAATTTCC | 52 |

The guide oligonucleotide sequences tested are shown in Table 5-19. These tables also show the editing results for certain guide oligonucleotide co-transfected with either ADAR1 or ADAR2 or both.

The following abbreviations are used to indicate modifications in the oligonucleotide sequences.

| Modification | Abbreviation |
|---|---|
| RNA | rN |
| DNA | dN |
| 2′-O-Methyl(2′-OMe) | mN |
| 2′-F-arabinose (FANA) | fN |
| 2′-F-RNA | FN |
| 2′-O-Methoxyethyl (MOE) | MN |
| Locked Nucleic Acids (LNA) | LN |
| Abasic | dS |
| RNA-Abasic | rS |
| 2′-OMe-Abasic | mS |
| 5-Methyl-rC | rmC |
| 5-Methyl-dC | dmC |
| 5-Methyl-2′-OMe-C | mmC |
| (S)-Glycol Nucleic Acid (S-GNA) | sgN |
| (R)-Glycol Nucleic Acid (R-GNA) | rgN |
| DNA α-anomer | αN |
| Arabinose | aN |
| 2′-OMe-Arabinose | amN |
| Flexible Nucleic Acid (FNA) | fxN |
| Serineol Nucleic Acid | sN |
| β-D-homoDNA | hdN |
| β-D-HNA | hN |
| Phosphorothioate internucleoside | * |
| linkage | |

TABLE 5
SERPINA1 Antisense-gRNA

| | | | Editing Data | | | | | | | |
|---|---|---|---|---|---|---|---|---|---|---|
| | SEQ | | ADAR2 % Editing | | | | ADAR1 p110 % Editing | | | |
| Seq ID | ID NO | Guide Oligonucleotide Sequences | 100 nM | SD | 10 nM | SD | 100 nM | SD | 10 nM | SD |
| | | Human SERPINA1 E342K Antisense-gRNA-with Varied Lengths | | | | | | | | |
| KB-018-001 | 53 | 5′-mC*mU*mA*mA*mAmAmAmCmAmUmGmGmCmCmCmCmAmGmCm | 42.8 | 1.4 | 33.4 | 2.4 | 21.0 | 3.3 | 6.6 | 0.9 |
| | | AmGmCmUmUmCmAmGmUmCmCmCmUmUmUmCdTdCdGmUmCmG | | | | | | | | |
| | | mAmUmGmGmU*mC*mA*mG*mC-3′ | | | | | | | | |
| KB-018-002 | 54 | 5′-mA*mA*mC*mA*mUmGmGmCmCmCmCmAmGmCmAmGmCmUmUm | 37.7 | 11.4 | 35.5 | 3.1 | | | | |
| | | CmAmGmUmCmCmCmUmUmUmCdTdCdGmUmCmGmAmUmGmGmU* | | | | | | | | |
| | | mC*mA*mG*mC-3′ | | | | | | | | |
| KB-018-003 | 55 | 5′-mG*mG*mC*mC*mCmCmAmGmCmAmGmCmUmUmCmAmGmUmCm | 40.9 | 4.8 | 15.4 | 1.2 | | | | |
| | | CmCmUmUmUmCdTdCdGmUmCmGmAmUmGmGmU*mC*mA*mG*mC- | | | | | | | | |
| | | 3′ | | | | | | | | |
| KB-018-004 | 56 | 5′-mC*mA*mG*mC*mAmGmCmUmUmCmAmGmUmCmCmCmUmUmUm | 31.1 | 4.4 | 13.3 | 4.2 | | | | |
| | | CdTdCdGmUmCmGmAmUmGmGmU*mC*mA*mG*mC-3′ | | | | | | | | |
| KB-018-005 | 57 | 5′-mA*mG*mC*mU*mUmCmAmGmUmCmCmCmUmUmUmCdTdCdGmU | 19.6 | 4.0 | 3.8 | 1.0 | | | | |
| | | mCmGmAmUmGmGmU*mC*mA*mG*mC-3′ | | | | | | | | |
| KB-018-006 | 58 | 5′-mC*mU*mU*mC*mAmGmUmCmCmCmUmUmUmCdTdCdGmUmCmG | 9.1 | 1.2 | 1.7 | 0.4 | | | | |
| | | mAmUmGmGmU*mC*mA*mG*mC-3′ | | | | | | | | |
| KB-018-007 | 59 | 5′-mC*mA*mG*mU*mCmCmCmUmUmUmCdTdCdGmUmCmGmAmUmG | 3.8 | 0.5 | 1.3 | 0.6 | | | | |
| | | mG*mU*mC*mA*mG-3′ | | | | | | | | |
| KB-018-008 | 60 | 5′-mA*mG*mU*mC*mCmCmUmUmUmCdTdCdGmUmCmGmAmUmG*m | 3.5 | 0.2 | 1.3 | 0.1 | | | | |
| | | G*mU*mC*mA-3′ | | | | | | | | |
| | | Human SERPINA1 E432K Antisense-gRNA with | | | | | | | | |
| | | varied lengths and FANA modifications | | | | | | | | |
| KB-018-026 | 61 | 5′-mC*mU*mA*mA*mAmAmAmCmAmUmGmGmCmCmCmCmAmGmCm | 67.3 | 2.3 | 32.6 | 2.9 | 27.9 | 7.3 | 8.3 | 1.8 |
| | | AmGmCmUmUmCmAmGmUmCmCmCmUmUmUmCfUfCfGmUmCmGm | | | | | | | | |
| | | AmUmGmGmU*mC*mA*mG*mC-3′ | | | | | | | | |
| KB-018-364 | 62 | 5′-mA*mA*mC*mA*mUmGmGmCmCmCmCmAmGmCmAmGmCmUmUm | 67.4 | 2.3 | 29.8 | 3.8 | | | | |
| | | CmAmGmUmCmCmCmUmUmUmCfUfCfGmUmCmGmAmUmGmGmU* | | | | | | | | |
| | | mC*mA*mG*mC-3′ | | | | | | | | |
| KB-018-365 | 63 | 5′-mG*mG*mC*mC*mCmCmAmGmCmAmGmCmUmUmCmAmGmUmCm | 53.5 | 4.6 | 13.2 | 2.9 | | | | |
| | | CmCmUmUmUmCfUfCfGmUmCmGmAmUmGmGmU*mC*mA*mG*mC-3′ | | | | | | | | |
| KB-018-366 | 64 | 5′-mC*mA*mG*mC*mAmGmCmUmUmCmAmGmUmCmCmCmUmUmUm | 40.1 | 7.4 | 10.3 | 2.2 | | | | |
| | | CfUfCfGmUmCmGmAmUmGmGmU*mC*mA*mG*mC-3′ | | | | | | | | |
| KB-018-367 | 65 | 5′-mA*mG*mC*mU*mUmCmAmGmUmCmCmCmUmUmUmCfUfCfGmUm | 33.7 | 12.7 | 5.9 | 3.2 | | | | |
| | | CmGmAmUmGmGmU*mC*mA*mG*mC-3′ | | | | | | | | |

TABLE 6
SERPINA1 Triplet Modifications

| | | | Editing Data | | | | | | | |
|---|---|---|---|---|---|---|---|---|---|---|
| | SEQ | | ADAR2 % Editing | | | | ADAR1 p110 % Editing | | | |
| Seq ID | ID NO | Guide Oligonucleotide Sequences | 100 nM | SD | 10 nM | SD | 100 nM | SD | 10 nM | SD |
| | | SERPINA1-E342K Antisense-gRNA with DNA/RNA Triplet | | | | | | | | |
| KB-018-074 | 66 | 5′-mC*mU*mA*mA*mAmAmAmCmAmUmGmGmCmCmCmCmAmG | | | | | 17.7 | 2.8 | 3.5 | 3.3 |
| | | mCmAmGmCmUmUmCmAmGmUmCmCmCmUmUmUmCrUrCrGm | | | | | | | | |
| | | UmCmGmAmUmGmGmU*mC*mA*mG*mC-3′ | | | | | | | | |
| KB-018-075 | 67 | 5′-mC*mU*mA*mA*mAmAmAmCmAmUmGmGmCmCmCmCmAmG | | | | | 20.4 | 1.7 | 7.0 | 0.6 |
| | | mCmAmGmCmUmUmCmAmGmUmCmCmCmUmUmUmCrUdCrGm | | | | | | | | |
| | | UmCmGmAmUmGmGmU*mC*mA*mG*mC-3′ | | | | | | | | |
| KB-018-015 | 68 | 5′-mC*mU*mA*mA*mAmAmAmCmAmUmGmGmCmCmCmCmAmG | 26.5 | 4.5 | 22.5 | 3.1 | 21.4 | 0.8 | 5.4 | 1.4 |
| | | mCmAmGmCmUmUmCmAmGmUmCmCmCmUmUmUmCdTdCrG | | | | | | | | |
| | | mUmCmGmAmUmGmGmU*mC*mA*mG*mC-3′ | | | | | | | | |
| KB-018-016 | 69 | 5′-mC*mU*mA*mA*mAmAmAmCmAmUmGmGmCmCmCmCmAmG | 20.5 | 2.5 | 7.3 | 10.3 | 18.3 | 5.5 | 5.8 | 0.8 |
| | | mCmAmGmCmUmUmCmAmGmUmCmCmCmUmUmUmCrUdCdG | | | | | | | | |
| | | mUmCmGmAmUmGmGmU*mC*mA*mG*mC-3′ | | | | | | | | |
| KB-018-017 | 70 | 5′-mC*mU*mA*mA*mAmAmAmCmAmUmGmGmCmCmCmCmAmG | 28.1 | 3.5 | 22.1 | 3.1 | 36.4 | 0.7 | 10.9 | 0.4 |
| | | mCmAmGmCmUmUmCmAmGmUmCmCmCmUmUmUmCrUrCdGm | | | | | | | | |
| | | UmCmGmAmUmGmGmU*mC*mA*mG*mC-3′ | | | | | | | | |
| KB-018-093 | 71 | 5′-mC*mU*mA*mA*mAmAmAmCmAmUmGmGmCmCmCmCmAmG | | | | | | | | |
| | | mCmAmGmCmUmUmCmAmGmUmCmCmCmUmUmUmCrUrCrGm | | | | | | | | |
| | | UmCmGmAmUmGmGmU*mC*mA*mG*mC-3′ | | | | | | | | |
| | | SERPINA1-E342K Antisense-gRNA with DNA/2-OMe | | | | | | | | |
| | | modifications in Triplet | | | | | | | | |
| KB-018-018 | 72 | 5′-mC*mU*mA*mA*mAmAmAmCmAmUmGmGmCmCmCmCmAmG | 41.2 | | 27.1 | | | | | |
| | | mCmAmGmCmUmUmCmAmGmUmCmCmCmUmUmUmCdTdCmG | | | | | | | | |
| | | mUmCmGmAmUmGmGmU*mC*mA*mG*mC-3′ | | | | | | | | |
| KB-018-019 | 73 | 5′-mC*mU*mA*mA*mAmAmAmCmAmUmGmGmCmCmCmCmAmG | 25.7 | | 13.5 | | | | | |
| | | mCmAmGmCmUmUmCmAmGmUmCmCmCmUmUmUmCdTmCm | | | | | | | | |
| | | GmUmCmGmAmUmGmGmU*mC*mA*mG*mC-3′ | | | | | | | | |
| KB-018-020 | 74 | 5′-mC*mU*mA*mA*mAmAmAmCmAmUmGmGmCmCmCmCmAmG | 41.6 | | 28.8 | | | | | |
| | | mCmAmGmCmUmUmCmAmGmUmCmCmCmUmUmUmCdTmCdG | | | | | | | | |
| | | mUmCmGmAmUmGmGmU*mC*mA*mG*mC-3′ | | | | | | | | |
| KB-018-021 | 75 | 5′-mC*mU*mA*mA*mAmAmAmCmAmUmGmGmCmCmCmCmAmG | 24.0 | | 11.9 | | | | | |
| | | mCmAmGmCmUmUmCmAmGmUmCmCmCmUmUmUmCmUmCd | | | | | | | | |
| | | GmUmCmGmAmUmGmGmU*mC*mA*mG*mC-3′ | | | | | | | | |
| KB-018-022 | 76 | 5′-mC*mU*mA*mA*mAmAmAmCmAmUmGmGmCmCmCmCmAmG | 49.2 | | 10.7 | | | | | |
| | | mCmAmGmCmUmUmCmAmGmUmCmCmCmUmUmUmCmUdCdG | | | | | | | | |
| | | mUmCmGmAmUmGmGmU*mC*mA*mG*mC-3′ | | | | | | | | |
| KB-018-023 | 77 | 5′-mC*mU*mA*mA*mAmAmAmCmAmUmGmGmCmCmCmCmAmG | 25.4 | | 21.2 | | | | | |
| | | mCmAmGmCmUmUmCmAmGmUmCmCmCmUmUmUmCmUdCm | | | | | | | | |
| | | GmUmCmGmAmUmGmGmU*mC*mA*mG*mC-3′ | | | | | | | | |
| KB-018-010 | 78 | 5′-mC*mU*mA*mA*mAmAmAmCmAmUmGmGmCmCmCmCmAmG | 8.1 | 1.0 | | | | | | |
| | | mCmAmGmCmUmUmCmAmGmUmCmCmCmUmUmUmCmUmCm | | | | | | | | |
| | | GmUmCmGmAmUmGmGmU*mC*mA*mG*mC-3′ | | | | | | | | |
| | | SERPINA1-E342K Antisense-gRNA with FANA modifications | | | | | | | | |
| | | in Triplet | | | | | | | | |
| KB-018-024 | 79 | 5′-mC*mU*mA*mA*mAmAmAmCmAmUmGmGmCmCmCmCmAmG | 58.3 | 4.2 | 25.0 | 2.0 | 23.8 | 3.3 | 7.3 | 0.9 |
| | | mCmAmGmCmUmUmCmAmGmUmCmCmCmUmUmUmCdTdCfG | | | | | | | | |
| | | mUmCmGmAmUmGmGmU*mC*mA*mG*mC-3′ | | | | | | | | |
| KB-018-025 | 80 | 5′-mC*mU*mA*mA*mAmAmAmCmAmUmGmGmCmCmCmCmAmG | 63.8 | 5.4 | 23.3 | 4.2 | 23.8 | 3.6 | 6.6 | 1.8 |
| | | mCmAmGmCmUmUmCmAmGmUmCmCmCmUmUmUmCdTfCfGm | | | | | | | | |
| | | UmCmGmAmUmGmGmU*mC*mA*mG*mC-3′ | | | | | | | | |
| KB-018-026 | 81 | 5′-mC*mU*mA*mA*mAmAmAmCmAmUmGmGmCmCmCmCmAmG | 64.7 | 1.2 | 34.4 | 1.2 | 27.9 | 7.3 | 8.3 | 1.8 |
| | | mCmAmGmCmUmUmCmAmGmUmCmCmCmUmUmUmCfUfCfGm | | | | | | | | |
| | | UmCmGmAmUmGmGmU*mC*mA*mG*mC-3′ | | | | | | | | |
| KB-018-027 | 82 | 5′-mC*mU*mA*mA*mAmAmAmCmAmUmGmGmCmCmCmCmAmG | 62.7 | 2.9 | | | 30.1 | 2.0 | | |
| | | mCmAmGmCmUmUmCmAmGmUmCmCmCmUmUmUmCdTfCdG | | | | | | | | |
| | | mUmCmGmAmUmGmGmU*mC*mA*mG*mC-3′ | | | | | | | | |
| KB-018-028 | 83 | 5′-mC*mU*mA*mA*mAmAmAmCmAmUmGmGmCmCmCmCmAmG | 55.1 | 2.2 | 20.3 | 0.8 | 26.4 | 3.1 | 10.2 | 0.7 |
| | | mCmAmGmCmUmUmCmAmGmUmCmCmCmUmUmUmCfUfCdGm | | | | | | | | |
| | | UmCmGmAmUmGmGmU*mC*mA*mG*mC-3′ | | | | | | | | |
| KB-018-029 | 84 | 5′-mC*mU*mA*mA*mAmAmAmCmAmUmGmGmCmCmCmCmAmG | 55.0 | 6.2 | 27.2 | 1.5 | 18.4 | 1.3 | 7.2 | 0.7 |
| | | mCmAmGmCmUmUmCmAmGmUmCmCmCmUmUmUmCfUdCdG | | | | | | | | |
| | | mUmCmGmAmUmGmGmU*mC*mA*mG*mC-3′ | | | | | | | | |
| KB-018-030 | 85 | 5′-mC*mU*mA*mA*mAmAmAmCmAmUmGmGmCmCmCmCmAmG | 50.6 | 4.9 | 18.8 | 2.3 | 30.4 | 1.2 | 10.5 | 0.5 |
| | | mCmAmGmCmUmUmCmAmGmUmCmCmCmUmUmUmCfUdCfGm | | | | | | | | |
| | | UmCmGmAmUmGmGmU*mC*mA*mG*mC-3′ | | | | | | | | |
| KB-018-031 | 91 | 5′-mC*mU*mA*mA*mAmAmAmCmAmUmGmGmCmCmCmCmAmG | 27.4 | 3.1 | 15.3 | | 11.4 | 1.1 | 6.4 | 1.4 |
| | | mCmAmGmCmUmUmCmAmGmUmCmCmCmUmUmUmCmUfCmG | | | | | | | | |
| | | mUmCmGmAmUmGmGmU*mC*mA*mG*mC-3′ | | | | | | | | |
| KB-018-032 | 92 | 5′-mC*mU*mA*mA*mAmAmAmCmAmUmGmGmCmCmCmCmAmG | 37.4 | 4.4 | 16.1 | 5.8 | 13.4 | | 6.4 | 1.5 |
| | | mCmAmGmCmUmUmCmAmGmUmCmCmCmUmUmUmCfUfCmG | | | | | | | | |
| | | mUmCmGmAmUmGmGmU*mC*mA*mG*mC-3′ | | | | | | | | |
| KB-018-033 | 93 | 5′-mC*mU*mA*mA*mAmAmAmCmAmUmGmGmCmCmCmCmAmG | 55.3 | 1.5 | 21.3 | 2.1 | 23.9 | 1.2 | 11.9 | 0.8 |
| | | mCmAmGmCmUmUmCmAmGmUmCmCmCmUmUmUmCmUfCfG | | | | | | | | |
| | | mUmCmGmAmUmGmGmU*mC*mA*mG*mC-3′ | | | | | | | | |
| KB-018-203 | 94 | 5′-mC*mU*mA*mA*mAmAmAmCmAmUmGmGmCmCmCmCmAmG | 53.2 | 2.8 | 18.5 | 1.1 | 20.2 | 2.2 | 8.8 | 2.9 |
| | | mCmAmGmCmUmUmCmAmGmUmCmCmCmUmUmUmCfUmCfG | | | | | | | | |
| | | mUmCmGmAmUmGmGmU*mC*mA*mG*mC-3′ | | | | | | | | |
| KB-018-204 | 95 | 5′-mC*mU*mA*mA*mAmAmAmCmAmUmGmGmCmCmCmCmAmG | 46.9 | 1.4 | 15.2 | 1.9 | 21.5 | 2.0 | 5.2 | 1.0 |
| | | mCmAmGmCmUmUmCmAmGmUmCmCmCmUmUmUmCdTmCfG | | | | | | | | |
| | | mUmCmGmAmUmGmGmU*mC*mA*mG*mC-3′ | | | | | | | | |
| KB-018-697 | 96 | 5′-mC*mU*mA*mA*mAmAmAmCmAmUmGmGmCmCmCmCmAmG | 24.0 | 0.8 | 14.8 | 0.0 | 19.8 | 1.1 | 10.8 | 1.5 |
| | | mCmAmGmCmUmUmCmAmGmUmCmCmCmUmUmUmCfUfCfGfU | | | | | | | | |
| | | mCmGmAmUmGmGmU*mC*mA*mG*mC-3′ | | | | | | | | |
| KB-018-698 | 97 | 5′-mC*mU*mA*mA*mAmAmAmCmAmUmGmGmCmCmCmCmAmG | 72.9 | 1.5 | 33.0 | 2.2 | 36.8 | 1.2 | 11.6 | 0.9 |
| | | mCmAmGmCmUmUmCmAmGmUmCmCmCmUmUmUfCfUfCfGmU | | | | | | | | |
| | | mCmGmAmUmGmGmU*mC*mA*mG*mC-3′ | | | | | | | | |
| KB-018-699 | 98 | 5′-mC*mU*mA*mA*mAmAmAmCmAmUmGmGmCmCmCmCmAmG | 39.8 | 1.6 | 18.5 | 2.5 | 15.2 | 0.3 | 6.3 | 1.3 |
| | | mCmAmGmCmUmUmCmAmGmUmCmCmCmUmUmUfCfUfCfGfU | | | | | | | | |
| | | mCmGmAmUmGmGmU*mC*mA*mG*mC-3′ | | | | | | | | |
| KB-018-704 | 99 | 5′-mC*mU*mA*mA*mAmAmAmCmAmUmGmGmCmCmCmCmAmG | 49.8 | 1.6 | 22.2 | 6.0 | 16.7 | 1.6 | 7.9 | 2.2 |
| | | mCmAmGmCmUmUmCmAmGmUmCmCmCmUmUmUmCdGfCdG | | | | | | | | |
| | | mUmCmGmAmUmGmGmU*mC*mA*mG*mC-3′ | | | | | | | | |
| KB-018-705 | 100 | 5′-mC*mU*mA*mA*mAmAmAmCmAmUmGmGmCmCmCmCmAmG | 62.2 | 3.3 | 32.8 | 0.3 | 18.9 | 0.4 | 7.1 | 0.5 |
| | | mCmAmGmCmUmUmCmAmGmUmCmCmCmUmUmUmCdGfCfGm | | | | | | | | |
| | | UmCmGmAmUmGmGmU*mC*mA*mG*mC-3′ | | | | | | | | |
| KB-018-706 | 101 | 5′-mC*mU*mA*mA*mAmAmAmCmAmUmGmGmCmCmCmCmAmG | 53.0 | 1.7 | 27.3 | 2.7 | 12.4 | 1.5 | 5.0 | 1.2 |
| | | mCmAmGmCmUmUmCmAmGmUmCmCmCmUmUmUmCfGdCfGm | | | | | | | | |
| | | UmCmGmAmUmGmGmU*mC*mA*mG*mC-3′ | | | | | | | | |
| KB-018-707 | 102 | 5′-mC*mU*mA*mA*mAmAmAmCmAmUmGmGmCmCmCmCmAmG | 48.2 | 1.1 | 22.5 | 3.0 | 14.9 | 1.5 | 5.7 | 1.5 |
| | | mCmAmGmCmUmUmCmAmGmUmCmCmCmUmUmUmCdGdCfG | | | | | | | | |
| | | mUmCmGmAmUmGmGmU*mC*mA*mG*mC-3′ | | | | | | | | |
| KB-018-708 | 103 | 5′-mC*mU*mA*mA*mAmAmAmCmAmUmGmGmCmCmCmCmAmG | 39.6 | 1.1 | 15.4 | 1.4 | 8.6 | 0.9 | 5.5 | 0.3 |
| | | mCmAmGmCmUmUmCmAmGmUmCmCmCmUmUmUmCmGfCfG | | | | | | | | |
| | | mUmCmGmAmUmGmGmU*mC*mA*mG*mC-3′ | | | | | | | | |
| KB-018-709 | 104 | 5′-mC*mU*mA*mA*mAmAmAmCmAmUmGmGmCmCmCmCmAmG | 63.1 | 4.5 | 31.5 | 3.2 | 24.8 | 1.5 | 8.1 | 1.4 |
| | | mCmAmGmCmUmUmCmAmGmUmCmCmCmUmUmUmCdGdCdlm | | | | | | | | |
| | | UmCmGmAmUmGmGmU*mC*mA*mG*mC-3′ | | | | | | | | |
| KB-018-710 | 105 | 5′-mC*mU*mA*mA*mAmAmAmCmAmUmGmGmCmCmCmCmAmG | 74.3 | 2.0 | 36.1 | 3.4 | 39.0 | 3.8 | 14.7 | 0.5 |
| | | mCmAmGmCmUmUmCmAmGmUmCmCmCmUmUmUmCdldCdlm | | | | | | | | |
| | | UmCmGmAmUmGmGmU*mC*mA*mG*mC-3′ | | | | | | | | |
| KB-018-711 | 106 | 5′-mC*mU*mA*mA*mAmAmAmCmAmUmGmGmCmCmCmCmAmG | 65.5 | 3.2 | 41.3 | 3.2 | 24.6 | 1.7 | 9.6 | 1.1 |
| | | mCmAmGmCmUmUmCmAmGmUmCmCmCmUmUmUmCdldCfGm | | | | | | | | |
| | | UmCmGmAmUmGmGmU*mC*mA*mG*mC-3′ | | | | | | | | |
| KB-018-712 | 107 | 5′-mC*mU*mA*mA*mAmAmAmCmAmUmGmGmCmCmCmCmAmG | 58.8 | 0.0 | 28.6 | 4.1 | 9.5 | 1.8 | 5.0 | 1.1 |
| | | mCmAmGmCmUmUmCmAmGmUmCmCmCmUmUmUmCmldCfGm | | | | | | | | |
| | | UmCmGmAmUmGmGmU*mC*mA*mG*mC-3′ | | | | | | | | |
| KB-018-713 | 108 | 5′-mC*mU*mA*mA*mAmAmAmCmAmUmGmGmCmCmCmCmAmG | 70.5 | 1.4 | 30.0 | 2.4 | 22.4 | 2.8 | 6.9 | 1.7 |
| | | mCmAmGmCmUmUmCmAmGmUmCmCmCmUmUmUmCdlfCfGm | | | | | | | | |
| | | UmCmGmAmUmGmGmU*mC*mA*mG*mC-3′ | | | | | | | | |
| KB-018-714 | 109 | 5′-mC*mU*mA*mA*mAmAmAmCmAmUmGmGmCmCmCmCmAmG | 58.4 | 0.7 | 22.4 | 0.5 | 14.7 | 1.3 | 5.5 | 0.5 |
| | | mCmAmGmCmUmUmCmAmGmUmCmCmCmUmUmUmCdTdmCf | | | | | | | | |
| | | GmUmCmGmAmUmGmGU*mC*mA*mG*mC-3′ | | | | | | | | |
| KB-018-715 | 110 | 5′-mC*mU*mA*mA*mAmAmAmCmAmUmGmGmCmCmCmCmAmG | 50.9 | 2.2 | 21.4 | 1.6 | 12.4 | 1.4 | 4.5 | 0.5 |
| | | mCmAmGmCmUmUmCmAmGmUmCmCmCmUmUmUmCdGdmCf | | | | | | | | |
| | | GmUmCmGmAmUmGmGmU*mC*mA*mG*mC-3′ | | | | | | | | |
| KB-018-716 | 111 | 5′-mC*mU*mA*mA*mAmAmAmCmAmUmGmGmCmCmCmCmAmG | 67.9 | 2.3 | 29.4 | 0.6 | 7.6 | 1.4 | 4.1 | 0.8 |
| | | mCmAmGmCmUmUmCmAmGmUmCmCmCmUmUmUmCfUdmCfG | | | | | | | | |
| | | mUmCmGmAmUmGmGmU*mC*mA*mG*mC-3′ | | | | | | | | |
| KB-018-717 | 112 | 5′-mC*mU*mA*mA*mAmAmAmCmAmUmGmGmCmCmCmCmAmG | 54.6 | 1.0 | 19.7 | 0.8 | | | | |
| | | mCmAmGmCmUmUmCmAmGmUmCmCmCmUmUmUmCmlfCfGm | | | | | | | | |
| | | UmCmGmAmUmGmGmU*mC*mA*mG*mC-3′ | | | | | | | | |
| KB-018-723 | 113 | 5′-mC*mU*mA*mA*mAmAmAmCmAmUmGmGmCmCmCmCmAmG | 24.0 | 0.7 | 11.6 | 2.3 | | | | |
| | | mCmAmGmCmUmUmCmAmGmUmCmCmCmUmUmUmCfGmCfG | | | | | | | | |
| | | mUmCmGmAmUmGmGmU*mC*mA*mG*mC-3′ | | | | | | | | |
| | | SERPINA1 Antisense-gRNA with (S)- and (R)-GNA | | | | | | | | |
| | | modifications in Triplet | | | | | | | | |
| KB-018-001 | 114 | 5′-mC*mU*mA*mA*mAmAmAmCmAmUmGmGmCmCmCmCmAmG | 54.5 | 2.9 | 57.0 | 2.5 | 21.0 | 3.3 | 6.6 | 0.9 |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-205 | 115 | 5′-mC*mU*mA*mA*mAmAmAmCmAmUmGmGmCmCmCmCmAmG | 45.1 | 4.0 | 34.1 | 1.7 | 15.6 | 2.3 | 4.6 | 0.8 |
| | | mCmAmGmCmUmUmCmAmGmUmCmCmCmUmUmUmCsgTdCd | | | | | | | | |
| | | GmUmCmGmAmUmGmGmU*mC*mA*mG*mC-3′ | | | | | | | | |
| KB-018-206 | 116 | 5′-mC*mU*mA*mA*mAmAmAmCmAmUmGmGmCmCmCmCmAmG | 24.0 | 3.4 | 15.3 | 2.4 | 7.3 | 2.0 | 4.1 | 1.3 |
| | | mCmAmGmCmUmUmCmAmGmUmCmCmCmUmUmUmCdTsgCd | | | | | | | | |
| | | GmUmCmGmAmUmGmGmU*mC*mA*mG*mC-3′ | | | | | | | | |
| KB-018-207 | 117 | 5′-mC*mU*mA*mA*mAmAmAmCmAmUmGmGmCmCmCmCmAmG | 15.6 | 5.6 | 13.2 | 3.0 | 7.4 | 1.3 | 6.1 | 2.3 |
| | | mCmAmGmCmUmUmCmAmGmUmCmCmCmUmUmUmCdTdCsg | | | | | | | | |
| | | GmUmCmGmAmUmGmGmU*mC*mA*mG*mC-3′ | | | | | | | | |
| KB-018-208 | 118 | 5′-mC*mU*mA*mA*mAmAmAmCmAmUmGmGmCmCmCmCmAmG | 1.9 | 1.0 | 1.5 | 0.8 | 3.4 | 1.4 | 4.0 | 2.5 |
| | | mCmAmGmCmUmUmCmAmGmUmCmCmCmUmUmUmCsgTsgCs | | | | | | | | |
| | | gGmUmCmGmAmUmGmGmU*mC*mA*mG*mC-3′ | | | | | | | | |
| KB-018-209 | 119 | 5′-mC*mU*mA*mA*mAmAmAmCmAmUmGmGmCmCmCmCmAmG | 9.1 | 0.1 | 9.1 | 0.8 | 4.7 | 1.6 | 3.0 | 1.6 |
| | | mCmAmGmCmUmUmCmAmGmUmCmCmCmUmUmUmCdTsgCsg | | | | | | | | |
| | | GmUmCmGmAmUmGmGmU*mC*mA*mG*mC-3′ | | | | | | | | |
| KB-018-210 | 120 | 5′-mC*mU*mA*mA*mAmAmAmCmAmUmGmGmCmCmCmCmAmG | 9.1 | 3.3 | 6.7 | 2.4 | 15.6 | 8.4 | 2.1 | 0.6 |
| | | mCmAmGmCmUmUmCmAmGmUmCmCmCmUmUmUmCsgTsgCd | | | | | | | | |
| | | GmUmCmGmAmUmGmGmU*mC*mA*mG*mC-3′ | | | | | | | | |
| KB-018-211 | 121 | 5′-mC*mU*mA*mA*mAmAmAmCmAmUmGmGmCmCmCmCmAmG | 10.7 | 1.3 | 7.4 | 1.2 | 3.2 | 1.6 | 6.2 | 4.8 |
| | | mCmAmGmCmUmUmCmAmGmUmCmCmCmUmUmUmCsgTdCsg | | | | | | | | |
| | | GmUmCmGmAmUmGmGmU*mC*mA*mG*mC-3′ | | | | | | | | |
| KB-018-212 | 122 | 5′-mC*mU*mA*mA*mAmAmAmCmAmUmGmGmCmCmCmCmAmG | 10.8 | 0.6 | 4.9 | 2.0 | 5.8 | 4.0 | 3.3 | 2.0 |
| | | mCmAmGmCmUmUmCmAmGmUmCmCmCmUmUmUmCmUsgCm | | | | | | | | |
| | | GmUmCmGmAmUmGmGmU*mC*mA*mG*mC-3′ | | | | | | | | |
| KB-018-213 | 123 | 5′-mC*mU*mA*mA*mAmAmAmCmAmUmGmGmCmCmCmCmAmG | 3.3 | 2.2 | 2.7 | 1.6 | 5.1 | 1.2 | | |
| | | mCmAmGmCmUmUmCmAmGmUmCmCmCmUmUmUmCsgTsgC | | | | | | | | |
| | | mGmUmCmGmAmUmGmGmU*mC*mA*mG*mC-3′ | | | | | | | | |
| KB-018-214 | 124 | 5′-mC*mU*mA*mA*mAmAmAmCmAmUmGmGmCmCmCmCmAmG | 10.9 | 1.6 | 5.9 | 1.1 | 6.2 | 1.5 | 7.4 | 3.5 |
| | | mCmAmGmCmUmUmCmAmGmUmCmCmCmUmUmUmCsgTmCs | | | | | | | | |
| | | gGmUmCmGmAmUmGmGmU*mC*mA*mG*mC-3′ | | | | | | | | |
| KB-018-215 | 125 | 5′-mC*mU*mA*mA*mAmAmAmCmAmUmGmGmCmCmCmCmAmG | 26.8 | 2.4 | 25.9 | 2.1 | 5.1 | 1.2 | | |
| | | mCmAmGmCmUmUmCmAmGmUmCmCmCmUmUmUmCdTrgCdG | | | | | | | | |
| | | mUmCmGmAmUmGmGmU*mC*mA*mG*mC-3′ | | | | | | | | |
| KB-018-216 | 126 | 5′-mC*mU*mA*mA*mAmAmAmCmAmUmGmGmCmCmCmCmAmG | 2.5 | 2.5 | 9.1 | 0.9 | 6.2 | 1.5 | 7.4 | 3.5 |
| | | mCmAmGmCmUmUmCmAmGmUmCmCmCmUmUmUmCmUrgCm | | | | | | | | |
| | | GmUmCmGmAmUmGmGmU*mC*mA*mG*mC-3′ | | | | | | | | |
| | | SERPINA1 E342K Antisense-gRNA with alpha-anomer | | | | | | | | |
| | | modifications in Triplet | | | | | | | | |
| KB-018-001 | 127 | 5′-mC*mU*mA*mA*mAmAmAmCmAmUmGmGmCmCmCmCmAmG | 52.4 | 9.3 | 16.9 | 2.5 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-590 | 128 | 5′-mC*mU*mA*mA*mAmAmAmCmAmUmGmGmCmCmCmCmAmG | 35.2 | 2.0 | 21.3 | 0.5 | | | | |
| | | mCmAmGmCmUmUmCmAmGmUmCmCmCmUmUmUmCaTdCdG | | | | | | | | |
| | | mUmCmGmAmUmGmGmU*mC*mA*mG*mC-3′ | | | | | | | | |
| KB-018-217 | 129 | 5′-mC*mU*mA*mA*mAmAmAmCmAmUmGmGmCmCmCmCmAmG | 18.1 | 0.2 | 7.1 | 0.7 | | | | |
| | | mCmAmGmCmUmUmCmAmGmUmCmCmCmUmUmUmCdTaCdG | | | | | | | | |
| | | mUmCmGmAmUmGmGmU*mC*mA*mG*mC-3′ | | | | | | | | |
| KB-018-591 | 130 | 5′-mC*mU*mA*mA*mAmAmAmCmAmUmGmGmCmCmCmCmAmG | 11.4 | 0.5 | 4.7 | 1.2 | | | | |
| | | mCmAmGmCmUmUmCmAmGmUmCmCmCmUmUmUmCdTdCaG | | | | | | | | |
| | | mUmCmGmAmUmGmGmU*mC*mA*mG*mC-3′ | | | | | | | | |
| KB-018-592 | 131 | 5′-mC*mU*mA*mA*mAmAmAmCmAmUmGmGmCmCmCmCmAmG | 20.1 | 13.1 | 8.3 | 6.5 | | | | |
| | | mCmAmGmCmUmUmCmAmGmUmCmCmCmUmUmUmCaTaCaG | | | | | | | | |
| | | mUmCmGmAmUmGmGmU*mC*mA*mG*mC-3′ | | | | | | | | |
| KB-018-593 | 132 | 5′-mC*mU*mA*mA*mAmAmAmCmAmUmGmGmCmCmCmCmAmG | 26.1 | 3.2 | 18.4 | 0.7 | | | | |
| | | mCmAmGmCmUmUmCmAmGmUmCmCmCmUmUmUmCdTaCaG | | | | | | | | |
| | | mUmCmGmAmUmGmGmU*mC*mA*mG*mC-3′ | | | | | | | | |
| KB-018-594 | 133 | 5′-mC*mU*mA*mA*mAmAmAmCmAmUmGmGmCmCmCmCmAmG | 12.7 | 0.6 | 12.0 | 2.6 | | | | |
| | | mCmAmGmCmUmUmCmAmGmUmCmCmCmUmUmUmCaTaCdG | | | | | | | | |
| | | mUmCmGmAmUmGmGmU*mC*mA*mG*mC-3′ | | | | | | | | |
| KB-018-595 | 134 | 5′-mC*mU*mA*mA*mAmAmAmCmAmUmGmGmCmCmCmCmAmG | 10.0 | 4.6 | 9.5 | 2.3 | | | | |
| | | mCmAmGmCmUmUmCmAmGmUmCmCmCmUmUmUmCaTdCaG | | | | | | | | |
| | | mUmCmGmAmUmGmGmU*mC*mA*mG*mC-3′ | | | | | | | | |
| KB-018-596 | 135 | 5′-mC*mU*mA*mA*mAmAmAmCmAmUmGmGmCmCmCmCmAmG | 21.3 | 2.5 | 5.1 | 1.6 | | | | |
| | | mCmAmGmCmUmUmCmAmGmUmCmCmCmUmUmUmCaTmCaG | | | | | | | | |
| | | mUmCmGmAmUmGmGmU*mC*mA*mG*mC-3′ | | | | | | | | |
| | | SERPINA1 E342K Antisense-gRNA with Arabinose | | | | | | | | |
| | | modifications in Triplet | | | | | | | | |
| KB-018-001 | 136 | 5′-mC*mU*mA*mA*mAmAmAmCmAmUmGmGmCmCmCmCmAmG | 58.2 | 2.1 | 23.5 | 4.1 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-600 | 137 | 5′-mC*mU*mA*mA*mAmAmAmCmAmUmGmGmCmCmCmCmAmG | 43.4 | 4.3 | 32.5 | 3.3 | | | | |
| | | mCmAmGmCmUmUmCmAmGmUmCmCmCmUmUmUmCaUdCdG | | | | | | | | |
| | | *mC*mA*mG*mC-3′mUmCmGmAmUmGmGmU | | | | | | | | |
| KB-018-218 | 138 | 5′-mC*mU*mA*mA*mAmAmAmCmAmUmGmGmCmCmCmCmAmG | 66.1 | 2.3 | 29.6 | 2.2 | | | | |
| | | mCmAmGmCmUmUmCmAmGmUmCmCmCmUmUmUmCdTaCdG | | | | | | | | |
| | | mUmCmGmAmUmGmGmU*mC*mA*mG*mC-3′ | | | | | | | | |
| KB-018-601 | 139 | 5′-mC*mU*mA*mA*mAmAmAmCmAmUmGmGmCmCmCmCmAmG | 71.4 | 0.5 | 37.1 | 2.3 | | | | |
| | | mCmAmGmCmUmUmCmAmGmUmCmCmCmUmUmUmCdTdCaG | | | | | | | | |
| | | mUmCmGmAmUmGmGmU*mC*mA*mG*mC-3′ | | | | | | | | |
| KB-018-602 | 140 | 5′-mC*mU*mA*mA*mAmAmAmCmAmUmGmGmCmCmCmCmAmG | 49.2 | 5.6 | 24.1 | 6.5 | | | | |
| | | mCmAmGmCmUmUmCmAmGmUmCmCmCmUmUmUmCaUaCaG | | | | | | | | |
| | | mUmCmGmAmUmGmGmU*mC*mA*mG*mC-3′ | | | | | | | | |
| KB-018-603 | 141 | 5′-mC*mU*mA*mA*mAmAmAmCmAmUmGmGmCmCmCmCmAmG | 75.1 | 1.8 | 50.9 | 3.9 | | | | |
| | | mCmAmGmCmUmUmCmAmGmUmCmCmCmUmUmUmCdTaCaG | | | | | | | | |
| | | mUmCmGmAmUmGmGmU*mC*mA*mG*mC-3′ | | | | | | | | |
| KB-018-604 | 142 | 5′-mC*mU*mA*mA*mAmAmAmCmAmUmGmGmCmCmCmCmAmG | 34.1 | 1.6 | 26.5 | 3.8 | | | | |
| | | mCmAmGmCmUmUmCmAmGmUmCmCmCmUmUmUmCaUaCdG | | | | | | | | |
| | | mUmCmGmAmUmGmGmU*mC*mA*mG*mC-3′ | | | | | | | | |
| KB-018-605 | 143 | 5′-mC*mU*mA*mA*mAmAmAmCmAmUmGmGmCmCmCmCmAmG | 53.4 | 2.8 | 43.3 | 3.9 | | | | |
| | | mCmAmGmCmUmUmCmAmGmUmCmCmCmUmUmUmCaUdCaG | | | | | | | | |
| | | mUmCmGmAmUmGmGmU*mC*mA*mG*mC-3′ | | | | | | | | |
| KB-018-606 | 144 | 5′-mC*mU*mA*mA*mAmAmAmCmAmUmGmGmCmCmCmCmAmG | 51.7 | 7.0 | 22.5 | 6.2 | | | | |
| | | mCmAmGmCmUmUmCmAmGmUmCmCmCmUmUmUmCaUmCaG | | | | | | | | |
| | | mUmCmGmAmUmGmGmU*mC*mA*mG*mC-3′ | | | | | | | | |
| | | hSERPINA1 E342K Antisense-gRNA with Ara-2′-OMe | | | | | | | | |
| | | modifications in Triplet | | | | | | | | |
| KB-018-001 | 145 | 5′-mC*mU*mA*mA*mAmAmAmCmAmUmGmGmCmCmCmCmAmG | 52.4 | 9.3 | 16.9 | 2.5 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-617 | 146 | 5′-mC*mU*mA*mA*mAmAmAmCmAmUmGmGmCmCmCmCmAmG | 36.0 | 4.0 | 21.1 | 2.3 | | | | |
| | | mCmAmGmCmUmUmCmAmGmUmCmCmCmUmUmUmCamUdCd | | | | | | | | |
| | | GmUmCmGmAmUmGmGmU*mC*mA*mG*mC-3′ | | | | | | | | |
| KB-018-219 | 147 | 5′-mC*mU*mA*mA*mAmAmAmCmAmUmGmGmCmCmCmCmAmG | 24.4 | 0.3 | 9.3 | 0.6 | | | | |
| | | mCmAmGmCmUmUmCmAmGmUmCmCmCmUmUmUmCdTamCd | | | | | | | | |
| | | GmUmCmGmAmUmGmGmU*mC*mA*mG*mC-3′ | | | | | | | | |
| KB-018-618 | 148 | 5′-mC*mU*mA*mA*mAmAmAmCmAmUmGmGmCmCmCmCmAmG | 63.7 | 3.9 | 24.8 | | | | | |
| | | mCmAmGmCmUmUmCmAmGmUmCmCmCmUmUmUmCdTdCam | | | | | | | | |
| | | GmUmCmGmAmUmGmGmU*mC*mA*mG*mC-3′ | | | | | | | | |
| KB-018-619 | 149 | 5′-mC*mU*mA*mA*mAmAmAmCmAmUmGmGmCmCmCmCmAmG | 47.6 | 3.8 | 17.0 | 0.4 | | | | |
| | | mCmAmGmCmUmUmCmAmGmUmCmCmCmUmUmUmCamUamC | | | | | | | | |
| | | amGmUmCmGmAmUmGmGmU*mC*mA*mG*mC-3′ | | | | | | | | |
| KB-018-620 | 150 | 5′-mC*mU*mA*mA*mAmAmAmCmAmUmGmGmCmCmCmCmAmG | 47.8 | 0.1 | 23.7 | 3.4 | | | | |
| | | mCmAmGmCmUmUmCmAmGmUmCmCmCmUmUmUmCdTamCa | | | | | | | | |
| | | mGmUmCmGmAmUmGmGmU*mC*mA*mG*mC-3′ | | | | | | | | |
| KB-018-621 | 151 | 5′-mC*mU*mA*mA*mAmAmAmCmAmUmGmGmCmCmCmCmAmG | 16.1 | 1.4 | 5.8 | 0.8 | | | | |
| | | mCmAmGmCmUmUmCmAmGmUmCmCmCmUmUmUmCamUamC | | | | | | | | |
| | | dGmUmCmGmAmUmGmGmU*mC*mA*mG*mC-3′ | | | | | | | | |
| KB-018-622 | 152 | 5′-mC*mU*mA*mA*mAmAmAmCmAmUmGmGmCmCmCmCmAmG | 52.1 | 1.0 | 26.4 | 1.4 | | | | |
| | | mCmAmGmCmUmUmCmAmGmUmCmCmCmUmUmUmCamUdCa | | | | | | | | |
| | | mGmUmCmGmAmUmGmGmU*mC*mA*mG*mC-3′ | | | | | | | | |
| KB-018-623 | 153 | 5′-mC*mU*mA*mA*mAmAmAmCmAmUmGmGmCmCmCmCmAmG | 60.1 | 1.9 | 19.0 | 1.6 | | | | |
| | | mCmAmGmCmUmUmCmAmGmUmCmCmCmUmUmUmCamUmCa | | | | | | | | |
| | | mGmUmCmGmAmUmGmGmU*mC*mA*mG*mC-3′ | | | | | | | | |
| | | SERPINA1 E342K Antisense-gRNA with Abasic (dSpacer) | | | | | | | | |
| | | modifications in Triplet | | | | | | | | |
| KB-018-001 | 154 | 5′-mC*mU*mA*mA*mAmAmAmCmAmUmGmGmCmCmCmCmAmG | 58.8 | 2.0 | 24.9 | 1.8 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-634 | 155 | 5′-mC*mU*mA*mA*mAmAmAmCmAmUmGmGmCmCmCmCmAmG | 27.4 | 2.2 | 15.4 | 0.4 | | | | |
| | | mCmAmGmCmUmUmCmAmGmUmCmCmCmUmUmUmCdSdCdG | | | | | | | | |
| | | mUmCmGmAmUmGmGmU*mC*mA*mG*mC-3′ | | | | | | | | |
| KB-018-220 | 156 | 5′-mC*mU*mA*mA*mAmAmAmCmAmUmGmGmCmCmCmCmAmG | 23.8 | 1.4 | 9.6 | 1.2 | | | | |
| | | mCmAmGmCmUmUmCmAmGmUmCmCmCmUmUmUmCdTdSdG | | | | | | | | |
| | | mUmCmGmAmUmGmGmU*mC*mA*mG*mC-3′ | | | | | | | | |
| KB-018-635 | 157 | 5′-mC*mU*mA*mA*mAmAmAmCmAmUmGmGmCmCmCmCmAmG | 15.7 | 1.4 | 6.8 | 1.5 | | | | |
| | | mCmAmGmCmUmUmCmAmGmUmCmCmCmUmUmUmCdTdCdS | | | | | | | | |
| | | mUmCmGmAmUmGmGmU*mC*mA*mG*mC-3′ | | | | | | | | |
| KB-018-636 | 158 | 5′-mC*mU*mA*mA*mAmAmAmCmAmUmGmGmCmCmCmCmAmG | 10.1 | 1.7 | 7.6 | 1.2 | | | | |
| | | mCmAmGmCmUmUmCmAmGmUmCmCmCmUmUmUmCdSdSdS | | | | | | | | |
| | | mUmCmGmAmUmGmGmU*mC*mA*mG*mC-3′ | | | | | | | | |
| KB-018-637 | 159 | 5′-mC*mU*mA*mA*mAmAmAmCmAmUmGmGmCmCmCmCmAmG | 24.0 | 2.4 | 10.6 | 1.2 | | | | |
| | | mCmAmGmCmUmUmCmAmGmUmCmCmCmUmUmUmCdTdSdS | | | | | | | | |
| | | mUmCmGmAmUmGmGmU*mC*mA*mG*mC-3′ | | | | | | | | |
| KB-018-638 | 160 | 5′-mC*mU*mA*mA*mAmAmAmCmAmUmGmGmCmCmCmCmAmG | 25.4 | 1.9 | 16.7 | 1.7 | | | | |
| | | mCmAmGmCmUmUmCmAmGmUmCmCmCmUmUmUmCdSdSdG | | | | | | | | |
| | | mUmCmGmAmUmGmGmU*mC*mA*mG*mC-3′ | | | | | | | | |
| KB-018-639 | 161 | 5′-mC*mU*mA*mA*mAmAmAmCmAmUmGmGmCmCmCmCmAmG | 5.7 | 0.5 | 3.1 | 0.5 | | | | |
| | | mCmAmGmCmUmUmCmAmGmUmCmCmCmUmUmUmCdSdCdS | | | | | | | | |
| | | mUmCmGmAmUmGmGmU*mC*mA*mG*mC-3′ | | | | | | | | |
| KB-018-640 | 162 | 5′-mC*mU*mA*mA*mAmAmAmCmAmUmGmGmCmCmCmCmAmG | 6.9 | 2.0 | 7.7 | 2.5 | | | | |
| | | mCmAmGmCmUmUmCmAmGmUmCmCmCmUmUmUmCdSmCdS | | | | | | | | |
| | | mUmCmGmAmUmGmGmU*mC*mA*mG*mC-3′ | | | | | | | | |
| | | Antisense-gRNA with 2′-OH-Abasic (rSpacer) | | | | | | | | |
| | | modifications in Triplet | | | | | | | | |
| KB-018-001 | 163 | 5′-mC*mU*mA*mA*mAmAmAmCmAmUmGmGmCmCmCmCmAmG | 48.7 | 4.2 | 25.3 | 6.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-641 | 164 | 5′-mC*mU*mA*mA*mAmAmAmCmAmUmGmGmCmCmCmCmAmG | 21.4 | 2.6 | 20.8 | 13.4 | | | | |
| | | mCmAmGmCmUmUmCmAmGmUmCmCmCmUmUmUmCrSdCdG | | | | | | | | |
| | | mUmCmGmAmUmGmGmU*mC*mA*mG*mC-3′ | | | | | | | | |
| KB-018-642 | 165 | 5′-mC*mU*mA*mA*mAmAmAmCmAmUmGmGmCmCmCmCmAmG | 12.8 | 6.9 | 19.0 | 10.5 | | | | |
| | | mCmAmGmCmUmUmCmAmGmUmCmCmCmUmUmUmCdTrSdG | | | | | | | | |
| | | mUmCmGmAmUmGmGmU*mC*mA*mG*mC-3′ | | | | | | | | |
| KB-018-643 | 166 | 5′-mC*mU*mA*mA*mAmAmAmCmAmUmGmGmCmCmCmCmAmG | 8.6 | 4.2 | 8.7 | 7.7 | | | | |
| | | mCmAmGmCmUmUmCmAmGmUmCmCmCmUmUmUmCdTdCrSm | | | | | | | | |
| | | UmCmGmAmUmGmGmU*mC*mA*mG*mC-3′ | | | | | | | | |
| KB-018-644 | 167 | 5′-mC*mU*mA*mA*mAmAmAmCmAmUmGmGmCmCmCmCmAmG | 27.7 | 4.9 | 24.5 | 3.4 | | | | |
| | | mCmAmGmCmUmUmCmAmGmUmCmCmCmUmUmUmCrSrSrSm | | | | | | | | |
| | | UmCmGmAmUmGmGmU*mC*mA*mG*mC-3′ | | | | | | | | |
| KB-018-645 | 168 | 5′-mC*mU*mA*mA*mAmAmAmCmAmUmGmGmCmCmCmCmAmG | 28.4 | 11.1 | 20.6 | 15.7 | | | | |
| | | mCmAmGmCmUmUmCmAmGmUmCmCmCmUmUmUmCdTrSrSm | | | | | | | | |
| | | UmCmGmAmUmGmGmU*mC*mA*mG*mC-3′ | | | | | | | | |
| KB-018-646 | 169 | 5′-mC*mU*mA*mA*mAmAmAmCmAmUmGmGmCmCmCmCmAmG | 16.9 | 13.2 | 10.7 | 7.7 | | | | |
| | | mCmAmGmCmUmUmCmAmGmUmCmCmCmUmUmUmCrSrSdGm | | | | | | | | |
| | | UmCmGmAmUmGmGmU*mC*mA*mG*mC-3′ | | | | | | | | |
| KB-018-647 | 170 | 5′-mC*mU*mA*mA*mAmAmAmCmAmUmGmGmCmCmCmCmAmG | 7.1 | 6.9 | 6.8 | 7.6 | | | | |
| | | mCmAmGmCmUmUmCmAmGmUmCmCmCmUmUmUmCrSdCrSm | | | | | | | | |
| | | UmCmGmAmUmGmGmU*mC*mA*mG*mC-3′ | | | | | | | | |
| KB-018-648 | 171 | 5′-mC*mU*mA*mA*mAmAmAmCmAmUmGmGmCmCmCmCmAmG | 29.2 | 2.5 | 23.5 | 4.6 | | | | |
| | | mCmAmGmCmUmUmCmAmGmUmCmCmCmUmUmUmCrSmCrS | | | | | | | | |
| | | mUmCmGmAmUmGmGmU*mC*mA*mG*mC-3′ | | | | | | | | |
| | | SERPINA1 E342K Antisense-gRNA with 2′-OMe-Abasic | | | | | | | | |
| | | (mSpacer) modifications in Triplet | | | | | | | | |
| KB-018-001 | 172 | 5′-mC*mU*mA*mA*mAmAmAmCmAmUmGmGmCmCmCmCmAmG | 54.4 | 8.7 | 15.7 | 2.6 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-650 | 173 | 5′-mC*mU*mA*mA*mAmAmAmCmAmUmGmGmCmCmCmCmAmG | 25.3 | 0.6 | 8.4 | 1.4 | | | | |
| | | mCmAmGmCmUmUmCmAmGmUmCmCmCmUmUmUmCmSdCdG | | | | | | | | |
| | | mUmCmGmAmUmGmGmU*mC*mA*mG*mC-3′ | | | | | | | | |
| KB-018-651 | 174 | 5′-mC*mU*mA*mA*mAmAmAmCmAmUmGmGmCmCmCmCmAmG | 15.8 | 1.3 | 5.3 | 1.9 | | | | |
| | | mCmAmGmCmUmUmCmAmGmUmCmCmCmUmUmUmCdTmSdG | | | | | | | | |
| | | mUmCmGmAmUmGmGmU*mC*mA*mG*mC-3′ | | | | | | | | |
| KB-018-652 | 175 | 5′-mC*mU*mA*mA*mAmAmAmCmAmUmGmGmCmCmCmCmAmG | 13.3 | 0.3 | 4.9 | 3.0 | | | | |
| | | mCmAmGmCmUmUmCmAmGmUmCmCmCmUmUmUmCdTdCmS | | | | | | | | |
| | | mUmCmGmAmUmGmGmU*mC*mA*mG*mC-3′ | | | | | | | | |
| KB-018-653 | 176 | 5′-mC*mU*mA*mA*mAmAmAmCmAmUmGmGmCmCmCmCmAmG | 14.6 | 2.6 | 13.8 | 4.3 | | | | |
| | | mCmAmGmCmUmUmCmAmGmUmCmCmCmUmUmUmCmSmSm | | | | | | | | |
| | | SmUmCmGmAmUmGmGmU*mC*mA*mG*mC-3′ | | | | | | | | |
| KB-018-654 | 177 | 5′-mC*mU*mA*mA*mAmAmAmCmAmUmGmGmCmCmCmCmAmG | 25.7 | 1.6 | 7.7 | 0.3 | | | | |
| | | mCmAmGmCmUmUmCmAmGmUmCmCmCmUmUmUmCdTmSmS | | | | | | | | |
| | | mUmCmGmAmUmGmGmU*mC*mA*mG*mC-3′ | | | | | | | | |
| KB-018-655 | 178 | 5′-mC*mU*mA*mA*mAmAmAmCmAmUmGmGmCmCmCmCmAmG | 20.8 | 1.0 | 8.7 | 3.4 | | | | |
| | | mCmAmGmCmUmUmCmAmGmUmCmCmCmUmUmUmCmSmSd | | | | | | | | |
| | | GmUmCmGmAmUmGmGmU*mC*mA*mG*mC-3′ | | | | | | | | |
| KB-018-656 | 179 | 5′-mC*mU*mA*mA*mAmAmAmCmAmUmGmGmCmCmCmCmAmG | 2.6 | 1.2 | 0.6 | 0.9 | | | | |
| | | mCmAmGmCmUmUmCmAmGmUmCmCmCmUmUmUmCmSdCmS | | | | | | | | |
| | | mUmCmGmAmUmGmGmU*mC*mA*mG*mC-3′ | | | | | | | | |
| KB-018-657 | 180 | 5′-mC*mU*mA*mA*mAmAmAmCmAmUmGmGmCmCmCmCmAmG | 13.8 | 2.2 | 9.6 | 1.1 | | | | |
| | | mCmAmGmCmUmUmCmAmGmUmCmCmCmUmUmUmCmSmCm | | | | | | | | |
| | | SmUmCmGmAmUmGmGmU*mC*mA*mG*mC-3′ | | | | | | | | |
| | | SERPINA1 E342K Antisense-gRNA: Triplet expansion | | | | | | | | |
| KB-018-724 | 181 | 5′-mC*mU*mA*mA*mAmAmAmCmAmUmGmGmCmCmCmCmAmG | 25.8 | 1.0 | 12.2 | 1.0 | 7.8 | 0.7 | 3.8 | 0.7 |
| | | mCmAmGmCmUmUmCmAmGmUmCmCmCmUmUmUmCdTdCdG | | | | | | | | |
| | | dTmCmGmAmUmGmGmU*mC*mA*mG*mC-3′ | | | | | | | | |
| KB-018-725 | 182 | 5′-mC*mU*mA*mA*mAmAmAmCmAmUmGmGmCmCmCmCmAmG | 8.4 | 0.7 | 5.1 | 0.6 | 6.4 | 1.9 | 3.3 | 0.7 |
| | | mCmAmGmCmUmUmCmAmGmUmCmCmCmUmUmUmCdTdCdG | | | | | | | | |
| | | sgTmCmGmAmUmGmGmU*mC*mA*mG*mC-3′ | | | | | | | | |
| KB-018-726 | 183 | 5′-mC*mU*mA*mA*mAmAmAmCmAmUmGmGmCmCmCmCmAmG | 47.2 | 1.8 | 20.0 | 0.7 | 18.1 | 0.3 | 5.5 | 0.8 |
| | | mCmAmGmCmUmUmCmAmGmUmCmCmCmUmUmUdCdTdCdG | | | | | | | | |
| | | mUmCmGmAmUmGmGmU*mC*mA*mG*mC-3′ | | | | | | | | |
| KB-018-727 | 184 | 5′-mC*mU*mA*mA*mAmAmAmCmAmUmGmGmCmCmCmCmAmG | 24.7 | 2.0 | 10.1 | 0.6 | 8.6 | 2.1 | 3.3 | 1.1 |
| | | mCmAmGmCmUmUmCmAmGmUmCmCmCmUmUmUdCdTdCdGd | | | | | | | | |
| | | TmCmGmAmUmGmGmU*mC*mA*mG*mC-3′ | | | | | | | | |
| KB-018-728 | 185 | 5′-mC*mU*mA*mA*mAmAmAmCmAmUmGmGmCmCmCmCmAmG | 62.7 | 1.8 | 23.7 | 9.9 | 17.1 | 2.7 | 4.5 | 1.0 |
| | | mCmAmGmCmUmUmCmAmGmUmCmCmCmUmUmUsgCdTdCdG | | | | | | | | |
| | | mUmCmGmAmUmGmGmU*mC*mA*mG*mC-3′ | | | | | | | | |

TABLE 7
SERPINA1 Modifications in Flanking Sequence

| | | | Editing Data | | | | | | | |
|---|---|---|---|---|---|---|---|---|---|---|
| | SEQ | | ADAR2 % Editing | | | | ADAR1 p110 % Editing | | | |
| Seq ID | ID NO | Guide Oligonucleotide Sequences | 100 nM | SD | 10 nM | SD | 100 nM | SD | 10 nM | SD |
| | | SERPINA1 E342K Antisense-gRNA with 2′-O-MOE modification | | | | | | | | |
| KB-018-001 | 186 | 5′-mC*mU*mA*mA*mAmAmAmCmAmUmGmGmCmCmCmCmAmG | 39.8 | 6.1 | 21.2 | 2.0 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-451 | 187 | 5′-MC*mU*mA*mA*mAmAmAmCmAmUmGmGmCmCmCmCmAmG | 62.3 | 3.8 | 41.5 | 6.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-452 | 188 | 5′-mC*MU*mA*mA*mAmAmAmCmAmUmGmGmCmCmCmCmAmG | 47.8 | 2.0 | 36.4 | 5.1 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-453 | 189 | 5′-mC*mU*MA*mA*mAmAmAmCmAmUmGmGmCmCmCmCmAm | 59.1 | 3.7 | 41.3 | 8.4 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-454 | 190 | 5′-mC*mU*mA*MA*mAmAmAmCmAmUmGmGmCmCmCmCmAm | 64.2 | 6.5 | 37.3 | 4.5 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-455 | 191 | 5′-mC*mU*mA*mA*MAmAmAmCmAmUmGmGmCmCmCmCmAm | 59.8 | 4.7 | 39.8 | 5.8 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-456 | 192 | 5′-mC*mU*mA*mA*mAMAmAmCmAmUmGmGmCmCmCmCmAm | 54.3 | 0.1 | 39.3 | 6.3 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-457 | 193 | 5′-mC*mU*mA*mA*mAmAMAmCmAmUmGmGmCmCmCmCmAm | 47.2 | 1.7 | 28.1 | 1.8 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-458 | 194 | 5′-mC*mU*mA*mA*mAmAmAMCmAmUmGmGmCmCmCmCmAmG | 57.8 | 1.3 | 34.0 | 3.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-459 | 195 | 5′-mC*mU*mA*mA*mAmAmAmCMAmUmGmGmCmCmCmCmAm | 59.0 | 2.8 | 49.7 | | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-460 | 196 | 5′-mC*mU*mA*mA*mAmAmAmCmAMUmGmGmCmCmCmCmAmG | 55.2 | 3.6 | 36.7 | 6.6 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-461 | 197 | 5′-mC*mU*mA*mA*mAmAmAmCmAmUMGmGmCmCmCmCmAmG | 57.0 | 3.9 | 35.6 | 3.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-462 | 198 | 5′-mC*mU*mA*mA*mAmAmAmCmAmUmGMGmCmCmCmCmAmG | 57.2 | 2.7 | | | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-463 | 199 | 5′-mC*mU*mA*mA*mAmAmAmCmAmUmGmGMCmCmCmCmAmG | 53.1 | 6.6 | 28.8 | 4.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-464 | 200 | 5′-mC*mU*mA*mA*mAmAmAmCmAmUmGmGmCMCmCmCmAmG | 54.0 | 2.3 | 27.6 | 2.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-465 | 201 | 5′-mC*mU*mA*mA*mAmAmAmCmAmUmGmGmCmCMCmCmAmG | 42.7 | 2.2 | 32.1 | 1.0 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-466 | 202 | 5′-mC*mU*mA*mA*mAmAmAmCmAmUmGmGmCmCmCMCmAmG | 53.7 | 1.8 | 24.9 | 3.5 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-467 | 203 | 5′-mC*mU*mA*mA*mAmAmAmCmAmUmGmGmCmCmCmCMAm | 58.8 | 1.5 | 28.1 | 2.5 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-468 | 204 | 5′-mC*mU*mA*mA*mAmAmAmCmAmUmGmGmCmCmCmCmAMG | 55.6 | 0.9 | 35.8 | 3.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-469 | 205 | 5′-mC*mU*mA*mA*mAmAmAmCmAmUmGmGmCmCmCmCmAmG | 53.2 | 2.8 | 39.8 | 3.6 | | | | |
| | | MCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-470 | 206 | 5′-mC*mU*mA*mA*mAmAmAmCmAmUmGmGmCmCmCmCmAmG | 54.7 | 3.1 | 34.8 | 4.5 | | | | |
| | | mCMAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-471 | 207 | 5′-mC*mU*mA*mA*mAmAmAmCmAmUmGmGmCmCmCmCmAmG | 54.4 | 1.4 | 36.0 | 3.7 | | | | |
| | | mCmAMGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-472 | 208 | 5′-mC*mU*mA*mA*mAmAmAmCmAmUmGmGmCmCmCmCmAmG | 53.2 | 1.0 | 34.0 | 2.3 | | | | |
| | | mCmAmGMCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-473 | 209 | 5′-mC*mU*mA*mA*mAmAmAmCmAmUmGmGmCmCmCmCmAmG | 37.2 | 5.0 | 20.9 | 2.3 | | | | |
| | | mCmAmGmCMUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-474 | 210 | 5′-mC*mU*mA*mA*mAmAmAmCmAmUmGmGmCmCmCmCmAmG | 31.5 | 4.2 | 16.4 | 0.6 | | | | |
| | | mCmAmGmCmUMUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-475 | 211 | 5′-mC*mU*mA*mA*mAmAmAmCmAmUmGmGmCmCmCmCmAmG | 46.4 | 4.6 | 25.3 | 0.8 | | | | |
| | | mCmAmGmCmUmUMCmAmGmUmCmCmCmUmUmUmCdTdCdG | | | | | | | | |
| | | mUmCmGmAmUmGmGmU*mC*mA*mG*mC-3′ | | | | | | | | |
| KB-018-476 | 212 | 5′-mC*mU*mA*mA*mAmAmAmCmAmUmGmGmCmCmCmCmAmG | 42.9 | 9.2 | 25.9 | 1.1 | | | | |
| | | mCmAmGmCmUmUmCMAmGmUmCmCmCmUmUmUmCdTdCdG | | | | | | | | |
| | | mUmCmGmAmUmGmGmU*mC*mA*mG*mC-3′ | | | | | | | | |
| KB-018-477 | 213 | 5′-mC*mU*mA*mA*mAmAmAmCmAmUmGmGmCmCmCmCmAmG | 52.5 | 3.4 | 47.8 | 2.7 | | | | |
| | | mCmAmGmCmUmUmCmAMGmUmCmCmCmUmUmUmCdTdCdG | | | | | | | | |
| | | mUmCmGmAmUmGmGmU*mC*mA*mG*mC-3′ | | | | | | | | |
| KB-018-478 | 214 | 5′-mC*mU*mA*mA*mAmAmAmCmAmUmGmGmCmCmCmCmAmG | 46.1 | 3.3 | 22.9 | 1.6 | | | | |
| | | mCmAmGmCmUmUmCmAmGMUmCmCmCmUmUmUmCdTdCdG | | | | | | | | |
| | | mUmCmGmAmUmGmGmU*mC*mA*mG*mC-3′ | | | | | | | | |
| KB-018-479 | 215 | 5′-mC*mU*mA*mA*mAmAmAmCmAmUmGmGmCmCmCmCmAmG | 44.5 | 0.3 | 19.8 | 2.0 | | | | |
| | | mCmAmGmCmUmUmCmAmGmUMCmCmCmUmUmUmCdTdCdG | | | | | | | | |
| | | mUmCmGmAmUmGmGmU*mC*mA*mG*mC-3′ | | | | | | | | |
| KB-018-480 | 216 | 5′-mC*mU*mA*mA*mAmAmAmCmAmUmGmGmCmCmCmCmAmG | 42.9 | 4.1 | 38.6 | 4.9 | | | | |
| | | mCmAmGmCmUmUmCmAmGmUmCMCmCmUmUmUmCdTdCdG | | | | | | | | |
| | | mUmCmGmAmUmGmGmU*mC*mA*mG*mC-3′ | | | | | | | | |
| KB-018-481 | 217 | 5′-mC*mU*mA*mA*mAmAmAmCmAmUmGmGmCmCmCmCmAmG | 46.8 | 2.1 | 25.1 | 1.3 | | | | |
| | | mCmAmGmCmUmUmCmAmGmUmCmCMCmUmUmUmCdTdCdG | | | | | | | | |
| | | mUmCmGmAmUmGmGmU*mC*mA*mG*mC-3′ | | | | | | | | |
| KB-018-482 | 218 | 5′-mC*mU*mA*mA*mAmAmAmCmAmUmGmGmCmCmCmCmAmG | 53.0 | 2.8 | 26.1 | 1.9 | | | | |
| | | mCmAmGmCmUmUmCmAmGmUmCmCmCMUmUmUmCdTdCdG | | | | | | | | |
| | | mUmCmGmAmUmGmGmU*mC*mA*mG*mC-3′ | | | | | | | | |
| KB-018-483 | 219 | 5′-mC*mU*mA*mA*mAmAmAmCmAmUmGmGmCmCmCmCmAmG | 53.2 | 4.1 | 25.2 | 1.7 | | | | |
| | | mCmAmGmCmUmUmCmAmGmUmCmCmCmUMUmUmCdTdCdG | | | | | | | | |
| | | mUmCmGmAmUmGmGmU*mC*mA*mG*mC-3′ | | | | | | | | |
| KB-018-484 | 220 | 5′-mC*mU*mA*mA*mAmAmAmCmAmUmGmGmCmCmCmCmAmG | 49.1 | 6.0 | 29.2 | 1.4 | | | | |
| | | mCmAmGmCmUmUmCmAmGmUmCmCmCmUmUMUmCdTdCdG | | | | | | | | |
| | | mUmCmGmAmUmGmGmU*mC*mA*mG*mC-3′ | | | | | | | | |
| KB-018-485 | 221 | 5′-mC*mU*mA*mA*mAmAmAmCmAmUmGmGmCmCmCmCmAmG | 49.8 | 1.5 | 26.0 | 2.2 | | | | |
| | | mCmAmGmCmUmUmCmAmGmUmCmCmCmUmUmUMCdTdCdG | | | | | | | | |
| | | mUmCmGmAmUmGmGmU*mC*mA*mG*mC-3′ | | | | | | | | |
| KB-018-486 | 222 | 5′-mC*mU*mA*mA*mAmAmAmCmAmUmGmGmCmCmCmCmAmG | 11.9 | 0.9 | 6.7 | 0.5 | | | | |
| | | mCmAmGmCmUmUmCmAmGmUmCmCmCmUmUmUmCdTdCdG | | | | | | | | |
| | | MUmCmGmAmUmGmGmU*mC*mA*mG*mC-3′ | | | | | | | | |
| KB-018-487 | 223 | 5′-mC*mU*mA*mA*mAmAmAmCmAmUmGmGmCmCmCmCmAmG | 13.8 | 0.8 | 5.0 | 1.3 | | | | |
| | | mCmAmGmCmUmUmCmAmGmUmCmCmCmUmUmUmCdTdCdG | | | | | | | | |
| | | mUMCmGmAmUmGmGmU*mC*mA*mG*mC-3′ | | | | | | | | |
| KB-018-488 | 224 | 5′-mC*mU*mA*mA*mAmAmAmCmAmUmGmGmCmCmCmCmAmG | 50.4 | 2.8 | 24.5 | 1.7 | | | | |
| | | mCmAmGmCmUmUmCmAmGmUmCmCmCmUmUmUmCdTdCdG | | | | | | | | |
| | | mUmCMGmAmUmGmGmU*mC*mA*mG*mC-3′ | | | | | | | | |
| KB-018-489 | 225 | 5′-mC*mU*mA*mA*mAmAmAmCmAmUmGmGmCmCmCmCmAmG | 53.8 | 1.8 | 24.7 | 2.1 | | | | |
| | | mCmAmGmCmUmUmCmAmGmUmCmCmCmUmUmUmCdTdCdG | | | | | | | | |
| | | mUmCmGMAmUmGmGmU*mC*mA*mG*mC-3′ | | | | | | | | |
| KB-018-490 | 226 | 5′-mC*mU*mA*mA*mAmAmAmCmAmUmGmGmCmCmCmCmAmG | 49.7 | 1.1 | 17.7 | 0.3 | | | | |
| | | mCmAmGmCmUmUmCmAmGmUmCmCmCmUmUmUmCdTdCdG | | | | | | | | |
| | | mUmCmGmAMUmGmGmU*mC*mA*mG*mC-3′ | | | | | | | | |
| KB-018-491 | 227 | 5′-mC*mU*mA*mA*mAmAmAmCmAmUmGmGmCmCmCmCmAmG | 58.1 | 0.1 | 23.9 | 0.8 | | | | |
| | | mCmAmGmCmUmUmCmAmGmUmCmCmCmUmUmUmCdTdCdG | | | | | | | | |
| | | mUmCmGmAmUMGmGmU*mC*mA*mG*mC-3′ | | | | | | | | |
| KB-018-492 | 228 | 5′-mC*mU*mA*mA*mAmAmAmCmAmUmGmGmCmCmCmCmAmG | 58.3 | 0.9 | 26.4 | 1.0 | | | | |
| | | mCmAmGmCmUmUmCmAmGmUmCmCmCmUmUmUmCdTdCdG | | | | | | | | |
| | | mUmCmGmAmUmGMGmU*mC*mA*mG*mC-3′ | | | | | | | | |
| KB-018-493 | 229 | 5′-mC*mU*mA*mA*mAmAmAmCmAmUmGmGmCmCmCmCmAmG | 52.8 | 0.7 | 25.4 | 4.1 | | | | |
| | | mCmAmGmCmUmUmCmAmGmUmCmCmCmUmUmUmCdTdCdG | | | | | | | | |
| | | mUmCmGmAmUmGmGMU*mC*mA*mG*mC-3′ | | | | | | | | |
| KB-018-494 | 230 | 5′-mC*mU*mA*mA*mAmAmAmCmAmUmGmGmCmCmCmCmAmG | 52.0 | 0.7 | 26.4 | 2.4 | | | | |
| | | mCmAmGmCmUmUmCmAmGmUmCmCmCmUmUmUmCdTdCdG | | | | | | | | |
| | | mUmCmGmAmUmGmGmU*MC*mA*mG*mC-3′ | | | | | | | | |
| KB-018-495 | 231 | 5′-mC*mU*mA*mA*mAmAmAmCmAmUmGmGmCmCmCmCmAmG | 49.1 | 0.3 | 26.9 | 2.0 | | | | |
| | | mCmAmGmCmUmUmCmAmGmUmCmCmCmUmUmUmCdTdCdG | | | | | | | | |
| | | mUmCmGmAmUmGmGmU*mC*MA*mG*mC-3′ | | | | | | | | |
| KB-018-496 | 232 | 5′-mC*mU*mA*mA*mAmAmAmCmAmUmGmGmCmCmCmCmAmG | 47.3 | 2.5 | 28.4 | 2.6 | | | | |
| | | mCmAmGmCmUmUmCmAmGmUmCmCmCmUmUmUmCdTdCdG | | | | | | | | |
| | | mUmCmGmAmUmGmGmU*mC*mA*MG*mC-3′ | | | | | | | | |
| KB-018-497 | 233 | 5′-mC*mU*mA*mA*mAmAmAmCmAmUmGmGmCmCmCmCmAmG | 56.2 | 0.8 | 26.7 | 3.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*MC-3′ | | | | | | | | |
| | | SERPINA1 E342K Antisense-gRNA with 2′-O-MOE modifications | | | | | | | | |
| KB-018-001 | 234 | 5′-mC*mU*mA*mA*mAmAmAmCmAmUmGmGmCmCmCmCmAmG | 55.1 | 3.9 | 36.3 | 0.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-378 | 235 | 5′-MA*MA*MC*MA*MUMGMGMCMCMCMCMAMGMCMAMGMCM | 0.0 | 0.0 | 0.0 | 0.0 | | | | |
| | | UMUMCMAMGMUMCMCMCMUMUMUMCdTdCdGMUMCMGMAM | | | | | | | | |
| | | UMGMGMU*MC*MA*MG*MC-3′ | | | | | | | | |
| KB-018-1261 | 236 | 5′-MC*MU*MA*MA*MAMAMAMCMAMUMGMGMCMCMCMCMAMG | 0.0 | 0.0 | 0.0 | 0.0 | | | | |
| | | MCMAMGMCMUMUMCMAMGMUMCMCMCMUMUMUMCdTdCdG | | | | | | | | |
| | | mUmCMGMAMUMGMGMU*MC*MA*MG*MC-3′ | | | | | | | | |
| KB-018-1262 | 237 | 5′-MC*MU*MA*MA*MAMAMAMCMAMUMGMGMCMCmCMCMAMG | 0.0 | 0.0 | 0.0 | 0.0 | | | | |
| | | MCMAMGMCmUmUMCMAMGMUMCMCMCMUMUMUMCdTdCdG | | | | | | | | |
| | | mUmCMGMAMUMGMGMU*MC*MA*MG*MC-3′ | | | | | | | | |
| KB-018-1263 | 238 | 5′-MC*MU*MA*MA*MAMAMAMCMAMUMGMGMCMCmCMCMAMG | 27.4 | 3.0 | 0.0 | 0.0 | | | | |
| | | MCMAMGMCmUmUmCmAMGmUmCmCmCMUMUMUMCdTdCdG | | | | | | | | |
| | | mUmCMGMAMUMGMGMU*MC*MA*MG*MC-3′ | | | | | | | | |
| KB-018-1264 | 239 | 5′-MC*mU*MA*MA*MAMAmAMCMAMUMGMGMCMCmCMCMAMG | 49.1 | 7.1 | 57.2 | 0.0 | | | | |
| | | MCMAMGMCmUmUmCmAMGmUmCmCmCMUMUmUmCdTdCdG | | | | | | | | |
| | | mUmCmGmAmUMGMGMU*MC*mA*mG*MC-3′ | | | | | | | | |
| KB-018-1289 | 240 | 5′-MC*MU*MA*MA*MAMAMAMCMAMUMGMGMCMCmCMCMAMG | | | | | | | | |
| | | MCMAMGMCmUmUmCmAmGmUmCmCmCmUmUmUmCdTdCdG | | | | | | | | |
| | | mUmCmGmAmUMGMGMU*MC*MA*MG*MC-3′ | | | | | | | | |
| | | SERPINA1 E342K Antisense-gRNA with 2′-F modification | | | | | | | | |
| KB-018-001 | 241 | 5′-mC*mU*mA*mA*mAmAmAmCmAmUmGmGmCmCmCmCmAmG | 55.3 | 2.8 | 29.1 | 6.4 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-403 | 242 | 5′-mC*FU*mA*mA*mAmAmAmCmAmUmGmGmCmCmCmCmAmG | 59.6 | 2.6 | 42.3 | 0.6 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-404 | 243 | 5′-mC*mU*FA*mA*mAmAmAmCmAmUmGmGmCmCmCmCmAmG | 62.3 | 1.7 | 43.7 | 5.1 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-405 | 244 | 5′-mC*mU*mA*FA*mAmAmAmCmAmUmGmGmCmCmCmCmAmG | 59.8 | 4.7 | 44.3 | 3.8 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-406 | 245 | 5′-mC*mU*mA*mA*FAmAmAmCmAmUmGmGmCmCmCmCmAmG | 59.0 | 4.4 | 45.9 | 5.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-407 | 246 | 5′-mC*mU*mA*mA*mAFAmAmCmAmUmGmGmCmCmCmCmAmG | 56.1 | 6.1 | 40.1 | 8.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-408 | 247 | 5′-mC*mU*mA*mA*mAmAFAmCmAmUmGmGmCmCmCmCmAmG | 59.3 | 0.9 | 37.1 | 3.9 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-409 | 248 | 5′-mC*mU*mA*mA*mAmAmAFCmAmUmGmGmCmCmCmCmAmG | 58.6 | 2.0 | 38.4 | 4.6 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-410 | 249 | 5′-mC*mU*mA*mA*mAmAmAmCFAmUmGmGmCmCmCmCmAmG | 55.2 | 8.3 | 40.4 | 2.4 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-411 | 250 | 5′-mC*mU*mA*mA*mAmAmAmCmAFUmGmGmCmCmCmCmAmG | 59.2 | 3.0 | 39.8 | 2.8 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-412 | 251 | 5′-mC*mU*mA*mA*mAmAmAmCmAmUFGmGmCmCmCmCmAmG | 57.8 | 9.1 | 45.6 | 6.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-413 | 252 | 5′-mC*mU*mA*mA*mAmAmAmCmAmUmGFGmCmCmCmCmAmG | 56.3 | 7.1 | 44.0 | 12.4 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-414 | 253 | 5′-mC*mU*mA*mA*mAmAmAmCmAmUmGmGFCmCmCmCmAmG | 57.1 | 5.3 | 34.2 | 5.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-415 | 254 | 5′-mC*mU*mA*mA*mAmAmAmCmAmUmGmGmCFCmCmCmAmG | 60.4 | 4.1 | 34.3 | 3.4 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-416 | 255 | 5′-mC*mU*mA*mA*mAmAmAmCmAmUmGmGmCmCFCmCmAmG | 64.1 | 3.5 | 36.7 | 7.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-417 | 256 | 5′-mC*mU*mA*mA*mAmAmAmCmAmUmGmGmCmCmCFCmAmG | 62.1 | 1.3 | 35.2 | 1.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-418 | 257 | 5′-mC*mU*mA*mA*mAmAmAmCmAmUmGmGmCmCmCmCFAmG | 55.9 | 0.9 | 31.3 | 1.5 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-419 | 258 | 5′-mC*mU*mA*mA*mAmAmAmCmAmUmGmGmCmCmCmCmAFG | 43.7 | 12.5 | 44.6 | 13.0 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-420 | 259 | 5′-mC*mU*mA*mA*mAmAmAmCmAmUmGmGmCmCmCmCmAmG | 57.9 | 2.7 | 47.8 | 5.2 | | | | |
| | | FCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-421 | 260 | 5′-mC*mU*mA*mA*mAmAmAmCmAmUmGmGmCmCmCmCmAmG | 56.1 | 3.1 | 46.3 | 8.9 | | | | |
| | | mCFAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-422 | 261 | 5′-mC*mU*mA*mA*mAmAmAmCmAmUmGmGmCmCmCmCmAmG | 58.0 | 2.4 | 46.4 | 2.1 | | | | |
| | | mCmAFGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-423 | 262 | 5′-mC*mU*mA*mA*mAmAmAmCmAmUmGmGmCmCmCmCmAmG | 53.8 | 1.7 | 16.0 | 7.0 | | | | |
| | | mCmAmGFCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-424 | 263 | 5′-mC*mU*mA*mA*mAmAmAmCmAmUmGmGmCmCmCmCmAmG | 45.4 | 17.8 | 45.7 | 5.7 | | | | |
| | | mCmAmGmCFUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-425 | 264 | 5′-mC*mU*mA*mA*mAmAmAmCmAmUmGmGmCmCmCmCmAmG | 75.2 | 5.0 | 65.4 | 2.6 | | | | |
| | | mCmAmGmCmUFUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-426 | 265 | 5′-mC*mU*mA*mA*mAmAmAmCmAmUmGmGmCmCmCmCmAmG | 62.6 | 4.3 | 50.7 | 1.3 | | | | |
| | | mCmAmGmCmUmUFCmAmGmUmCmCmCmUmUmUmCdTdCdG | | | | | | | | |
| | | mUmCmGmAmUmGmGmU*mC*mA*mG*mC-3′ | | | | | | | | |
| KB-018-427 | 266 | 5′-mC*mU*mA*mA*mAmAmAmCmAmUmGmGmCmCmCmCmAmG | 58.4 | 0.9 | 37.3 | 2.6 | | | | |
| | | mCmAmGmCmUmUmCFAmGmUmCmCmCmUmUmUmCdTdCdG | | | | | | | | |
| | | mUmCmGmAmUmGmGmU*mC*mA*mG*mC-3′ | | | | | | | | |
| KB-018-428 | 267 | 5′-mC*mU*mA*mA*mAmAmAmCmAmUmGmGmCmCmCmCmAmG | 63.4 | 2.6 | 45.0 | 4.6 | | | | |
| | | mCmAmGmCmUmUmCmAFGmUmCmCmCmUmUmUmCdTdCdG | | | | | | | | |
| | | mUmCmGmAmUmGmGmU*mC*mA*mG*mC-3′ | | | | | | | | |
| KB-018-429 | 268 | 5′-mC*mU*mA*mA*mAmAmAmCmAmUmGmGmCmCmCmCmAmG | 56.3 | 3.2 | 32.0 | 1.8 | | | | |
| | | mCmAmGmCmUmUmCmAmGFUmCmCmCmUmUmUmCdTdCdG | | | | | | | | |
| | | mUmCmGmAmUmGmGmU*mC*mA*mG*mC-3′ | | | | | | | | |
| KB-018-430 | 269 | 5′-mC*mU*mA*mA*mAmAmAmCmAmUmGmGmCmCmCmCmAmG | 61.2 | 2.5 | 42.2 | 2.6 | | | | |
| | | mCmAmGmCmUmUmCmAmGmUFCmCmCmUmUmUmCdTdCdG | | | | | | | | |
| | | mUmCmGmAmUmGmGmU*mC*mA*mG*mC-3′ | | | | | | | | |
| KB-018-431 | 270 | 5′-mC*mU*mA*mA*mAmAmAmCmAmUmGmGmCmCmCmCmAmG | 59.8 | 1.8 | 42.4 | 5.7 | | | | |
| | | mCmAmGmCmUmUmCmAmGmUmCFCmCmUmUmUmCdTdCdG | | | | | | | | |
| | | mUmCmGmAmUmGmGmU*mC*mA*mG*mC-3′ | | | | | | | | |
| KB-018-432 | 271 | 5′-mC*mU*mA*mA*mAmAmAmCmAmUmGmGmCmCmCmCmAmG | 60.5 | 4.9 | 51.5 | 16.3 | | | | |
| | | mCmAmGmCmUmUmCmAmGmUmCmCFCmUmUmUmCdTdCdG | | | | | | | | |
| | | mUmCmGmAmUmGmGmU*mC*mA*mG*mC-3′ | | | | | | | | |
| KB-018-433 | 272 | 5′-mC*mU*mA*mA*mAmAmAmCmAmUmGmGmCmCmCmCmAmG | 56.6 | 2.9 | 39.5 | 4.0 | | | | |
| | | mCmAmGmCmUmUmCmAmGmUmCmCmCFUmUmUmCdTdCdG | | | | | | | | |
| | | mUmCmGmAmUmGmGmU*mC*mA*mG*mC-3′ | | | | | | | | |
| KB-018-434 | 273 | 5′-mC*mU*mA*mA*mAmAmAmCmAmUmGmGmCmCmCmCmAmG | 54.5 | 2.8 | 35.4 | 6.0 | | | | |
| | | mCmAmGmCmUmUmCmAmGmUmCmCmCmUFUmUmCdTdCdG | | | | | | | | |
| | | mUmCmGmAmUmGmGmU*mC*mA*mG*mC-3′ | | | | | | | | |
| KB-018-435 | 274 | 5′-mC*mU*mA*mA*mAmAmAmCmAmUmGmGmCmCmCmCmAmG | 55.7 | 5.4 | 38.4 | 17.2 | | | | |
| | | mCmAmGmCmUmUmCmAmGmUmCmCmCmUmUFUmCdTdCdG | | | | | | | | |
| | | mUmCmGmAmUmGmGmU*mC*mA*mG*mC-3′ | | | | | | | | |
| KB-018-436 | 275 | 5′-mC*mU*mA*mA*mAmAmAmCmAmUmGmGmCmCmCmCmAmG | 62.0 | 3.7 | 34.8 | 7.8 | | | | |
| | | mCmAmGmCmUmUmCmAmGmUmCmCmCmUmUmUFCdTdCdG | | | | | | | | |
| | | mUmCmGmAmUmGmGmU*mC*mA*mG*mC-3′ | | | | | | | | |
| KB-018-437 | 276 | 5′-mC*mU*mA*mA*mAmAmAmCmAmUmGmGmCmCmCmCmAmG | 42.0 | 3.9 | 25.9 | 3.7 | | | | |
| | | mCmAmGmCmUmUmCmAmGmUmCmCmCmUmUmUmCFUdCd | | | | | | | | |
| | | GmUmCmGmAmUmGmGmU*mC*mA*mG*mC-3′ | | | | | | | | |
| KB-018-438 | 277 | 5′-mC*mU*mA*mA*mAmAmAmCmAmUmGmGmCmCmCmCmAmG | 32.7 | 2.0 | 23.1 | 5.4 | | | | |
| | | mCmAmGmCmUmUmCmAmGmUmCmCmCmUmUmUmCdTFCdG | | | | | | | | |
| | | mUmCmGmAmUmGmGmU*mC*mA*mG*mC-3′ | | | | | | | | |
| KB-018-439 | 278 | 5′-mC*mU*mA*mA*mAmAmAmCmAmUmGmGmCmCmCmCmAmG | 30.5 | 1.3 | 20.1 | 6.8 | | | | |
| | | mCmAmGmCmUmUmCmAmGmUmCmCmCmUmUmUmCdTdCFG | | | | | | | | |
| | | mUmCmGmAmUmGmGmU*mC*mA*mG*mC-3′ | | | | | | | | |
| KB-018-440 | 279 | 5′-mC*mU*mA*mA*mAmAmAmCmAmUmGmGmCmCmCmCmAmG | 68.7 | 4.9 | 59.5 | 3.5 | | | | |
| | | mCmAmGmCmUmUmCmAmGmUmCmCmCmUmUmUmCdTdCdG | | | | | | | | |
| | | FUmCmGmAmUmGmGmU*mC*mA*mG*mC-3′ | | | | | | | | |
| KB-018-441 | 280 | 5′-mC*mU*mA*mA*mAmAmAmCmAmUmGmGmCmCmCmCmAmG | 56.4 | 3.6 | 33.2 | 10.5 | | | | |
| | | mCmAmGmCmUmUmCmAmGmUmCmCmCmUmUmUmCdTdCdG | | | | | | | | |
| | | mUFCmGmAmUmGmGmU*mC*mA*mG*mC-3′ | | | | | | | | |
| KB-018-442 | 281 | 5′-mC*mU*mA*mA*mAmAmAmCmAmUmGmGmCmCmCmCmAmG | 56.9 | 3.0 | 38.4 | 12.5 | | | | |
| | | mCmAmGmCmUmUmCmAmGmUmCmCmCmUmUmUmCdTdCdG | | | | | | | | |
| | | mUmCFGmAmUmGmGmU*mC*mA*mG*mC-3′ | | | | | | | | |
| KB-018-443 | 282 | 5′-mC*mU*mA*mA*mAmAmAmCmAmUmGmGmCmCmCmCmAmG | 54.8 | 2.6 | 33.7 | 11.4 | | | | |
| | | mCmAmGmCmUmUmCmAmGmUmCmCmCmUmUmUmCdTdCdG | | | | | | | | |
| | | mUmCmGFAmUmGmGmU*mC*mA*mG*mC-3′ | | | | | | | | |
| KB-018-444 | 283 | 5′-mC*mU*mA*mA*mAmAmAmCmAmUmGmGmCmCmCmCmAmG | 51.8 | 5.1 | 39.7 | 22.0 | | | | |
| | | mCmAmGmCmUmUmCmAmGmUmCmCmCmUmUmUmCdTdCdG | | | | | | | | |
| | | mUmCmGmAFUmGmGmU*mC*mA*mG*mC-3′ | | | | | | | | |
| KB-018-445 | 284 | 5′-mC*mU*mA*mA*mAmAmAmCmAmUmGmGmCmCmCmCmAmG | 51.7 | 3.7 | 33.5 | 10.9 | | | | |
| | | mCmAmGmCmUmUmCmAmGmUmCmCmCmUmUmUmCdTdCdG | | | | | | | | |
| | | mUmCmGmAmUFGmGmU*mC*mA*mG*mC-3′ | | | | | | | | |
| KB-018-446 | 285 | 5′-mC*mU*mA*mA*mAmAmAmCmAmUmGmGmCmCmCmCmAmG | 58.1 | 3.2 | 29.7 | 4.8 | | | | |
| | | mCmAmGmCmUmUmCmAmGmUmCmCmCmUmUmUmCdTdCdG | | | | | | | | |
| | | mUmCmGmAmUmGFGmU*mC*mA*mG*mC-3′ | | | | | | | | |
| KB-018-447 | 286 | 5′-mC*mU*mA*mA*mAmAmAmCmAmUmGmGmCmCmCmCmAmG | 56.3 | 6.2 | 39.8 | 0.4 | | | | |
| | | mCmAmGmCmUmUmCmAmGmUmCmCmCmUmUmUmCdTdCdG | | | | | | | | |
| | | mUmCmGmAmUmGmGFU*mC*mA*mG*mC-3′ | | | | | | | | |
| KB-018-448 | 287 | 5′-mC*mU*mA*mA*mAmAmAmCmAmUmGmGmCmCmCmCmAmG | 52.2 | 4.2 | 19.8 | 4.9 | | | | |
| | | mCmAmGmCmUmUmCmAmGmUmCmCmCmUmUmUmCdTdCdG | | | | | | | | |
| | | mUmCmGmAmUmGmGmU*FC*mA*mG*mC-3′ | | | | | | | | |
| KB-018-449 | 288 | 5′-mC*mU*mA*mA*mAmAmAmCmAmUmGmGmCmCmCmCmAmG | 58.9 | 4.1 | 32.7 | 9.4 | | | | |
| | | mCmAmGmCmUmUmCmAmGmUmCmCmCmUmUmUmCdTdCdG | | | | | | | | |
| | | mUmCmGmAmUmGmGmU*mC*FA*mG*mC-3′ | | | | | | | | |
| KB-018-450 | 289 | 5′-mC*mU*mA*mA*mAmAmAmCmAmUmGmGmCmCmCmCmAmG | 55.6 | 8.4 | 32.6 | 10.3 | | | | |
| | | mCmAmGmCmUmUmCmAmGmUmCmCmCmUmUmUmCdTdCdG | | | | | | | | |
| | | mUmCmGmAmUmGmGmU*mC*mA*FG*mC-3′ | | | | | | | | |
| | | SERPINA1 E342K Antisense-gRNA with 2′-F Triplet | | | | | | | | |
| | | Modifications | | | | | | | | |
| KB-018-700 | 290 | 5′-mC*mU*mA*mA*mAmAmAmCmAmUmGmGmCmCmCmCmAmG | 10.3 | 0.4 | 7.0 | 0.4 | 20.6 | 2.6 | 11.0 | 0.8 |
| | | mCmAmGmCmUmUmCmAmGmUmCmCmCmUmUmUmCFUFCm | | | | | | | | |
| | | GmUmCmGmAmUmGmGmU*mC*mA*mG*mC-3′ | | | | | | | | |
| KB-018-701 | 291 | 5′-mC*mU*mA*mA*mAmAmAmCmAmUmGmGmCmCmCmCmAmG | 7.7 | 0.5 | 5.8 | 0.4 | 14.4 | 2.8 | 7.3 | 1.7 |
| | | mCmAmGmCmUmUmCmAmGmUmCmCmCmUmUmUmCmUFCm | | | | | | | | |
| | | GmUmCmGmAmUmGmGmU*mC*mA*mG*mC-3′ | | | | | | | | |
| KB-018-702 | 292 | 5′-mC*mU*mA*mA*mAmAmAmCmAmUmGmGmCmCmCmCmAmG | 13.6 | 1.3 | 9.8 | 1.8 | 15.6 | 1.2 | 9.8 | 1.8 |
| | | mCmAmGmCmUmUmCmAmGmUmCmCmCmUmUmUmCFUmCF | | | | | | | | |
| | | GmUmCmGmAmUmGmGmU*mC*mA*mG*mC-3′ | | | | | | | | |
| KB-018-703 | 293 | 5′-mC*mU*mA*mA*mAmAmAmCmAmUmGmGmCmCmCmCmAmG | 9.9 | 0.5 | 7.1 | 2.1 | 10.0 | 2.3 | 6.9 | 1.7 |
| | | mCmAmGmCmUmUmCmAmGmUmCmCmCmUmUmUmCmUmCF | | | | | | | | |
| | | GmUmCmGmAmUmGmGmU*mC*mA*mG*mC-3′ | | | | | | | | |
| KB-018-718 | 294 | 5′-mC*mU*mA*mA*mAmAmAmCmAmUmGmGmCmCmCmCmAmG | 13.7 | 0.2 | 6.0 | 1.3 | 6.9 | 0.9 | 4.7 | 1.3 |
| | | mCmAmGmCmUmUmCmAmGmUmCmCmCmUmUmUmCFUFCF | | | | | | | | |
| | | GmUmCmGmAmUmGmGmU*mC*mA*mG*mC-3′ | | | | | | | | |
| KB-018-719 | 295 | 5′-mC*mU*mA*mA*mAmAmAmCmAmUmGmGmCmCmCmCmAmG | 12.7 | 1.7 | 6.0 | 0.8 | 7.6 | 0.8 | 4.0 | 0.6 |
| | | mCmAmGmCmUmUmCmAmGmUmCmCmCmUmUmUmCdTFCFG | | | | | | | | |
| | | mUmCmGmAmUmGmGmU*mC*mA*mG*mC-3′ | | | | | | | | |
| KB-018-720 | 296 | 5′-mC*mU*mA*mA*mAmAmAmCmAmUmGmGmCmCmCmCmAmG | 15.6 | 0.3 | 5.5 | 0.8 | 7.9 | 1.4 | 4.3 | 0.7 |
| | | mCmAmGmCmUmUmCmAmGmUmCmCmCmUmUmUmCFUdCF | | | | | | | | |
| | | GmUmCmGmAmUmGmGmU*mC*mA*mG*mC-3′ | | | | | | | | |
| KB-018-721 | 297 | 5′-mC*mU*mA*mA*mAmAmAmCmAmUmGmGmCmCmCmCmAmG | 23.7 | 1.5 | 10.0 | 1.3 | 10.2 | 1.1 | 4.6 | 0.9 |
| | | mCmAmGmCmUmUmCmAmGmUmCmCmCmUmUmUmCFUFCd | | | | | | | | |
| | | GmUmCmGmAmUmGmGmU*mC*mA*mG*mC-3′ | | | | | | | | |
| KB-018-437 | 298 | 5′-mC*mU*mA*mA*mAmAmAmCmAmUmGmGmCmCmCmCmAmG | 42.9 | 2.0 | 17.7 | 1.3 | 18.3 | 0.9 | 7.3 | 1.1 |
| | | mCmAmGmCmUmUmCmAmGmUmCmCmCmUmUmUmCFUdCd | | | | | | | | |
| | | GmUmCmGmAmUmGmGmU*mC*mA*mG*mC-3′ | | | | | | | | |
| KB-018-438 | 299 | 5′-mC*mU*mA*mA*mAmAmAmCmAmUmGmGmCmCmCmCmAmG | 30.6 | 2.0 | 11.7 | 1.0 | 11.2 | 1.9 | | |
| | | mCmAmGmCmUmUmCmAmGmUmCmCmCmUmUmUmCdTFCdG | | | | | | | | |
| | | mUmCmGmAmUmGmGmU*mC*mA*mG*mC-3′ | | | | | | | | |
| KB-018-439 | 300 | 5′-mC*mU*mA*mA*mAmAmAmCmAmUmGmGmCmCmCmCmAmG | 28.5 | 4.3 | 11.2 | 1.3 | 12.7 | 0.9 | 4.6 | 1.2 |
| | | mCmAmGmCmUmUmCmAmGmUmCmCmCmUmUmUmCdTdCFG | | | | | | | | |
| | | mUmCmGmAmUmGmGmU*mC*mA*mG*mC-3′ | | | | | | | | |
| KB-018-440 | 301 | 5′-mC*mU*mA*mA*mAmAmAmCmAmUmGmGmCmCmCmCmAmG | 10.8 | 0.9 | 5.0 | 0.4 | 9.9 | 0.6 | 5.4 | 0.3 |
| | | mCmAmGmCmUmUmCmAmGmUmCmCmCmUmUmUmCmUFCF | | | | | | | | |
| | | GmUmCmGmAmUmGmGmU*mC*mA*mG*mC-3′ | | | | | | | | |
| | | SERPINA1 E342K Antisense-gRNA: 2′-F and FANA | | | | | | | | |
| | | modifications | | | | | | | | |
| KB-018-001 | 302 | 5′-mC*mU*mA*mA*mAmAmAmCmAmUmGmGmCmCmCmCmAmG | 55.1 | 3.9 | 36.3 | 0.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-1265 | 303 | 5′-mC*mU*mA*mA*mAmAmAmCmAmUmGmGmCmCmCmCmAmG | 72.3 | 10.3 | 53.9 | 1.6 | | | | |
| | | mCmAmGmCmUFUmCmAmGmUmCmCmCmUmUmUmCdTdCdG | | | | | | | | |
| | | FUmCmGmAmUmGmGmU*mC*mA*mG*mC-3′ | | | | | | | | |
| KB-018-1266 | 304 | 5′-mC*mU*mA*mA*mAmAmAmCmAmUmGmGmCmCFCmCmAmG | 77.7 | 3.3 | 55.7 | 2.7 | | | | |
| | | mCmAmGmCmUFUmCmAmGmUmCmCmCmUmUmUmCdTdCdG | | | | | | | | |
| | | FUmCmGmAmUmGmGmU*mC*mA*mG*mC-3′ | | | | | | | | |
| KB-018-1267 | 305 | 5′-mC*mU*mA*mA*mAmAmAmCmAmUmGmGmCmCFCmCmAmG | 78.3 | 2.4 | 55.6 | 3.1 | | | | |
| | | mCmAmGmCmUFUmCmAmGmUmCmCmCmUmUmUFCdTdCdG | | | | | | | | |
| | | FUmCmGmAmUmGmGmU*mC*mA*mG*mC-3′ | | | | | | | | |
| KB-018-1268 | 306 | 5′-mC*mU*mA*mA*mAmAmAmCmAmUmGmGmCmCFCmCmAmG | 70.7 | 18.5 | 61.3 | 7.5 | | | | |
| | | mCmAmGmCFUFUmCmAmGmUmCmCmCmUmUmUFCdTdCdGF | | | | | | | | |
| | | UmCmGmAmUmGmGmU*mC*mA*mG*mC-3′ | | | | | | | | |
| KB-018-697 | 307 | 5′-mC*mU*mA*mA*mAmAmAmCmAmUmGmGmCmCmCmCmAmG | 28.9 | 2.3 | 0.0 | 0.0 | | | | |
| | | mCmAmGmCmUmUmCmAmGmUmCmCmCmUmUmUmCfUfCfGf | | | | | | | | |
| | | UmCmGmAmUmGmGmU*mC*mA*mG*mC-3′ | | | | | | | | |
| KB-018-699 | 308 | 5′-mC*mU*mA*mA*mAmAmAmCmAmUmGmGmCmCmCmCmAmG | 44.7 | 3.1 | 29.1 | 5.6 | | | | |
| | | mCmAmGmCmUmUmCmAmGmUmCmCmCmUmUmUfCfUfCfGfU | | | | | | | | |
| | | mCmGmAmUmGmGmU*mC*mA*mG*mC-3′ | | | | | | | | |
| | | SERPINA1 E342K Antisense-gRNA with 2′-O-MOE and 2′-F | | | | | | | | |
| | | modifications | | | | | | | | |
| KB-018-001 | 309 | 5′-mC*mU*mA*mA*mAmAmAmCmAmUmGmGmCmCmCmCmAmG | 55.1 | 3.9 | 36.3 | 0.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-1269 | 310 | 5′-MC*MU*MA*MA*MAMAMAMCMAMUMGMGMCMCMCMCMAMG | 44.8 | 2.9 | 0.0 | 0.0 | | | | |
| | | MCMAMGMCMUMUMCMAMGMUMCMCMCMUMUMUMCdTdCdG | | | | | | | | |
| | | FUFCMGMAMUMGMGMU*MC*MA*MG*MC-3′ | | | | | | | | |
| KB-018-1270 | 311 | 5′-MC*MU*MA*MA*MAMAMAMCMAMUMGMGMCMCFCMCMAMG | 34.5 | 0.0 | 0.0 | 0.0 | | | | |
| | | MCMAMGMCmUFUMCMAMGMUMCMCMCMUMUMUFCdTdCdG | | | | | | | | |
| | | FUFCMGMAMUMGMGMU*MC*MA*MG*MC-3′ | | | | | | | | |
| KB-018-1271 | 312 | 5′-MC*MU*MA*MA*MAMAMAMCMAMUMGMGMCMCFCMCMAMG | 48.3 | 4.2 | 28.9 | 3.7 | | | | |
| | | MCMAMGMCmUFUmCmAMGmUmCmCmCMUMUMUFCdTdCdG | | | | | | | | |
| | | FUmCMGMAMUMGMGMU*MC*MA*MG*MC-3′ | | | | | | | | |
| KB-018-1272 | 313 | 5′-MC*mU*MA*MA*MAMAmAMCMAMUMGMGMCMCFCMCMAMG | 50.8 | 15.3 | 44.8 | 14.9 | | | | |
| | | MCMAMGMCmUFUmCmAMGmUmCmCmCMUMUmUFCdTdCdG | | | | | | | | |
| | | FUmCmGmAmUMGMGMU*MC*mA*mG*MC-3′ | | | | | | | | |
| KB-018-1290 | 314 | 5′-MC*MU*MA*MA*MAMAMAMCMAMUMGMGMCMCFCMCMAMG | 0.0 | 0.0 | 0.0 | 0.0 | | | | |
| | | MCMAMGMCmUFUmCmAmGmUmCmCmCmUmUmUFCdTdCdG | | | | | | | | |
| | | FUmCmGmAmUMGMGMU*MC*MA*MG*MC-3′ | | | | | | | | |
| KB-018-1273 | 315 | 5′-MC*MU*MA*MA*MAMAMAMCMAMUMGMGMCMCfCMCMAMG | | | | | | | | |
| | | MCMAMGMCMUfUMCMAMGMUMCMCMCMUMUMUMCfUfCfGfU | | | | | | | | |
| | | fCMGMAMUMGMGMU*MC*MA*MG*MC-3′ | | | | | | | | |
| KB-018-1274 | 316 | 5′-MC*mU*MA*MA*MAMAmAMCMAMUMGMGMCMCfCMCMAMG | | | | | | | | |
| | | MCMAMGMCmUfUmCmAMGmUmCmCmCMUMUmUfCfUfCfGfU | | | | | | | | |
| | | mCmGmAmUMGMGMU*MC*mA*mG*MC-3′ | | | | | | | | |

TABLE 8
Triplet Mismatches (underlined)

| | | | Editing Data | | | | | | | |
|---|---|---|---|---|---|---|---|---|---|---|
| | | | ADAR2 % | | | | ADAR1 p110 | | | |
| | SEQ | | Editing | | | | % Editing | | | |
| | ID | | 100 | | 10 | | 100 | | 10 | |
| Seq ID | NO | Guide Oligonucleotide Sequences | nM | SD | nM | SD | nM | SD | nM | SD |
| KB-018-001 | 317 | 5′-mC*mU*mA*mA*mAmAmAmCmAmUmGmGmCmCmCmCmAmGm | 44.7 | 1.7 | 33.8 | 0.0 | | | | |
| | | CmAmGmCmUmUmCmAmGmUmCmCmCmUmUmUmCdTdCdGmU | | | | | | | | |
| | | mCmGmAmUmGmGmU*mC*mA*mG*mC-3′ | | | | | | | | |
| KB-018-012 | 318 | 5′-mC*mU*mA*mA*mAmAmAmCmAmUmGmGmCmCmCmCmAmGm | 9.3 | 1.5 | 5.0 | 0.5 | | | | |
| | | CmAmGmCmUmUmCmAmGmUmCmCmCmUmUmUmCdTdTdGmU | | | | | | | | |
| | | mCmGmAmUmGmGmU*mC*mA*mG*mC-3′ | | | | | | | | |
| KB-018-174 | 319 | 5′-mC*mU*mA*mA*mAmAmAmCmAmUmGmGmCmCmCmCmAmGm | 26.0 | 0.5 | 11.6 | 1.3 | | | | |
| | | CmAmGmCmUmUmCmAmGmUmCmCmCmUmUmUmCdTdGdGmU | | | | | | | | |
| | | mCmGmAmUmGmGmU*mC*mA*mG*mC-3′ | | | | | | | | |
| KB-018-175 | 320 | 5′-mC*mU*mA*mA*mAmAmAmCmAmUmGmGmCmCmCmCmAmGm | 14.3 | 0.7 | 8.6 | 1.4 | | | | |
| | | CmAmGmCmUmUmCmAmGmUmCmCmCmUmUmUmCdTdAdGmU | | | | | | | | |
| | | mCmGmAmUmGmGmU*mC*mA*mG*mC-3′ | | | | | | | | |
| KB-018-176 | 321 | 5′-mC*mU*mA*mA*mAmAmAmCmAmUmGmGmCmCmCmCmAmGm | 25.1 | 0.7 | 8.9 | 1.5 | | | | |
| | | CmAmGmCmUmUmCmAmGmUmCmCmCmUmUmUmCdGdCdGmU | | | | | | | | |
| | | mCmGmAmUmGmGmU*mC*mA*mG*mC-3′ | | | | | | | | |
| KB-018-177 | 322 | 5′-mC*mU*mA*mA*mAmAmAmCmAmUmGmGmCmCmCmCmAmGm | 37.8 | 4.3 | 22.5 | 1.7 | | | | |
| | | CmAmGmCmUmUmCmAmGmUmCmCmCmUmUmUmCdIdCdGmU | | | | | | | | |
| | | mCmGmAmUmGmGmU*mC*mA*mG*mC-3′ | | | | | | | | |
| KB-018-178 | 323 | 5′-mC*mU*mA*mA*mAmAmAmCmAmUmGmGmCmCmCmCmAmGm | 17.1 | 1.7 | 12.9 | 1.8 | | | | |
| | | CmAmGmCmUmUmCmAmGmUmCmCmCmUmUmUmCdAdCdGmU | | | | | | | | |
| | | mCmGmAmUmGmGmU*mC*mA*mG*mC-3′ | | | | | | | | |
| KB-018-179 | 324 | 5′-mC*mU*mA*mA*mAmAmAmCmAmUmGmGmCmCmCmCmAmGm | 20.6 | 0.8 | 15.0 | 0.9 | | | | |
| | | CmAmGmCmUmUmCmAmGmUmCmCmCmUmUmUmCdCdCdGmU | | | | | | | | |
| | | mCmGmAmUmGmGmU*mC*mA*mG*mC-3′ | | | | | | | | |
| KB-018-180 | 325 | 5′-mC*mU*mA*mA*mAmAmAmCmAmUmGmGmCmCmCmCmAmGm | 11.5 | 1.5 | 6.4 | 1.6 | | | | |
| | | CmAmGmCmUmUmCmAmGmUmCmCmCmUmUmUmCdTdCdAmU | | | | | | | | |
| | | mCmGmAmUmGmGmU*mC*mA*mG*mC-3′ | | | | | | | | |
| KB-018-181 | 326 | 5′-mC*mU*mA*mA*mAmAmAmCmAmUmGmGmCmCmCmCmAmGm | 6.0 | 0.5 | 2.6 | 0.5 | | | | |
| | | CmAmGmCmUmUmCmAmGmUmCmCmCmUmUmUmCdTdCdCmU | | | | | | | | |
| | | mCmGmAmUmGmGmU*mC*mA*mG*mC-3′ | | | | | | | | |
| KB-018-182 | 327 | 5′-mC*mU*mA*mA*mAmAmAmCmAmUmGmGmCmCmCmCmAmGm | 7.7 | 1.0 | 6.0 | 0.7 | | | | |
| | | CmAmGmCmUmUmCmAmGmUmCmCmCmUmUmUmCdTdCdTmU | | | | | | | | |
| | | mCmGmAmUmGmGmU*mC*mA*mG*mC-3′ | | | | | | | | |
| KB-018-183 | 328 | 5′-mC*mU*mA*mA*mAmAmAmCmAmUmGmGmCmCmCmCmAmGm | 2.9 | 0.2 | 2.4 | 0.1 | | | | |
| | | CmAmGmCmUmUmCmAmGmUmCmCmCmUmUmUmCdGdCdCmU | | | | | | | | |
| | | mCmGmAmUmGmGmU*mC*mA*mG*mC-3′ | | | | | | | | |

TABLE 9
Flanking Sequence Mismatches (underlined)

| | | | Editing Data | | | | | | | |
|---|---|---|---|---|---|---|---|---|---|---|
| | | | ADAR2 % | | | | ADAR1 p110 % | | | |
| | SEQ | | Editing | | | | Editing | | | |
| | ID | | 100 | | 10 | | 100 | | 10 | |
| Seq ID | NO | Guide Oligonucleotide Sequences | nM | SD | nM | SD | nM | SD | nM | SD |
| | | SERPINA1 E342K Antisense-gRNAs with single mismatch | | | | | | | | |
| KB-018-001 | 329 | 5′-mC*mU*mA*mA*mAmAmAmCmAmUmGmGmCmCmCmCmAmG | 57.8 | 4.1 | 26.8 | 3.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-110 | 330 | 5′-mC*mU*mA*mA*mAmAmAmCmAmUmGmGmCmCmCmCmAmG | 46.5 | 0.9 | 31.1 | 3.7 | | | | |
| | | mCmAmGmCmUmUmCmAmGmUmCmCmCmCmUmUmCdTdCdG | | | | | | | | |
| | | mUmCmGmAmUmGmGmU*mC*mA*mG*mC-3′ | | | | | | | | |
| KB-018-559 | 331 | 5′-mC*mU*mA*mA*mAmAmAmCmAmUmGmGmCmCmCmCmAmG | 63.2 | 1.4 | 40.3 | 1.1 | | | | |
| | | mCmAmGmCmUmUmCmAmGmUmCmCmGmUmUmUmCdTdCd | | | | | | | | |
| | | GmUmCmGmAmUmGmGmU*mC*mA*mG*mC-3′ | | | | | | | | |
| KB-018-560 | 332 | 5′-mC*mU*mA*mA*mAmAmAmCmAmUmGmGmCmCmCmCmAmG | 55.0 | 2.8 | 32.7 | 3.1 | | | | |
| | | mCmAmGmCmUmUmCmAmGmUmCmGmCmUmUmUmCdTdCd | | | | | | | | |
| | | GmUmCmGmAmUmGmGmU*mC*mA*mG*mC-3′ | | | | | | | | |
| KB-018-561 | 333 | 5′-mC*mU*mA*mA*mAmAmAmCmAmUmGmGmCmCmCmCmAmG | 60.3 | 10.0 | 29.5 | 6.6 | | | | |
| | | mCmAmGmCmUmUmCmAmGmUmGmCmCmUmUmUmCdTdCd | | | | | | | | |
| | | GmUmCmGmAmUmGmGmU*mC*mA*mG*mC-3′ | | | | | | | | |
| KB-018-111 | 334 | 5′-mC*mU*mA*mA*mAmAmAmCmAmUmGmGmCmCmCmCmAmG | 53.3 | 5.4 | 31.3 | 5.0 | | | | |
| | | mCmAmGmCmUmUmCmAmGmCmCmCmCmUmUmUmCdTdCdG | | | | | | | | |
| | | mUmCmGmAmUmGmGmU*mC*mA*mG*mC-3′ | | | | | | | | |
| KB-018-562 | 335 | 5′-mC*mU*mA*mA*mAmAmAmCmAmUmGmGmCmCmCmCmAmG | 56.0 | 1.4 | 38.4 | 1.2 | | | | |
| | | mCmAmGmCmUmUmCmAmCmUmCmCmCmUmUmUmCdTdCdG | | | | | | | | |
| | | mUmCmGmAmUmGmGmU*mC*mA*mG*mC-3′ | | | | | | | | |
| KB-018-563 | 336 | 5′-mC*mU*mA*mA*mAmAmAmCmAmUmGmGmCmCmCmCmAmG | 51.8 | 1.5 | 33.0 | 1.1 | | | | |
| | | mCmAmGmCmUmUmCmUmGmUmCmCmCmUmUmUmCdTdCd | | | | | | | | |
| | | GmUmCmGmAmUmGmGmU*mC*mA*mG*mC-3′ | | | | | | | | |
| KB-018-564 | 337 | 5′-mC*mU*mA*mA*mAmAmAmCmAmUmGmGmCmCmCmCmAmG | 41.2 | 7.3 | 21.6 | 5.5 | | | | |
| | | mCmAmGmCmUmUmGmAmGmUmCmCmCmUmUmUmCdTdCd | | | | | | | | |
| | | GmUmCmGmAmUmGmGmU*mC*mA*mG*mC-3′ | | | | | | | | |
| KB-018-112 | 338 | 5′-mC*mU*mA*mA*mAmAmAmCmAmUmGmGmCmCmCmCmAmG | 48.9 | 4.3 | 25.0 | 3.0 | | | | |
| | | mCmAmGmCmUmCmCmAmGmUmCmCmCmUmUmUmCdTdCdG | | | | | | | | |
| | | mUmCmGmAmUmGmGmU*mC*mA*mG*mC-3′ | | | | | | | | |
| KB-018-113 | 339 | 5′-mC*mU*mA*mA*mAmAmAmCmAmUmGmGmCmCmCmCmAmG | 54.3 | 2.6 | 26.2 | 1.6 | | | | |
| | | mCmAmGmCmCmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-565 | 340 | 5′-mC*mU*mA*mA*mAmAmAmCmAmUmGmGmCmCmCmCmAmG | 56.8 | 0.7 | 21.9 | 1.1 | | | | |
| | | mCmAmGmGmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-566 | 341 | 5′-mC*mU*mA*mA*mAmAmAmCmAmUmGmGmCmCmCmCmAmG | 58.5 | 3.7 | 31.2 | 1.6 | | | | |
| | | mCmAmCmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-172 | 342 | 5′-mC*mU*mA*mA*mAmAmAmCmAmUmGmGmCmCmCmCmAmG | 59.9 | 1.7 | 27.8 | 1.5 | | | | |
| | | mCmCmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-567 | 343 | 5′-mC*mU*mA*mA*mAmAmAmCmAmUmGmGmCmCmCmCmAmG | 56.9 | 3.4 | 32.6 | 2.1 | | | | |
| | | mGmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-568 | 344 | 5′-mC*mU*mA*mA*mAmAmAmCmAmUmGmGmCmCmCmCmAmC | 50.6 | 2.8 | 30.1 | 2.2 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-569 | 345 | 5′-mC*mU*mA*mA*mAmAmAmCmAmUmGmGmCmCmCmCmUm | 58.8 | 1.7 | 28.4 | 3.0 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-570 | 346 | 5′-mC*mU*mA*mA*mAmAmAmCmAmUmGmGmCmCmCmGmAm | 58.8 | 3.5 | 33.0 | 0.6 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-571 | 347 | 5′-mC*mU*mA*mA*mAmAmAmCmAmUmGmGmCmCmGmCmAm | 61.3 | 1.2 | 30.9 | 1.1 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-572 | 348 | 5′-mC*mU*mA*mA*mAmAmAmCmAmUmGmGmCmGmCmCmAm | 64.4 | 1.0 | 30.5 | 1.7 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-573 | 349 | 5′-mC*mU*mA*mA*mAmAmAmCmAmUmGmGmGmCmCmCmAm | 58.8 | 1.9 | 32.2 | 1.5 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-574 | 350 | 5′-mC*mU*mA*mA*mAmAmAmCmAmUmGmCmCmCmCmCmAmG | 58.0 | 2.1 | 28.1 | 1.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-575 | 351 | 5′-mC*mU*mA*mA*mAmAmAmCmAmUmCmGmCmCmCmCmAmG | 53.1 | 1.4 | 27.0 | 0.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-114 | 352 | 5′-mC*mU*mA*mA*mAmAmAmCmAmCmGmGmCmCmCmCmAm | 50.5 | 3.3 | 25.1 | 1.9 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-576 | 353 | 5′-mC*mU*mA*mA*mAmAmAmCmUmUmGmGmCmCmCmCmAm | 60.8 | 3.2 | 30.9 | 3.5 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-577 | 354 | 5′-mC*mU*mA*mA*mAmAmAmGmAmUmGmGmCmCmCmCmAm | 58.5 | 4.0 | 31.2 | 3.9 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-578 | 355 | 5′-mC*mU*mA*mA*mAmAmUmCmAmUmGmGmCmCmCmCmAm | 61.4 | 2.5 | 28.6 | 2.6 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-579 | 356 | 5′-mC*mU*mA*mA*mAmUmAmCmAmUmGmGmCmCmCmCmAm | 57.7 | 2.2 | 26.5 | 2.3 | | | | |
| | | GmCmAmGmCmUmUmCmAmGmUmCmCmCmUmUmUmCdTdCd | | | | | | | | |
| | | GmUmCmGmAmUmGmGmU*mC*mA*mG*mC-3′ | | | | | | | | |
| | | SERPINA1 E342K Antisense-gRNAs with two mismatches | | | | | | | | |
| KB-018-001 | 357 | 5′-mC*mU*mA*mA*mAmAmAmCmAmUmGmGmCmCmCmCmAmG | 45.3 | 3.6 | 32.5 | 1.5 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-093 | 358 | 5′-mC*mU*mA*mA*mAmAmAmCmAmUmGmGmCmCmCmCmAmG | 26.4 | 5.7 | 17.2 | 4.1 | | | | |
| | | mCmAmGmCmUmUmCmAmGmUmCmCmCmUmUmUmCrUrCrG | | | | | | | | |
| | | mUmCmGmAmUmGmGmU*mC*mA*mG*mC-3′ | | | | | | | | |
| KB-018-108 | 359 | 5′-mC*mU*mA*mA*mAmAmAmCmAmUmGmGmCmCmCmCmAmG | | | | | | | | |
| | | mCmAmGmCmUmUmCmAmGmUmGmCmCmUmCmUmCdTdCd | | | | | | | | |
| | | GmUmCmGmAmUmGmGmU*mC*mA*mG*mC-3′ | | | | | | | | |
| KB-018-094 | 360 | 5′-mC*mU*mA*mA*mAmAmAmCmAmUmGmGmCmCmCmCmAmG | 35.4 | 6.8 | 27.3 | 2.7 | | | | |
| | | mCmAmAmCmUmUmGmAmGmUmCmCmCmUmUmUmCdTdCd | | | | | | | | |
| | | GmUmCmGmAmUmGmGmU*mC*mA*mG*mC-3′ | | | | | | | | |
| KB-018-095 | 361 | 5′-mC*mU*mA*mA*mAmAmAmCmAmUmGmGmCmCmCmCmAmG | 13.7 | 3.0 | 9.0 | 1.4 | | | | |
| | | mCmAmAmCmUmUmGmAmGmUmCmCmCmUmUmUmCrUrCrG | | | | | | | | |
| | | mUmCmGmAmUmGmGmU*mC*mA*mG*mC-3′ | | | | | | | | |
| KB-018-096 | 362 | 5′-mC*mU*mA*mA*mAmAmAmCmAmUmGmGmCmCmCmCmAmG | 23.8 | 3.6 | 13.8 | 2.2 | | | | |
| | | mCmAmAmCmUmUmGmAmGmUmCmCmCmUmUmUmCmUdCd | | | | | | | | |
| | | GmUmCmGmAmUmGmGmU*mC*mA*mG*mC-3′ | | | | | | | | |
| KB-018-097 | 363 | 5′-mC*mU*mA*mA*mAmAmAmCmAmUmGmGmCmCmCmCmAmG | 15.9 | 2.6 | 9.3 | 1.6 | | | | |
| | | mCmAmAmCmUmUmGmAmGmUmCmCmCmUmUmUmCmUrCrG | | | | | | | | |
| | | mUmCmGmAmUmGmGmU*mC*mA*mG*mC-3′ | | | | | | | | |
| KB-018-098 | 364 | 5′-mA*mU*mG*mG*mCmCmCmCmAmGmCmAmAmCmUmUmGm | 27.9 | 2.4 | 14.6 | 3.7 | | | | |
| | | AmGmUmCmCmCmUmUmUmCdTdCdGmUmCmGmAmU*mG*mG | | | | | | | | |
| | | *mU*mC-3′ | | | | | | | | |
| KB-018-099 | 365 | 5′-mA*mU*mG*mG*mCmCmCmCmAmGmCmAmAmCmUmUmGm | 7.7 | 1.5 | 2.4 | 0.9 | | | | |
| | | AmGmUmCmCmCmUmUmUmCrUrCrGmUmCmGmAmU*mG*mG* | | | | | | | | |
| | | mU*mC-3′ | | | | | | | | |
| KB-018-100 | 366 | 5′-mC*mA*mU*mG*mGmCmCmCmCmAmGmCmGmGmCmUmCm | 18.5 | 3.1 | 7.4 | 1.6 | | | | |
| | | CmAmGmUmCmCmCmUmUmUmCdTdCdGmUmCmGmAmU*mG* | | | | | | | | |
| | | mG*mU*mC-3′ | | | | | | | | |
| KB-018-101 | 367 | 5′-mC*mA*mU*mG*mGmCmCmCmCmAmGmCmGmGmCmUmCm | 13.2 | 2.4 | 5.2 | 1.1 | | | | |
| | | CmAmGmUmCmCmCmUmUmUmCrUrCrGmUmCmGmAmU*mG* | | | | | | | | |
| | | mG*mU*mC-3′ | | | | | | | | |
| KB-018-109 | 368 | 5′-mC*mU*mA*mA*mAmAmAmCmAmUmGmGmCmCmCmCmAmG | | | | | | | | |
| | | mGmAmGmCmCmUmCmAmGmUmCmCmCmUmUmUmCdTdCd | | | | | | | | |
| | | GmUmCmGmAmUmGmGmU*mC*mA*mG*mC-3′ | | | | | | | | |
| KB-018-102 | 369 | 5′-mC*mU*mA*mA*mAmAmAmCmAmUmGmGmCmCmCmGmAm | 59.6 | 3.2 | 47.2 | 3.6 | | | | |
| | | GmCmCmGmCmUmUmCmAmGmUmCmCmCmUmUmUmCdTdC | | | | | | | | |
| | | dGmUmCmGmAmUmGmGmU*mC*mA*mG*mC-3′ | | | | | | | | |
| KB-018-103 | 370 | 5′-mC*mU*mA*mA*mAmAmAmCmAmUmGmGmCmCmCmGmAm | 30.0 | 3.0 | 19.5 | 3.8 | | | | |
| | | GmCmCmGmCmUmUmCmAmGmUmCmCmCmUmUmUmCrUrCr | | | | | | | | |
| | | GmUmCmGmAmUmGmGmU*mC*mA*mG*mC-3′ | | | | | | | | |
| KB-018-104 | 371 | 5′-mC*mU*mA*mA*mAmAmAmCmAmUmGmGmCmCmCmGmAm | 30.0 | 10.2 | 19.6 | 6.6 | | | | |
| | | GmCmCmGmCmUmUmCmAmGmUmCmCmCmUmUmUmCmUdC | | | | | | | | |
| | | dGmUmCmGmAmUmGmGmU*mC*mA*mG*mC-3′ | | | | | | | | |
| KB-018-105 | 372 | 5′-mC*mU*mA*mA*mAmAmAmCmAmUmGmGmCmCmCmGmAm | 30.4 | 4.4 | 19.2 | 4.0 | | | | |
| | | GmCmCmGmCmUmUmCmAmGmUmCmCmCmUmUmUmCmUrCr | | | | | | | | |
| | | GmUmCmGmAmUmGmGmU*mC*mA*mG*mC-3′ | | | | | | | | |
| KB-018-106 | 373 | 5′-mA*mA*mA*mC*mAmUmGmGmCmCmCmGmAmGmCmCmGm | 48.9 | 2.3 | 31.0 | 2.6 | | | | |
| | | CmUmUmCmAmGmUmCmCmCmUmUmUmCdTdCdGmUmCmGm | | | | | | | | |
| | | AmU*mG*mG*mU*mC-3′ | | | | | | | | |
| KB-018-107 | 374 | 5′-mA*mA*mA*mC*mAmUmGmGmCmCmCmGmAmGmCmCmGm | 18.4 | 1.7 | 6.1 | 0.7 | | | | |
| | | CmUmUmCmAmGmUmCmCmCmUmUmUmCrUrCrGmUmCmGm | | | | | | | | |
| | | AmU*mG*mG*mU*mC-3′ | | | | | | | | |
| | | SERPINA1 E342K Antisense-gRNA with FANA triplet | | | | | | | | |
| | | modifications | | | | | | | | |
| KB-018-102 | 375 | 5′-mC*mU*mAkmA*mAmAmAmCmAmUmGmGmCmCmCmGmAm | 48.2 | 4.6 | 34.6 | 12.3 | | | | |
| | | GmCmCmGmCmUmUmCmAmGmUmCmCmCmUmUmUmCdTdC | | | | | | | | |
| | | dGmUmCmGmAmUmGmGmU*mC*mAkmG*mC-3′ | | | | | | | | |
| KB-018-195 | 376 | 5′-mC*mU*mAkmA*mAmAmAmCmAmUmGmGmCmCmCmGmAm | 62.5 | 2.5 | 42.8 | 0.5 | | | | |
| | | GmCmCmGmCmUmUmCmAmGmUmCmCmCmUmUmUmCfUdCd | | | | | | | | |
| | | GmUmCmGmAmUmGmGmU*mC*mAkmG*mC-3′ | | | | | | | | |
| KB-018-196 | 377 | 5′-mC*mU*mAkmA*mAmAmAmCmAmUmGmGmCmCmCmGmAm | 65.8 | 3.6 | 53.4 | 2.4 | | | | |
| | | GmCmCmGmCmUmUmCmAmGmUmCmCmCmUmUmUmCdTfCd | | | | | | | | |
| | | GmUmCmGmAmUmGmGmU*mC*mAkmG*mC-3′ | | | | | | | | |
| KB-018-197 | 378 | 5′-mC*mU*mAkmA*mAmAmAmCmAmUmGmGmCmCmCmGmAm | 69.3 | 5.2 | 53.7 | 4.5 | | | | |
| | | GmCmCmGmCmUmUmCmAmGmUmCmCmCmUmUmUmCdTdCf | | | | | | | | |
| | | GmUmCmGmAmUmGmGmU*mC*mAkmG*mC-3′ | | | | | | | | |
| KB-018-198 | 379 | 5′-mC*mU*mAkmA*mAmAmAmCmAmUmGmGmCmCmCmGmAm | 62.7 | 10.1 | 53.6 | 2.9 | | | | |
| | | GmCmCmGmCmUmUmCmAmGmUmCmCmCmUmUmUmCfUfCf | | | | | | | | |
| | | GmUmCmGmAmUmGmGmU*mC*mAkmG*mC-3′ | | | | | | | | |
| KB-018-199 | 380 | 5′-mC*mU*mAkmA*mAmAmAmCmAmUmGmGmCmCmCmGmAm | 70.1 | 2.0 | 57.7 | 2.9 | | | | |
| | | GmCmCmGmCmUmUmCmAmGmUmCmCmCmUmUmUmCdTfCf | | | | | | | | |
| | | GmUmCmGmAmUmGmGmU*mC*mAkmG*mC-3′ | | | | | | | | |
| KB-018-200 | 381 | 5′-mC*mU*mAkmA*mAmAmAmCmAmUmGmGmCmCmCmGmAm | 63.0 | 1.6 | 37.5 | 9.0 | | | | |
| | | GmCmCmGmCmUmUmCmAmGmUmCmCmCmUmUmUmCfUfCd | | | | | | | | |
| | | GmUmCmGmAmUmGmGmU*mC*mAkmG*mC-3′ | | | | | | | | |
| KB-018-368 | 382 | 5′-mC*mU*mAkmA*mAmAmAmCmAmUmGmGmCmCmCmGmAm | | | | | | | | |
| | | GmCmCmGmCmUmUmCmAmGmUmCmCmCmUmUmUmCfUdCf | | | | | | | | |
| | | GmUmCmGmAmUmGmGmU*mC*mAkmG*mC-3′ | | | | | | | | |
| KB-018-201 | 383 | 5′-mC*mU*mAkmA*mAmAmAmCmAmUmGmGmCmCmCmGmAm | 58.8 | 2.3 | 33.6 | 13.9 | | | | |
| | | GmCmCmGmCmUmUmCmAmGmUmCmCmCmUmUmUmCfUmCf | | | | | | | | |
| | | GmUmCmGmAmUmGmGmU*mC*mAkmG*mC-3′ | | | | | | | | |
| KB-018-202 | 384 | 5′-mC*mU*mAkmA*mAmAmAmCmAmUmGmGmCmCmCmGmAm | 51.7 | 3.6 | 39.2 | 0.4 | | | | |
| | | GmCmCmGmCmUmUmCmAmGmUmCmCmCmUmUmUmCdTmC | | | | | | | | |
| | | fGmUmCmGmAmUmGmGmU*mC*mAkmG*mC-3′ | | | | | | | | |
| KB-018-243 | 385 | 5′-mC*mU*mAkmA*mAmAmAmCmAmUmGmGmCmCmCmGmAm | | | | | | | | |
| | | GmCmCmGmCmUmUmCmAmGmUmCmCmCmUmUmUmCdTdCf | | | | | | | | |
| | | GmUmCmGmAmUmGmGmU*mC*mAkmG*mC-3′ | | | | | | | | |
| | | SERPINA1 E342K Antisense-gRNA with alpha-anomer | | | | | | | | |
| | | modifications: | | | | | | | | |
| KB-018-221 | 386 | 5′-mC*mU*mAkmA*mAmAmAmCmAmUmGmGmCmCmCmGmAm | 7.6 | 0.7 | 3.5 | 1.0 | | | | |
| | | GmCmCmGmCmUmUmCmAmGmUmCmCmCmUmUmUmCdTαC | | | | | | | | |
| | | dGmUmCmGmAmUmGmGmU*mC*mAkmG*mC-3′ | | | | | | | | |
| KB-018-597 | 387 | 5′-mC*mU*mAkmA*mAmAmAmCmAmUmGmGmCmCmCmGmAm | 1.6 | 0.6 | 2.7 | 1.0 | | | | |
| | | GmCmCmGmCmUmUmCmAmGmUmCmCmCmUmUmUmCαTαC | | | | | | | | |
| | | αGmUmCmGmAmUmGmGmU*mC*mAkmG*mC-3′ | | | | | | | | |
| KB-018-598 | 388 | 5′-mC*mU*mAkmA*mAmAmAmCmAmUmGmGmCmCmCmGmAm | 3.4 | 0.3 | 1.6 | 1.3 | | | | |
| | | GmCmCmGmCmUmUmCmAmGmUmCmCmCmUmUmUmCαTdC | | | | | | | | |
| | | αGmUmCmGmAmUmGmGmU*mC*mAkmG*mC-3′ | | | | | | | | |
| KB-018-599 | 389 | 5′-mC*mU*mAkmA*mAmAmAmCmAmUmGmGmCmCmCmGmAm | 2.5 | 1.5 | 2.0 | 0.8 | | | | |
| | | GmCmCmGmCmUmUmCmAmGmUmCmCmCmUmUmUmCαTmC | | | | | | | | |
| | | αGmUmCmGmAmUmGmGmU*mC*mAkmG*mC-3′ | | | | | | | | |
| | | SERPINA1 E342K Antisense-gRNA with Arabinose | | | | | | | | |
| | | modifications: | | | | | | | | |
| KB-018-222 | 390 | 5′-mC*mU*mAkmA*mAmAmAmCmAmUmGmGmCmCmCmGmAm | 18.6 | 0.5 | 7.8 | 0.1 | | | | |
| | | GmCmCmGmCmUmUmCmAmGmUmCmCmCmUmUmUmCdTaCd | | | | | | | | |
| | | GmUmCmGmAmUmGmGmU*mC*mAkmG*mC-3′ | | | | | | | | |
| KB-018-607 | 391 | 5′-mC*mU*mAkmA*mAmAmAmCmAmUmGmGmCmCmCmGmAm | 31.1 | 0.6 | 13.9 | 0.0 | | | | |
| | | GmCmCmGmCmUmUmCmAmGmUmCmCmCmUmUmUmCaUaCa | | | | | | | | |
| | | GmUmCmGmAmUmGmGmU*mC*mAkmG*mC-3′ | | | | | | | | |
| KB-018-608 | 392 | 5′-mC*mU*mAkmA*mAmAmAmCmAmUmGmGmCmCmCmGmAm | 61.9 | 0.3 | 22.2 | 4.7 | | | | |
| | | GmCmCmGmCmUmUmCmAmGmUmCmCmCmUmUmUmCaUdC | | | | | | | | |
| | | aGmUmCmGmAmUmGmGmU*mC*mAkmG*mC-3′ | | | | | | | | |
| KB-018-609 | 393 | 5′-mC*mU*mAkmA*mAmAmAmCmAmUmGmGmCmCmCmGmAm | 62.0 | 0.5 | 25.6 | 0.0 | | | | |
| | | GmCmCmGmCmUmUmCmAmGmUmCmCmCmUmUmUmCaUmC | | | | | | | | |
| | | aGmUmCmGmAmUmGmGmU*mC*mAkmG*mC-3′ | | | | | | | | |
| | | hSERPINA1 E342K Antisense-gRNA with Ara-2′-OMe | | | | | | | | |
| | | modifications: | | | | | | | | |
| KB-018-223 | 394 | 5′-mC*mU*mAkmA*mAmAmAmCmAmUmGmGmCmCmCmGmAm | 18.6 | 0.5 | 7.8 | 0.1 | | | | |
| | | GmCmCmGmCmUmUmCmAmGmUmCmCmCmUmUmUmCdTam | | | | | | | | |
| | | CdGmUmCmGmAmUmGmGmWmC*mAkmG*mC-3′ | | | | | | | | |
| KB-018-624 | 395 | 5′-mC*mU*mAkmA*mAmAmAmCmAmUmGmGmCmCmCmGmAm | 31.1 | 0.6 | 13.9 | 0.0 | | | | |
| | | GmCmCmGmCmUmUmCmAmGmUmCmCmCmUmUmUmCamUa | | | | | | | | |
| | | mCamGmUmCmGmAmUmGmGmU*mC*mAkmG*mC-3′ | | | | | | | | |
| KB-018-625 | 396 | 5′-mC*mU*mAkmA*mAmAmAmCmAmUmGmGmCmCmCmGmAm | 61.9 | 0.3 | 22.2 | 4.7 | | | | |
| | | GmCmCmGmCmUmUmCmAmGmUmCmCmCmUmUmUmCamUd | | | | | | | | |
| | | CamGmUmCmGmAmUmGmGmU*mC*mAkmG*mC-3′ | | | | | | | | |
| KB-018-626 | 397 | 5′-mC*mU*mAkmA*mAmAmAmCmAmUmGmGmCmCmCmGmAm | 62.0 | 0.5 | 25.6 | 0.0 | | | | |
| | | GmCmCmGmCmUmUmCmAmGmUmCmCmCmUmUmUmCamUm | | | | | | | | |
| | | CamGmUmCmGmAmUmGmGmU*mC*mAkmG*mC-3′ | | | | | | | | |
| | | hSERPINA1 E342K Antisense-gRNA with Abasic (dSpacer) | | | | | | | | |
| | | modifications: | | | | | | | | |
| KB-018-224 | 398 | 5′-mC*mU*mAkmA*mAmAmAmCmAmUmGmGmCmCmCmGmAm | 13.6 | 2.2 | 6.2 | 0.7 | | | | |
| | | GmCmCmGmCmUmUmCmAmGmUmCmCmCmUmUmUmCdTdSd | | | | | | | | |
| | | GmUmCmGmAmUmGmGmU*mC*mAkmG*mC-3′ | | | | | | | | |
| | | hSERPINA1 E342K Antisense-gRNA with 2′-OH-Abasic | | | | | | | | |
| | | (rSpacer) modifications: | | | | | | | | |
| KB-018-649 | 399 | 5′-mC*mU*mAkmA*mAmAmAmCmAmUmGmGmCmCmCmGmAm | 18.9 | 1.9 | 10.1 | 1.0 | | | | |
| | | GmCmCmGmCmUmUmCmAmGmUmCmCmCmUmUmUmCdTrSd | | | | | | | | |
| | | GmUmCmGmAmUmGmGmU*mC*mA*mG*mC-3′ | | | | | | | | |
| | | SERPINA1 E342K Antisense-gRNA with 2′-OMe-Abasic | | | | | | | | |
| | | (mSpacer) modifications: | | | | | | | | |
| KB-018-658 | 400 | 5′-mC*mU*mAkmA*mAmAmAmCmAmUmGmGmCmCmCmGmAm | 13.7 | 2.5 | 7.4 | 1.2 | | | | |
| | | GmCmCmGmCmUmUmCmAmGmUmCmCmCmUmUmUmCdTmS | | | | | | | | |
| | | dGmUmCmGmAmUmGmGmU*mC*mA*mG*mC-3′ | | | | | | | | |
| KB-018-188 | 401 | 5′-mC*mU*mAkmA*mAmAmAmCmAmUmGmGmCmCmCmGmAm | | | | | | | | |
| | | GmCmCmGmCmUmUmCmAmGmUmCmCmCmUmUmUmCdIdCd | | | | | | | | |
| | | GmUmCmGmAmUmGmGmU*mC*mA*mG*mC-3′ | | | | | | | | |

TABLE 10
Mismatches in Both Flanking Sequence and Triplet (underlined)

| | | | Editing Data | | | | | | | |
|---|---|---|---|---|---|---|---|---|---|---|
| | | | ADAR2 % | | | | ADAR1 p110 % | | | |
| | SEQ | | Editing | | | | Editing | | | |
| | ID | | 100 | | 10 | | 100 | | 10 | |
| Seq ID | NO | Guide Oligonucleotide Sequences | nM | SD | nM | SD | nM | SD | nM | SD |
| KB-018-102 | 402 | 5′-mC*mU*mAkmA*mAmAmAmCmAmUmGmGmCmCmCmGmAm | 56.8 | 2.3 | 31.3 | 1.8 | | | | |
| | | GmCmCmGmCmUmUmCmAmGmUmCmCmCmUmUmUmCdTdC | | | | | | | | |
| | | dGmUmCmGmAmUmGmGmU*mC*mAkmG*mC-3′ | | | | | | | | |
| KB-018-184 | 403 | 5′-mC*mU*mAkmA*mAmAmAmCmAmUmGmGmCmCmCmGmAm | 7.8 | 0.6 | 2.8 | 0.6 | | | | |
| | | GmCmCmGmCmUmUmCmAmGmUmCmCmCmUmUmUmCdTdTd | | | | | | | | |
| | | GmUmCmGmAmUmGmGmU*mC*mAkmG*mC-3′ | | | | | | | | |
| KB-018-185 | 404 | 5′-mC*mU*mAkmA*mAmAmAmCmAmUmGmGmCmCmCmGmAm | 47.0 | 2.7 | 30.4 | 1.9 | | | | |
| | | GmCmCmGmCmUmUmCmAmGmUmCmCmCmUmUmUmCdTdG | | | | | | | | |
| | | dGmUmCmGmAmUmGmGmU*mC*mAkmG*mC-3′ | | | | | | | | |
| KB-018-186 | 405 | 5′-mC*mU*mAkmA*mAmAmAmCmAmUmGmGmCmCmCmGmAm | 23.4 | 2.5 | 16.3 | 0.2 | | | | |
| | | GmCmCmGmCmUmUmCmAmGmUmCmCmCmUmUmUmCdTdA | | | | | | | | |
| | | dGmUmCmGmAmUmGmGmU*mC*mAkmG*mC-3′ | | | | | | | | |
| KB-018-187 | 406 | 5′-mC*mU*mAkmA*mAmAmAmCmAmUmGmGmCmCmCmGmAm | 39.2 | 2.6 | 16.4 | | | | | |
| | | GmCmCmGmCmUmUmCmAmGmUmCmCmCmUmUmUmCdGdC | | | | | | | | |
| | | dGmUmCmGmAmUmGmGmU*mC*mAkmG*mC-3′ | | | | | | | | |
| KB-018-188 | 407 | 5′-mC*mU*mAkmA*mAmAmAmCmAmUmGmGmCmCmCmGmAm | 64.0 | 1.5 | 29.8 | 1.3 | | | | |
| | | GmCmCmGmCmUmUmCmAmGmUmCmCmCmUmUmUmCdIdCd | | | | | | | | |
| | | GmUmCmGmAmUmGmGmU*mC*mAkmG*mC-3′ | | | | | | | | |
| KB-018-189 | 408 | 5′-mC*mU*mAkmA*mAmAmAmCmAmUmGmGmCmCmCmGmAm | 25.0 | 1.3 | 11.5 | 2.0 | | | | |
| | | GmCmCmGmCmUmUmCmAmGmUmCmCmCmUmUmUmCdAdC | | | | | | | | |
| | | dGmUmCmGmAmUmGmGmU*mC*mAkmG*mC-3′ | | | | | | | | |
| KB-018-190 | 409 | 5′-mC*mU*mAkmA*mAmAmAmCmAmUmGmGmCmCmCmGmAm | 44.6 | 1.1 | 29.5 | 1.0 | | | | |
| | | GmCmCmGmCmUmUmCmAmGmUmCmCmCmUmUmUmCdCdC | | | | | | | | |
| | | dGmUmCmGmAmUmGmGmU*mC*mAkmG*mC-3′ | | | | | | | | |
| KB-018-191 | 410 | 5′-mC*mU*mAkmA*mAmAmAmCmAmUmGmGmCmCmCmGmAm | 16.7 | 1.1 | 9.8 | 0.5 | | | | |
| | | GmCmCmGmCmUmUmCmAmGmUmCmCmCmUmUmUmCdTdC | | | | | | | | |
| | | dAmUmCmGmAmUmGmGmWmC*mAkmG*mC-3′ | | | | | | | | |
| KB-018-192 | 411 | 5′-mC*mU*mAkmA*mAmAmAmCmAmUmGmGmCmCmCmGmAm | 10.5 | 0.3 | 6.5 | 0.6 | | | | |
| | | GmCmCmGmCmUmUmCmAmGmUmCmCmCmUmUmUmCdTdC | | | | | | | | |
| | | dCmUmCmGmAmUmGmGmU*mC*mAkmG*mC-3′ | | | | | | | | |
| KB-018-193 | 412 | 5′-mC*mU*mAkmA*mAmAmAmCmAmUmGmGmCmCmCmGmAm | 7.4 | 1.5 | 2.6 | 0.1 | | | | |
| | | GmCmCmGmCmUmUmCmAmGmUmCmCmCmUmUmUmCdTdC | | | | | | | | |
| | | dTmUmCmGmAmUmGmGmU*mC*mAkmG*mC-3′ | | | | | | | | |
| KB-018-194 | 413 | 5′-mC*mU*mAkmA*mAmAmAmCmAmUmGmGmCmCmCmGmAm | 1.3 | 0.4 | 0.3 | 0.3 | | | | |
| | | GmCmCmGmCmUmUmCmAmGmUmCmCmCmUmUmUmCdGdC | | | | | | | | |
| | | dCmUmCmGmAmUmGmGmU*mC*mAkmG*mC-3′ | | | | | | | | |

TABLE 11
Mismatches and 2′-O-MOE Modifications in Flanking Sequence

| | | | Editing Data | | | | | | | |
|---|---|---|---|---|---|---|---|---|---|---|
| | | | ADAR2 % | | | | ADAR1 p110 % | | | |
| | SEQ | | Editing | | | | Editing | | | |
| | ID | | 100 | | 10 | | 100 | | 10 | |
| Seq ID | NO | Guide Oligonucleotide Sequences | nM | SD | nM | SD | nM | SD | nM | SD |
| | | SERPINA1 E342K Anti sense-gRNA with 2′-O-MOE | | | | | | | | |
| | | modifications | | | | | | | | |
| KB-018-102 | 414 | 5′-mC*mU*mAkmAkmAmAmAmCmAmUmGmGmCmCmCmGmAm | 42.0 | 4.7 | 16.4 | 2.1 | | | | |
| | | GmCmCmGmCmUmUmCmAmGmUmCmCmCmUmUmUmCdTdC | | | | | | | | |
| | | dGmUmCmGmAmUmGmGmU*mC*mAkmG*mC-3′ | | | | | | | | |
| KB-018-225 | 415 | 5′-MC*MU*MA*MA*mAmAmAmCmAmUmGmGmCmCmCmGmAm | 54.6 | 3.4 | 26.6 | 2.5 | | | | |
| | | GmCmCmGmCmUmUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-226 | 416 | 5′-MC*MU*MA*MA*mAmAmAMCMAMUmGmGmCmCmCmGmAm | 58.6 | 3.9 | 27.1 | 1.2 | | | | |
| | | GmCmCmGmCmUmUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-227 | 417 | 5′-MC*MU*MA*MA*mAmAmAmCmAmUmGmGMCMCMCmGmAm | 49.4 | 1.1 | 25.8 | 0.2 | | | | |
| | | GmCmCmGmCmUmUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-228 | 418 | 5′-MC*MU*MA*MA*mAmAmAmCmAmUmGmGmCmCmCmGMAM | 45.7 | 0.8 | 28.4 | | | | | |
| | | GMCmCmGmCmUmUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-229 | 419 | 5′-MC*MU*MA*MA*mAmAmAmCmAmUmGmGmCmCmCmGmAm | 28.3 | 0.4 | 19.2 | 0.4 | | | | |
| | | GmCmCmGMCMUMUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-230 | 420 | 5′-MC*MU*MA*MA*mAmAmAmCmAmUmGmGmCmCmCmGmAm | 33.4 | 1.8 | 29.0 | 1.3 | | | | |
| | | GmCmCmGmCmUmUmCmAmGMUMCMCmCmUmUmUmCdTdC | | | | | | | | |
| | | dGmUmCmGmAmUmGmGmU*MC*MA*MG*MC-3′ | | | | | | | | |
| KB-018-231 | 421 | 5′-MC*MU*MA*MA*mAmAmAmCmAmUmGmGmCmCmCmGmAm | 31.1 | 0.8 | 34.6 | 1.6 | | | | |
| | | GmCmCmGmCmUmUmCmAmGmUmCmCmCmUMUMUMCdTdC | | | | | | | | |
| | | dGmUmCmGmAmUmGmGmU*MC*MA*MG*MC-3′ | | | | | | | | |
| KB-018-232 | 422 | 5′-MC*MU*MA*MA*mAmAmAmCmAmUmGmGmCmCmCmGmAm | 2.5 | 0.0 | 0.3 | 0.3 | | | | |
| | | GmCmCmGmCmUmUmCmAmGmUmCmCmCmUmUmUmCdTdC | | | | | | | | |
| | | dGMUMCMGmAmUmGmGmU*MC*MA*MG*MC-3′ | | | | | | | | |
| KB-018-233 | 423 | 5′-MC*MU*MA*MA*mAmAmAmCmAmUmGmGMCMCMCMGMAM | 48.1 | 0.3 | 21.6 | 1.9 | | | | |
| | | GMCMCMGMCMUmUmCmAmGmUmCmCmCmUmUmUmCdTdC | | | | | | | | |
| | | dGmUmCmGmAmUmGmGmU*MC*MA*MG*MC-3′ | | | | | | | | |
| KB-018-234 | 424 | 5′-MC*MU*MA*MA*mAmAmAmCmAmUmGmGmCmCmCmGmAm | 1.2 | 0.8 | 1.4 | 0.1 | | | | |
| | | GmCmCmGmCmUmUmCmAmGmUmCmCmCmUMUMUMCdTdC | | | | | | | | |
| | | dGMUMCMGmAmUmGmGmU*MC*MA*MG*MC-3′ | | | | | | | | |
| | | SERPINA1 E342K shorter Antisense-gRNA with site -specific | | | | | | | | |
| | | 2′-MOE modifications | | | | | | | | |
| KB-018-106 | 425 | 5′-mA*mA*mA*mC*mAmUmGmGmCmCmCmGmAmGmCmCmG | 42.7 | 1.0 | 15.1 | 0.5 | | | | |
| | | mCmUmUmCmAmGmUmCmCmCmUmUmUmCdTdCdGmUmCm | | | | | | | | |
| | | GmAmU*mG*mG*mU*mC-3′ | | | | | | | | |
| KB-018-235 | 426 | 5′-MA*MA*MA*MC*mAmUmGmGmCmCmCmGmAmGmCmCmG | 28.8 | 0.8 | 12.7 | 0.1 | | | | |
| | | mCmUmUmCmAmGmUmCmCmCmUmUmUmCdTdCdGmUmCm | | | | | | | | |
| | | GmAmU*MG*MG*MU*MC-3′ | | | | | | | | |
| KB-018-236 | 427 | 5′-MA*MA*MA*MC*mAmUmGmGmCMCMCMGMAMGMCMCMG | 14.8 | 0.3 | 8.8 | 0.5 | | | | |
| | | MCmUmUmCmAmGmUmCmCmCmUmUmUmCdTdCdGmUmCm | | | | | | | | |
| | | GmAmU*MG*MG*MU*MC-3′ | | | | | | | | |
| KB-018-237 | 428 | 5′-MA*MA*MA*MC*mAmUmGmGmCMCMCMGMAMGMCMCMG | 0.3 | 0.0 | 0.1 | 0.1 | | | | |
| | | MCmUmUmCmAmGmUmCmCmCmUmUMUMCdTdCdGMUMCm | | | | | | | | |
| | | GmAmU*MG*MG*MU*MC-3′ | | | | | | | | |

TABLE 12
Partial Modified gRNA Sequence

| | | | Editing Data | | | | | | | |
|---|---|---|---|---|---|---|---|---|---|---|
| | | | ADAR2 % | | | | ADAR1 p110 | | | |
| | SEQ | | Editing | | | | % Editing | | | |
| | ID | | 100 | | 10 | | 100 | | 10 | |
| Seq ID | NO | Guide Oligonucleotide Sequences | nM | SD | nM | SD | nM | SD | nM | SD |
| | | SERPINA1 E342K Antisense-gRNA with partial flanking | | | | | | | | |
| | | sequence modifications | | | | | | | | |
| KB-018-001 | 429 | 5′-mC*mU*mA*mA*mAmAmAmCmAmUmGmGmCmCmCmCmAmG | 30.9 | 3.6 | 11.2 | 1.7 | | | | |
| | | mCmAmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-748 | 430 | 5′-rC*rU*rA*rA*rArArArCrArUrGrGrCrCrCrCrArGrCrArGrCrU | 34.9 | 1.9 | 9.2 | 3.6 | | | | |
| | | rUrCrArGrUrCmCmCmUmUmUmCdTdCdGmUmCmGmAmUmGmGmU*mC | | | | | | | | |
| | | *mA*mG*mC-3′ | | | | | | | | |
| KB-018-749 | 431 | 5′-aC*aU*aA*aA*aAaAaAaCaAaUaGaGaCaCaCaCaAaGaCaAaGaC | 3.5 | 1.1 | 1.0 | 0.9 | | | | |
| | | aUaUaCaAaGaUaCmCmCmUmUmUmCdTdCdGmUmCmGmAmUm | | | | | | | | |
| | | GmGmU*mC*mA*mG*mC-3′ | | | | | | | | |
| KB-018-750 | 432 | 5′-rC*rU*rAkrAkrArArArCrArUrGrGrCrCrCrCrArGrCrArGrCrU | | | | | | | | |
| | | rUrCrArGrUrCmCmCmUmUmUmCfUfCfGmUmCmGmAmUmGmGmU*mC* | | | | | | | | |
| | | mA*mG*mC-3′ | | | | | | | | |
| KB-018-751 | 433 | 5′-mC*mU*mArArArArAmCrAmUrGrGmCmCmCmCrArGmCrArGmC | 25.3 | 0.7 | 6.4 | 2.5 | | | | |
| | | mUmUmCrArGmUmCmCmCmUmUmUmCdTdCdGmUmCmGmAm | | | | | | | | |
| | | UmGmGmU*mC*mA*mG*mC-3′ | | | | | | | | |
| KB-018-752 | 434 | 5′-mC*mU*mArArArArAmCrAmUrGrGmCmCmCmCrArGmCrArGmC | 32.0 | 7.1 | 4.0 | 0.9 | | | | |
| | | mUmUmCrArGmUmCmCmCmUmUmUmCfUfCfGmUmCmGmAmU | | | | | | | | |
| | | mGmGmU*mC*mA*mG*mC-3′ | | | | | | | | |
| KB-018-753 | 435 | 5′-LC*LT*LArArArArArCrArUrGrGrCrCrCrCrArGrCrArGrCrUrU | | | | | | | | |
| | | rCrArGrUrCmCLCmUmUmUmCdTdCdGmUmCmGmALTmGmGmUmCL | | | | | | | | |
| | | A*LG*LC-3′ | | | | | | | | |
| KB-018-754 | 436 | 5′-rC*rU*rA*rA*rArArArCrArUrGrGrCrCrCrCrArGrCrArGrCrU | | | | | | | | |
| | | rUrCrArGrUrCmCmCmUmUmUmCdTdCdGmUmCmGmAmUmGmGmU*mC | | | | | | | | |
| | | *mA*mG*rC-PO-GaINAc-3′ | | | | | | | | |
| KB-018-755 | 437 | 5′-rC*rA*rU*rG*rGrCrCrCrCrArGrCrArGrCrUrUrCrArGrUrCmC | 30.6 | 5.3 | 8.8 | 3.5 | | | | |
| | | mCmUmUmUmCdTdCdGmUmCmGmAmUmGmGmU*mC*mA*mG*mC-3′ | | | | | | | | |
| KB-018-756 | 438 | 5′-rC*rA*rU*rG*rGrCrCrCrCrArGrCrArGrCrUrUrCrArGrUrCmC | 22.6 | 3.9 | 7.1 | 1.9 | | | | |
| | | LCmUmUmUmCdTdCdGmUmCmGmALT*mG*mG*LT*mC-3′ | | | | | | | | |
| KB-018-102 | 439 | 5′-mC*mU*mA*mA*mAmAmAmCmAmUmGmGmCmCmCmGmAmG | 8.9 | 1.5 | 5.1 | 1.9 | | | | |
| | | mCmCmGmCmUmUmCmAmGmUmCmCmCmUmUmUmCdTdCdG | | | | | | | | |
| | | mUmCmGmAmUmGmGmU*mC*mA*mG*mC-3′ | | | | | | | | |
| KB-018-757 | 440 | 5′-rC*rU*rA*rA*rArArArCrArUrGrGrCrCrCrGrArGrCrCrGrCrU | 10.7 | 1.6 | 4.0 | 0.1 | | | | |
| | | rUrCrArGrUrCmCmCmUmUmUmCdTdCdGmUmCmGmAmUmGmGmU*mC | | | | | | | | |
| | | *mA*mG*mC-3′ | | | | | | | | |
| KB-018-758 | 441 | 5′-mC*mU*mArArArArAmCrAmUrGrGmCmCmCrGrArGmCmCrGmC | 6.2 | 0.4 | 3.3 | 0.6 | | | | |
| | | mUmUmCrArGmUmCmCmCmUmUmUmCdTdCdGmUmCmGmAm | | | | | | | | |
| | | UmGmGmU*mC*mA*mG*mC-3′ | | | | | | | | |
| KB-018-759 | 442 | 5′-rC*rU*rA*rA*rArArArCrArUrGrGrCrCrCrGrArGrCrCrGrCrU | | | | | | | | |
| | | rUrCrArGrUrCmCmCmUmUmUmCdTdCdGmUmCmGmAmUmGmGmU*mC | | | | | | | | |
| | | *mA*mG*rC-PO-GaINAc-3′ | | | | | | | | |

TABLE 13
Site-Specific Internal Phosphorothioate (PS) Modifications

| | | | Editing Data | | | | | | | |
|---|---|---|---|---|---|---|---|---|---|---|
| | SEQ | | ADAR2 % Editing | | | | ADAR1 p110 % Editing | | | |
| Seq ID | ID NO | Guide Oligonucleotide Sequences | 100 nM | SD | 10 nM | SD | 100 nM | SD | 10 nM | SD |
| | | SERPINA1 E342K Anti sense-gRNA with site-specific PS | | | | | | | | |
| | | modification | | | | | | | | |
| KB-018-380 | 443 | 5′-mA*mA*mC*mA*mUmGmGmCmCmCmC*mAmGmC*mAmGmCm | 46.3 | 40.6 | 51.6 | 54.4 | | | | |
| | | UmU*mC*mAmGmU*mCmCmCmUmUmU*mCdTdCdGmU*mCmGm | | | | | | | | |
| | | AmUmGmGmU*mC*mA*mG*mC-3′ | | | | | | | | |
| KB-018-381 | 444 | 5′-mC*mU*mA*mA*mAmAmAmC*mAmUmGmGmCmCmCmC*mAmG | 32.0 | 30.3 | 38.0 | | | | | |
| | | mC*mAmGmCmUmU*mC*mAmGmU*mCmCmCmUmUmU*mC*dT*d | | | | | | | | |
| | | C*dG*mU*mCmGmAmUmGmGmU*mC*mA*mG*mC-3′ | | | | | | | | |
| KB-018-382 | 445 | 5′-mC*mU*mA*mA*mAmAmAmC*mAmUmGmGmCmCmCmC*mAmG | 29.3 | 36.6 | 36.6 | 34.8 | | | | |
| | | mC*mAmGmCmUmU*mC*mAmGmU*mC*mC*mC*mU*mUmU*mCdT | | | | | | | | |
| | | dCdG*mU*mC*mG*mA*mU*mG*mG*mU*mC*mA*mG*mC-3′ | | | | | | | | |
| KB-018-383 | 446 | 5′-mC*mU*mA*mA*mAmAmAmC*mAmUmGmGmCmCmCmC*mAmG | | 31.4 | 26.0 | 22.1 | | | | |
| | | mC*mAmGmCmUmU*mC*mAmGmU*mC*mC*mC*mU*mUmU*mC*d | | | | | | | | |
| | | rdC*dG*mU*mC*mG*mA*mU*mG*mG*mU*mC*mA*mG*mC-3′ | | | | | | | | |
| KB-018-384 | 447 | 5′-mC*mU*mA*mA*mAmAmAmCmAmUmGmGmCmCmCmCmAmGm | 43.2 | 41.4 | 30.0 | 31.2 | | | | |
| | | CmAmGmCmUmUmCmAmGmUmC*mC*mCmUmUmUmCdTdC*dG* | | | | | | | | |
| | | mU*mC*mG*mAmUmGmGmU*mC*mA*mG*mC-3′ | | | | | | | | |
| KB-018-385 | 448 | 5′-mC*mU*mA*mA*mAmAmAmC*mAmUmGmGmCmCmCmC*mAmG | 53.9 | 54.5 | 38.5 | 40.5 | | | | |
| | | mC*mAmGmCmUmU*mC*mAmGmU*mC*mC*mCmUmUmU*mCdTd | | | | | | | | |
| | | C*dG*mU*mC*mG*mA*mUmGmGmU*mC*mA*mG*mC-3′ | | | | | | | | |
| KB-018-386 | 449 | 5′-mC*mU*mA*mA*mAmAmAmC*mAmUmGmGmCmCmCmC*mAmG | 47.6 | 47.7 | | | | | | |
| | | mC*mAmGmCmUmU*mC*mAmGmU*mC*mC*mCmUmUmU*mC*dT* | | | | | | | | |
| | | dC*dG*mU*mC*mG*mA*mUmGmGmU*mC*mA*mG*mC-3′ | | | | | | | | |
| KB-018-373 | 450 | 5′-mC*mU*mA*mA*mA*mA*mA*mC*mA*mU*mG*mG*mC*mC*mC*m | 40.0 | 33.1 | 28.7 | 24.6 | | | | |
| | | C*mA*mG*mC*mA*mG*mC*mU*mU*mC*mA*mG*mU*mC*mC*mC*m | | | | | | | | |
| | | U*mU*mU*mC*dT*dC*dG*mU*mC*mG*mA*mU*mG*mG*mU*mC*mA | | | | | | | | |
| | | *mG*mC-3′ | | | | | | | | |
| | | SERPINA1 E342K with RNA, 2′-F, 2′-OMe, 2′-MOE and PS | | | | | | | | |
| | | modifications | | | | | | | | |
| KB-018-001 | 451 | 5′-mC*mU*mA*mA*mAmAmAmCmAmUmGmGmCmCmCmCmAmGm | 54.9 | 6.3 | 26.8 | 8.8 | | | | |
| | | CmAmGmCmUmUmCmAmGmUmCmCmCmUmUmUmCdTdCdGmU | | | | | | | | |
| | | mCmGmAmUmGmGmU*mC*mA*mG*mC-3′ | | | | | | | | |
| KB1-08-373 | 452 | 5′-mC*mU*mA*mA*mA*mA*mA*mC*mA*mU*mG*mG*mC*mC*mC*m | 35.0 | 1.7 | 22.3 | 3.2 | | | | |
| | | C*mA*mG*mC*mA*mG*mC*mU*mU*mC*mA*mG*mU*mC*mC*mC*m | | | | | | | | |
| | | U*mU*mU*mC*dT*dC*dG*mU*mC*mG*mA*mU*mG*mG*mU*mC*mA | | | | | | | | |
| | | *mG*mC-3′ | | | | | | | | |
| KB-018-374 | 453 | 5′-rC*rU*rA*rA*rArArArCrArUrGrGrCrCrCrCrArGrCrArGrCrUrUrCrArGr | 37.2 | 2.1 | 14.9 | 2.9 | | | | |
| | | UrCrCrCrUrUrUrCrUrCrGrUrCrGrArUrGrGrU*rC*rA*rG*rC-3′ | | | | | | | | |
| KB-018-375 | 454 | 5′-rC*rU*rA*rA*rA*rA*rA*rC*rA*rU*rG*rG*rC*rC*rC*rC*rA*rG*rC*rA*rG* | 57.3 | 4.7 | 39.9 | 3.4 | | | | |
| | | rC*rU*rU*rC*rA*rG*rU*rC*rC*rC*rU*rU*rU*rC*rU*rC*rG*rU*rC*rG*rA*r | | | | | | | | |
| | | U*rG*rG*rU*rC*rA*rG*rC-3′ | | | | | | | | |
| KB-018-376 | 455 | 5′-FA*FA*FC*FA*FUFGFGFCFCFCFCFAFGFCFAFGFCFUFUFCFAF | 35.9 | 12.4 | 27.7 | 1.3 | | | | |
| | | GFUFCFCFCFUFUFUFCdTdCdGFUFCFGFAFUFGFGFU*FC*FA*FG | | | | | | | | |
| | | *FC-3′ | | | | | | | | |
| KB-018-377 | 456 | 5′-FA*FA*FC*FA*FU*FG*FG*FC*FC*FC*FC*FA*FG*FC*FA*FG*FC*F | 46.2 | 7.1 | 32.2 | 2.3 | | | | |
| | | U*FU*FC*FA*FG*FU*FC*FC*FC*FU*FU*FU*FC*dT*dC*dG*FU*FC*F | | | | | | | | |
| | | G*FA*FU*FG*FG*FU*FC*FA*FG*FC-3′ | | | | | | | | |
| KB-018-378 | 457 | 5′-MA*MA*MC*MA*MUMGMGMCMCMCMCMAMGMCMAMGMCMU | 4.9 | 1.7 | 3.4 | 1.3 | | | | |
| | | MUMCMAMGMUMCMCMCMUMUMUMCdTdCdGMUMCMGMAMUM | | | | | | | | |
| | | GMGMU*MC*MA*MG*MC-3′ | | | | | | | | |
| KB-018-379 | 458 | 5′-MA*MA*MC*MA*MU*MG*MG*MC*MC*MC*MC*MA*MG*MC*MA*M | 10.2 | 1.8 | 8.8 | 4.5 | | | | |
| | | G*MC*MU*MU*MC*MA*MG*MU*MC*MC*MC*MU*MU*MU*MC*dT*dC | | | | | | | | |
| | | *dG*MU*MC*MG*MA*MU*MG*MG*MU*MC*MA*MG*MC-3′ | | | | | | | | |

TABLE 14
Additional SERPINA1 antisense-gRNAs

| | | | Editing Data | | | | | | | |
|---|---|---|---|---|---|---|---|---|---|---|
| | SEQ | | ADAR2 % Editing | | | | ADAR1 p110 % Editing | | | |
| Seq ID | ID NO | Guide Oligonucleotide Sequences | 100 nM | SD | 10 nM | SD | 100 nM | SD | 10 nM | SD |
| | | SERPINA1 Antisense-gRNA:Reverse Constructs | | | | | | | | |
| KB-018-001 | 459 | 5′-mC*mU*mA*mA*mAmAmAmCmAmUmGmGmCmCmCmCmAmGm | 54.9 | 6.3 | 26.8 | 8.8 | | | | |
| | | CmAmGmCmUmUmCmAmGmUmCmCmCmUmUmUmCdTdCdGmU | | | | | | | | |
| | | mCmGmAmUmGmGmU*mC*mA*mG*mC-3′ | | | | | | | | |
| KB-018-397 | 460 | 5′-mU*mC*mA*mG*mUmCmCmCmUmUmUmCdTdCdGmUmCmGm | 40.7 | 4.7 | 26.2 | 7.8 | | | | |
| | | AmUmGmGmUmCmAmGmCmAmCmAmGmCmCmUmUmAmUmGm | | | | | | | | |
| | | CmAmCmGmGmCmCmU*mU*mG*mG*mA-3′ | | | | | | | | |
| KB-018-094 | 461 | 5′-mC*mU*mA*mA*mAmAmAmCmAmUmGmGmCmCmCmCmAmGm | 46.9 | 7.8 | 26.4 | 3.9 | | | | |
| | | CmAmAmCmUmUmGmAmGmUmCmCmCmUmUmUmCdTdCdGmU | | | | | | | | |
| | | mCmGmAmUmGmGmU*mC*mA*mG*mC-3′ | | | | | | | | |
| KB-018-102 | 462 | 5′-mC*mU*mA*mA*mAmAmAmCmAmUmGmGmCmCmCmGmAmG | 40.7 | 4.7 | 26.2 | 7.8 | | | | |
| | | mCmCmGmCmUmUmCmAmGmUmCmCmCmUmUmUmCdTdCdGm | | | | | | | | |
| | | UmCmGmAmUmGmGmU*mC*mA*mG*mC-3′ | | | | | | | | |
| KB-018-398 | 463 | 5′-mU*mC*mA*mG*mUmCmCmCmUmUmUmCdTdCdGmUmCmGm | 36.4 | 4.9 | 27.0 | 8.3 | | | | |
| | | AmUmGmGmUmCmAmAmCmAmCmCmGmCmCmUmUmAmUmGm | | | | | | | | |
| | | CmAmCmGmGmCmCmU*mU*mG*mG*mA-3′ | | | | | | | | |
| KB-018-399 | 464 | 5′-mU*mC*mA*mG*mUmCmCmCmUmUmUmCdTdCdGmUmCmGm | 41.6 | 8.2 | 20.0 | 15.2 | | | | |
| | | AmUmGmGmUmCmAmGmCmAmCmCmGmCmCmGmUmAmUmGm | | | | | | | | |
| | | CmAmCmGmGmCmCmU*mU*mG*mG*mA-3′ | | | | | | | | |
| KB-018-400 | 465 | 5′-mU*mC*mA*mG*mUmCmCmCmUmUmUmCdTdCdGmUmCmGm | 39.0 | 5.2 | 26.9 | 4.4 | | | | |
| | | AmUmGmGmUmCmAmGmCmAmCmAmAmCmCmUmGmAmUmGm | | | | | | | | |
| | | CmAmCmGmGmCmCmU*mU*mG*mG*mA-3′ | | | | | | | | |

TABLE 15
SERPINA1 antisense gRNAs of different lengths

| | | | Editing Data | | | | | |
|---|---|---|---|---|---|---|---|---|
| | SEQ | | ADAR2 % Editing | | | | | |
| Seq ID | ID NO | Guide Oligonucleotide Sequences | 100 nM | SD | 11 nM | SD | 10 nM | SD |
| | | SERPINA1 E342K GluR-gRNA with different antisense lengths and | | | | | | |
| | | modifications | | | | | | |
| KB-018-034 | 466 | 5′-rG*rU*rG*rG*rArArUrArGrUrArUrArArCrArArUrArUrGrCrUrArArArUrGrUrUrGr | 43.0 | 7.8 | 47.4 | 9.1 | | |
| | | UrUrArUrArGrUrArUrCrCrCrAmC-mCmUmUmUmCdTdCdGmUmCmGmAmUm | | | | | | |
| | | GmGmU*mC*mA*mG*mC-3′ | | | | | | |
| KB-018-035 | 467 | 5′-mG*mU*mG*mG*mAmAmUmAmGmUmAmUmAmAmCmAmAmUmAmUmG | 44.6 | 18.1 | 45.2 | 18.9 | | |
| | | mCmUmAmAmAmUmGmUmUmGmUmUmAmUmAmGmUmAmUmCmCmCm | | | | | | |
| | | A- | | | | | | |
| | | mC-mCmUmUmUmCdTdCdGmUmCmGmAmUmGmGmU*mC*mA*mG*mC-3′ | | | | | | |
| KB-018-036 | 468 | 5′-rG*rU*rG*rG*rArArUrArGrUrArUrArArCrArArUrArUrGrCrUrArArArUrGrUrUrGr | 45.2 | 10.3 | 43.4 | 10.3 | | |
| | | UrUrArUrArGrUrArUrCrCrCrAmC-mCmCmUmUmUmCdTdCdGmUmCmGmAm | | | | | | |
| | | UmGmG*mU*mC*mA*mG-3′ | | | | | | |
| KB-018-037 | 469 | 5′-mG*mU*mG*mG*mAmAmUmAmGmUmAmUmAmAmCmAmAmUmAmUmG | 37.9 | 15.2 | 40.1 | 13.6 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmG*mU*mC*mA*mG-3′ | | | | | | |
| KB-018-038 | 470 | 5′-rG*rU*rG*rG*rArArUrArGrUrArUrArArCrArArUrArUrGrCrUrArArArUrGrUrUrGr | 34.8 | 11.3 | 35.6 | 10.9 | | |
| | | UrUrArUrArGrUrArUrCrCrCrAmC-mCmUmUmUmCdTdCdGmUmCmGmAmUm | | | | | | |
| | | GmG*mU*mC*mA*mG-3′ | | | | | | |
| KB-018-039 | 471 | 5′-rG*rU*rG*rG*rArArUrArGrUrArUrArArCrArArUrArUrGrCrUrArArArUrGrUrUrGr | 45.5 | 18.4 | 41.1 | 15.6 | | |
| | | UrUrArUrArGrUrArUrCrCrCrAmC-mCmCmUmUmUmCdTdCdGmUmCmGmAm | | | | | | |
| | | UmG*mG*mU*mC*mA-3′ | | | | | | |
| KB-018-040 | 472 | 5′-rG*rU*rG*rG*rArArUrArGrUrArUrArArCrArArUrArUrGrCrUrArArArUrGrUrUrGr | 37.7 | 12.4 | 22.3 | 5.8 | | |
| | | UrUrArUrArGrUrArUrCrCrCrAmC-mCmUmUmUmCdTdCdGmUmCmGmAmUm | | | | | | |
| | | G*mG*mU*mC*mA-3′ | | | | | | |
| KB-018-041 | 473 | 5′-rG*rU*rG*rG*rArArUrArGrUrArUrArArCrArArUrArUrGrCrUrArArArUrGrUrUrGr | 35.2 | 9.1 | 40.8 | 14.3 | | |
| | | UrUrArUrArGrUrArUrCrCrCrAmC-mCmCmUmUmUmCdTdCdGmUmCmGmAm | | | | | | |
| | | U*mG*mG*mU*mC-3′ | | | | | | |
| KB-018-042 | 474 | 5′-rG*rU*rG*rG*rArArUrArGrUrArUrArArCrArArUrArUrGrCrUrArArArUrGrUrUrGr | 46.0 | 3.9 | 42.1 | 18.6 | | |
| | | UrUrArUrArGrUrArUrCrCrCrAmC-mCmUmUmUmCdTdCdGmUmCmGmAmU* | | | | | | |
| | | mG*mG*mU*mC-3′ | | | | | | |
| KB-018-043 | 475 | 5′-mG*mU*mG*mG*mAmAmUmAmGmUmAmUmAmAmCmAmAmUmAmUmG | 38.4 | 19.9 | 42.1 | 17.2 | | |
| | | mCmUmAmAmAmUmGmUmUmGmUmUmAmUmAmGmUmAmUmCmCmCm | | | | | | |
| | | AmC-mCmUmUmUmCdTdCdGmUmCmGmAmU*mG*mG*mU*mC-3′ | | | | | | |
| KB-018-044 | 476 | 5′-rG*rU*rG*rG*rArArUrArGrUrArUrArArCrArArUrArUrGrCrUrArArArUrGrUrUrGr | 41.7 | 5.4 | 34.5 | 9.6 | | |
| | | UrUrArUrArGrUrArUrCrCrCrAmC-mCmCmUmUmUmCdTdCdGmUmCmGmA* | | | | | | |
| | | mU*mG*mG*mU-3′ | | | | | | |
| KB-018-045 | 477 | 5′-mG*mU*mG*mG*mAmAmUmAmGmUmAmUmAmAmCmAmAmUmAmUmG | 42.0 | 16.8 | 36.4 | 14.6 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmA*mU*mG*mG*mU-3′ | | | | | | |
| | | SERPINA1 E342K GluR-gRNA with varying lengths 5′ of triplet | | | | | | |
| KB-018-115 | 478 | 5′-mG*mU*mG*mG*mAmAmUmAmGmUmAmUmAmAmCmAmAmUmAmUmG | 3.0 | 0.6 | 2.0 | 0.3 | | |
| | | mCmUmAmAmAmUmGmUmUmGmUmUmAmUmAmGmUmAmUmCmCmCm | | | | | | |
| | | AmC-mUmCdTdCdGmUmCmGmAmUmGmGmU*mC*mA*mG*mC-3 | | | | | | |
| KB-018-116 | 479 | 5′-mG*mU*mG*mG*mAmAmUmAmGmUmAmUmAmAmCmAmAmUmAmUmG | 16.7 | 1.9 | 11.0 | 0.9 | | |
| | | mCmUmAmAmAmUmGmUmUmGmUmUmAmUmAmGmUmAmUmCmCmCm | | | | | | |
| | | AmC-mUmUmCdTdCdGmUmCmGmAmUmGmGmU*mC*mA*mG*mC-3 | | | | | | |
| KB-018-117 | 480 | 5′-mG*mU*mG*mG*mAmAmUmAmGmUmAmUmAmAmCmAmAmUmAmUmG | 49.1 | 3.2 | 37.1 | 3.1 | | |
| | | mCmUmAmAmAmUmGmUmUmGmUmUmAmUmAmGmUmAmUmCmCmCm | | | | | | |
| | | AmC-mUmUmUmCdTdCdGmUmCmGmAmUmGmGmU*mC*mA*mG*mC-3 | | | | | | |
| KB-018-035 | 481 | 5′-mG*mU*mG*mG*mAmAmUmAmGmUmAmUmAmAmCmAmAmUmAmUmG | 59.2 | 0.6 | 47.7 | 1.0 | | |
| | | mCmUmAmAmAmUmGmUmUmGmUmUmAmUmAmGmUmAmUmCmCmCm | | | | | | |
| | | A-mC-mCmUmUmUmCdTdCdGmUmCmGmAmUmGmGmU*mC*mA*mG*mC- | | | | | | |
| | | 3′ | | | | | | |
| KB-018-118 | 482 | 5′-mG*mU*mG*mG*mAmAmUmAmGmUmAmUmAmAmCmAmAmUmAmUmG | 47.9 | 5.6 | 47.6 | 3.5 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmGmU*mC*mA*mG*m | | | | | | |
| | | C-3 | | | | | | |
| KB-018-119 | 483 | 5′-mG*mU*mG*mG*mAmAmUmAmGmUmAmUmAmAmCmAmAmUmAmUmG | 49.3 | 1.5 | 31.1 | 7.5 | | |
| | | mCmUmAmAmAmUmGmUmUmGmUmUmAmUmAmGmUmAmUmCmCmCm | | | | | | |
| | | AmC-mCmCmCmUmUmUmCdTdCdGmUmCmGmAmUmGmGmU*mC*mA*m | | | | | | |
| | | G*mC-3 | | | | | | |
| KB-018-120 | 484 | 5′-mG*mU*mG*mG*mAmAmUmAmGmUmAmUmAmAmCmAmAmUmAmUmG | 46.1 | 1.4 | 34.5 | 1.8 | | |
| | | mCmUmAmAmAmUmGmUmUmGmUmUmAmUmAmGmUmAmUmCmCmCm | | | | | | |
| | | AmC-mUmCmCmCmUmUmUmCdTdCdGmUmCmGmAmUmGmGmU*mC*mA | | | | | | |
| | | *mG*mC-3 | | | | | | |
| KB-018-121 | 485 | 5′-mG*mU*mG*mG*mAmAmUmAmGmUmAmUmAmAmCmAmAmUmAmUmG | 18.6 | 6.1 | 23.3 | 1.4 | | |
| | | mCmUmAmAmAmUmGmUmUmGmUmUmAmUmAmGmUmAmUmCmCmCm | | | | | | |
| | | AmC-mGmUmCmCmCmUmUmUmCdTdCdGmUmCmGmAmUmGmGmU*mC* | | | | | | |
| | | mA*mG*mC-3 | | | | | | |
| | | SERPINA1 E342K GluR-gRNA with varying lengths 3′ of triplet | | | | | | |
| KB-018-122 | 486 | 5′-mG*mU*mG*mG*mAmAmUmAmGmUmAmUmAmAmCmAmAmUmAmUmG | 14.6 | 1.1 | 8.4 | 0.8 | | |
| | | mCmUmAmAmAmUmGmUmUmGmUmUmAmUmAmGmUmAmUmCmCmCm | | | | | | |
| | | AmC-mCmCmUmUmUmCdTdCdGmU*mC*mG*mA*mU-3 | | | | | | |
| KB-018-123 | 487 | 5′-mG*mU*mG*mG*mAmAmUmAmGmUmAmUmAmAmCmAmAmUmAmUmG | 20.9 | 2.8 | 21.2 | 0.7 | | |
| | | mCmUmAmAmAmUmGmUmUmGmUmUmAmUmAmGmUmAmUmCmCmCm | | | | | | |
| | | AmC-mCmCmUmUmUmCdTdCdGmUmC*mG*mA*mU*mG-3 | | | | | | |
| KB-018-124 | 488 | 5′-mG*mU*mG*mG*mAmAmUmAmGmUmAmUmAmAmCmAmAmUmAmUmG | 40.2 | 1.9 | 33.5 | 1.3 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmG*mA*mU*mG*mG-3 | | | | | | |
| KB-018-045 | 489 | 5′-mG*mU*mG*mG*mAmAmUmAmGmUmAmUmAmAmCmAmAmUmAmUmG | 48.6 | 1.1 | 25.8 | 9.7 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmA*mU*mG*mG*mU-3 | | | | | | |
| KB-018-125 | 490 | 5′-mG*mU*mG*mG*mAmAmUmAmGmUmAmUmAmAmCmAmAmUmAmUmG | 52.4 | 1.9 | 45.7 | 1.6 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmU*mG*mG*mU*mC-3 | | | | | | |
| KB-018-126 | 491 | 5′-mG*mU*mG*mG*mAmAmUmAmGmUmAmUmAmAmCmAmAmUmAmUmG | 1.4 | 0.3 | 1.5 | 0.4 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmG*mG*mU*mC*mA-3 | | | | | | |
| KB-018-037 | 492 | 5′-mG*mU*mG*mG*mAmAmUmAmGmUmAmUmAmAmCmAmAmUmAmUmG | 51.7 | 3.0 | 30.0 | 4.5 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmG*mU*mC*mA*mG- | | | | | | |
| | | 3 | | | | | | |
| KB-018-128 | 493 | 5′-mG*mU*mG*mG*mAmAmUmAmGmUmAmUmAmAmCmAmAmUmAmUmG | 57.6 | 0.7 | 53.5 | 1.4 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmGmU*mC*mA*mG*m | | | | | | |
| | | C-3 | | | | | | |
| KB-018-129 | 494 | 5′-mG*mU*mG*mG*mAmAmUmAmGmUmAmUmAmAmCmAmAmUmAmUmG | 59.8 | 0.6 | 46.8 | 1.1 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmGmUmC*mA*mG*m | | | | | | |
| | | C*mA-3 | | | | | | |
| KB-018-130 | 495 | 5′-mG*mU*mG*mG*mAmAmUmAmGmUmAmUmAmAmCmAmAmUmAmUmG | 62.7 | 1.8 | 50.6 | 3.6 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmGmUmCmA*mG*mC | | | | | | |
| | | *mA*mC-3 | | | | | | |
| KB-018-131 | 496 | 5′-mG*mU*mG*mG*mAmAmUmAmGmUmAmUmAmAm0mAmAmUmAmUmG | 67.0 | 1.7 | 53.1 | 3.1 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmUmGmGmUmCmAmG*mC* | | | | | | |
| | | mA*mC*mA-3 | | | | | | |

TABLE 16
GluR Motif With Different Lengths and Modifications

| | | | Editing Data | | | | | |
|---|---|---|---|---|---|---|---|---|
| | | | ADAR2 % Editing | | | | | |
| | SEQ ID | | | | | | | |
| Seq ID | NO | Guide Oligonucleotide Sequences | 100 nM | SD | 11 nM | SD | 10 nM | SD |
| | | SERPINA1 E342K GluR-gRNA with modifications | | | | | | |
| KB-018-081 | 497 | 5′-rGrGrUrGrArArGrArGrGrArGrArArCrArArUrArUrGrCrUrArArArUrGrUrUrGr | 21.8 | 1.0 | 18.5 | 0.8 | | |
| | | UrUrCrUrCrGrUrCrUrCrCrArCrCmCmCmUmUmUmCrUrCrGmUmCmGmAm | | | | | | |
| | | U*mG*mG*mU*mC-3′ | | | | | | |
| KB-018-082 | 498 | 5′-mG*mG*mUrGrArArGrArGrGrArGrArAmCrArAmUrAmUrGmCmUrArArAm | 16.8 | 3.4 | 12.3 | 3.7 | | |
| | | UrGmUmUrGmUmUmCmUmCrGmUmCmUmCmCrAmCmCmCmCmUmUm | | | | | | |
| | | UmCrUrCrGmUmCmGmAmU*mG*mG*mU*mC-3′ | | | | | | |
| KB-018-083 | 499 | 5′-mG*mG*mUrGrArArGrArGrGrArGrArAmCrArAmUrAmUrGmCmUrArArAm | 16.9 | 1.4 | 15.9 | 1.0 | | |
| | | UrGmUmUrGmUmUmCmUmCrGmUmCmUmCmCrAmCmCmCmCmUmUm | | | | | | |
| | | UmCmUrCrGmUmCmGmAmU*mG*mG*mU*mC-3′ | | | | | | |
| KB-018-084 | 500 | 5′-mG*mG*mUrGrArArGrArGrGrArGrArAmCrArAmUrAmUrGmCmUrArArAm | 33.8 | 0.7 | 27.1 | 2.2 | | |
| | | UrGmUmUrGmUmUmCmUmCrGmUmCmUmCmCrAmCmCmCmCmUmUm | | | | | | |
| | | UmCdTdCdGmUmCmGmAmU*mG*mG*mU*mC-3′ | | | | | | |
| KB-018-085 | 501 | 5′-rGrGrUrGrUrCrGrArGrArArGrArGrGrArGrArArCrArArUrArUrGrCrUrArArAr | 6.2 | 2.2 | 7.0 | 1.9 | | |
| | | UrGrUrUrGrUrUrCrUrCrGrUrCrUrCrCrUrCrGrArCrArCrCmCmCmUmUmUmC | | | | | | |
| | | rUrCrGmUmCmGmAmU*mG*mG*mU*mC-3′ | | | | | | |
| KB-018-086 | 502 | 5′-mG*mG*mUrGmUmCrGrArGrArArGrArGrGrArGrArAmCrArAmUrAmUrGm | 27.0 | 1.4 | 26.6 | 2.4 | | |
| | | CmUrArArAmUrGmUmUrGmUmUmCmUmCrGmUmCmUmCmCmUmCrGrA | | | | | | |
| | | mCrAmCmCmCmCmUmUmUmCrUrCrGmUmCmGmAmU*mG*mG*mU*mC- | | | | | | |
| | | 3′ | | | | | | |
| KB-018-087 | 503 | 5′-mG*mG*mUrGmUmCrGrArGrArArGrArGrGrArGrArAmCrArAmUrAmUrGm | 27.7 | 1.3 | 25.2 | 1.8 | | |
| | | CmUrArArAmUrGmUmUrGmUmUmCmUmCrGmUmCmUmCmCmUmCrGrA | | | | | | |
| | | mCrAmCmCmCmCmUmUmUmCmUrCrGmUmCmGmAmU*mG*mG*mU*mC- | | | | | | |
| | | 3′ | | | | | | |
| KB-018-088 | 504 | 5′-mG*mG*mUrGmUmCrGrArGrArArGrArGrGrArGrArAmCrArAmUrAmUrGm | 42.2 | 0.5 | 37.5 | 0.9 | | |
| | | CmUrArArAmUrGmUmUrGmUmUmCmUmCrGmUmCmUmCmCmUmCrGrA | | | | | | |
| | | mCrAmCmCmCmCmUmUmUmCdTdCdGmUmCmGmAmU*mG*mG*mU*m | | | | | | |
| | | C-3′ | | | | | | |
| KB-018-089 | 505 | 5′-mG*mG*mUrGmUmCrGrArGrArArGrArGrGrArGrArAmCrArAmUrAmUrGm | | | | | | |
| | | CmUrArArAmUrGmUmUrGmUmUmCmUmCrGmUmCmUmCmCmUmCrGrA | | | | | | |
| | | mCrAmCmCrCrArUrGrGrCrCrCrCrArGrCrArGrCrUrUrCrArGrUrCmCLCmUm | | | | | | |
| | | UmUmCmUrCrGmUmCmGmALT*mG*mG*LT*mC-3′ | | | | | | |
| KB-018-760 | 506 | 5′-mG*mG*mUrGrArArGrArGrGrArGrArAmCrArAmUrAmUrGmCmUrArArAm | | | | | | |
| | | UrGmUmUrGmUmUmCmUmCrGmUmCmUmCmCrAmCmC- | | | | | | |
| | | rCrArUrGrGrCrCrCrCrArGrCrArGrCrUrUrCrArGrUrCmCLCmUmUmUmCdTd | | | | | | |
| | | CdGmUmCmGmALT*mG*mG*LT*mC-3′ | | | | | | |
| KB-018-761 | 507 | 5′-mG*mG*mUrGmUmCrGrArGrArArGrArGrGrArGrArAmCrArAmUrAmUrGm | | | | | | |
| | | CmUrArArAmUrGmUmUrGmUmUmCmUmCrGmUmCmUmCmCmUmCrGrA | | | | | | |
| | | mCrAmCmC-rCrArUrGrGrCrCrCrCrArGrCrArGrCrUrUrCrArGrUrCmCLCmUm | | | | | | |
| | | UmUmCdTdCdGmUmCmGmALT*mG*mG*LT*mC-3′ | | | | | | |
| KB-018-1303 | 508 | 5′-mG*mG*mUrGmUmCrGrArGrArArGrArGrGrArGrArAmCrArAmUrAmUrGm | | | | | | |
| | | CmUrArArAmUrGmUmUrGmUmUmCmUmCrGmUmCmUmCmCmUmCrGrA | | | | | | |
| | | mCrAmCmC-mCmAmUmGmGmCmCmCmCmAmGmCmAmGmCmUmUmC | | | | | | |
| | | mAmGmUmCmCLCmUmUmUmCdTdCdGmUmCmGmALT*mG*mG*LT*mC- | | | | | | |
| | | 3′ | | | | | | |
| | | SERPINA1 E342K GluR-gRNA with different GluR motifs and | | | | | | |
| | | modifications | | | | | | |
| KB-018-041 | 509 | 5′-rG*rU*rG*rG*rArArUrArGrUrArUrArArCrArArUrArUrGrCrUrArArArUrGrUrUr | 54.6 | 1.3 | 33.7 | 1.2 | | |
| | | GrUrUrArUrArGrUrArUrCrCrCrAmC- | | | | | | |
| | | mCmCmUmUmUmCdTdCdGmUmCmGmAmU*mG*mG*mU*mC-3′ | | | | | | |
| KB-018-125 | 510 | 5′-mG*mU*mG*mG*mAmAmUmAmGmUmAmUmAmAmCmAmAmUmAmUm | 52.4 | 1.9 | 45.7 | 1.6 | | |
| | | GmCmUmAmAmAmUmGmUmUmGmUmUmAmUmAmGmUmAmUmCmCm | | | | | | |
| | | CmAmC-mCmCmUmUmUmCdTdCdGmUmCmGmAmU*mG*mG*mU*mC-3 | | | | | | |
| KB-018-132 | 511 | 5′-rG*rG*rU*rG*rArArGrArGrGrArGrArArCrArArUrArUrGrCrUrArArArUrGrUrUr | 52.7 | 1.7 | 46.1 | 0.9 | | |
| | | GrUrUrCrUrCrGrUrCrUrCrCrArCrC- | | | | | | |
| | | mCmCmUmUmUmCdTdCdGmUmCmGmAmU*mG*mG*mU*mC-3′ | | | | | | |
| KB-018-084 | 512 | 5′-mG*mG*mUrGrArArGrArGrGrArGrArAmCrArAmUrAmUrGmCmUrArArAm | 31.4 | 2.1 | 20.8 | 1.1 | | |
| | | UrGmUmUrGmUmUmCmUmCrGmUmCmUmCmCrAmCmC- | | | | | | |
| | | mCmCmUmUmUmCdTdCdGmUmCmGmAmU*mG*mG*mU*mC-3′ | | | | | | |
| KB-018-133 | 513 | 5′-mG*mG*mU*mG*mAmAmGmAmGmGmAmGmAmAmCmAmAmUmAmUm | 59.8 | 1.5 | 45.7 | 2.4 | | |
| | | GmCmUmAmAmAmUmGmUmUmGmUmUmCmUmCmGmUmCmUmCmCm | | | | | | |
| | | AmCmC-mCmCmUmUmUmCdTdCdGmUmCmGmAmU*mG*mG*mU*mC-3′ | | | | | | |
| KB-018-134 | 514 | 5′-mG*mG*FU*mG*mAmAmGmAmGmGmAmGmAmAFCmAmAFUmAFUmG | 60.9 | 0.6 | 56.6 | 2.0 | | |
| | | FCFUmAmAmAFUmGFUFUmGFUFUFCFUFCmGFUFCFUFCFCmAFCFC- | | | | | | |
| | | mCmCmUmUmUmCdTdCdGmUmCmGmAmU*mG*mG*mU*mC-3′ | | | | | | |
| KB-018-135 | 515 | 5′-FGFGmUFGFAFAFGFAFGFGFAFGFAFAmCFAFAmUFAmUFGmCmUFA | 20.4 | 2.1 | 18.9 | 1.6 | | |
| | | FAFAmUFGmUmUFGmUmUmCmUmCFGmUmCmUmCmCFAmCmC- | | | | | | |
| | | mCmCmUmUmUmCdTdCdGmUmCmGmAmU*mG*mG*mU*mC-3′ | | | | | | |
| KB-018-136 | 516 | 5′-rG*rG*rU*rG*rUrCrGrArGrArArGrArGrGrArGrArArCrArArUrArUrGrCrUrArAr | 45.6 | 2.4 | 44.4 | 3.0 | | |
| | | ArUrGrUrUrGrUrUrCrUrCrGrUrCrUrCrCrUrCrGrArCrArCrC- | | | | | | |
| | | mCmCmUmUmUmCdTdCdGmUmCmGmAmU*mG*mG*mU*mC-3′ | | | | | | |
| KB-018-088 | 517 | 5′-mG*mG*mUrGmUmCrGrArGrArArGrArGrGrArGrArAmCrArAmUrAmUrGm | 37.0 | 3.4 | 31.6 | 4.0 | | |
| | | CmUrArArAmUrGmUmUrGmUmUmCmUmCrGmUmCmUmCmCmUmCrGrA | | | | | | |
| | | mCrAmCmC- | | | | | | |
| | | mCmCmUmUmUmCdTdCdGmUmCmGmAmU*mG*mG*mU*mC-3′ | | | | | | |
| KB-018-137 | 518 | 5′-mG*mG*mU*mG*mUmCmGmAmGmAmAmGmAmGmGmAmGmAmAmC | 42.5 | 3.0 | 39.4 | 2.5 | | |
| | | mAmAmUmAmUmGmCmUmAmAmAmUmGmUmUmGmUmUmCmUmCmG | | | | | | |
| | | mUmCmUmCmCmUmCmGmAmCmAmCmC- | | | | | | |
| | | mCmCmUmUmUmCdTdCdGmUmCmGmAmU*mG*mG*mU*mC-3′ | | | | | | |
| KB-018-138 | 519 | 5′-mG*mG*FU*mG*FUFCmGmAmGmAmAmGmAmGmGmAmGmAmAFCmA | 28.9 | 2.2 | 22.3 | 2.2 | | |
| | | mAFUmAFUmGFCFUmAmAmAFUmGFUFUmGFUFUFCFUFCmGFUFCFUF | | | | | | |
| | | CFCFUFCmGmAFCmAFCFC-mCmCmUmUmUmCdTdCdGmUmCmGmAm | | | | | | |
| | | U*mG*mG*mU*mC-3′ | | | | | | |
| KB-018-139 | 520 | 5′-FG*FG*mU*FG*mUmCFGFAFGFAFAFGFAFGFGFAFGFAFAmCFAFAmU | 41.2 | 3.4 | 29.7 | 2.4 | | |
| | | FAmUFGmCmUFAFAFAmUFGmUmUFGmUmUmCmUmCFGmUmCmUmC | | | | | | |
| | | mCmUmCFGFAmCFAmCmC-mCmCmUmUmUmCdTdCdGmUmCmGmAm | | | | | | |
| | | U*mG*mG*mU*mC-3′ | | | | | | |

TABLE 17
Linker Between GluR and Antisense Motifs

| | | | Editing Data | | | | | |
|---|---|---|---|---|---|---|---|---|
| | SEQ ID | | ADAR2 % Editing | | | | | |
| Seq ID | NO | Guide Oligonucleotide Sequences | 100 nM | SD | 11 nM | SD | 10 nM | SD |
| | | SERPINA1 E342K GluR-gRNA with C3-linker between GluR & | | | | | | |
| | | antisense gRNA | | | | | | |
| KB-018-125 | 521 | 5′-mG*mU*mG*mG*mAmAmUmAmGmUmAmUmAmAmCmAmAmUmAmUmG | 48.6 | 1.1 | 25.8 | 9.7 | | |
| | | mCmUmAmAmAmUmGmUmUmGmUmUmAmUmAmGmUmAmUmCmCmCm | | | | | | |
| | | AmC-mCmCmUmUmUmCdTdCdGmUmCmGmAmU*mG*mG*mU*mC-3 | | | | | | |
| KB-018-140 | 522 | 5′-mG*mU*mG*mG*mA*mAmUmAmGmUmAmUmAmAmCmAmAmUmAmUmG | 21.7 | 1.9 | 21.5 | 3.2 | | |
| | | mCmUmAmAmAmUmGmUmUmGmUmUmAmUmAmGmUmAmUmCmCmCm | | | | | | |
| | | AmC-C3-mCmCmUmUmUmCdTdCdGmUmCmGmAmU*mG*mG*mU*mC-3′ | | | | | | |
| KB-018-141 | 523 | 5′-mG*mG*mU*mG*mAmAmGmAmGmGmAmGmAmAmCmAmAmUmAmUmG | | | | | | |
| | | mCmUmAmAmAmUmGmUmUmGmUmUmCmUmCmGmUmCmUmCmCmAm | | | | | | |
| | | CmC-C3-mCmCmUmUmUmCdTdCdGmUmCmGmAmU*mG*mG*mU*mC-3′ | | | | | | |
| KB-018-142 | 524 | 5′-mG*mG*mU*mG*mUmCmGmAmGmAmAmGmAmGmGmAmGmAmAmCmA | | | | | | |
| | | mAmUmAmUmGmCmUmAmAmAmUmGmUmUmGmUmUmCmUmCmGmUm | | | | | | |
| | | CmUmCmCmUmCmGmAmCmAmCmC-C3-mCmCmUmUmUmCdTdCdGmUm | | | | | | |
| | | CmGmAmU*mG*mG*mU*mC-3′ | | | | | | |

TABLE 18
GluR-21ZNA: Triplet Modifications

| | | | Editing Data | | | | | |
|---|---|---|---|---|---|---|---|---|
| | SEQ | | ADAR2 % Editing | | | | | |
| Seq ID | ID NO | Guide Oligonucleotide Sequences | 100 nM | SD | 11 nM | SD | 10 nM | SD |
| | | SERPINA1 E342K GluR-gRNA with 2′-OMe modification | | | | | | |
| KB-018-125 | 525 | 5′-mG*mU*mG*mG*mAmAmUmAmGmUmAmUmAmAmCmAmAmUmAmUmGmC | 59.2 | 1.4 | 50.8 | 2.7 | | |
| | | mUmAmAmAmUmGmUmUmGmUmUmAmUmAmGmUmAmUmCmCmCmAmC-m | | | | | | |
| | | CmCmUmUmUmCdTdCdGmUmCmGmAmU*mG*mG*mU*mC-3 | | | | | | |
| KB-018-146 | 526 | 5′-mG*mU*mG*mG*mAmAmUmAmGmUmAmUmAmAmCmAmAmUmAmUmGmC | 47.8 | 1.8 | 41.3 | 3.6 | | |
| | | mUmAmAmAmUmGmUmUmGmUmUmAmUmAmGmUmAmUmCmCmCmAmC-m | | | | | | |
| | | CmCmUmUmUmCmUdCdGmUmCmGmAmU*mG*mG*mU*mC-3 | | | | | | |
| KB-018-147 | 527 | 5′-mG*mU*mG*mG*mAmAmUmAmGmUmAmUmAmAmCmAmAmUmAmUmGmC | 30.1 | 10.3 | 34.5 | 2.0 | | |
| | | mUmAmAmAmUmGmUmUmGmUmUmAmUmAmGmUmAmUmCmCmCmAmC-m | | | | | | |
| | | CmCmUmUmUmCdTmCdGmUmCmGmAmU*mG*mG*mU*mC-3 | | | | | | |
| KB-018-148 | 528 | 5′-mG*mU*mG*mG*mAmAmUmAmGmUmAmUmAmAmCmAmAmUmAmUmGmC | 33.2 | 2.2 | 29.5 | 2.3 | | |
| | | mUmAmAmAmUmGmUmUmGmUmUmAmUmAmGmUmAmUmCmCmCmAmC-m | | | | | | |
| | | CmCmUmUmUmCdTdCmGmUmCmGmAmU*mG*mG*mU*mC-3 | | | | | | |
| KB-018-149 | 529 | 5′-mG*mU*mG*mG*mAmAmUmAmGmUmAmUmAmAmCmAmAmUmAmUmGmC | 7.0 | 1.9 | 6.5 | 0.7 | | |
| | | mUmAmAmAmUmGmUmUmGmUmUmAmUmAmGmUmAmUmCmCmCmAmC-m | | | | | | |
| | | CmCmUmUmUmCmUmCmGmUmCmGmAmU*mG*mG*mU*mC-3 | | | | | | |
| KB-018-150 | 530 | 5′-mG*mU*mG*mG*mAmAmUmAmGmUmAmUmAmAmCmAmAmUmAmUmGmC | 14.3 | 5.0 | 13.9 | 1.7 | | |
| | | mUmAmAmAmUmGmUmUmGmUmUmAmUmAmGmUmAmUmCmCmCmAmC-m | | | | | | |
| | | CmCmUmUmUmCdTmCmGmUmCmGmAmU*mG*mG*mU*mC-3 | | | | | | |
| KB-018-151 | 531 | 5′-mG*mU*mG*mG*mAmAmUmAmGmUmAmUmAmAmCmAmAmUmAmUmGmC | 16.2 | 1.9 | 15.2 | 1.8 | | |
| | | mUmAmAmAmUmGmUmUmGmUmUmAmUmAmGmUmAmUmCmCmCmAmC-m | | | | | | |
| | | CmCmUmUmUmCmUmCdGmUmCmGmAmU*mG*mG*mU*mC-3 | | | | | | |
| KB-018-152 | 532 | 5′-mG*mU*mG*mG*mAmAmUmAmGmUmAmUmAmAmCmAmAmUmAmUmGmC | 20.2 | 2.3 | 14.8 | 1.7 | | |
| | | mUmAmAmAmUmGmUmUmGmUmUmAmUmAmGmUmAmUmCmCmCmAmC-m | | | | | | |
| | | CmCmUmUmUmCmUdCmGmUmCmGmAmU*mG*mG*mU*mC-3 | | | | | | |
| | | SERPINA1 E342K GluR-gRNA with 2′-FANA modification | | | | | | |
| KB-018-125 | 533 | 5′-mG*mU*mG*mG*mAmAmUmAmGmUmAmUmAmAmCmAmAmUmAmUmGmC | 59.2 | 1.4 | 50.8 | 2.7 | | |
| | | mUmAmAmAmUmGmUmUmGmUmUmAmUmAmGmUmAmUmCmCmCmAmC-m | | | | | | |
| | | CmCmUmUmUmCdTdCdGmUmCmGmAmU*mG*mG*mU*mC-3 | | | | | | |
| KB-018-153 | 534 | 5′-mG*mU*mG*mG*mAmAmUmAmGmUmAmUmAmAmCmAmAmUmAmUmGmC | 57.1 | 2.0 | 51.9 | 2.4 | | |
| | | mUmAmAmAmUmGmUmUmGmUmUmAmUmAmGmUmAmUmCmCmCmAmC-m | | | | | | |
| | | CmCmUmUmUmCfUdCdGmUmCmGmAmU*mG*mG*mU*mC-3 | | | | | | |
| KB-018-154 | 535 | 5′-mG*mU*mG*mG*mAmAmUmAmGmUmAmUmAmAmCmAmAmUmAmUmGmC | 63.1 | 1.0 | 51.5 | 2.9 | | |
| | | mUmAmAmAmUmGmUmUmGmUmUmAmUmAmGmUmAmUmCmCmCmAmC-m | | | | | | |
| | | CmCmUmUmUmCdTfCdGmUmCmGmAmU*mG*mG*mU*mC-3 | | | | | | |
| KB-018-155 | 536 | 5′-mG*mU*mG*mG*mAmAmUmAmGmUmAmUmAmAmCmAmAmUmAmUmGmC | 47.7 | 10.8 | 53.9 | 2.2 | | |
| | | mUmAmAmAmUmGmUmUmGmUmUmAmUmAmGmUmAmUmCmCmCmAmC-m | | | | | | |
| | | CmCmUmUmUmCdTdCfGmUmCmGmAmU*mG*mG*mU*mC-3 | | | | | | |
| KB-018-156 | 537 | 5′-mG*mU*mG*mG*mAmAmUmAmGmUmAmUmAmAmCmAmAmUmAmUmGmC | 59.4 | 0.9 | 48.8 | 2.6 | | |
| | | mUmAmAmAmUmGmUmUmGmUmUmAmUmAmGmUmAmUmCmCmCmAmC-m | | | | | | |
| | | CmCmUmUmUmCfUfCfGmUmCmGmAmU*mG*mG*mU*mC-3 | | | | | | |
| KB-018-157 | 538 | 5′-mG*mU*mG*mG*mAmAmUmAmGmUmAmUmAmAmCmAmAmUmAmUmGmC | 7.3 | 0.5 | 2.3 | 0.5 | | |
| | | mUmAmAmAmUmGmUmUmGmUmUmAmUmAmGmUmAmUmCmCmCmAmC-m | | | | | | |
| | | CmCmUmUmUmCdTfCfGmUmCmGmAmU*mG*mG*mU*mC-3 | | | | | | |
| KB-018-158 | 539 | 5′-mG*mU*mG*mG*mAmAmUmAmGmUmAmUmAmAmCmAmAmUmAmUmGmC | 56.3 | 1.2 | 44.1 | 1.8 | | |
| | | mUmAmAmAmUmGmUmUmGmUmUmAmUmAmGmUmAmUmCmCmCmAmC-m | | | | | | |
| | | CmCmUmUmUmCfUfCdGmUmCmGmAmU*mG*mG*mU*mC-3 | | | | | | |
| KB-018-159 | 540 | 5′-mG*mU*mG*mG*mAmAmUmAmGmUmAmUmAmAmCmAmAmUmAmUmGmC | 56.8 | 1.5 | 43.6 | 1.7 | | |
| | | mUmAmAmAmUmGmUmUmGmUmUmAmUmAmGmUmAmUmCmCmCmAmC-m | | | | | | |
| | | CmCmUmUmUmCfUdCfGmUmCmGmAmU*mG*mG*mU*mC-3 | | | | | | |
| KB-018-160 | 541 | 5′-mG*mU*mG*mG*mAmAmUmAmGmUmAmUmAmAmCmAmAmUmAmUmGmC | 11.5 | 2.5 | 9.5 | 0.0 | | |
| | | mUmAmAmAmUmGmUmUmGmUmUmAmUmAmGmUmAmUmCmCmCmAmC-m | | | | | | |
| | | CmCmUmUmUmCmUfCmGmUmCmGmAmU*mG*mG*mU*mC-3 | | | | | | |
| KB-018-161 | 542 | 5′-mG*mU*mG*mG*mAmAmUmAmGmUmAmUmAmAmCmAmAmUmAmUmGmC | 21.3 | 2.5 | 17.6 | 0.0 | | |
| | | mUmAmAmAmUmGmUmUmGmUmUmAmUmAmGmUmAmUmCmCmCmAmC-m | | | | | | |
| | | CmCmUmUmUmCfUfCmGmUmCmGmAmU*mG*mG*mU*mC-3 | | | | | | |
| KB-018-162 | 543 | 5′-mG*mU*mG*mG*mAmAmUmAmGmUmAmUmAmAmCmAmAmUmAmUmGmC | 33.1 | 3.7 | 24.2 | 3.4 | | |
| | | mUmAmAmAmUmGmUmUmGmUmUmAmUmAmGmUmAmUmCmCmCmAmC-m | | | | | | |
| | | CmCmUmUmUmCmUfCfGmUmCmGmAmU*mG*mG*mU*mC-3 | | | | | | |
| KB-018-163 | 544 | 5′-mG*mU*mG*mG*mAmAmUmAmGmUmAmUmAmAmCmAmAmUmAmUmGmC | 31.9 | 2.3 | 26.5 | 2.7 | | |
| | | mUmAmAmAmUmGmUmUmGmUmUmAmUmAmGmUmAmUmCmCmCmAmC-m | | | | | | |
| | | CmCmUmUmUmCfUmCfGmUmCmGmAmU*mG*mG*mU*mC-3 | | | | | | |
| | | SERPINA1 E342K GluR-gRN with (S)- and (R)-GNA modifications | | | | | | |
| KB-018-125 | 545 | 5′-mG*mU*mG*mG*mAmAmUmAmGmUmAmUmAmAmCmAmAmUmAmUmGmC | 28.3 | 0.7 | 17.2 | 4.1 | | |
| | | mUmAmAmAmUmGmUmUmGmUmUmAmUmAmGmUmAmUmCmCmCmAmC-m | | | | | | |
| | | CmCmUmUmUmCdTdCdGmUmCmGmAmU*mG*mG*mU*mC-3 | | | | | | |
| KB-018-164 | 546 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCsgTdCdGmUmCmGmAmU*mG*mG*mU*mC-3 | | | | | | |
| KB-018-165 | 547 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCdTsgCdGmUmCmGmAmU*mG*mG*mU*mC-3 | | | | | | |
| KB-018-166 | 548 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCdTdCsgGmUmCmGmAmU*mG*mG*mU*mC-3 | | | | | | |
| KB-018-167 | 549 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCsgUsgCsgGmUmCmGmAmU*mG*mG*mU*mC-3 | | | | | | |
| KB-018-168 | 550 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCdTsgCsgGmUmCmGmAmU*mG*mG*mU*mC-3 | | | | | | |
| KB-018-169 | 551 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCsgUsgCdGmUmCmGmAmU*mG*mG*mU*mC-3 | | | | | | |
| KB-018-170 | 552 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCsgUdCsgGmUmCmGmAmU*mG*mG*mU*mC-3 | | | | | | |
| KB-018-171 | 553 | 5′-mG*mU*mG*mG*mAmAmUmAmGmUmAmUmAmAmCmAmAmUmAmUmGmC | 0.0 | 0.0 | 0.0 | 0.0 | | |
| | | mUmAmAmAmUmGmUmUmGmUmUmAmUmAmGmUmAmUmCmCmCmAmC-m | | | | | | |
| | | CmCmUmUmUmCdTrgCdGmUmCmGmAmU*mG*mG*mU*mC-3 | | | | | | |
| | | SERPINA1 E342K GluR-gRNA with Arabinose modifications | | | | | | |
| KB-018-128 | 554 | 5′-mG*mU*mG*mG*mAmAmUmAmGmUmAmUmAmAmCmAmAmUmAmUmGmC | 47.6 | 5.8 | 36.8 | 6.1 | | |
| | | mUmAmAmAmUmGmUmUmGmUmUmAmUmAmGmUmAmUmCmCmCmAmC-m | | | | | | |
| | | CmCmUmUmUmCdTdCdGmUmCmGmAmUmGmGmU*mC*mA*mG*mC-3 | | | | | | |
| KB-018-610 | 555 | 5′-mG*mU*mG*mG*mAmAmUmAmGmUmAmUmAmAmCmAmAmUmAmUmGmC | 38.8 | 4.5 | 33.4 | 2.1 | | |
| | | mUmAmAmAmUmGmUmUmGmUmUmAmUmAmGmUmAmUmCmCmCmAmC-m | | | | | | |
| | | CmCmUmUmUmCaUdCdGmUmCmGmAmUmGmGmU*mC*mA*mG*mC-3 | | | | | | |
| KB-018-611 | 556 | 5′-mG*mU*mG*mG*mAmAmUmAmGmUmAmUmAmAmCmAmAmUmAmUmGmC | 55.5 | 2.3 | 39.7 | 4.3 | | |
| | | mUmAmAmAmUmGmUmUmGmUmUmAmUmAmGmUmAmUmCmCmCmAmC-m | | | | | | |
| | | CmCmUmUmUmCdTaCdGmUmCmGmAmUmGmGmU*mC*mA*mG*mC-3 | | | | | | |
| KB-018-612 | 557 | 5′-mG*mU*mG*mG*mAmAmUmAmGmUmAmUmAmAmCmAmAmUmAmUmGmC | 59.4 | 3.4 | 38.7 | 7.7 | | |
| | | mUmAmAmAmUmGmUmUmGmUmUmAmUmAmGmUmAmUmCmCmCmAmC-m | | | | | | |
| | | CmCmUmUmUmCdTdCaGmUmCmGmAmUmGmGmU*mC*mA*mG*mC-3 | | | | | | |
| KB-018-613 | 558 | 5′-mG*mU*mG*mG*mAmAmUmAmGmUmAmUmAmAmCmAmAmUmAmUmGmC | 39.3 | 2.7 | 31.2 | 5.4 | | |
| | | mUmAmAmAmUmGmUmUmGmUmUmAmUmAmGmUmAmUmCmCmCmAmC-m | | | | | | |
| | | CmCmUmUmUmCaUaCaGmUmCmGmAmUmGmGmU*mC*mA*mG*mC-3 | | | | | | |
| KB-018-614 | 559 | 5′-mG*mU*mG*mG*mAmAmUmAmGmUmAmUmAmAmCmAmAmUmAmUmGmC | 46.2 | 2.5 | 38.5 | 5.6 | | |
| | | mUmAmAmAmUmGmUmUmGmUmUmAmUmAmGmUmAmUmCmCmCmAmC-m | | | | | | |
| | | CmCmUmUmUmCdTaCaGmUmCmGmAmUmGmGmU*mC*mA*mG*mC-3 | | | | | | |
| KB-018-615 | 560 | 5′-mG*mU*mG*mG*mAmAmUmAmGmUmAmUmAmAmCmAmAmUmAmUmGmC | 27.1 | 0.7 | 22.3 | 4.8 | | |
| | | mUmAmAmAmUmGmUmUmGmUmUmAmUmAmGmUmAmUmCmCmCmAmC-m | | | | | | |
| | | CmCmUmUmUmCaUaCdGmUmCmGmAmUmGmGmU*mC*mA*mG*mC-3 | | | | | | |
| KB-018-616 | 561 | 5′-mG*mU*mG*mG*mAmAmUmAmGmUmAmUmAmAmCmAmAmUmAmUmGmC | 46.9 | 3.7 | 40.2 | 4.4 | | |
| | | mUmAmAmAmUmGmUmUmGmUmUmAmUmAmGmUmAmUmCmCmCmAmC-m | | | | | | |
| | | CmCmUmUmUmCaUdCaGmUmCmGmAmUmGmGmU*mC*mA*mG*mC-3 | | | | | | |
| | | SERPINA1 E342K GluR-gRNA with Ara-2′-OMe modifications | | | | | | |
| KB-018-128 | 562 | 5′-mG*mU*mG*mG*mAmAmUmAmGmUmAmUmAmAmCmAmAmUmAmUmGmC | 60.7 | 2.6 | 53.3 | 3.0 | | |
| | | mUmAmAmAmUmGmUmUmGmUmUmAmUmAmGmUmAmUmCmCmCmAmC-m | | | | | | |
| | | CmCmUmUmUmCdTdCdGmUmCmGmAmUmGmGmU*mC*mA*mG*mC-3 | | | | | | |
| KB-018-627 | 563 | 5′-mG*mU*mG*mG*mAmAmUmAmGmUmAmUmAmAmCmAmAmUmAmUmGmC | 41.0 | 0.9 | 39.1 | 3.6 | | |
| | | mUmAmAmAmUmGmUmUmGmUmUmAmUmAmGmUmAmUmCmCmCmAmC-m | | | | | | |
| | | CmCmUmUmUmCamUdCdGmUmCmGmAmUmGmGmU*mC*mA*mG*mC-3 | | | | | | |
| KB-018-628 | 564 | 5′-mG*mU*mG*mG*mAmAmUmAmGmUmAmUmAmAmCmAmAmUmAmUmGmC | 41.2 | 2.8 | 35.6 | 2.2 | | |
| | | mUmAmAmAmUmGmUmUmGmUmUmAmUmAmGmUmAmUmCmCmCmAmC-m | | | | | | |
| | | CmCmUmUmUmCdTamCdGmUmCmGmAmUmGmGmU*mC*mA*mG*mC-3 | | | | | | |
| KB-018-629 | 565 | 5′-mG*mU*mG*mG*mAmAmUmAmGmUmAmUmAmAmCmAmAmUmAmUmGmC | 56.5 | 2.4 | 50.8 | 3.3 | | |
| | | mUmAmAmAmUmGmUmUmGmUmUmAmUmAmGmUmAmUmCmCmCmAmC-m | | | | | | |
| | | CmCmUmUmUmCdTdCamGmUmCmGmAmUmGmGmU*mC*mA*mG*mC-3 | | | | | | |
| KB-018-630 | 566 | 5′-mG*mU*mG*mG*mAmAmUmAmGmUmAmUmAmAmCmAmAmUmAmUmGmC | 43.6 | 3.2 | 37.0 | 3.5 | | |
| | | mUmAmAmAmUmGmUmUmGmUmUmAmUmAmGmUmAmUmCmCmCmAmC-m | | | | | | |
| | | CmCmUmUmUmCamUamCamGmUmCmGmAmUmGmGmU*mC*mA*mG*mC-3 | | | | | | |
| KB-018-631 | 567 | 5′-mG*mU*mG*mG*mAmAmUmAmGmUmAmUmAmAmCmAmAmUmAmUmGmC | 42.6 | 0.9 | 40.9 | 4.9 | | |
| | | mUmAmAmAmUmGmUmUmGmUmUmAmUmAmGmUmAmUmCmCmCmAmC-m | | | | | | |
| | | CmCmUmUmUmCdTamCamGmUmCmGmAmUmGmGmU*mC*mA*mG*mC-3 | | | | | | |
| KB-018-632 | 568 | 5′-mG*mU*mG*mG*mAmAmUmAmGmUmAmUmAmAmCmAmAmUmAmUmGmC | 24.6 | 1.0 | 59.3 | 1.9 | | |
| | | mUmAmAmAmUmGmUmUmGmUmUmAmUmAmGmUmAmUmCmCmCmAmC-m | | | | | | |
| | | CmCmUmUmUmCamUamCdGmUmCmGmAmUmGmGmU*mC*mA*mG*mC-3 | | | | | | |
| KB-018-633 | 569 | 5′-mG*mU*mG*mG*mAmAmUmAmGmUmAmUmAmAmCmAmAmUmAmUmGmC | 59.3 | 1.9 | 47.0 | 5.2 | | |
| | | mUmAmAmAmUmGmUmUmGmUmUmAmUmAmGmUmAmUmCmCmCmAmC-m | | | | | | |
| | | CmCmUmUmUmCamUdCamGmUmCmGmAmUmGmGmU*mC*mA*mG*mC-3 | | | | | | |

TABLE 19
SERPINA1 GluR-gRNA: Reverse Constructs

| | | | Editing Data | | | | | |
|---|---|---|---|---|---|---|---|---|
| | SEQ | | ADAR2 % Editing | | | | | |
| | ID | | 100 | | 11 | | 10 | |
| Seq ID | NO | Guide Oligonucleotide Sequences | nM | SD | nM | SD | nM | SD |
| KB-018-035 | 570 | 5′-mG*mU*mG*mG*mAmAmUmAmGmUmAmUmAmAmCmAmAmUmAmUmGmC | 58.9 | 10.2 | | | 40.7 | 5.0 |
| | | mUmAmAmAmUmGmUmUmGmUmUmAmUmAmGmUmAmUmCmCmCmA-mCm | | | | | | |
| | | CmUmUmUmCdTdCdGmUmCmGmAmUmGmGmU*mC*mA*mG*mC-3′ | | | | | | |
| KB-018-401 | 571 | 5′-mU*mC*mA*mG*mUmCmCmCmUmUmUmCdTdCdGmUmCmGmAmUmGm-C | 15.2 | 5.2 | | | 13.0 | 3.2 |
| | | mAmCmCmCmUmAmUmGmAmUmAmUmUmGmUmUmGmUmAmAmAmUmCmG | | | | | | |
| | | mUmAmUmAmAmCmAmAmUmAmUmGmAmUmAmA*mG*mG*mU*mG-3′ | | | | | | |
| KB-018-402 | 572 | 5′-mU*mC*mA*mG*mUmCmCmCmUmUmUmCdTdCdGmUmCmGmAmUmGm-G | 12.6 | 1.2 | | | 8.3 | 1.5 |
| | | mUmGmGmAmAmUmAmGmUmAmUmAmAmCmAmAmUmAmUmGmCmUmAmA | | | | | | |
| | | mAmUmGmUmUmGmUmUmAmUmAmGmUmAmUmC*mC*mC*mA*mC-3′ | | | | | | |

### Example 2. The +2 Position of the Guide Oligonucleotide is Sensitive to Certain Nucleotide Modifications

Based on the data in Example 1, it was found that the selection of nucleotide at the +2 position of the triplet of the guide oligonucleotide (i.e., the first position 3′ of the traditional triplet, which is represented by X⁴ in the structure [Aₘ]-X¹-X²-X³-X⁴-[Bn], where X² is the editing site) can affect the editing rate of the target. Compounds KB-018-001, KB-018-724, KB-018-697, KB-018-725, KB-018-440, and KB-018-486 each have a different modified nucleotide at the +2 position (2′-OMe, DNA, FANA, SGNA, 2′-F, and MOE, respectively).

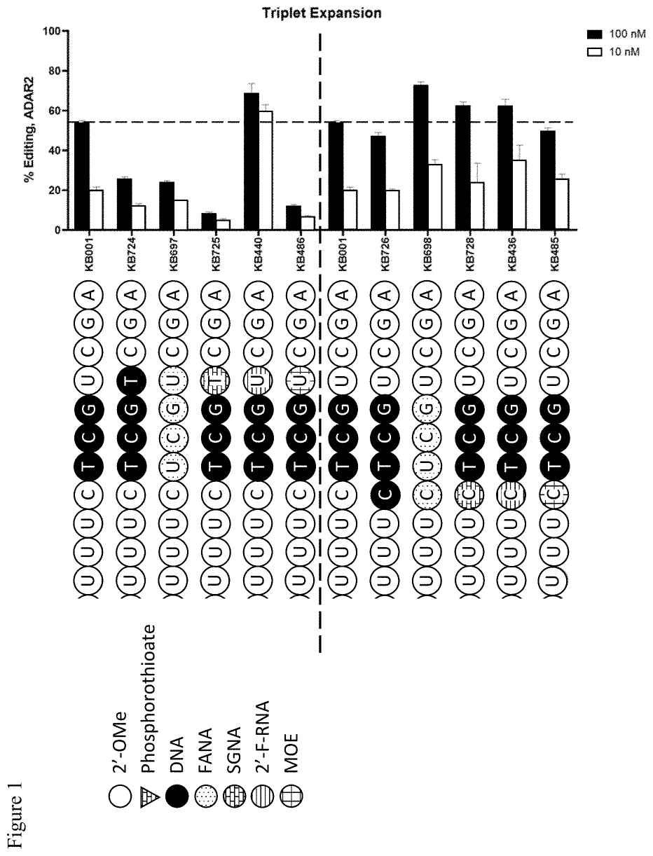

As shown in FIG. 1, the presence of DNA, FANA, S-GNA, or MOE nucleotides (each as defined herein) at the +2 location resulted in lower rates of editing compared to a 2′-OMe nucleotide. Further, improved editing was observed with a 2′-fluoro nucleotide compared to 2′-OMe nucleotide.

In contrast, the −2 position of the guide oligonucleotide (i.e., the first position 5′ of the traditional triplet) does not appear to be particularly sensitive to the same nucleotide modifications. As shown in FIG. 1, the presence of DNA, FANA, S-GNA, 2′-F, or MOE nucleotides (each as defined herein) at the −2 location did not significantly impact the rate of editing compared to 2′-OMe. The guide oligonucleotides with 2′-OMe, DNA, FANA, SGNA, 2′-F, and MOE in the −2 position are KB-018-001, KB-018-726, KB-018-698, KB-018-728, KB-018-436, and KB-018-485, respectively.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method of editing a SERPINA1 polynucleotide comprising a single nucleotide polymorphism (SNP) associated with alpha 1 antitrypsin deficiency which may result in hepatic failure or emphysema, the method comprising contacting the SERPINA1 polynucleotide with a guide oligonucleotide capable of effecting an adenosine deaminase acting on RNA (ADAR)-mediated adenosine to inosine alteration of a SNP associated with alpha 1 antitrypsin deficiency, thereby editing the SERPINA1 polynucleotide.

2.-9. (canceled)

10. A method of treating alpha 1 antitrypsin deficiency in a subject in need thereof, the method comprising
identifying a subject with a single nucleotide polymorphism (SNP) associated with alpha 1 antitrypsin deficiency in a SERPINA1 polynucleotide;
contacting the SERPINA1 polynucleotide in a cell of the subject with a guide oligonucleotide capable of effecting an adenosine deaminase acting on RNA (ADAR)-mediated adenosine to inosine alteration of the SNP associated with alpha 1 antitrypsin deficiency, thereby treating the subject.

11. A method of treating alpha 1 antitrypsin deficiency in a subject in need thereof, the method comprising
identifying a subject with a single nucleotide polymorphism (SNP) associated with alpha 1 antitrypsin deficiency in a SERPINA1 polynucleotide;
contacting the SERPINA1 polynucleotide in a cell with a guide oligonucleotide capable of effecting an adenosine deaminase acting on RNA (ADAR)-mediated adenosine to inosine alteration of the SNP associated with alpha 1 antitrypsin deficiency, and administering the cell to the subject, thereby treating the subject.

12.-18. (canceled)

19. The method of claim 1, wherein the guide oligonucleotide comprises the structure:
[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², or X³ is an alternative nucleotide.

20. The method of claim 1, wherein the guide oligonucleotide comprises the structure:
[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², or X³ has the structure of any one of Formula I-V: 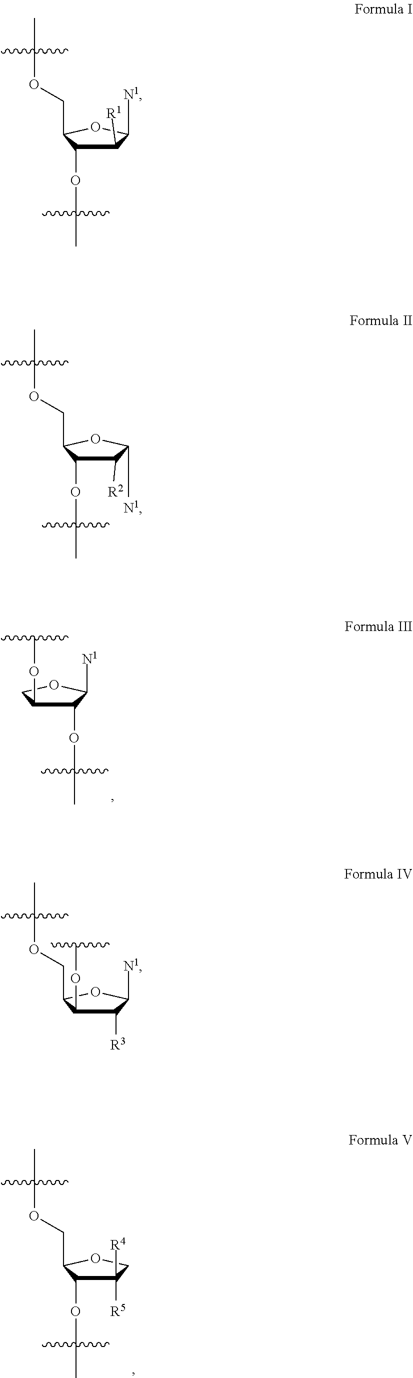
wherein N¹ is hydrogen or a nucleobase;
R¹ is hydroxy, halogen, or C₁-C₆ alkoxy;
R² is hydrogen, hydroxy, halogen, or C₁-C₆ alkoxy;
R³ is hydrogen, hydroxy, halogen, or C₁-C₆ alkoxy;
R⁴ is hydrogen, hydroxy, halogen, or C₁-C₆ alkoxy; and R⁵ is hydrogen, hydroxy, halogen, or C₁-C₆ alkoxy.

21.-64. (canceled)

65. The method of claim 1, wherein the guide oligonucleotide comprises the structure:
[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², or X³ has the structure of any one of Formula VI-XI: 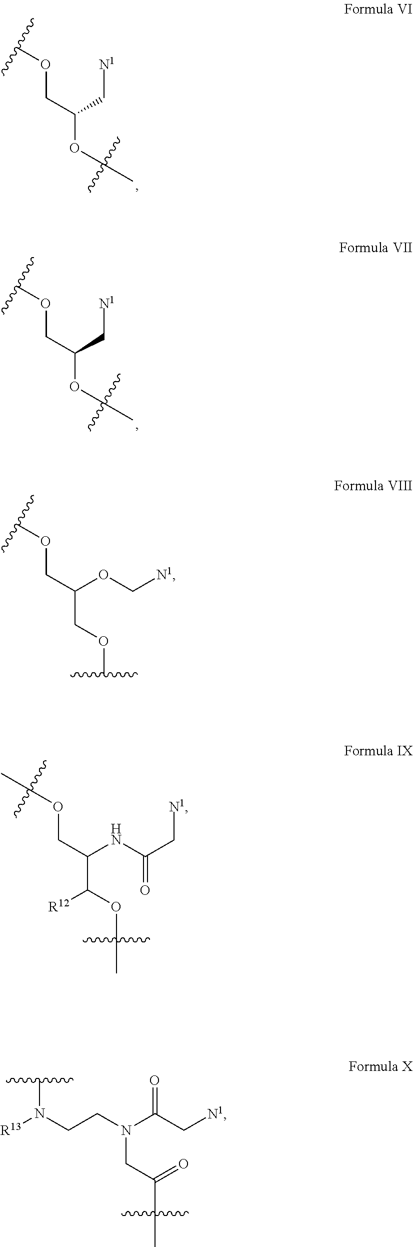 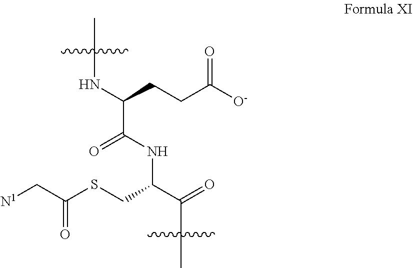
wherein N¹ is hydrogen or a nucleobase;
R¹² is hydrogen, hydroxy, fluoro, halogen, C₁-C₆ alkyl, C₁-C₆ heteroalkyl, or C₁-C₆ alkoxy;
R¹³ is hydrogen or C₁-C₆ alkyl.

66.-102. (canceled)

103. The method of claim 1, wherein the guide oligonucleotide comprises the structure:
[Aₘ]-X¹-X²-X³-[Bₙ]
wherein each of A and B is a nucleotide;
m and n are each, independently, an integer from 1 to 50;
X¹, X², and X³ are each, independently, a nucleotide, wherein at least one of X¹, X², and X³ has the structure of any one of Formula XII-XV: 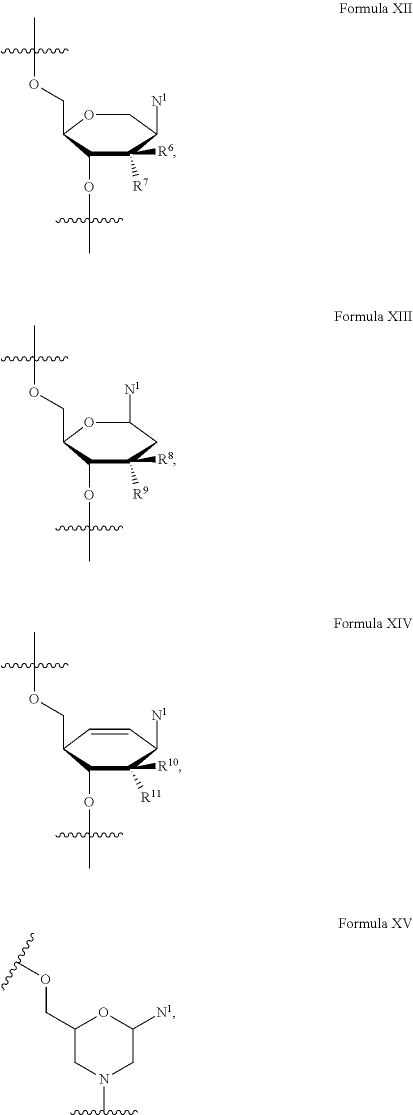
wherein N¹ is hydrogen or a nucleobase;
R⁶ is hydrogen, hydroxy, or halogen;
R⁷ is hydrogen, hydroxy, halogen, or C₁-C₆ alkoxy;
R⁸ is hydrogen or halogen;
R⁹ is hydrogen or hydroxy, halogen, or C₁-C₆ alkoxy;
R¹⁰ Is hydrogen or halogen; and
R¹¹ is hydrogen or hydroxy, halogen, or C₁-C₆ alkoxy.

104.-151. (canceled)

152. An oligonucleotide capable of effecting an adenosine deaminase acting on RNA (ADAR)-mediated adenosine to inosine alteration or a target RNA, wherein the oligonucleotide comprises the structure:
[Aₘ]-X¹-X²-X³-X⁴-[Bₙ]
wherein each of A and B is a nucleotide;
m and n are each, independently, an integer from 1 to 50;
X¹, X², and X³ are each a deoxyribonucleotide and X⁴ is a 2′-fluoronucleotide, wherein when the oligonucleotide is hybridized to the target RNA, X² is opposite the adenosine that is to be deaminated to inosine.

153. An oligonucleotide comprising the structure:
[Aₘ]-X¹-X²-X³-X⁴-[Bₙ]
wherein each of A and B is a nucleotide;
m and n are each, independently, an integer from 1 to 50;
X¹, X², and X³ are each, independently, a nucleotide, and X⁴ is selected from a 2′-O-methylnucleotide and a 2′-fluoronucleotide;
wherein at least one of X¹, X², or X³ has the structure of any one of Formula 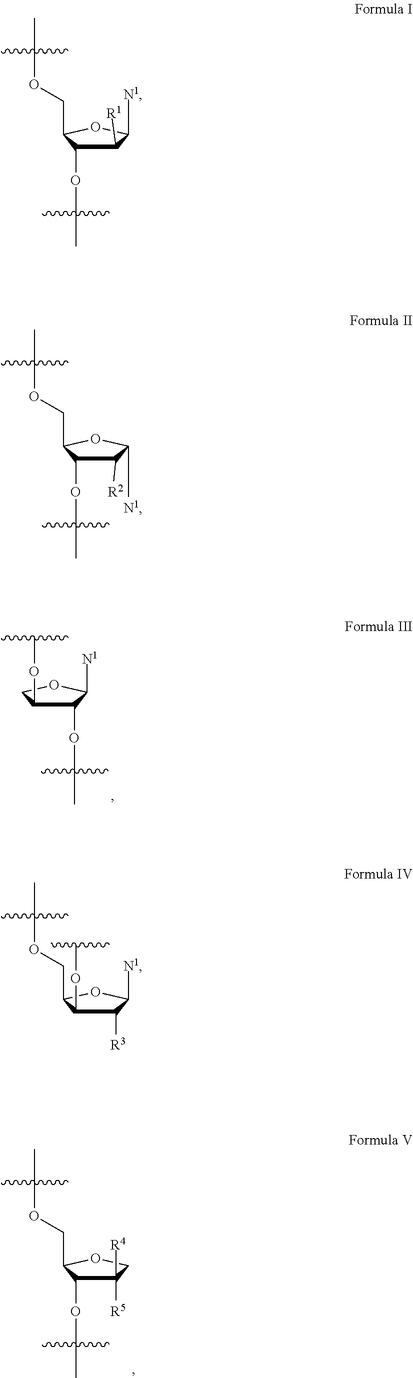
wherein N¹ is hydrogen or a nucleobase;
R¹ is hydroxy, halogen, or C₁-C₆ alkoxy;
R² is hydrogen, hydroxy, halogen, or C₁-C₆ alkoxy;
R³ is hydrogen, hydroxy, halogen, or C₁-C₆ alkoxy;
R⁴ is hydrogen, hydroxy, halogen, or C₁-C₆ alkoxy; and
R⁵ is hydrogen, hydroxy, halogen, or C₁-C₆ alkoxy.

154.-219. (canceled)

220. The oligonucleotide of claim 152, wherein the oligonucleotide capable of effecting an adenosine deaminase acting on RNA (ADAR)-mediated adenosine to inosine alteration of a SNP associated with alpha 1 antitrypsin deficiency.

221.-225. (canceled)

226. A conjugate comprising an oligonucleotide of claim 152 conjugated to a targeting moiety.

227. (canceled)

228. A complex comprising:
an oligonucleotide of claim 152; and
an mRNA,
wherein the oligonucleotide or conjugate and mRNA are hybridized to each other and the complex comprises a first mismatch at an adenosine of the mRNA.

229.-237. (canceled)

238. A method for deamination of an adenosine in an mRNA, the method comprising contacting a cell with an oligonucleotide of claim 152.

239. A method of treating a disorder in a subject in need thereof, the method comprising administering to the subject an effective amount of an oligonucleotide of claim 152.

240. A method of editing a SERPINA1 polynucleotide comprising a single nucleotide polymorphism (SNP) associated with alpha 1 antitrypsin deficiency which may result in hepatic failure or emphysema, the method comprising contacting the SERPINA1 polynucleotide with the oligonucleotide of claim 220, thereby editing the SERPINA1 polynucleotide.

241.-248. (canceled)

249. A method of treating alpha 1 antitrypsin deficiency in a subject in need thereof, the method comprising
identifying a subject with a single nucleotide polymorphism (SNP) associated with alpha 1 antitrypsin deficiency in a SERPINA1 polynucleotide;
contacting the SERPINA1 polynucleotide in a cell of the subject with the oligonucleotide of claim 220, thereby treating the subject.

250. A method of treating alpha 1 antitrypsin deficiency in a subject in need thereof, the method comprising
identifying a subject with a single nucleotide polymorphism (SNP) associated with alpha 1 antitrypsin deficiency in a SERPINA1 polynucleotide;
contacting the SERPINA1 polynucleotide in a cell with the oligonucleotide of claim 220, and
administering the cell to the subject, thereby treating the subject.

251.-257. (canceled)
